(19) United States
(12) Patent Application Publication
(10) Pub. No.: US 2018/0228797 A1
(43) Pub. Date: Aug. 16, 2018

(54) PHARMACEUTICAL COMPOSITIONS

(71) Applicant: CHARLESTON LABORATORIES, INC., Jupiter, FL (US)

(72) Inventors: Paul BOSSE, Jupiter, FL (US); John AMELING, Jupiter, FL (US); Bernard SCHACHTEL, Jupiter, FL (US); William KOZAREK, Jensen Beach, FL (US)

(21) Appl. No.: 15/951,020

(22) Filed: Apr. 11, 2018

Related U.S. Application Data
(63) Continuation of application No. PCT/US2016/056910, filed on Oct. 13, 2016.
(60) Provisional application No. 62/300,014, filed on Feb. 25, 2016.
(60) Provisional application No. 62/240,965, filed on Oct. 13, 2015.

(51) Int. Cl. A61K 31/485; A61P 29/00

(52) U.S. Cl. CPC A61K 31/485; A61K 9/0019; A61P 29/00

(57) ABSTRACT

Provided herein are methods and compositions for effective treatment or prevention of pain, which also reduce or eliminate adverse effects associated with an opioid analgesic. In addition, provided herein are methods and compositions for treatment or prevention of pain, and for reducing or preventing retching in a subject. Also provided herein are methods and compositions for treatment or prevention of pain, and for reducing or preventing sleep disturbances. Also provided herein are methods and compositions for treatment or prevention of pain, and for reducing or preventing a sensory or affective quality associated with the pain, such as agonizing pain, in a subject. Compositions described herein may include an opioid analgesic and an antiemetic. Compositions described herein may further include a non-opioid analgesic.

### CROSS-REFERENCE

This application is a continuation of PCT Application No. PCT/US2016/056910, filed Oct. 13, 2016, which claims the benefit of U.S. Provisional Application No. 62/240,965 filed Oct. 13, 2015, and U.S. Provisional Application No. 62/300,014 filed Feb. 25, 2016, each of which are incorporated herein by reference in their entirety.

### INCORPORATION BY REFERENCE

All publications, patents, and patent applications disclosed herein are incorporated by reference to the same extent as if each individual publication, patent, or patent application was specifically and individually indicated to be incorporated by reference. In the event of a conflict between a term disclosed herein and a term in an incorporated reference, the term herein controls.

### BACKGROUND

Available pain medications can have adverse effects, such as nausea, retching, vomiting, constipation, skin rashes, sedation, and sleep disturbance. As a result of such adverse effects, many subjects are unable to tolerate recommended dosages needed for effective pain relief because of adverse effects. Accordingly, there remains a need for effective therapeutics with reduced adverse effects.

In addition, to discern analgesic effects, most clinical investigators measure only evaluative end-points, and not affective or sensory qualities. There exists a need for additional types of measurement to assess therapeutic activity for preventing, reducing or treating various forms of pain.

### BRIEF SUMMARY

The present disclosure provides methods and compositions for effective pain treatment.

Provided herein are methods for providing treatment or reduction of agonizing pain in a subject, comprising: administering to a subject in need thereof a pharmaceutical composition, wherein the pharmaceutical composition comprises: an effective amount of an opioid analgesic to treat agonizing pain; and an effective amount of an antiemetic to reduce or prevent nausea associated the opioid analgesic or to reduce or prevent vomiting associated the opioid analgesic, wherein administration of the pharmaceutical composition provides for a reduction or prevention of agonizing pain in the subject. Further provided herein are methods wherein the agonizing pain is measured by the subject on a Likert-type agonizing pain scale. Further provided herein are methods wherein the method reduces severity of the agonizing pain. Further provided herein are methods wherein the method reduces occurrence of the agonizing pain. Further provided herein are methods wherein administration of the pharmaceutical composition provides for about 60% reduction of agonizing pain over 24 hours. Further provided herein are methods wherein administration of the pharmaceutical composition provides for about 30% reduction in pain intensity over 24 hours. Further provided herein are methods wherein administration of the pharmaceutical composition provides increased agonizing pain relief in the subject compared to administration of the opioid analgesic without the antiemetic. Further provided herein are methods wherein the pharmaceutical composition further comprises an effective amount of a non-opioid analgesic to treat pain. Further provided herein are methods wherein the pharmaceutical composition is a solid oral pharmaceutical composition. Provided herein are methods for providing pain relief and reducing or preventing an affective quality pain in a subject in need thereof, comprising: administering to a subject a pharmaceutical composition, wherein the pharmaceutical composition comprises: an effective amount of an opioid analgesic to treat pain; and an effective amount of an antiemetic to reduce or prevent nausea associated the opioid analgesic or to reduce or prevent vomiting associated the opioid analgesic, wherein administration of the pharmaceutical composition provides for a reduction or prevention of an affective quality in the subject. Further provided herein are methods wherein the affective quality is agonizing pain. Further provided herein are methods wherein the method provides the subject a decrease in pain occurrence. Further provided herein are methods wherein the method provides the subject a decrease in pain severity. Further provided herein are methods wherein the pain severity is reduced by more than 30% following first administration of the pharmaceutical composition. Further provided herein are methods wherein the subject has a reduction of at least 30% in severe pain over about 24 hours or more following administration of the pharmaceutical composition. Further provided herein are methods wherein the subject has a reduction of at least 10% in moderate to severe pain over about 48 hours or more following administration of the pharmaceutical composition. Further provided herein are methods wherein the subject has a reduction of at least 20% in moderate to severe pain over about 24 hours or more following administration of the pharmaceutical composition. Further provided herein are methods wherein the subject has a reduction in moderate to severe pain over about 4 to 6 hours or more following administration of the pharmaceutical composition. Further provided herein are methods wherein the subject has a reduction in moderate to severe pain over about 12 hours or more following administration of the pharmaceutical composition. Further provided herein are methods wherein the subject has a reduction in moderate to severe pain over about 2 or 3 doses following administration of the pharmaceutical composition. Further provided herein are methods wherein the subject has an increase of at least 20% in pain relief over about 12 hours or more following administration of the pharmaceutical composition. Further provided herein are methods wherein the subject has an increase of at least 20% in pain relief over about the first 2 or 3 doses or more following administration of the pharmaceutical composition. Further provided herein are methods wherein the subject has a reduction in affective, sensory, or evaluative qualities of pain over about 6 hours or more following initial administration of the pharmaceutical composition. Further provided herein are methods wherein the subject has a reduction in affective, sensory, or evaluative qualities of pain over two or more doses or about 12 hours, about 24 hours, or more following administration of the pharmaceutical composition. Further provided herein are methods wherein the subject has a reduction of at least 15% in affective, sensory, or evaluative qualities of pain over about 24 hours or more following administration of the pharmaceutical composition. Further provided herein are methods wherein the subject has a reduction of at least 50% in the occurrence of vomiting over about 24 hours or more following administration of the pharmaceutical composition. Further provided herein are methods wherein the subject is nausea-prone. Further provided herein are methods wherein the administration of the pharmaceutical composition further provides for reduction or prevention of opioid induced nausea or vomiting (OINV) in the subject. Further provided herein are methods wherein the subject has increased pain relief compared to a subject administered a pharmaceutical composition that comprises the opioid analgesic and the non-opioid analgesic without the antiemetic. Further provided herein are methods wherein the subject has increased pain relief during the first 6 hours post administration. Further provided herein are methods wherein the administration of the pharmaceutical composition further provides for reduction or prevention of opioid induced nausea or vomiting (OINV) in the subject. Further provided herein are methods wherein the subject has reduced severity of nausea or vomiting. Further provided herein are methods wherein the subject has reduced severity of nausea or vomiting over about 6 hours following initial administration of the pharmaceutical composition. Further provided herein are methods wherein the subject has reduced severity of nausea or vomiting over about 24 hours following initial administration of the pharmaceutical composition. Further provided herein are methods wherein the subject has reduced occurrence of nausea or vomiting. Further provided herein are methods wherein the subject has a reduction of at least 70% in the occurrence of ONIV over about 48 hours or more following administration of the pharmaceutical composition. Further provided herein are methods wherein the subject has a reduction of at least 50% in occurrence of ONIV for about 24 hours or more following administration of the pharmaceutical composition. Further provided herein are methods wherein reducing nausea or vomiting in the subject is in comparison to a subject administered a pharmaceutical composition that comprises the opioid analgesic and the non-opioid analgesic without the antiemetic. Further provided herein are methods wherein the method reduces occurrence or severity of nausea or vomiting over about 24 hours following administration of the pharmaceutical composition. Further provided herein are methods wherein the subject has a reduction of at least 50% in occurrence of moderate to severe nausea or vomiting over about 48 hours or more following administration of the pharmaceutical composition. Further provided herein are methods wherein the subject has a reduction of at least 60% in occurrence of vomiting over about 5 days or more following administration of the pharmaceutical composition. Further provided herein are methods wherein the subject has a reduction of at least 50% in doses of an antiemetic over about 48 hours or more following administration of the pharmaceutical composition. Further provided herein are methods wherein the subject has a reduction of at least 75% in doses of a parenteral antiemetic over about 48 hours or more following administration of the pharmaceutical composition. Further provided herein are methods wherein the subject has a reduction of at least 70% in doses of an antiemetic over about 5 days or more following administration of the pharmaceutical composition. Further provided herein are methods wherein the subject has a reduction of at least 80% in doses of an antiemetic over about 5 days or more following administration of the pharmaceutical composition. Further provided herein are methods wherein the subject has a reduction of at least 70% in severe nausea over about 5 days or more following administration of the pharmaceutical composition. Further provided herein are methods wherein the subject has a reduction of at least 40% in peak severity of nausea over about 5 days or more following administration of the pharmaceutical composition. Further provided herein are methods wherein the subject has an increase of 60% in the likelihood of no nausea, no vomiting, or no use of an antiemetic over about 5 days or more following administration of the pharmaceutical composition. Further provided herein are methods wherein the subject has an increase of 100% in the likelihood of no nausea, no vomiting, or no use of an antiemetic over about 5 days or more following administration of the pharmaceutical composition. Further provided herein are methods wherein the administration occurs more than once over about 24 hours or longer. Further provided herein are methods further comprising administering an antiemetic when awake. Further provided herein are methods further comprising assessing the subject by a Nausea Severity Scale (NIS). Further provided herein are methods further comprising assessing the subject by a Retching or Vomiting Index (RVI). Further provided herein are methods further comprising assessing the subject by a Vomiting Supplemental Medications (NVSM). Further provided herein are methods wherein the administration occurs every four to six hours. Further provided herein are methods wherein the administration occurs two to six times over the first 24 hours. Further provided herein are methods wherein the administration occurs one to six times after the first 24 hours. Further provided herein are methods wherein the administration occurs no more than six times every 24 hours. Further provided herein are methods wherein the administration occurs periodically over 1-28 days. Further provided herein are methods wherein the administration occurs periodically over 1-21 days. Further provided herein are methods wherein the administration occurs periodically over 1-14 days. Further provided herein are methods wherein the administration occurs periodically over 1-7 days. Further provided herein are methods wherein the administration occurs periodically over 1-5 days. Further provided herein are methods wherein the administration occurs periodically over 1-3 days. Further provided herein are methods wherein the administration occurs periodically over 1-2 days. Further provided herein are methods wherein the administration occurs periodically over 24 hours. Further provided herein are methods wherein the administration is an oral administration. Further provided herein are methods wherein the administration begins from 0 to 6 hours before a surgery. Further provided herein are methods wherein the administration begins from 0 to 6 hours after a surgery. Further provided herein are methods wherein the administration is from 0 to 6 hours after an injury. Further provided herein are methods wherein the subject is a post-operative subject. Further provided herein are methods wherein the subject is a post-discharge subject. Further provided herein are methods wherein the pain is pain from an operation or post-operative pain. Further provided herein are methods wherein the pain is acute pain. Further provided herein are methods wherein the pain is chronic pain. Further provided herein are methods wherein the pain is severe. Further provided herein are methods wherein the pain is moderate. Further provided herein are methods wherein the pain is moderate to severe. Further provided herein are methods wherein the pain is agonizing pain. Further provided herein are methods wherein the administration is prophylactic. Further provided herein are methods wherein the subject has one or more conditions or diseases. Further provided herein are methods wherein the one or more conditions or diseases comprise cancer, surgical procedure, acute physical injury, chronic physical injury, bone fracture, crush injury, spinal cord injury, inflammatory disease, non-inflammatory neuropathic condition, photophobia, or any combination thereof. Further provided herein are methods wherein the pharmaceutical composition does not cause increased sedation in comparison to a pharmaceutical composition with the same amount of the opioid analgesic, in the absence of the antiemetic. Further provided herein are methods wherein the pharmaceutical composition is a solid dosage form. Further provided herein are methods wherein the solid dosage form comprises an immediate-release matrix, wherein the immediate release matrix comprises an effective amount of the antiemetic. Further provided herein are methods wherein the solid dosage form comprises a controlled-release matrix, wherein the controlled-release matrix comprises an effective amount of the opioid analgesic. Further provided herein are methods wherein the controlled-release matrix comprises an effective amount of the non-opioid analgesic. Further provided herein are methods wherein the solid dosage form is a tablet, particle or capsule. Further provided herein are methods wherein the tablet is a multi-layer tablet. Further provided herein are methods wherein the tablet is a bi-layer tablet. Further provided herein are methods wherein the antiemetic is promethazine or a pharmaceutically acceptable salt thereof. Further provided herein are methods wherein the opioid analgesic is hydrocodone or a pharmaceutically acceptable salt thereof. Further provided herein are methods wherein the opioid analgesic is oxycodone or a pharmaceutically acceptable salt thereof. Further provided herein are methods wherein the non-opioid analgesic is acetaminophen or a pharmaceutically acceptable salt thereof. Further provided herein are methods wherein: the opioid analgesic is hydrocodone or a pharmaceutically acceptable salt thereof that is present in an amount of from about 6.5 mg to about 8.5 mg; and the antiemetic is promethazine or a pharmaceutically acceptable salt thereof that is present in an amount of from about 12.5 mg to about 25 mg. Further provided herein are methods wherein: the opioid analgesic is hydrocodone or a pharmaceutically acceptable salt thereof that is present in an amount of from about 6.5 mg to about 8.5 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg. Further provided herein are methods wherein: the opioid analgesic is oxycodone or a pharmaceutically acceptable salt thereof that is present in an amount of from about 5 mg to about 15 mg; and the antiemetic is promethazine or a pharmaceutically acceptable salt thereof that is present in an amount of from about 12.5 mg to about 25 mg. Further provided herein are methods wherein: the opioid analgesic is hydrocodone, or a pharmaceutically acceptable salt thereof that is present in an amount of from 5 mg to about 15 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg. Further provided herein are methods wherein: the antiemetic is promethazine or a pharmaceutically acceptable salt thereof that is present in amount of about 12.5 mg, and the opioid analgesic is oxycodone or a pharmaceutically acceptable salt thereof that is present in an amount of about 5.0 mg. Further provided herein are methods wherein: the antiemetic is promethazine hydrochloride that is present in an amount of about 12.5 mg; and the opioid analgesic is oxycodone hydrochloride that is present in an amount of about 5.0 mg. Further provided herein are methods wherein the non-opioid analgesic is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof. Further provided herein are methods wherein: the antiemetic is promethazine or a pharmaceutically acceptable salt thereof that is present in amount of about 12.5 mg, the opioid analgesic is hydrocodone or a pharmaceutically acceptable salt thereof that is present in an amount of about 7.5 mg; and the non-opioid analgesic is acetaminophen or a pharmaceutically acceptable salt thereof that is present in an amount of about 325 mg. Further provided herein are methods wherein: the antiemetic is promethazine hydrochloride that is present in an amount of about 12.5 mg; the opioid analgesic is hydrocodone bitartrate that is present in an amount of about 7.5 mg; and the non-opioid analgesic is acetaminophen that is present in an amount of about 325 mg. Further provided herein are methods wherein the subject is human. Further provided herein are methods wherein the pharmaceutical composition further comprises one or more excipients. Further provided herein are methods wherein the one or more excipients comprise an antioxidant agent, a binder, a coating material, a colorant agent, a diluent, a disintegrant, a dispersant, an emulsifying agent, a flavoring agent, a glidant, a lubricant, a pH modifying agent, a plasticizer, a preservative agent, a solubilizing agent, a stabilizer, a surfactant, a sweetening agent, a thickening agent, a pharmaceutically inert material, or any combination thereof. Further provided herein are methods wherein the antiemetic has a Tmax that is about 3-6 hours. Further provided herein are methods wherein the antiemetic has a Tmax that is about 20-100 minutes shorter than a Tmax of a corresponding standard-release antiemetic. Further provided herein are methods wherein the antiemetic has a Tmax that is about 3-5 hours, wherein the subject is fasted. Further provided herein are methods wherein the antiemetic has a Tmax of about 4 hours. Further provided herein are methods wherein the antiemetic has a Tmax that is about 20-80 minutes shorter than a Tmax of a corresponding standard-release antiemetic, wherein the subject is fasted. Further provided herein are methods wherein the antiemetic has a Tmax that is about 50 minutes shorter than a Tmax of a corresponding standard-release antiemetic. Further provided herein are methods wherein the antiemetic has a Tmax that is about 4-6 hours, wherein the subject is fed. Further provided herein are methods wherein the antiemetic has a Tmax of about 5 hours. Further provided herein are methods wherein the antiemetic has a Tmax that is about 40-100 minutes shorter than a Tmax of a corresponding standard-release antiemetic, wherein the subject is fed. Further provided herein are methods wherein the antiemetic has a Tmax that is about 70 minutes shorter than a Tmax of a corresponding standard-release antiemetic. Further provided herein are methods wherein the antiemetic has an about 20-200% greater absorption during in about two hours than a corresponding standard-release antiemetic. Further provided herein are methods wherein the antiemetic has an about 20-200% greater absorption in about 90 minutes than a corresponding standard-release antiemetic. Further provided herein are methods wherein the antiemetic has an about 20-200% greater absorption in about an hour than a corresponding standard-release antiemetic. Further provided herein are methods wherein the antiemetic has an about 20-100% greater absorption in about an hour than a corresponding standard-release antiemetic. Further provided herein are methods wherein the antiemetic has an about 60% greater absorption in about an hour than a corresponding standard-release antiemetic. Further provided herein are methods wherein the antiemetic has an about 20-60% greater absorption in about 45 minutes than a corresponding standard-release antiemetic. Further provided herein are methods wherein the antiemetic has an about 40% greater absorption in about 45 minutes than a corresponding standard-release antiemetic. Further provided herein are methods wherein the antiemetic has an about 20-60% greater absorption in about 30 minutes than a corresponding standard-release antiemetic. Further provided herein are methods wherein the antiemetic has an about 40% greater absorption in about 30 minutes than a corresponding standard-release antiemetic. Further provided herein are methods wherein the antiemetic is promethazine or a pharmaceutically acceptable salt thereof. Further provided herein are methods wherein the antiemetic is promethazine hydrochloride. Further provided herein are methods wherein the administration occurs multiple times per day. Further provided herein are methods wherein the administration occurs at least twice daily. Further provided herein are methods wherein the administration occurs 2 to 6 times daily.

Provided herein are methods for and reducing or preventing retching in a subject in need thereof, comprising: administering to a subject in need thereof a pharmaceutical composition, wherein the pharmaceutical composition comprises: an effective amount of an opioid analgesic to treat pain; and an effective amount of an antiemetic to reduce or prevent retching associated the opioid analgesic, wherein administration of the pharmaceutical composition provides for a reduction or prevention of retching in the subject. Further provided herein are methods wherein the pharmaceutical composition further comprises an effective amount of a non-opioid analgesic to treat pain. Further provided herein are methods wherein the pharmaceutical composition is a solid oral pharmaceutical composition. Further provided herein are methods wherein the administration of the pharmaceutical composition provides for prevention of retching. Further provided herein are methods wherein the method reduces severity of retching. Further provided herein are methods wherein the administration of the pharmaceutical composition provides for reduction in severity of retching. Further provided herein are methods wherein the administration of the pharmaceutical composition provides for reduction in severity of retching. Further provided herein are methods wherein administration of the pharmaceutical composition provides for less vomiting than observed for a subject administered the opioid without the antiemetic. Further provided herein are methods wherein administration of the pharmaceutical composition provides for less retching than observed for a subject administered the opioid without the antiemetic. Further provided herein are methods wherein administration of the pharmaceutical composition provides for three times less vomiting that observed for a subject administered the opioid without the antiemetic. Further provided herein are methods wherein administration of the pharmaceutical composition provides for two times less vomiting that observed for a subject administered the opioid without the antiemetic over about 5 days following administration of the pharmaceutical composition. Further provided herein are methods wherein the subject has a reduction of about 40% in occurrence of retching over about 5 days following administration of the pharmaceutical composition. Further provided herein are methods wherein the subject has a reduction of at least 35% in occurrence of retching or vomiting over about 5 days or more following administration of the pharmaceutical composition. Further provided herein are methods wherein the subject has a reduction of at least 45% in occurrence of retching or vomiting over about 5 days or more following administration of the pharmaceutical composition. Further provided herein are methods wherein the subject has a reduction of about 50% in occurrence of retching over about 5 days following administration of the pharmaceutical composition. Further provided herein are methods wherein administration of the pharmaceutical composition provides increased pain relief in the subject compared to administration of the opioid analgesic without the antiemetic. Further provided herein are methods wherein the method provides the subject a decrease in pain occurrence. Further provided herein are methods wherein the method provides the subject a decrease in pain severity. Further provided herein are methods wherein the pain severity is reduced by more than 30% following first administration of the pharmaceutical composition. Further provided herein are methods wherein the subject has a reduction of at least 30% in severe pain over about 24 hours or more following administration of the pharmaceutical composition. Further provided herein are methods wherein the subject has a reduction of at least 10% in moderate to severe pain over about 48 hours or more following administration of the pharmaceutical composition. Further provided herein are methods wherein the subject has a reduction of at least 20% in moderate to severe pain over about 24 hours or more following administration of the pharmaceutical composition. Further provided herein are methods wherein the subject has a reduction in moderate to severe pain over about 4 to 6 hours or more following administration of the pharmaceutical composition. Further provided herein are methods wherein the subject has a reduction in moderate to severe pain over about 12 hours or more following administration of the pharmaceutical composition. Further provided herein are methods wherein the subject has a reduction in moderate to severe pain over about 2 or 3 doses following administration of the pharmaceutical composition. Further provided herein are methods wherein the subject has an increase of at least 20% in pain relief over about 12 hours or more following administration of the pharmaceutical composition. Further provided herein are methods wherein the subject has an increase of at least 20% in pain relief over about the first 2 or 3 doses or more following administration of the pharmaceutical composition. Further provided herein are methods wherein the subject has a reduction in affective, sensory, or evaluative qualities of pain over about 6 hours or more following initial administration of the pharmaceutical composition. Further provided herein are methods wherein the subject has a reduction in affective, sensory, or evaluative qualities of pain over two or more doses or about 12 hours, about 24 hours, or more following administration of the pharmaceutical composition. Further provided herein are methods wherein the subject has a reduction of at least 15% in affective, sensory, or evaluative qualities of pain over about 24 hours or more following administration of the pharmaceutical composition. Further provided herein are methods wherein the subject has a reduction of at least 50% in the occurrence of vomiting over about 24 hours or more following administration of the pharmaceutical composition. Further provided herein are methods wherein the subject is nausea-prone. Further provided herein are methods wherein the subject is susceptible to opioid induced nausea or vomiting (OINV). Further provided herein are methods wherein the subject has increased pain relief compared to a subject administered a pharmaceutical composition that comprises the opioid analgesic and the non-opioid analgesic without the antiemetic. Further provided herein are methods wherein the subject has increased pain relief during the first 6 hours post administration. Further provided herein are methods wherein the administration of the pharmaceutical composition further provides for reduction or prevention of opioid induced nausea or vomiting (OINV) in the subject. Further provided herein are methods wherein the subject has reduced severity of nausea or vomiting. Further provided herein are methods wherein the subject has reduced severity of nausea or vomiting over about 6 hours following initial administration of the pharmaceutical composition. Further provided herein are methods wherein the subject has reduced severity of nausea or vomiting over about 24 hours following initial administration of the pharmaceutical composition. Further provided herein are methods wherein the subject has reduced occurrence of nausea or vomiting. Further provided herein are methods wherein the subject has a reduction of at least 70% in the occurrence of ONIV over about 48 hours or more following administration of the pharmaceutical composition. Further provided herein are methods wherein the subject has a reduction of at least 50% in occurrence of ONIV for about 24 hours or more following administration of the pharmaceutical composition. Further provided herein are methods wherein reducing nausea or vomiting in the subject is in comparison to a subject administered a pharmaceutical composition that comprises the opioid analgesic and the non-opioid analgesic without the antiemetic. Further provided herein are methods wherein the method reduces occurrence or severity of nausea or vomiting over about 24 hours following administration of the pharmaceutical composition. Further provided herein are methods wherein the subject has a reduction of at least 50% in occurrence of moderate to severe nausea or vomiting over about 48 hours or more following administration of the pharmaceutical composition. Further provided herein are methods wherein the subject has a reduction of at least 60% in occurrence of vomiting over about 5 days or more following administration of the pharmaceutical composition. Further provided herein are methods wherein the subject has a reduction of at least 50% in doses of an antiemetic over about 48 hours or more following administration of the pharmaceutical composition. Further provided herein are methods wherein the subject has a reduction of at least 75% in doses of a parenteral antiemetic over about 48 hours or more following administration of the pharmaceutical composition. Further provided herein are methods wherein the subject has a reduction of at least 70% in doses of an antiemetic over about 5 days or more following administration of the pharmaceutical composition. Further provided herein are methods wherein the subject has a reduction of at least 80% in doses of an antiemetic over about 5 days or more following administration of the pharmaceutical composition. Further provided herein are methods wherein the subject has a reduction of at least 70% in severe nausea over about 5 days or more following administration of the pharmaceutical composition. Further provided herein are methods wherein the subject has a reduction of at least 40% in peak severity of nausea over about 5 days or more following administration of the pharmaceutical composition. Further provided herein are methods wherein the subject has an increase of 60% in the likelihood of no nausea, no vomiting, or no use of an antiemetic over about 5 days or more following administration of the pharmaceutical composition. Further provided herein are methods wherein the subject has an increase of 100% in the likelihood of no nausea, no vomiting, or no use of an antiemetic over about 5 days or more following administration of the pharmaceutical composition. Further provided herein are methods wherein the administration occurs more than once over about 24 hours or longer. Further provided herein are methods further comprising administering an antiemetic when awake. Further provided herein are methods further comprising assessing the subject by a Nausea Severity Scale (NIS). Further provided herein are methods further comprising assessing the subject by a Retching or Vomiting Index (RVI). Further provided herein are methods further comprising assessing the subject by a Vomiting Supplemental Medications (NVSM). Further provided herein are methods wherein the administration occurs every four to six hours. Further provided herein are methods wherein the administration occurs two to six times over the first 24 hours. Further provided herein are methods wherein the administration occurs one to six times after the first 24 hours. Further provided herein are methods wherein the administration occurs no more than six times every 24 hours. Further provided herein are methods wherein the administration occurs periodically over 1-28 days. Further provided herein are methods wherein the administration occurs periodically over 1-21 days. Further provided herein are methods wherein the administration occurs periodically over 1-14 days. Further provided herein are methods wherein the administration occurs periodically over 1-7 days. Further provided herein are methods wherein the administration occurs periodically over 1-5 days. Further provided herein are methods wherein the administration occurs periodically over 1-3 days. Further provided herein are methods wherein the administration occurs periodically over 1-2 days. Further provided herein are methods wherein the administration occurs periodically over 24 hours. Further provided herein are methods wherein the administration is an oral administration. Further provided herein are methods wherein the administration begins from 0 to 6 hours before a surgery. Further provided herein are methods wherein the administration begins from 0 to 6 hours after a surgery. Further provided herein are methods wherein the administration is from 0 to 6 hours after an injury. Further provided herein are methods wherein the subject is a post-operative subject. Further provided herein are methods wherein the subject is a post-discharge subject. Further provided herein are methods wherein the pain is pain from an operation or post-operative pain. Further provided herein are methods wherein the pain is acute pain. Further provided herein are methods wherein the pain is chronic pain. Further provided herein are methods wherein the pain is severe. Further provided herein are methods wherein the pain is moderate. Further provided herein are methods wherein the pain is moderate to severe. Further provided herein are methods wherein the pain is agonizing pain. Further provided herein are methods wherein the administration is prophylactic. Further provided herein are methods wherein the subject has one or more conditions or diseases. Further provided herein are methods wherein the one or more conditions or diseases comprise cancer, surgical procedure, acute physical injury, chronic physical injury, bone fracture, crush injury, spinal cord injury, inflammatory disease, non-inflammatory neuropathic condition, photophobia, or any combination thereof. Further provided herein are methods wherein the pharmaceutical composition does not cause increased sedation in comparison to a pharmaceutical composition with the same amount of the opioid analgesic, in the absence of the antiemetic. Further provided herein are methods wherein the pharmaceutical composition is a solid dosage form. Further provided herein are methods wherein the solid dosage form comprises an immediate-release matrix, wherein the immediate release matrix comprises an effective amount of the antiemetic. Further provided herein are methods wherein the solid dosage form comprises a controlled-release matrix, wherein the controlled-release matrix comprises an effective amount of the opioid analgesic. Further provided herein are methods wherein the controlled-release matrix comprises an effective amount of the non-opioid analgesic. Further provided herein are methods wherein the solid dosage form is a tablet, particle or capsule. Further provided herein are methods wherein the tablet is a multi-layer tablet. Further provided herein are methods wherein the tablet is a bi-layer tablet. Further provided herein are methods wherein the antiemetic is promethazine or a pharmaceutically acceptable salt thereof. Further provided herein are methods wherein the opioid analgesic is hydrocodone or a pharmaceutically acceptable salt thereof. Further provided herein are methods wherein the opioid analgesic is oxycodone or a pharmaceutically acceptable salt thereof. Further provided herein are methods wherein the non-opioid analgesic is acetaminophen or a pharmaceutically acceptable salt thereof. Further provided herein are methods wherein: the opioid analgesic is hydrocodone or a pharmaceutically acceptable salt thereof that is present in an amount of from about 6.5 mg to about 8.5 mg; and the antiemetic is promethazine or a pharmaceutically acceptable salt thereof that is present in an amount of from about 12.5 mg to about 25 mg. Further provided herein are methods wherein: the opioid analgesic is hydrocodone or a pharmaceutically acceptable salt thereof that is present in an amount of from about 6.5 mg to about 8.5 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg. Further provided herein are methods wherein: the opioid analgesic is oxycodone or a pharmaceutically acceptable salt thereof that is present in an amount of from about 5 mg to about 15 mg; and the antiemetic is promethazine or a pharmaceutically acceptable salt thereof that is present in an amount of from about 12.5 mg to about 25 mg. Further provided herein are methods wherein: the opioid analgesic is hydrocodone, or a pharmaceutically acceptable salt thereof that is present in an amount of from 5 mg to about 15 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg. Further provided herein are methods wherein: the antiemetic is promethazine or a pharmaceutically acceptable salt thereof that is present in amount of about 12.5 mg, and the opioid analgesic is oxycodone or a pharmaceutically acceptable salt thereof that is present in an amount of about 5.0 mg. Further provided herein are methods wherein: the antiemetic is promethazine hydrochloride that is present in an amount of about 12.5 mg; and the opioid analgesic is oxycodone hydrochloride that is present in an amount of about 5.0 mg. Further provided herein are methods wherein the non-opioid analgesic is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof. Further provided herein are methods wherein: the antiemetic is promethazine or a pharmaceutically acceptable salt thereof that is present in amount of about 12.5 mg, the opioid analgesic is hydrocodone or a pharmaceutically acceptable salt thereof that is present in an amount of about 7.5 mg; and the non-opioid analgesic is acetaminophen or a pharmaceutically acceptable salt thereof that is present in an amount of about 325 mg. Further provided herein are methods wherein: the antiemetic is promethazine hydrochloride that is present in an amount of about 12.5 mg; the opioid analgesic is hydrocodone bitartrate that is present in an amount of about 7.5 mg; and the non-opioid analgesic is acetaminophen that is present in an amount of about 325 mg. Further provided herein are methods wherein the subject is human. Further provided herein are methods wherein the pharmaceutical composition further comprises one or more excipients. Further provided herein are methods wherein the one or more excipients comprise an antioxidant agent, a binder, a coating material, a colorant agent, a diluent, a disintegrant, a dispersant, an emulsifying agent, a flavoring agent, a glidant, a lubricant, a pH modifying agent, a plasticizer, a preservative agent, a solubilizing agent, a stabilizer, a surfactant, a sweetening agent, a thickening agent, a pharmaceutically inert material, or any combination thereof. Further provided herein are methods wherein the antiemetic has a Tmax that is about 3-6 hours. Further provided herein are methods wherein the antiemetic has a Tmax that is about 20-100 minutes shorter than a Tmax of a corresponding standard-release antiemetic. Further provided herein are methods wherein the antiemetic has a Tmax that is about 3-5 hours, wherein the subject is fasted. Further provided herein are methods wherein the antiemetic has a Tmax of about 4 hours. Further provided herein are methods wherein the antiemetic has a Tmax that is about 20-80 minutes shorter than a Tmax of a corresponding standard-release antiemetic, wherein the subject is fasted. Further provided herein are methods wherein the antiemetic has a Tmax that is about 50 minutes shorter than a Tmax of a corresponding standard-release antiemetic. Further provided herein are methods wherein the antiemetic has a Tmax that is about 4-6 hours, wherein the subject is fed. Further provided herein are methods wherein the antiemetic has a Tmax of about 5 hours. Further provided herein are methods wherein the antiemetic has a Tmax that is about 40-100 minutes shorter than a Tmax of a corresponding standard-release antiemetic, wherein the subject is fed. Further provided herein are methods wherein the antiemetic has a Tmax that is about 70 minutes shorter than a Tmax of a corresponding standard-release antiemetic. Further provided herein are methods wherein the antiemetic has an about 20-200% greater absorption during in about two hours than a corresponding standard-release antiemetic. Further provided herein are methods wherein the antiemetic has an about 20-200% greater absorption in about 90 minutes than a corresponding standard-release antiemetic. Further provided herein are methods wherein the antiemetic has an about 20-200% greater absorption in about an hour than a corresponding standard-release antiemetic. Further provided herein are methods wherein the antiemetic has an about 20-100% greater absorption in about an hour than a corresponding standard-release antiemetic. Further provided herein are methods wherein the antiemetic has an about 60% greater absorption in about an hour than a corresponding standard-release antiemetic. Further provided herein are methods wherein the antiemetic has an about 20-60% greater absorption in about 45 minutes than a corresponding standard-release antiemetic. Further provided herein are methods wherein the antiemetic has an about 40% greater absorption in about 45 minutes than a corresponding standard-release antiemetic. Further provided herein are methods wherein the antiemetic has an about 20-60% greater absorption in about 30 minutes than a corresponding standard-release antiemetic. Further provided herein are methods wherein the antiemetic has an about 40% greater absorption in about 30 minutes than a corresponding standard-release antiemetic. Further provided herein are methods wherein the antiemetic is promethazine or a pharmaceutically acceptable salt thereof. Further provided herein are methods wherein the antiemetic is promethazine hydrochloride. Further provided herein are methods wherein the administration occurs multiple times per day. Further provided herein are methods wherein the administration occurs at least twice daily. Further provided herein are methods wherein the administration occurs 2 to 6 times daily.

Provided herein are methods for providing pain relief and reducing or preventing sleep disturbance in a subject in need thereof, comprising administering to a subject a pharmaceutical composition, wherein the pharmaceutical composition comprises: an effective amount of an opioid analgesic to treat pain; and an effective amount of an antiemetic to reduce or prevent sleep disturbance associated the opioid analgesic, wherein administration of the pharmaceutical composition provides for a reduction or prevention of sleep disturbance in the subject. Further provided herein are methods wherein administration of the pharmaceutical composition provides for about 3.5% chance of sleep disturbance over a 48 hour period in the subject. Further provided herein are methods wherein the subject has a reduction of about 40% in occurrence of retching over about 5 days following administration of the pharmaceutical composition. Further provided herein are methods wherein the pharmaceutical composition further comprises an effective amount of a non-opioid analgesic to treat pain. Further provided herein are methods wherein the pharmaceutical composition is a solid oral pharmaceutical composition. Further provided herein are methods wherein the subject has a reduction of at least 70% in the occurrence of sleep disturbance over 2 nights or more following administration of the pharmaceutical composition. Further provided herein are methods wherein the sleep disturbance is measured by the number of awakenings in an observation period. Further provided herein are methods wherein the subject awakens due to nausea, retching, or vomiting. Further provided herein are methods wherein administration of the pharmaceutical composition provides for less sleep disturbance pain relief in the subject compared to administration of the opioid analgesic without the antiemetic. Further provided herein are methods wherein the method provides the subject a decrease in pain occurrence. Further provided herein are methods wherein the method provides the subject a decrease in pain severity. Further provided herein are methods wherein the pain severity is reduced by more than 30% following first administration of the pharmaceutical composition. Further provided herein are methods wherein the subject has a reduction of at least 30% in severe pain over about 24 hours or more following administration of the pharmaceutical composition. Further provided herein are methods wherein the subject has a reduction of at least 10% in moderate to severe pain over about 48 hours or more following administration of the pharmaceutical composition. Further provided herein are methods wherein the subject has a reduction of at least 20% in moderate to severe pain over about 24 hours or more following administration of the pharmaceutical composition. Further provided herein are methods wherein the subject has a reduction in moderate to severe pain over about 4 to 6 hours or more following administration of the pharmaceutical composition. Further provided herein are methods wherein the subject has a reduction in moderate to severe pain over about 12 hours or more following administration of the pharmaceutical composition. Further provided herein are methods wherein the subject has a reduction in moderate to severe pain over about 2 or 3 doses following administration of the pharmaceutical composition. Further provided herein are methods wherein the subject has an increase of at least 20% in pain relief over about 12 hours or more following administration of the pharmaceutical composition. Further provided herein are methods wherein the subject has an increase of at least 20% in pain relief over about the first 2 or 3 doses or more following administration of the pharmaceutical composition. Further provided herein are methods wherein the subject has a reduction in affective, sensory, or evaluative qualities of pain over about 6 hours or more following initial administration of the pharmaceutical composition. Further provided herein are methods wherein the subject has a reduction in affective, sensory, or evaluative qualities of pain over two or more doses or about 12 hours, about 24 hours, or more following administration of the pharmaceutical composition. Further provided herein are methods wherein the subject has a reduction of at least 15% in affective, sensory, or evaluative qualities of pain over about 24 hours or more following administration of the pharmaceutical composition. Further provided herein are methods wherein the subject has a reduction of at least 50% in the occurrence of vomiting over about 24 hours or more following administration of the pharmaceutical composition. Further provided herein are methods wherein the subject is nausea-prone. Further provided herein are methods wherein the subject is susceptible to opioid induced nausea or vomiting (OINV). Further provided herein are methods wherein the subject has increased pain relief compared to a subject administered a pharmaceutical composition that comprises the opioid analgesic and the non-opioid analgesic without the antiemetic. Further provided herein are methods wherein the subject has increased pain relief during the first 6 hours post administration. Further provided herein are methods wherein the administration of the pharmaceutical composition further provides for reduction or prevention of opioid induced nausea or vomiting (OINV) in the subject. Further provided herein are methods wherein the subject has reduced severity of nausea or vomiting. Further provided herein are methods wherein the subject has reduced severity of nausea or vomiting over about 6 hours following initial administration of the pharmaceutical composition. Further provided herein are methods wherein the subject has reduced severity of nausea or vomiting over about 24 hours following initial administration of the pharmaceutical composition. Further provided herein are methods wherein the subject has reduced occurrence of nausea or vomiting. Further provided herein are methods wherein the subject has a reduction of at least 70% in the occurrence of ONIV over about 48 hours or more following administration of the pharmaceutical composition. Further provided herein are methods wherein the subject has a reduction of at least 50% in occurrence of ONIV for about 24 hours or more following administration of the pharmaceutical composition. Further provided herein are methods wherein reducing nausea or vomiting in the subject is in comparison to a subject administered a pharmaceutical composition that comprises the opioid analgesic and the non-opioid analgesic without the antiemetic. Further provided herein are methods wherein the method reduces occurrence or severity of nausea or vomiting over about 24 hours following administration of the pharmaceutical composition. Further provided herein are methods wherein the subject has a reduction of at least 50% in occurrence of moderate to severe nausea or vomiting over about 48 hours or more following administration of the pharmaceutical composition. Further provided herein are methods wherein the subject has a reduction of at least 60% in occurrence of vomiting over about 5 days or more following administration of the pharmaceutical composition. Further provided herein are methods wherein the subject has a reduction of at least 50% in doses of an antiemetic over about 48 hours or more following administration of the pharmaceutical composition. Further provided herein are methods wherein the subject has a reduction of at least 75% in doses of a parenteral antiemetic over about 48 hours or more following administration of the pharmaceutical composition. Further provided herein are methods wherein the subject has a reduction of at least 70% in doses of an antiemetic over about 5 days or more following administration of the pharmaceutical composition. Further provided herein are methods wherein the subject has a reduction of at least 80% in doses of an antiemetic over about 5 days or more following administration of the pharmaceutical composition. Further provided herein are methods wherein the subject has a reduction of at least 70% in severe nausea over about 5 days or more following administration of the pharmaceutical composition. Further provided herein are methods wherein the subject has a reduction of at least 40% in peak severity of nausea over about 5 days or more following administration of the pharmaceutical composition. Further provided herein are methods wherein the subject has an increase of 60% in the likelihood of no nausea, no vomiting, or no use of an antiemetic over about 5 days or more following administration of the pharmaceutical composition. Further provided herein are methods wherein the subject has an increase of 100% in the likelihood of no nausea, no vomiting, or no use of an antiemetic over about 5 days or more following administration of the pharmaceutical composition. Further provided herein are methods wherein the administration occurs more than once over about 24 hours or longer. Further provided herein are methods further comprising administering an antiemetic when awake. Further provided herein are methods further comprising assessing the subject by a Nausea Severity Scale (NIS). Further provided herein are methods further comprising assessing the subject by a Retching or Vomiting Index (RVI). Further provided herein are methods further comprising assessing the subject by a Vomiting Supplemental Medications (NVSM). Further provided herein are methods wherein the administration occurs every four to six hours. Further provided herein are methods wherein the administration occurs two to six times over the first 24 hours. Further provided herein are methods wherein the administration occurs one to six times after the first 24 hours. Further provided herein are methods wherein the administration occurs no more than six times every 24 hours. Further provided herein are methods wherein the administration occurs periodically over 1-28 days. Further provided herein are methods wherein the administration occurs periodically over 1-21 days. Further provided herein are methods wherein the administration occurs periodically over 1-14 days. Further provided herein are methods wherein the administration occurs periodically over 1-7 days. Further provided herein are methods wherein the administration occurs periodically over 1-5 days. Further provided herein are methods wherein the administration occurs periodically over 1-3 days. Further provided herein are methods wherein the administration occurs periodically over 1-2 days. Further provided herein are methods wherein the administration occurs periodically over 24 hours. Further provided herein are methods wherein the administration is an oral administration. Further provided herein are methods wherein the administration begins from 0 to 6 hours before a surgery. Further provided herein are methods wherein the administration begins from 0 to 6 hours after a surgery. Further provided herein are methods wherein the administration is from 0 to 6 hours after an injury. Further provided herein are methods wherein the subject is a post-operative subject. Further provided herein are methods wherein the subject is a post-discharge subject. Further provided herein are methods wherein the pain is pain from an operation or post-operative pain. Further provided herein are methods wherein the pain is acute pain. Further provided herein are methods wherein the pain is chronic pain. Further provided herein are methods wherein the pain is severe. Further provided herein are methods wherein the pain is moderate. Further provided herein are methods wherein the pain is moderate to severe. Further provided herein are methods wherein the pain is agonizing pain. Further provided herein are methods wherein the administration is prophylactic. Further provided herein are methods wherein the subject has one or more conditions or diseases. Further provided herein are methods wherein the one or more conditions or diseases comprise cancer, surgical procedure, acute physical injury, chronic physical injury, bone fracture, crush injury, spinal cord injury, inflammatory disease, non-inflammatory neuropathic condition, photophobia, or any combination thereof. Further provided herein are methods wherein the pharmaceutical composition does not cause increased sedation in comparison to a pharmaceutical composition with the same amount of the opioid analgesic, in the absence of the antiemetic. Further provided herein are methods wherein the pharmaceutical composition is a solid dosage form. Further provided herein are methods wherein the solid dosage form comprises an immediate-release matrix, wherein the immediate release matrix comprises an effective amount of the antiemetic. Further provided herein are methods wherein the solid dosage form comprises a controlled-release matrix, wherein the controlled-release matrix comprises an effective amount of the opioid analgesic. Further provided herein are methods wherein the controlled-release matrix comprises an effective amount of the non-opioid analgesic. Further provided herein are methods wherein the solid dosage form is a tablet, particle or capsule. Further provided herein are methods wherein the tablet is a multi-layer tablet. Further provided herein are methods wherein the tablet is a bi-layer tablet. Further provided herein are methods wherein the antiemetic is promethazine or a pharmaceutically acceptable salt thereof. Further provided herein are methods wherein the opioid analgesic is hydrocodone or a pharmaceutically acceptable salt thereof. Further provided herein are methods wherein the opioid analgesic is oxycodone or a pharmaceutically acceptable salt thereof. Further provided herein are methods wherein the non-opioid analgesic is acetaminophen or a pharmaceutically acceptable salt thereof. Further provided herein are methods wherein: the opioid analgesic is hydrocodone or a pharmaceutically acceptable salt thereof that is present in an amount of from about 6.5 mg to about 8.5 mg; and the antiemetic is promethazine or a pharmaceutically acceptable salt thereof that is present in an amount of from about 12.5 mg to about 25 mg. Further provided herein are methods wherein: the opioid analgesic is hydrocodone or a pharmaceutically acceptable salt thereof that is present in an amount of from about 6.5 mg to about 8.5 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg. Further provided herein are methods wherein: the opioid analgesic is oxycodone or a pharmaceutically acceptable salt thereof that is present in an amount of from about 5 mg to about 15 mg; and the antiemetic is promethazine or a pharmaceutically acceptable salt thereof that is present in an amount of from about 12.5 mg to about 25 mg. Further provided herein are methods wherein: the opioid analgesic is hydrocodone, or a pharmaceutically acceptable salt thereof that is present in an amount of from 5 mg to about 15 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg. Further provided herein are methods wherein the non-opioid analgesic is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof. Further provided herein are methods wherein: the antiemetic is promethazine or a pharmaceutically acceptable salt thereof that is present in amount of about 12.5 mg, the opioid analgesic is hydrocodone or a pharmaceutically acceptable salt thereof that is present in an amount of about 7.5 mg; and the non-opioid analgesic is acetaminophen or a pharmaceutically acceptable salt thereof that is present in an amount of about 325 mg. Further provided herein are methods wherein: the antiemetic is promethazine hydrochloride that is present in an amount of about 12.5 mg; the opioid analgesic is hydrocodone bitartrate that is present in an amount of about 7.5 mg; and the non-opioid analgesic is acetaminophen that is present in an amount of about 325 mg. Further provided herein are methods wherein the subject is human. Further provided herein are methods wherein the pharmaceutical composition further comprises one or more excipients. Further provided herein are methods wherein the one or more excipients comprise an antioxidant agent, a binder, a coating material, a colorant agent, a diluent, a disintegrant, a dispersant, an emulsifying agent, a flavoring agent, a glidant, a lubricant, a pH modifying agent, a plasticizer, a preservative agent, a solubilizing agent, a stabilizer, a surfactant, a sweetening agent, a thickening agent, a pharmaceutically inert material, or any combination thereof. Further provided herein are methods wherein the antiemetic has a Tmax that is about 3-6 hours. Further provided herein are methods wherein the antiemetic has a Tmax that is about 20-100 minutes shorter than a Tmax of a corresponding standard-release antiemetic. Further provided herein are methods wherein the antiemetic has a Tmax that is about 3-5 hours, wherein the subject is fasted. Further provided herein are methods wherein the antiemetic has a Tmax of about 4 hours. Further provided herein are methods wherein the antiemetic has a Tmax that is about 20-80 minutes shorter than a Tmax of a corresponding standard-release antiemetic, wherein the subject is fasted. Further provided herein are methods wherein the antiemetic has a Tmax that is about 50 minutes shorter than a Tmax of a corresponding standard-release antiemetic. Further provided herein are methods wherein the antiemetic has a Tmax that is about 4-6 hours, wherein the subject is fed. Further provided herein are methods wherein the antiemetic has a Tmax of about 5 hours. Further provided herein are methods wherein the antiemetic has a Tmax that is about 40-100 minutes shorter than a Tmax of a corresponding standard-release antiemetic, wherein the subject is fed. Further provided herein are methods wherein the antiemetic has a Tmax that is about 70 minutes shorter than a Tmax of a corresponding standard-release antiemetic. Further provided herein are methods wherein the antiemetic has an about 20-200% greater absorption during in about two hours than a corresponding standard-release antiemetic. Further provided herein are methods wherein the antiemetic has an about 20-200% greater absorption in about 90 minutes than a corresponding standard-release antiemetic. Further provided herein are methods wherein the antiemetic has an about 20-200% greater absorption in about an hour than a corresponding standard-release antiemetic. Further provided herein are methods wherein the antiemetic has an about 20-100% greater absorption in about an hour than a corresponding standard-release antiemetic. Further provided herein are methods wherein the antiemetic has an about 60% greater absorption in about an hour than a corresponding standard-release antiemetic. Further provided herein are methods wherein the antiemetic has an about 20-60% greater absorption in about 45 minutes than a corresponding standard-release antiemetic. Further provided herein are methods wherein the antiemetic has an about 40% greater absorption in about 45 minutes than a corresponding standard-release antiemetic. Further provided herein are methods wherein the antiemetic has an about 20-60% greater absorption in about 30 minutes than a corresponding standard-release antiemetic. Further provided herein are methods wherein the antiemetic has an about 40% greater absorption in about 30 minutes than a corresponding standard-release antiemetic. Further provided herein are methods wherein the antiemetic is promethazine or a pharmaceutically acceptable salt thereof. Further provided herein are methods wherein the antiemetic is promethazine hydrochloride. Further provided herein are methods wherein the administration occurs multiple times per day. Further provided herein are methods wherein the administration occurs at least twice daily. Further provided herein are methods wherein the administration occurs 2 to 6 times daily.

### BRIEF DESCRIPTION OF THE DRAWINGS

FIG. 1 illustrates an exemplary dissolution profile of a bi-layer tablet.

FIGS. 2A-2C illustrate a manufacturing processes for pharmaceutical composition fabrication. FIG. 2A shows a process of making a layer of hydrocodone bitartrate and acetaminophen (APAP). FIG. 2B shows a process of making a layer of promethazine HCl.

FIG. 2C shows a process of compressing two layers into a bi-layer tablet.

FIG. 3 illustrates a friabilator apparatus utilized for friability measurements.

FIG. 4 illustrates tablet orientations in a plunger for hardness measurements.

FIG. 5 illustrates a range of values for pharmaceutical composition hardness (kp), thickness (mm), friability (%), and dissolution rates (Disso) of hydrocodone (HC), acetaminophen (APAP), and promethazine (PMZ).

FIG. 6 illustrates a correlation between pharmaceutical composition hardness and friability.

FIG. 7 illustrates a correlation between pharmaceutical composition thickness and friability.

FIG. 8 illustrates a correlation between pharmaceutical composition thickness and hardness.

FIG. 9 illustrates a correlation between pharmaceutical composition hardness and percent hydrocodone (HC) dissolution within 10 and 15 minutes.

FIG. 10 illustrates a correlation between pharmaceutical composition hardness and percent acetaminophen (APAP) dissolution within 10 and 15 minutes.

FIG. 11 illustrates a correlation between pharmaceutical composition hardness and percent promethazine (PMZ) dissolution within 10 and 15 minutes.

FIG. 12 illustrates a correlation between pharmaceutical composition thickness and percent hydrocodone (HC) dissolution within 10 and 15 minutes.

FIG. 13 illustrates a correlation between pharmaceutical composition thickness and percent acetaminophen (APAP) dissolution with 10 and 15 minutes.

FIG. 14 illustrates a correlation between pharmaceutical composition hardness and percent promethazine (PMZ) dissolution within 10 and 15 minutes.

FIGS. 15A-15B illustrate a linear scale of mean plasma concentrations (ng/mL) of hydrocodone from Formulation A and commercial hydrocodone within the first 4 hours (FIG. 15A) and 8 hours (FIG. 15B) following administration to treatment groups A (Formulation A—Fasted), B (Formulation A—Fed), C (Comparator—Fasted) and D (Comparator—Fed).

FIGS. 16A-16B illustrate a linear scale of mean plasma concentrations (μg/mL) of acetaminophen from Formulation A and commercial acetaminophen within the first 4 hours (FIG. 16A) and 8 hours (FIG. 16B) following administration to treatment groups A (Formulation A—Fasted), B (Formulation A—Fed), C (Comparator—Fasted) and D (Comparator—Fed).

FIGS. 17A-17B illustrate a linear scale of mean plasma concentrations (ng/mL) of promethazine from Formulation A and commercial promethazine within the first 4 hours (FIG. 17A) and 8 hours (FIG. 17B) following administration to treatment groups A (Formulation A—Fasted), B (Formulation A—Fed), C (Comparator—Fasted) and D (Comparator—Fed).

FIG. 18 illustrates the mean and standard deviation of summed pain intensity differences (on the Categorical Pain Intensity Scale) over 24 hours (SPID24).

FIG. 19 illustrates a Kaplan Meier survival curve of time to onset of perceptible pain reduction among subjects with moderate pain or greater than moderate pain relief in the first 6 hours.

FIG. 20 illustrates a Kaplan Meier survival curve of time to the second use of study medication from the first dose of study medication.

FIG. 21 illustrates a Kaplan Meier survival curve of time to first dose of supplemental analgesic medication from the first dose of study medication.

FIG. 22 illustrates the mean and standard deviation of summed nausea intensity differences on the Nausea Intensity Scale (NIS) over 24 hours.

FIG. 23 illustrates frequency of vomiting after the first dose over all 5 post-operation days.

FIG. 24 illustrates the percentage of subjects using supplementary medication for nausea/vomiting over 5 days.

FIG. 25 illustrates a Kaplan Meier survival curve of time to first dose of supplemental medication for nausea/vomiting from the first dose of study medication.

FIG. 26 illustrates a plot of co-primary OINV endpoint recording in subjects with OINV over 48 hours.

FIG. 27 illustrates a plot of secondary endpoint recording in subjects with OINV over 48 hours.

FIG. 28 illustrates a plot of nausea and severity endpoint recording in subjects over 48 hours.

FIG. 29 illustrates a plot of peak nausea severity endpoint recording in subjects over 48 hours.

FIG. 30 illustrates a plot of SPID24 recording for subjects with severe pain over 24 hours.

FIG. 31 illustrates a plot of relief of moderate or severe pain recording in subjects over the first 24 hours.

FIG. 32 illustrates a plot of percent change in overall QPI score over the first 24 hours in subjects.

FIG. 33 illustrates a plot of the percentage of subjects with repeat retching recorded over 5 days.

FIG. 34 illustrates a plot of the percentage of subjects with sleep disturbance during the first 2 nights of in-hospital observation.

### DETAILED DESCRIPTION

The disclosure is generally directed to compositions comprising multiple pharmaceutically active agents that are useful as therapeutics that alleviate, abate or eliminate one or more conditions in a subject in need thereof, as further described herein below.

The term “about” means the referenced numeric indication plus or minus 15% of that referenced numeric indication.

The term “subject” as used herein refers to a mammal (e.g., a human, mouse, rat, guinea pig, dog, cat, horse, cow, pig, or non-human primate, such as a monkey, chimpanzee or baboon). In a particular instance the subject is a human subject.

The term “controlled-release” or “controlled release” refers to release of at least one pharmaceutically active agent in a formulation or component of a formulation (e.g., a layer in a tablet, a particulate in a multi-particulate composition, etc.) at a time later than immediately after contact with a dissolution fluid or administration to a subject. Typically, the active agent exhibits a lag time in quantifiable dissolution. A controlled-release formulation or component of a formulation has a slower release of the at least one pharmaceutically active agent than a pharmaceutically active agent in an immediate-release formulation or component of a formulation. A controlled-release formulation can begin its release and continue that release over an extended period of time. A rate of release in a controlled-release formulation can be constant, can increase or decrease over time, can be pulsed, can be continuous or intermittent, and the like.

The term “immediate-release” or “immediate release” refers to the release of at least one pharmaceutically active agent in a formulation or component of a formulation (e.g., a layer in a tablet, a particulate in a multi-particulate composition, etc.) quickly after contact with a dissolution fluid or administration to a subject.

The term “supplemental antiemetic” refers to an antiemetic used as rescue medication taken after a target composition (e.g., an opioid analgesic) intended for treatment of a condition, such as pain, is administered to a subject in need thereof. The term “supplemental analgesic” refers to an analgesic used as rescue medication taken after a target composition (e.g., an opioid analgesic) intended for treatment of a condition, such as pain, is administered to a subject in need thereof.

The present disclosure provides methods and compositions for effective pain treatment. The term “pain” as used herein to all types of pain, in particular moderate to severe pain. Pain includes agonizing pain, neuropathic pain, post-operative pain, chronic lower back pain, cluster headaches, herpes neuralgia, phantom limb pain, central pain, dental pain, neuropathic pain, visceral pain, surgical pain, bone injury pain, pain during labor and delivery, pain resulting from burns, post partum pain, migraine, angina pain, genitourinary tract-related pain including cystitis and nociceptive pain. In some instances, the pain is chronic or acute (“chronic pain” or “acute pain”). The term “post-operative pain” as used herein refers to a subject's pain after surgery. In some aspects, provided herein are methods for reducing or preventing pain in a subject, comprising administering to a subject in need thereof a pharmaceutical composition that comprises an immediate-release matrix comprising an antiemetic and a controlled-release matrix comprising and opioid analgesic, wherein the subject experiences increased pain relief compared to a subject administered with a composition of the same opioid analgesic. In some instances, the pharmaceutical composition that comprises the immediate-release matrix comprising and the controlled-release matrix comprising the opioid analgesic has a synergistic effect to provide increased pain relief to a subject in need thereof. In some instances, controlled-release matrix further comprises a non-opioid analgesic. In some instances, the pain is agonizing pain.

The term “affective” as used herein with regard to an indicator or descriptor of pain refers to a measurement of pain that is based on the pain experience of a subject. Tension, fear, and autonomic properties may be considered in an affective measurement of pain. Exemplary affective measurements of pain include, without limitation, “agonizing” and “annoying”-agonizing being an extreme pain experience.

The term “sensory” as used herein with regard to an indicator or descriptor of pain refers to a measurement of how the subject feels the pain. Temporal, spatial, pressure, thermal, among other properties, may be considered in a sensory measurement of pain. Exemplary sensory measurements of pain include, without limitation, aching, throbbing, hot, heavy, pulling, sharp, radiating, pressing, swollen, tight, stabbing, and stinging.

The term “evaluative” as used herein with regard to an indicator or descriptor of pain refers to a measurement of the overall intensity of the pain experience. Exemplary evaluative measurements of pain include, without limitation, aching and hurting.

In some instances, a “standard-release” agent (e.g., standard-release antiemetic) refers to an agent commercially available at the time of filing this application (e.g., commercially available promethazine hydrochloride), such as those used as a comparison point in Example 7. In some embodiments, a standard-release antiemetic is PHENERGAN. In some embodiments, a standard-release opioid analgesic is VICOPROFEN. In some embodiments, a standard-release non-opioid analgesic is ULTRACET.

In another aspect, provided herein are methods for treating, reducing, or preventing pain in a subject, comprising administering to a subject in need thereof a pharmaceutical composition that comprises an immediate-release antiemetic and a controlled-release opioid analgesic, wherein the subject experiences increased pain relief and decreased nausea or vomiting compared to a subject administered with a composition of the same opioid analgesic. In another aspect, provided herein are methods for treating, reducing, or preventing pain in a subject, comprising administering to a subject a pharmaceutical composition that comprises an immediate-release antiemetic and a controlled-release opioid analgesic, wherein the subject experiences a reduced need for a supplemental antiemetic or a supplemental analgesic compared to a subject administered with a composition of the same opioid analgesic. In another aspect, provided herein are methods for treating, reducing, or preventing pain in a subject, comprising periodically administering to the subject for one or more days a pharmaceutical composition that comprises an immediate-release antiemetic and a controlled-release opioid analgesic, wherein the subject experiences less frequent or less intense nausea or vomiting compared to a subject periodically administered with a composition of the same opioid analgesic for one or more days. In some instances, the subject is a human. In some instances, the pain is agonizing pain, e.g., an “affective” descriptor of extremely painful, significantly distressful, and/or most bothersome pain, in Quality of Pain Index (QPI). In some instances, the pain treated herein is chronic pain. In some instances, the pain is moderate to severe. In some instances, the pain treated herein is moderate. In some instances, the pain treated herein is severe. In some instances, on a scale of 0 to 100 mm, where 0 mm=no pain, and 100 mm=severe pain, moderate pain is about 50 mm to about 70 mm, severe pain is about 70 mm to about 100 mm, and moderate to severe pain is about 50 mm to about 100 mm. In some instances, a subject treated for pain is a post-operative subject who has undergone a recent surgical procedure. In some instances, a subject the subject treated for pain is a post-discharge subject who has been recently discharged from a hospital or surgical care unit. In some instances, the pain is caused by surgery. In some instances, the pain is operative pain. In some instances, the pain continues after surgery is complete. In some instances the pain is post-operative or post-discharge pain. In some instances, the pain treated herein is caused by a disease or condition. In some instances, the one or more conditions or diseases comprise cancer, acute physical injury, chronic physical injury, bone fracture, crush injury, spinal cord injury, inflammatory disease, non-inflammatory neuropathic condition, or any combination thereof.

In some instances, an incidence of nausea or vomiting is prevented or reduced after the administration of a pharmaceutical composition disclosed herein. In some instances, the incidence of the nausea or vomiting is reduced by about 10-25%, about 10-50%, or about 50-80%, or about 70-80% after the administration of the pharmaceutical composition to a subject in need thereof, in comparison to administration with the composition of the same opioid analgesic. In some instances, an intensity of nausea or vomiting is reduced after the administration of the pharmaceutical composition to a subject in need thereof. In some instances, the intensity of the nausea or vomiting is reduced from (severe to moderate) to (mild to none) after the administration of the pharmaceutical composition to a subject in need thereof. In some instances, the intensity of the nausea or vomiting is reduced from severe to moderate, from severe to mild, or from severe to none, after the administration of the pharmaceutical composition to a subject in need thereof. In some instances, the intensity of the nausea or vomiting is reduced from moderate to mild, or from moderate to none, after the administration of the pharmaceutical composition to a subject in need thereof. In some instances, nausea can be measured or quantified using the Nausea Intensity Scale (NIS). In some instances, nausea can be measured on a scale of 0 to 10, where 0 is no nausea and 10 is severe nausea. In some instances, nausea is measured by soliciting feedback from a subject. In some instances, nausea can be measured as the time from administration of the opioid analgesic to the time of the first episode of nausea. In some instances, nausea can be measured as the time from administration to the time of delivery of a rescue therapy.

In some instances, vomiting can be measured or quantified using the Vomiting Frequency Scale (VFS). In some instances, vomiting can be measured based on a question, “how often did you vomit over the past hour?” where 0=no vomiting, 1=one time to vomit, 2=two times to vomit, 3=three or more times to vomit. In some instances, vomiting is measured by soliciting feedback from a subject. In some instances, vomiting is measured as the time from administration to the time of the first episode of vomiting.

In some instances, administration of a pharmaceutical compound disclosed herein is repeated every 4-6 hours to a subject in need thereof. In some instances, the administration is repeated every 8 hours. In some instances, the administration is repeated for 1-5 days. In some instances, nausea or vomiting of a subject is prevented or reduced within 24, 48, 72, 96, or 120 hours after the administration of a pharmaceutical composition disclosed herein to a subject in need thereof.

In some instances, compositions disclosed herein comprise a controlled-release opioid analgesic. In some instances, the a controlled-release opioid analgesic comprises hydrocodone, oxycodone, oripavine, dihydromorphine, hydromorphinol, nicomorphine, dipropanoylmorphine, diacetyldihydromorphine, desomorphine, methyldesorphine, heterocodeine, benzylmorphine, dihydroheterocodeine, myrophine, pentamorphone, etorphine, acetyldihydrocodeine, nicocodeine, nicodicodeine, alphamethylfentanyl, carfentanil, parafluorofentanyl, thiofentanyl, anileridine, benzethidine, difenoxin, diphenoxylate, etoxeridine, furethidine, morpheridine, pheneridine, phenoperidine, piminodine, allylprodine, loperamide, dextropropoxyphene, dihydroetorphine, acetorphine, levophenacylmorphan, phenomorphan, drotebanol, dipipanone, normethadone, phenadoxone, dimepheptanol, levacetylmethadol, dextromoramide, diethylthiambutene, dimethylthiambutene, ethylmethylthiambutene, dextropropoxyphene, dimenoxadol, tilidine, ethoheptazine, proheptazine, piritramide, etonitazene, tapentadol, tramadol, a pharmaceutically acceptable salt of any one of the foregoing, or any combination thereof.

In some instances, compositions disclosed herein comprise an immediate-release antiemetic. In some instances, the immediate-release antiemetic comprises promethazine, aprepitant, dronabinol, perphenazine, palonosetron, metoclopromide, domperidone, prochlorperazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, thiethylperazine, thioproperazine, tropisetron, droperidol, haloperidol, diphenhydramine, midazolam, lorazepam, hyoscine, dexamethasone, emetrol, a pharmaceutically acceptable salt of any one of the foregoing, or any combination thereof. In some instances, the immediate-release antiemetic is promethazine or a pharmaceutically acceptable salt thereof.

In some instances, a controlled-release opioid analgesic included in a composition described herein comprises hydrocodone, oxycodone, or a pharmaceutically acceptable salt of any one of the foregoing, or any combination thereof and the immediate-release antiemetic comprises promethazine or a pharmaceutically acceptable salt thereof and ondansetron or a pharmaceutically acceptable salt thereof. In some instances, a pharmaceutical composition comprises from about 6.5 mg to about 8.5 mg of hydrocodone, oxycodone, or a pharmaceutically acceptable salt of any one of the foregoing, or any combination thereof and from about 11 mg to about 14 mg of promethazine or a pharmaceutically acceptable salt thereof.

In some instances, a pharmaceutical composition comprises a controlled-release non-opioid analgesic and a controlled-release non-opioid analgesic. In some instances, the controlled-release non-opioid analgesic comprises acetaminophen, acetylsalicylic acid, amoxiprin, benorilate, choline magnesium salicylate, diflunisal, faislamine, methyl salicylate, magnesium salicylate, diclofenac, aceclofenac, acemetacin, bromfenac, etodolac, indometacin, nabumetone, sulindac, tolmetin, ibuprofen, carprofen, fenbuprofen, flubiprofen, ketaprofen, ketorolac, loxoprofen, naproxen, suprofen, mefenamic acid, meclofenamic acid, piroxicam, lomoxicam, meloxicam, tenoxicam, phenylbutazone, azapropazone, metamizole, oxyphenbutazone, sulfinprazone, a pharmaceutically acceptable salt of any one of the foregoing, or any combination thereof. In some instances, the controlled-release non-opioid analgesic is acetaminophen or a pharmaceutically acceptable salt thereof. In some instances, the controlled-release non-opioid analgesic is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof.

In some instances, a pharmaceutical composition comprises two or more immediate-release antiemetics. In some instances, the two or more immediate-release antiemetics comprise promethazine or a pharmaceutically acceptable salt thereof and ondansetron or a pharmaceutically acceptable salt thereof. In some instances, a pharmaceutical composition comprises an opioid antagonist or an abuse deterrent agent. In some instances, the opioid antagonist agent or abuse deterrent agent comprises nalmefene, naloxone, naltrexone, cyclazacine, levallorphan, niacin, a pharmaceutically acceptable salt of any one of the foregoing, or any combination thereof. In some instances, the pharmaceutical composition further comprises the abuse deterrent agent that is formulated as a gel-forming agent comprising a pharmaceutically acceptable polymer. In some instances, the pharmaceutically acceptable polymer is capable of forming a viscous gel upon contact with a solvent, wherein the viscous gel resists crushing and snorting. In some instances, the pharmaceutically acceptable polymer comprises polyethylene oxide, polyvinyl alcohol, hydroxypropyl methyl cellulose, carbomers, or any combination thereof.

In some instances, a pharmaceutical composition disclosed herein is formulated as a tablet, capsule, or lollipop. In some instances, the pharmaceutical composition is formulated with a controlled-release enteric coating. In some instances, the pharmaceutical composition is formulated as the tablet that is a bi-layer tablet, a two layer tablet, a multi-layer tablet, a tannate tablet, an oral disintegrating tablet, an effervescent tablet, or any combination thereof. In some instances, the pharmaceutical composition is formulated as the capsule that is a soft gelatin capsule or a hard gelatin capsule. In some instances, the capsule has micro drilled holes. In some instances, the capsule is formulated with an immediate-release powder. In some instances, the capsule is formulated with one or more controlled-release or modified-release particulates. In some instances, the particulate is a bead, a sphere, or a pellet. In some instances, the tablet or capsule further comprises an inner dosage and an outer dosage, the latter being in the form of an envelope over the former. In some instances, the inner dosage and outer dosage components are separated by an enteric layer.

In some instances, a pharmaceutical composition disclosed herein comprises one or more excipients. In some instances, the one or more excipients comprise an antioxidant agent, a binder, a coating material, a colorant agent, a diluent, a disintegrant, a dispersant, an emulsifying agent, a flavoring agent, a glidant, a lubricant, a pH modifying agent, a plasticizer, a preservative agent, a solubilizing agent, a stabilizer, a surfactant, a sweetening agent, a thickening agent, a pharmaceutically inert material, or any combination thereof. In some instances, the one or more excipients comprise the antioxidant agent that is a flavonoid, an anthocyanidin, an anthocyanin, a proanthocyanidin, or any combination thereof. In some instances, the one or more excipients comprise the binder that is hydroxypropylcellulose, methylcellulose, corn starch, pregelatinized starch, hydroxypropyl methylcellulose, hydroxypropyl starch, glucose, dextrose, sucrose, lactose, sorbitol, polyvinyl alcohol, polyethylene glycol, acacia, tragacanth, sodium alginate, polymethacrylate, polyvinylpyrrolidone, povidone, dextrin, pullulane, agar, gelatin, tragacanth, macrogol, or any combination thereof. In some instances, the one or more excipients comprise the coating material that is hydroxypropyl methyl cellulose 2910, aminoalkyl methacrylate copolymer E, polyvinylacetal diethylaminoacetate, macrogol 6000, titanium oxide, or any combination thereof. In some instances, the one or more excipients comprise the colorant agent that is food red dye No. 2, food red dye No. 3, food yellow dye No. 4, food yellow dye No. 5, food blue dye No. 1, food blue dye No. 2, water-insoluble lake dye, beta-carotene, chlorophyll, iron oxide red, D&C Red No. 33, FD&C Red No. 3, FD&C Red No. 40, D&C Yellow No. 10, C Yellow No. 6, or any combination thereof. In some instances, the one or more excipients comprise the diluent that is cellulose, microcrystalline cellulose, dry starch, hydrolyzed starch, corn starch, cyclodextrin, powdered sugar, lactose, D-mannitol, aluminum hydroxide gel, precipitated calcium carbonate, carbonate, magnesium aluminometasilicate, dibasic calcium phosphate, sodium chloride, silicon dioxide, titanium dioxide, titanium oxide, dicalcium phosphate dihydrate, calcium sulfate, alumina, kaolin, talc, or any combination thereof. In some instances, the one or more excipients comprise the disintegrant that is alginic acid, crosslinked polyvinylpyrrolidone, croscarmellose sodium, potassium starch glycolate, sodium starch glycolate, clay, cellulose, starch, gum, or any combination thereof. In some instances, the one or more excipients comprise the emulsifying agent that is gelatin, egg yolk, casein, cholesterol, acacia, tragacanth, chondrus, pectin, methyl cellulose, carbomer, cetostearyl alcohol, cetyl alcohol, or any combination thereof. In some instances, the one or more excipients comprise the flavoring agent that is natural fruit, artificial fruit, artificial banana, artificial strawberry, artificial pineapple, or any combination thereof. In some instances, the one or more excipients comprise the lubricant that is mineral oil, magnesium stearate, calcium stearate, stearic acid, glyceryl behenate, polyethylene glycol, talc, or any combination thereof. In some instances, the one or more excipients comprise the pH buffering agent that is gluconate, lactate, citrate, citric acid, acetate, phosphate, potassium phosphate, sodium phosphate, benzoate, sodium benzoate, carbonate salt, or any combination thereof. In some instances, the one or more excipients comprise the plasticizer that is triethyl citrate, triacetin, macrogol 6000, or any combination thereof. In some instances, the one or more excipients comprise the preservative agent that is sodium benzoate, paraoxybenzoic acid ester, methyl paraben, ethyl paraben, butyl paraben, propyl paraben, chlorobutanol, benzyl alcohol, phenylethylalcohol, dehydroacetic acid, sorbic acid, benzalkonium chloride, benzethonium chloride, phenol, phenylmercuric nitrate, thimerosal, or any combination thereof. In some instances, the one or more excipients comprise the solubilizing agent that is ethyl alcohol, glycerin, D-mannitol, trehalose, benzyl benzoate, trisaminomethane, cholesterol, triethanolamine, sodium carbonate, sodium citrate, sodium salicylate, sodium acetate, ethanol, isopropanol, t-butanol, phenol, cresol, benzyl alcohol, propylene glycol, polypropylene glycol, polyethylene glycol, or any combination thereof. In some instances, the one or more excipients comprise the stabilizer that is ethanol, glycerin, polyethylene glycol, propylene glycol, polypropylene glycol, hydroxypropyl methyl cellulose, hydroxymethylcellulose, or any combination thereof. In some instances, the one or more excipients comprise the surfactant that is polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, lauromacrogol, poloxamer, or any combination thereof. In some instances, the one or more excipients comprise the sweetening agent that is sorbitol, saccharin, acesulfame, acesulfame potassium, sucralose, xylitol, maltitol, sucrose, aspartame, fructose, neotame, glycerin, sodium saccharate, glycyrrhizin dipotassium, acesulfame K, mannitol, invert sugar, liquid sugar, or any combination thereof. In some instances, the one or more excipients comprise the thickening agent that is acacia, alginic acid bentonite, carbomer, carboxymethylcellulose calcium, carboxymethylcellulose sodium, cetostearyl alcohol, methyl cellulose, ethylcellulose, glycerin, gelatin guar gum, hydroxyethyl cellulose, hydroxymethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum, or any combination thereof.

In some instances, a controlled-release opioid analgesic has a Tmax that is about 1.4-2.0 hours. In some instances, the controlled-release opioid analgesic has a Tmax that is about: 1.4, 1.45, 1.5, 1.55, 1.6, 1.65, 1.7, 1.75, 1.8, 1.85, 1.9, 1.95, or 2 hours. In some instances, the controlled-release opioid analgesic has a Tmax that is about 5-60 minutes longer than a corresponding Tmax of the composition of the same opioid analgesic. In some instances, the controlled-release opioid analgesic has a Tmax that is about 10-30 minutes longer than a corresponding Tmax of the composition of the same opioid analgesic. In some instances, the controlled-release opioid analgesic has a Tmax that is about: 5-10 minutes, 10-20 minutes, 20-30 minutes, 30-40 minutes, 40-50 minutes, or 50-60 minutes longer than a corresponding Tmax of the composition of the same opioid analgesic. In some instances, the controlled-release opioid analgesic has a Tmax that is about: 5, 10, 15, 20, 25, 30, 35, 40, 45, 50, 55, or 60 minutes longer than a corresponding Tmax of the composition of the same opioid analgesic.

In some instances, an immediate-release antiemetic has a Tmax that is about 3.5 to 4.3 hours. In some instances, the immediate-release antiemetic has a Tmax that is about: 3.5, 3.6, 3.7, 3.75, 3.8, 3.85, 3.9, 3.95, 4, 4.1, 4.2, or 4.3 hours. In some instances, the immediate-release antiemetic has a Tmax that is about 30-120 minutes shorter than a Tmax of a corresponding standard-release antiemetic. In some instances, the immediate-release antiemetic has a Tmax that is about 50-100 minutes shorter than a Tmax of a corresponding standard-release antiemetic. In some instances, the immediate-release antiemetic has a Tmax that is about 60-90 minutes shorter than a Tmax of a corresponding standard-release antiemetic. In some instances, the immediate-release antiemetic has a Tmax that is about: 30-40 minutes, 40-50 minutes, 50-60 minutes, 60-70 minutes, 70-80 minutes, 80-90 minutes, 90-100 minutes, 100-110 minutes, or 110-120 minutes shorter than a Tmax of a corresponding standard-release antiemetic in median times. In some instances, the immediate-release antiemetic has a Tmax that is about: 30, 35, 40, 45, 50, 55, 60, 65, 70, 75, 80, 85, 90, 95, 100, 105, 110, 115, or 120 minutes shorter than a Tmax of a corresponding standard-release antiemetic in median times. In some instances, the immediate-release antiemetic has a Tmax that is about: 70, 71, 72, 73, 74, 75, 76, 77, 78, 79, or 80 minutes shorter than a Tmax of a corresponding standard-release antiemetic in median times.

In some instances, a controlled-release non-opioid analgesic has a Tmax that is about 0.9 to 1.1 hours. In some instances, the controlled-release non-opioid analgesic has a Tmax that is about: 0.9, 0.92, 0.94, 0.96, 0.98, 1, 1.02, 1.04, 1.06, 1.08, or 1.1 hours. In some instances, the controlled-release non-opioid analgesic has a Tmax that is about 5-30 minutes longer than a corresponding Tmax of the composition of the same opioid analgesic and the same non-opioid analgesic. In some instances, the controlled-release non-opioid analgesic has a Tmax that is about 10-25 minutes longer than of a corresponding Tmax of the composition of the same opioid analgesic and the same non-opioid analgesic. In some instances, the controlled-release non-opioid analgesics has a Tmax that is about 10-15 minutes longer than a corresponding Tmax of the composition of the same opioid analgesic and the same non-opioid analgesic. In some instances, the controlled-release non-opioid analgesic has a Tmax that is about: 5-10 minutes, 10-15 minutes, 15-20 minutes, 20-25 minutes, or 25-30 minutes longer than a corresponding Tmax of the composition of the same opioid analgesic and the same non-opioid analgesic. In some instances, the controlled-release non-opioid analgesic has a Tmax that is about: 5, 10, 15, 20, 25, or 30 minutes longer than a corresponding Tmax of the composition of the same opioid analgesic and the same non-opioid analgesic. In some instances, the controlled-release non-opioid analgesic has a Tmax that is about: 6, 7, 8, 9, 10, 11, 12, 13, 14, 15, 16, 17, 18, or 19 minutes longer than a corresponding Tmax of the composition of the same opioid analgesic and the same non-opioid analgesic.

In some instances, a pharmaceutical composition disclosed herein is a solid composition. In some instances, a pharmaceutical composition disclosed herein is a liquid composition. In some instances, a pharmaceutical composition disclosed herein is formulated as a patch.

### Pharmaceutical Agents

### Opioid Analgesics

In some instances, a pharmaceutical composition as disclosed herein comprises one or more opioid analgesics. Opioid analgesics include, without limitation, an opiate, an endogenous opioid, an opium alkaloid, an active opiate metabolite, an opioid peptide, an opioid from the morphine family, a semi-synthetic opioid, a synthetic opioid, and a pharmaceutically acceptable salt of any one of the foregoing. Opioid analgesics also include any combination of those mentioned above.

Exemplary endogenous opioids include endorphins, enkephalins, dynorphins, or endomorphins. Opium alkaloids, naturally occurring in opium, can include codeine, morphine, thebaine, oripavine, papaveretum, or a pharmaceutically acceptable salt of any one of the foregoing, or any combination thereof.

Exemplary opioids from the morphine family include: a) 2,4-dinitrophenylmorphine, 4,5-α-Epoxy-17-methyl-6-methylenemorphinan-3-ol (6-MDDM), dihydromorphine, hydromorphinol, N-phenethylnormorphine, 3,6,14-trihydroxy-4,5α-epoxy-17-(2-phenylethyl) morphinan (RAM-378), or a pharmaceutically acceptable salt of any one of the foregoing, or any combination thereof; b) esters of morphine including diacetylmorphine, nicomorphine, dipropanoylmorphine, diacetyldihydromorphine, acetylpropionylmorphine, desomorphine, methyldesorphine, dibenzoylmorphine, dihydroheroin, or a pharmaceutically acceptable salt of any one of the foregoing, or any combination thereof; c) ethers of morphine including dihydrocodeine, ethylmorphine, heterocodeine, or a pharmaceutically acceptable salt of any one of the foregoing, or any combination thereof; d) codeine-dionine family members including[(4R,4aR,7R,7aR,12bS)-9-methoxy-3-methyl-2,4,4a,7,7a, 13-hexahydro-1H-4,12-methanobenzofuro[3,2-e]isoquinoline-7-yl] acetate (6-Monoacetylcodeine or 6-MAC), benzylmorphine, codeine methylbromide, dihydroheterocodeine, pholcodine, myrophine, or a pharmaceutically acceptable salt of any one of the foregoing, or any combination thereof; and e) morphinones and morphols including 14-cinnamoyloxycodeinone, 14-ethoxymetopon, 14-methoxymetopon, 3-Hydroxy-14-(3-phenylpropoxy)-5-methyl-7,8-dihydro-4,5α-epoxy-17-methylmorphinan-6-one (14-Phenylpropoxymetopon or PPOM), 7-spiroindanyloxymorphone, acetylmorphone, codeinone, codoxime, thebacon, metopon, morphinone, pentamorphone, or a pharmaceutically acceptable salt of any one of the foregoing, or any combination thereof.

Exemplary active opiate metabolites include (2S,3S,4S,5R,6R)-6-[[(4R,4aR,7S,7aR,12bS)-9-hydroxy-3-methyl-2,4,4a,7,7a, 13-hexahydro-1H-4,12-methanobenzofuro[3,2-e]isoquinoline-7-yl]oxy]-3,4,5-trihydroxyoxane-2-carboxylic acid (morphine-6-glucuronide or M6G), 3-hydroxy-6-acetyl-(5α,6α)-7,8-Didehydro-4,5-epoxy-17-methylmorphinan (6-monoacetylmorphine or 6-MAM), norcodeine, normorphine, morphine-N-oxide, or a pharmaceutically acceptable salt of any one of the foregoing, or any combination thereof.

Exemplary opioid peptides include adrenorphin, amidorphin, casomorphin, (2R)-2-[[(2S)-2-[[2-[[(2R)-2-[[(2S)-2-amino-3-(4-hydroxyphenyl) propanoyl] amino] propanoyl] amino] acetyl] amino]-3-phenylpropanoyl] amino]-4-methylpentanoic acid (DADLE), (2S)-2-amino-N-[(2R)-1-[[2-[[(2S)-1-(2-hydroxyethylamino)-1-oxo-3-phenylpropan-2-yl]-methylamino]-2-oxoethyl]amino]-1-oxopropan-2-yl]-3-(4-hydroxyphenyl) propanamide (DAMGO), dermorphin, morphiceptin, nociceptin, octreotide, opiorphin, L-tyrosyl-N-{[(3-methylbutyl)amino]acetyl}-D-alaninamide (TRIMU 5), or a pharmaceutically acceptable salt of any one of the foregoing, or any combination thereof.

Exemplary semi-synthetic opioids include etorphine, hydrocodone, hydromorphone, oxycodone, oxymorphone, ethylmorphine, chloromorphide, 14-hydroxydihydrocodeine, acetyldihydrocodeine, nicocodeine, nicodicodeine, oxymorphazone, 1-iodomorphine, or a pharmaceutically acceptable salt of any one of the foregoing, or any combination thereof.

Exemplary synthetic opioids include: a) anilidopiperidines including alphamethylfentanyl, 3-allylfentanyl, 3-methylfentanyl, 3-methylthiofentanyl, 4-phenylfentanyl, alfentanil, α-methylacetylfentanyl, α-methylfentanyl, α-methylthiofentanyl, β-hydroxyfentanyl, β-hydroxythiofentanyl, β-methylfentanyl, brifentanil, carfentanil, fentanyl, lofentanil, mirfentanil, ocfentanil, ohmefentanyl, parafluorofentanyl, phenaridine, remifentanil, sufentanil, thiofentanyl, trefentanil, or a pharmaceutically acceptable salt of any one of the foregoing, or any combination thereof; b) 4-phenylpiperidines including 4-fluoromeperidine, allylnorpethidine, anileridine, benzethidine, carperidine, difenoxin, diphenoxylate, etoxeridine, furethidine, hydroxypethidine, morpheridine, oxpheneridine, pethidine, pheneridine, phenoperidine, piminodine, properidine, allylprodine, 1-methyl-4-phenyl-4-propionoxypiperidine (Desmethylprodine or MPPP), 4-phenyl-1-(2-phenylethyl)piperidin-4-yl acetate (PEPAP), α-prodine, prosidol, trimeperidine, acetoxyketobemidone, droxypropine, ketobemidone, methylketobemidone, propylketobemidone, loperamide, or a pharmaceutically acceptable salt of any one of the foregoing, or any combination thereof; c) diphenylpropylamine derivatives including propoxyphene, dextroporpoxyphene, or a pharmaceutically acceptable salt of any one of the foregoing, or any combination thereof; d) orvinols and oripavine derivatives including dihydroetorphine, etorphine, (2R)-2-((4R,7S,7aR,12bS,14R)-7,9-dimethoxy-3-methyl-1,2,3,4,7,7a-hexahydro-7,4a-ethano-4,12-methanobenzofuro[3,2-e]isoquinolin-14-yl)-4-phenylbutan-2-ol (7-PET), acetorphine, N-cyclopropylmethyl-[7α, 8α,2′,3′]-cyclohexano-1′[S]-hydroxy-6,14-endo-ethenotetrahydronororipavine (BU-48), norbuprenorphine, or a pharmaceutically acceptable salt of any one of the foregoing, or any combination thereof; e) morphinan derivative including levorphanol, levomethorphan, levophenacylmorphan, norlevorphanol, phenomorphan, furethylnorlevorphanol, drotebanol, or a pharmaceutically acceptable salt of any one of the foregoing, or any combination thereof; f) allosteric modulators including cannabidiol, tetrahydrocannabinol, or a pharmaceutically acceptable salt of any one of the foregoing, or any combination thereof; g) open chain opioids including dipipanone, methadone, normethadone, phenadoxone, dimepheptanol, dextromoramide, levomoramide, racemoramide, diethylthiambutene, dimethylthiambutene, ethylmethylthiambutene, piperidylthiambutene, pyrrolidinylthiambutene, thiambutene, tipepidine, dextropropoxyphene, dimenoxadol, dioxaphetyl butyrate, levopropoxyphene, norpropoxyphene, diampromide, phenampromide, methiodone, isoaminile, lefetamine, 6-(3,4-dihydro-1′H,2H-spiro[naphthalene-1,4′-piperidin]-1′-yl)-4,4-diphenylhexan-3-one (R-4066), or a pharmaceutically acceptable salt of any one of the foregoing, or any combination thereof; and h) various others including menthol, tilidine, ethoheptazine, metheptazine, metethoheptazine, proheptazine, bezitramide, piritramide, clonitazene, etonitazene, 7-hydroxymitragynine, akuammine, eseroline, hodgkinsine, mitragynine, pericine, 4-[(R)-[(2S,5R)-2,5-dimethyl-4-prop-2-enylpiperazin-1-yl]-(3-hydroxyphenyl) methyl]-N,N-diethylbenzamide (BW373U86), 4-((αS)-α-((2S,5R)-2,5-dimethyl-4-(3-fluorobenzyl)-1-piperazinyl) benzyl)-N,N-diethylbenzamide (DPI-221), 4-[(R)-[(2S,5R)-2,5-dimethyl-4-benzylpiperazin-1-yl]-(3-hydroxyphenyl) methyl]-N,N-diethylbenzamide (DPI-287), (+)-3-((αR)-α-((2S,5R)-4-Allyl-2,5-dimethyl-1-piperazinyl)-3-hydroxybenzyl)-N-(3-fluorophenyl)-N-methylbenzamide (DPI-3290), 4-[(R)-[(2S,5R)-4-allyl-2,5-dimethylpiperazin-1-yl](3-methoxyphenyl) methyl]-NN-diethylbenzamide (SNC-80), 3,4-dichloro-N-[(1-dimethylamino) cyclohexylmethyl] benzamide (AH-7921), azaprocin, bromadol, 2-(3,4-dichlorophenyl)-1-[(2S)-2-(pyrrolidin-1-ylmethyl) piperidin-1-yl] ethanone (BRL-52537), bromadoline, trans-4-(p-Bromophenyl)-4-(dimethylamino)-1-(2-(thiophen-2-yl)ethyl) cyclohexanol (thiobromadol or C-8813), doxpicomine, enadoline, faxeladol, methyl 4-[2-(3,4-dichlorophenyl) acetyl]-3-(pyrrolidin-1-ylmethyl) piperazine-1-carboxylate (GR-89696), herkinorin, 2-(3,4-dichlorophenyl)-N-methyl-N-[(1S)-1-phenyl-2-pyrrolidin-1-ylethyl]acetamide (ICI-199,441), 2-(3-[1-([2-(3,4-dichlorophenyl) acetyl]-methylamino)-2-pyrrolidin-1-ylethyl] phenoxy) acetic acid (ICI-204,448), 2-(3,4-dichlorophenyl)-N-[(2S)-1-(2,5-dihydropyrrol-1-yl)-3-methylbutan-2-yl]-N-methylacetamide (LPK-26), methopholine, 8-chloro-11-piperazin-1-yl-5H-dibenzo[b,e][1,4] diazepine (N-desmethylclozapine or norclozapine or NDMC), methyl [8-(1-naphthylmethyl)-4-oxo-1-phenyl-1,3,8-triazaspiro[4.5]dec-3-yl)acetate (NNC 63-0532), nortilidine, O-desmethyltramadol, prodilidine, 8-[(1S,3aS)-2,3,3a,4,5,6-hexahydro-1H-phenalen-1-yl]-1-phenyl-1,3,8-triazaspiro[4.5]decan-4-one (Ro64-6198), 6-[(dimethylamino)methyl]-3-ethoxy-21-fluoro-20-oxopregna-3,5-dien-17-yl acetate (SC-17599), N,N-diethyl-4-((8-phenethyl-8-azabicyclo[3.2.1]oct-3-ylidene) phenylmethyl) benzamide (RWJ-394674), 3-[(4aS,12aR)-11-amino-2-methyl-1,3,4,5,12,12a-hexahydropyrido[3,4-b]acridin-4a(2H)-yl] phenol (SB-205,607 or TAN-67), tapentadol, tifluadom, tramadol, trimebutine, 2-(3,4-dichlorophenyl)-N-methyl-N-[(1R,2R)-2-pyrrolidin-1-ylcyclohexyl] acetamide (U-50488), N-methyl-2-phenyl-N-[(5R,7S,8S)-7-(pyrrolidin-1-yl)-1-oxaspiro[4.5]dec-8-yl] acetamide (U-69593), (E)-4-chloro-N-(1-(4-nitrophenethyl)piperidin-2-ylidene) benzenesulfonamide (W-18), 1-[1-(1-methylcyclooctyl)-4-piperidinyl]-2-[(3R)-3-piperidinyl]-1H-benzimidazole (MCOPPB), or a pharmaceutically acceptable salt of any one of the foregoing, or any combination thereof.

In some instances, the opioid analgesic includes hydrocodone, oxycodone, propoxyphene, fentanyl, acetyldihydrocodeinone, diamorphine, codeine, pethidine, alfentanil, codeine, hydromorphone, levorphanol, meperidine, methadone, morphine sulfate, oxymorphone, remifentanil, sufentanil, tramadol, tapentadol, or a pharmaceutically acceptable salt of any one of the foregoing, or any combination thereof.

### Non-Opioid Analgesics

A pharmaceutical composition disclosed herein comprises one or more non-opioid analgesics. Exemplary non-opioid analgesics can include a non-steroidal anti-inflammatory drug (NSAID) such as a salicylate (including for example, amoxiprin, benorilate, choline magnesium salicylate, diflunisal, faislamine, methyl salicylate, magnesium salicylate), an arylalkanoic acid (including, for example, diclofenac, aceclofenac, acemetacin, bromfenac, etodolac, indometacin, nabumetone, sulindac, tolmetin), a profen (including, for example, ibuprofen, carprofen, fenbuprofen, flubiprofen, ketaprofen, ketorolac, loxoprofen, naproxen, suprofen), a fenamic acid (including, for example, mefenamic acid, meclofenamic acid), an oxicam (including, for example, piroxicam, lomoxicam, meloxicam, tenoxicam), a pyrazolidine derivative (including, for example, phenylbutazone, azapropazone, metamizole, oxyphenbutazone, or sulfinprazone), or a pharmaceutically acceptable salt of any one of the foregoing, or any combination thereof.

In some instances, a non-opioid analgesic includes a Cox-2 inhibitor. Exemplary Cox-2 inhibitors include valdecoxib, celecoxib, rofecoxib or a pharmaceutically acceptable salt of any one of the foregoing, or any combination thereof. In some instances, the non-opioid analgesic can be a local analgesic. Exemplary local analgesics include lidocaine, mexiletine, or a pharmaceutically acceptable salt of any one of the foregoing, or any combination thereof. In some instances, the non-opioid analgesic can be an anti-depressant. Exemplary anti-depressants include amitriptyline, carbamazepine, gabapentin, pregabalin, amoxapine, clomipramine, desipramine, dosulepin, doxepin, imipramine, iprindole, lofepramine, nortriptyline, opipramol, protryptyline, trimipramine, or a pharmaceutically acceptable salt of any one of the foregoing, or any combination thereof. In some instances, the non-opioid analgesic can be an atypical analgesic. Exemplary atypical analgesics include orphenadrine, cyclobenzaprine, scopolamine, atropine, gabapentin, or a pharmaceutically acceptable salt of any one of the foregoing, or any combination thereof. In some instances, the non-opioid analgesic can be a psychotropic agent. Exemplary psychotropic agents include tetrahydrocannabinol or a pharmaceutically acceptable salt thereof. In some instances, the non-opioid analgesic can be an NMDA receptor antagonist. Exemplary NMDA receptor antagonists include ketamine, amantadine, dextromethorphan, dextrorphan, ibogaine, phencyclidine, riluzole, tiletamine, memantine, dizocilpine, patiganel, remacimide, or a pharmaceutically acceptable salt of any one of the foregoing, or any combination thereof. In some instances, the non-opioid analgesic can be an α2-adrenoreceptor agonist. Exemplary α2-adrenoreceptor agonists include clonidine or a pharmaceutically acceptable salt of any one of the foregoing, or any combination thereof. In some instances, the non-opioid analgesic is acetaminophen or a pharmaceutically acceptable salt thereof.

### Antiemetics

A pharmaceutical composition disclosed herein can comprise one or more antiemetics. In some instances, the antiemetic is an antihistamine. Exemplary antihistamines include promethazine, dolasetron, granisetron, ondansetron, tropisetron, palonosetron, domperidone, droperidol, haloperidol, chlorpromazine, prochloperazine, metoclopramide, alizapride, cyclizine, diphenhydramine, dimenhydrinate, meclizine, hydroxyzine, cannabis, dronabinol, nabilone, midazolam, lorazepam, hyoscine, dexamethasone, trimethobenzamide, emetrol, or propofol, or pharmaceutically acceptable salt of any one of the foregoing, or any combination thereof.

In some instances, an antihistamine comprises an H1 agonist or H1 antagonist. Exemplary H1 agonists or partial agonists include 2-(m-fluorophenyl)-histamine or a pharmaceutically acceptable salt thereof. Exemplary H1 antagonists can include azelastine, buclizine, carbinoxamine, cetrizine, clemastine, cyproheptadine, desloratidine, dimenhydrinate, diphenhydramine, emedastine, fexofenadine, hydroxyzine, ketotifen, levocabastine, olopatadine, phenindamine, promethazine, chlorphenamine, scopolamine, mepyramine, terfenadine, astemizole, triprolidine or a pharmaceutically acceptable salt of any one of the foregoing, or any combination thereof.

Additional exemplary antagonists include ethanolamines such as carbinoxamine, dimenhydrinate, diphenhydramine, doxylamine, or a pharmaceutically acceptable salt of any one of the foregoing, or any combination thereof; ethylaminediamines such as pyrilamine, tripelennamine, or a pharmaceutically acceptable salt of any one of the foregoing, or any combination thereof; piperazine derivatives such as dydroxyzine, cyclizine, fexofenadine, meclizine, or a pharmaceutically acceptable salt of any one of the foregoing, or any combination thereof; alkylamines such as brompheniramine, chlorpheniramine, or a pharmaceutically acceptable salt of any one of the foregoing, or any combination thereof; and miscellaneous antagonists such as cyproheptadine, loratadine, cetrizine, or a pharmaceutically acceptable salt of any one of the foregoing, or any combination thereof.

In some instances, an antihistamine includes an H2 agonist or H2 antagonist. Exemplary H2 agonists include dimaprit, impromidine, amthamine, or a pharmaceutically acceptable salt of any one of the foregoing, or any combination thereof. Exemplary H2 antagonists (useful in the treatment of gastric acid secretion) include cimetidine, ranitidine, nizatidine, famotidine, or a pharmaceutically acceptable salt of any one of the foregoing, or any combination thereof.

In some instances, an antihistamine includes an H3 agonist or H3 antagonist. Exemplary H3 agonists include R-alpha-methylhistamine, imetit, immepip, or a pharmaceutically acceptable salt of any one of the foregoing, or any combination thereof. Exemplary H3 antagonists include thioperamide, iodophenpropit, clobenpropit, or a pharmaceutically acceptable salt of any one of the foregoing, or any combination thereof.

In some instances, an antihistamine includes an H4 agonist or H4 antagonist. In some instances, an antihistamine includes an H4 agonists and H4 antagonists. Exemplary H4 agonists include clobenpropit, imetit, clozapine, or a pharmaceutically acceptable salt of any one of the foregoing, or any combination thereof. Exemplary H4 antagonists include thioperamide or a pharmaceutically acceptable salt thereof.

In some instances, an agent useful for preventing or suppressing an adverse effect includes an H1 antagonist. Exemplary H1 antagonists include azelastine, brompheniramine, buclizine, carbinoxamine, cetrizine, chlorpheniramine, clemastine, cyclizine, cyproheptadine, desloratidine, dimenhydrinate, diphenhydramine, emedastine, fexofenadine, hydroxyzine, ketotifen, levocabastine, loratadine, meclizine, olopatadine, phenindamine, promoathazine, or a pharmaceutically acceptable salt of any one of the foregoing, or any combination thereof.

In other instances, exemplary antiemetics can include aprepitant, dronabinol, perphenazine, palonosetron, trimethyobenzamide, metoclopromide, domperidone, prochlorperazine, promethazine, promethazine HCl, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt of any one of the foregoing, or any combination thereof.

### Barbiturate Agents

In some instances, a pharmaceutical composition disclosed herein comprises a barbiturate active agent. Exemplary barbiturate agents include allobarbital, alphenal, amobarbital, aprobarbital, barbexaclone, barbital, brallobarbital, butabarbital, butalbital, butobarbital, butallylonal, crotylbarbital, cyclobarbital, cyclopal, ethallobarbital, febarbamate, heptabarbital, hexethal, hexobarbital, mephobarbital, metharbital, methohexital, methylphenobarbital, narcobarbital, nealbarbital, pentobarbital, primidone, probarbital, propallylonal, proxibarbal, proxibarbital, reposal, secbutabarbital, secobarbital, sigmodal, talbutal, thialbarbital, thiamylal, thiobarbital, thiobutabarbital, thiopental, valofane, vinbarbital, vinylbital, 1,3-dimethoxymethyl 5,5-diphenyl-barbituric acid (DMMDPB), 1-monomethoxymethyl 5,5-diphenylbarbituric acid (MMMDPB), a diphenyl-barbituric acid (DPB) and their precursors, derivatives and analogs, or a pharmaceutically acceptable salt of any one of the foregoing, or any combination thereof.

### Stimulants and Irritants

In some instances, a pharmaceutical composition disclosed herein comprises a stimulant agent. Exemplary stimulant agents include aminophylline, caffeine, dyphlline, oxitriphylline, theophhylline, amphetamine, benzphetamine, dextroamphetamine, diethylpropion, mazindol, methamphetamine, methylphenidate, dexmethylphenidate, pemoline, sibutramine, modafinil, atomoxetine, phendimetrizine, phenteramine, adrafinil, phenylpropanolamine, pseudoephedrine, synephrine, amphetaminil, furfenorex, or a pharmaceutically acceptable salt of any one of the foregoing, or any combination thereof. In some instances, pharmaceutical compositions can comprise a stimulant agent that provides an anti-sedative effect.

In some instances, a stimulant agent comprises an amphetamine. Exemplary amphetamines include methamphetamine, levoamphetamine, dextroamphetamine, 3,5-methyloxy amphetamine, 2,5-dimethoxy-4-methylthioamphetamine, 2,5-dimethoxy-4-ethylthioamphetamine, 2,5-dimethoxy-4-(i)-propylthioamphetamine, 2,5-dimethoxy-4-phenylthioamphetamine, 2,5-dimethoxy-4-(n)-propylthioamphetamine, brolamfetamine, 2,5-dimethoxy-4-iodoamphetamine, 2,5-dimethoxy-4-methylamphetamine, 2,5-dimethoxy-4-butyl-amphetamine, 3,4-dimethyl-2,5 dimethoxyamphetamine, 2-phenylethylamine, propylamphetamine, methylphenidate, lisdexamfetamine, ethylamphetamine, MDMA (3,4-methylenedioxy-N-methylamphetamine), MDEA (3,4-methylenedioxy-N-ethylamphetamine), PMA (p-methoxyamphetamine), DMA (2-(2,4-dimethoxy-phenyl)-1-methyl-ethylamine), benzphetamine, 4-FMP (para-fluoroamphetamine), or 4-MTA (4-methylthioamphetamine), or a pharmaceutically acceptable salt of any one of the foregoing, or any combination thereof.

In some instances, a stimulant agent comprises a laxative. Exemplary laxatives include anthracenedione, triphenylmethane, ricinoleic acid, or a pharmaceutically acceptable salt of any one of the foregoing, or any combination thereof.

In some instances, a stimulant comprises an anthracenedione. Exemplary anthracenediones include dantron (1,8-dihydroxyanthraquinone), emodine (6-methyl-1,3,8-trihydroxyanthraquinone), aloe emodin (1,8-Dihydroxy-3-(hydroxymethyl)-9,10-anthracenedione), a senna glycoside, or a pharmaceutically acceptable salt of any one of the foregoing, or any combination thereof.

In some instances, a stimulant comprises a triphenylmethane. Exemplary triphenylmethanes include bisacodyl (4,4′-(pyridin-2-ylmethylene)bis(4,1-phenylene) diacetate), phenolphthalein, or a pharmaceutically acceptable salt of any one of the foregoing, or any combination thereof.

### Anti-Tussive Agents

In some instances, a pharmaceutical composition disclosed herein comprises an antitussive agent. Exemplary antitussive agents include dextromethorphan, dextrorphan, noscapine, ethyl morphine, codeine, camphor, menthol, theobromine, guaifenesin, dihydrocodein, hydrocodone, pholcodine, or a pharmaceutically acceptable salt of any one of the foregoing, or any combination thereof.

### Beta Blockers

In some instances, a pharmaceutical composition disclosed herein comprises one or more beta blockers. Exemplary beta blockers include acebutolol, arotinolol, atenolol, betaxolol, bisoprolol, butoxamine, carvedilol, carteolol, esmolol, carteolol, carvedilol, labetalol, levobunolol, mepindolol, metoprolol, nebivolol, nadolol, oxprenolol, penbutolol, propranolol, pindolol, sotalol, timolol or a pharmaceutically acceptable salt of any one of the foregoing, or any combination thereof. In some instances, the beta blocker can be propranolol or a pharmaceutically acceptable salt thereof.

### Serotonin Receptor Agonists

In some instances, a pharmaceutical composition disclosed herein comprises one or more serotonin receptor agonists. Exemplary serotonin receptor agonists include buspirone, mescaline, psilocybin, cisapride, lysergic acid diethylamide, or a pharmaceutically acceptable salt of any one of the foregoing, or any combination thereof.

### Vasoconstrictors

In some instances, a pharmaceutical composition disclosed herein comprises one or more vasoconstrictors. Exemplary vasoconstrictors include isometheptene mucate, amphetamines, antihistamines, cocaine, caffeine, pseudoephedrine, ergine, methylphenidate, psilocybin, stimulants such as amphakines (e.g., drugs effective to glutagatergic AMPA receptors and benzoylpiperidine derivatives) or a pharmaceutically acceptable salt of any one of the foregoing, or any combination thereof.

### Anti-Platelet Agents

In some instances, a pharmaceutical composition disclosed herein comprises one or more anti-platelet agents. Exemplary anti-platelet agents include acetylsalycyclic acid, clopidogrel, ticlopidine, cilostazol, abciximab, eptifibatide, tirofiban defibrotide, dipyridamole, or a pharmaceutically acceptable salt of any one of the foregoing, or any combination thereof.

### Anti-Convulsants

In some instances, a pharmaceutical composition disclosed herein comprises one or more anti-convulsants. Exemplary anti-convulsants include topiramate, divaprex, phenobarbital, methlyphenobarbital, metharbital, barbexaclone, stiripentol, clobazam, clonazepam, clorazepate, diazepam, midazolam, lorazepam, nitrazepam, temazepam, nimetazepam, potassium bromide, felbamate, carbamazepine, oxcarbazepine, vigabatrin, progabide, tiagabine, gabapentin, pregabalin, ethotoin, phenytoin, mephenytoin, fosphenytoin, paramethadione, trimethadione, ethadione, beclaminde, primidone, brivaracetam, levetiracetam, seletracetam, ethsuximide, phesuximide, mesuximide, acetazolamide, sulthiame, methazolamide, zonisamide, lamotrigine, pheneturide, phenacemide, valpromide, valnoctamide, or a pharmaceutically acceptable salt of any one of the foregoing, or any combination thereof.

### Ergots

In some instances, a pharmaceutical composition disclosed herein comprises one or more ergots. Exemplary ergots include ergotamine, methysergide, zonisamide, or a pharmaceutically acceptable salt of any one of the foregoing, or any combination thereof.

### Calcitonin-Gene-Related Peptide (CGRP) Receptor Antagonists

In some instances, a pharmaceutical composition disclosed herein comprises one or more calcitonin-gene-related peptide (CGRP) receptor antagonists. Exemplary CGRP include MK-0974, CGRP8-37, BIBN 4096 BS, quinine, nitrobenzamide, 4-oxobutanamides, cyclopropane derivatives, benzimidazolinyl piperidine, or a pharmaceutically acceptable salt of any one of the foregoing, or any combination thereof.

### Laxatives

In some instances, pharmaceutical compositions disclosed herein comprise one or more laxatives. Exemplary laxatives include a bulk-producing agent, a stool softener, a lubricant, a hydrating agent, a stimulant, an irritant, a serotonin agonist, a chloride channel activator, or any combination thereof. In some instances, the laxative comprises a bulk-producing agent. Exemplary bulk-producing agents include polycarbophil calcium, methyl cellulose, a soluble dietary fibre, an insoluble dietary fibre, or a pharmaceutically acceptable salt of any one of the foregoing, or any combination thereof. Exemplary soluble and insoluble dietary fibers include bran, gamkaraya, sterculia, psyllium husk, or combinations thereof. In some instances, the laxative comprises a stool softener. Exemplary stool softeners include dioctyl calcium sulfosuccinate (docusate calcium), dioctyl sodium sulfosuccinate (docusate sodium, DSS), dioctyl potassium sulfosuccinate (docusate potassium), or a pharmaceutically acceptable salt of any one of the foregoing, or any combination thereof. In some instances, the laxative comprises a lubricant. An exemplary lubricant is mineral oil. In some instances, the laxative comprises a hydrating agent. Exemplary hydrating agents include a saline laxative, a hyperosmotic agent, or a pharmaceutically acceptable salt of any one of the foregoing, or any combination thereof. Exemplary saline laxatives include sodium chloride, sodium bicarbonate, potassium chloride, sodium sulfate, sodium phosphate, potassium sodium tartrate, magnesium citrate, magnesium hydroxide, magnesium sulfate, or a pharmaceutically acceptable salt of any one of the foregoing, or any combination thereof. Exemplary hyperosmotic agents include sorbitol, lactulose, polyethylene glycol, glycerin, or a pharmaceutically acceptable salt of any one of the foregoing, or any combination thereof. In some instances, the laxative comprises a stimulant or irritant. Exemplary stimulants or irritants include an anthracenedione, a triphenylmethane, a castor oil, a ricinoleic acid, or a pharmaceutically acceptable salt of any one of the foregoing, or any combination thereof. Exemplary anthracenediones include dantron (1,8-dihydroxyanthraquinone), emodine (6-methyl-1,3,8-trihydroxyanthraquinone), aloe emodin (1,8-Dihydroxy-3-(hydroxymethyl)-9,10-anthracenedione), a senna glycoside, or a pharmaceutically acceptable salt of any one of the foregoing, or any combination thereof. Exemplary triphenylmethanes include bisacodyl (4,4′-(pyridin-2-ylmethylene)bis(4,1-phenylene) diacetate), phenolphthalein, or a pharmaceutically acceptable salt of any one of the foregoing, or any combination thereof. In some instances, the laxative comprises a serotonin agonist. Exemplary serotonin agonists include tegaserod, cisapride, prucalopride, or a pharmaceutically acceptable salt of any one of the foregoing, or any combination thereof. In some instances, the laxative comprises a chloride channel activator. Exemplary chloride channel activators include lubiprostone or a pharmaceutically acceptable salt thereof. In some instances, a pharmaceutical composition disclosed herein includes an amount of a laxative effective in reducing or eliminating constipation in a subject in need thereof.

### Excipients/Carriers/Additives

In some instances, a pharmaceutical composition disclosed herein comprises one or more excipients, carriers, or additives. Exemplary excipients, carriers, or additives can include antioxidant agents, binders, coating materials, colorant agents, diluents, disintegrants, dispersants, emulsifying agents, flavoring agents, glidants, lubricants, pH modifying agents (e.g., buffering agents), plasticizers, preservative agents, solubilizing agents, stabilizers or stabilizing agents, surfactants, sweetening agents, thickening agents, or pharmaceutically inert materials. In some instances, excipients can comprise nontoxic auxiliary substances.

Exemplary antioxidants can include flavonoids, anthocyanidins, anthocyanins, proanthocyanidins, or combinations thereof. In some instances, one or more antioxidants can be included in the liquid dosage form. In some instances, antioxidants help provide long term stability to liquid compositions, e.g., at ambient conditions for at least about one month, at least about 3 months, at least about 24 months, or longer, depending on the type and concentration of antioxidant used and depending on other components of the storage microenvironment, such as pH, buffering agent, etc.

Exemplary binders include celluloses such as hydroxypropylcellulose, methylcellulose, and hydroxypropyl methylcellulose; starches such as corn starch, pregelatinized starch, and hydroxpropyl starch; sugars such as glucose, dextrose, sucrose, lactose and sorbitol; alcohols such as polyvinyl alcohol and polyethylene glycol; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders can impact cohesive qualities to a tablet formulation, or a particle formulation in a capsule. Tablets can remain intact after compression by including a binder in the pharmaceutical composition.

Exemplary coating materials include hydroxypropyl methyl cellulose 2910, aminoalkyl methacrylate copolymer E, polyvinylacetal diethylaminoacetate, macrogol 6000, titanium oxide, or combinations thereof. Exemplary plasticizers include triethyl citrate, triacetin, macrogol 6000, or combinations thereof.

Exemplary colorant agents include one or more synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above synthetic organic food additives, etc.), natural pigments (e.g., beta-carotene, chlorophyll, iron oxide red, etc.), or combinations thereof. Other suitable colorant agents can include D&C Red No. 33, FD&C Red No. 3, FD&C Red No. 40, D&C Yellow No. 10, and C Yellow No. 6, or any combination of these or the above colorants. A colorant agent, when included in the liquid dosage form, can be provided in an amount sufficient to provide the pharmaceutical compositions with a more aesthetic and/or distinctive appearance.

Exemplary diluents include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose; D-mannitol; inorganic diluents such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Diluents, also terms “fillers”, can increase the bulk of a tablet so that a practical size is provided for compression.

Exemplary disintegrants include starches, alginic acid, crosslinked polymers such as, e.g., crosslinked polyvinylpyrrolidone, croscarmellose sodium, potassium or sodium starch glycolate, clays, celluloses, starches, gums, or combinations thereof. Disintegrants can facilitate tablet disintegration after administration, or following contact with dissolution fluid, or as measured in an in vitro dissolution study.

Exemplary emulsifying agents include gelatin, egg yolk, casein, cholesterol, acacia, tragacanth, chondrus, pectin, methyl cellulose, carbomer, cetostearyl alcohol, cetyl alcohol, or combinations thereof. Emulsifying agents can be included in the liquid dosage form in an amount sufficient to facilitate more uniform dispersion of one or more active ingredients or other pharmaceutically acceptable excipient that is not generally soluble in the liquid.

Exemplary glidants include silicon dioxide, talc, dried aluminum hydroxide gel, magnesium silicate, or combinations thereof. Exemplary lubricants include magnesium stearate, calcium stearate, stearic acid, glyceryl behenate, polyethylene glycol, talc, or combinations thereof. Lubricants can also facilitate tablet manufacture.

Exemplary buffering agents include gluconate, lactate, citrate, acetate, phosphate, benzoate, carbonate salts, or combinations thereof. The buffering agent can be present in an amount sufficient to buffer the pH of the solution and minimize degradation of the active ingredients. Some buffering agents can also modulate active ingredient solubility in the liquid dosage form. The pH can be adjusted with a combination of two or more of these buffering agents, e.g. citric acid and sodium benzoate. The buffering agent can be present as a buffer solution. In some instances, the buffering agent can include a phosphate, such as a potassium phosphate or sodium phosphate, or any combination thereof.

Exemplary preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form.

Exemplary solubilizing agents include an alcohol, e.g., 95% ethyl alcohol, a glycol, glycerin, D-mannitol, trehalose, benzyl benzoate, trisaminomethane, cholesterol, triethanolamine, sodium carbonate, sodium citrate, sodium salicylate, sodium acetate, or combinations thereof. Exemplary alcohols can include ethanol, isopropanol, t-butanol, phenol, cresol, a benzyl alcohol, or any combination thereof. Exemplary glycols include C2-20 alkenes functionalized with a glycol, including propylene glycol, polypropylene glycol, polyethylene glycol, etc., or any combination thereof. Solubilizing agents can be included in the liquid dosage form, e.g., in an amount sufficient to facilitate greater or more rapid dissolution of one or more active ingredients or other excipients. A solubilizing agent can be included in an amount of about 1 volume percent to 20 volume percent (v/v), or about 4 volume percent to 15 volume percent (v/v), based on the total volume of the solution. Exemplary amounts of solubilizing agent include about 7 volume percent to 12 volume percent (v/v) based on the total volume of the solution.

Exemplary stabilizing agents include one or more liquid excipients such as ethanol or glycerin; one or more glycols, such as polyethylene glycol, e.g., PEG-400, propylene glycol, or polypropylene glycol; a cellulose-based component, such as hydroxypropyl methylcellulose (HPMC) or hydroxymethylcellulose (HMC); or combinations thereof. Stabilizers can inhibit or retard drug decompositions reactions including oxidative reactions. A stabilizing agent can include any suitable monohydroxy phenol component or polyhydroxy phenol component, or any combination thereof. Such stabilizing agents can also function as antioxidant agents, or antimicrobial agents. Stabilizing agent(s) can be included in the liquid dosage form. Thus, it should be understood that certain solubilizing agents can function effectively as a stabilizing agent. For example, propylene glycol can function as both a solubilizing agent and as a stabilizing agent.

Exemplary surfactants include sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, lauromacrogol, or combinations thereof. Surfactants can also be anionic, cationic, amphoteric, or nonionic.

Exemplary sweetening agents include sorbitol, saccharin, acesulfame, e.g., acesulfame potassium, sucralose, xylitol, maltitol, sucrose, aspartame, fructose, neotame, glycerin, sodium saccharate, glycyrrhizin dipotassium, acesulfame K, mannitol, invert sugar, or combinations thereof. In some instances, a sweetening agent, such as one or more sucralose-containing components or saccharin-containing components, can be added to the pharmaceutical composition to modify the taste of the pharmaceutical composition. In some instances, a viscous sweetener such as one or more of a sorbitol solution, a syrup (sucrose solution), or high-fructose corn syrup can increase viscosity and retard sedimentation. In some instances, the sweetening agent can include an acesulfame-containing, sucralose-containing, or saccharin-containing component. The sweetening agent can include glycerin, saccharin, liquid sugar (sucrose solution), or any combination thereof. In some instances, a sweetening agent can be present in an amount sufficient to minimize or mask any off-flavors in the taste of the active agents (e.g., opioid analgesic, non-opioid analgesic, antiemetic, laxative, barbiturate, etc), and also to minimize or mask any other off-flavor components included in the pharmaceutical composition.

In some instances, a sweetening agent is present in an amount of about 0.1 volume percent to 85 volume percent (v/v), based on the total volume of the solution. In one example, the sweetening agent is present in an amount of about 5 volume percent to 70 volume percent (v/v), based on the total volume of the solution. Exemplary amounts of glycerin can include about 2 volume percent to 18 volume percent (v/v), or about 5 volume percent to 10 volume percent (v/v). Exemplary amounts of liquid sugar can include about 40 volume percent to 75 volume percent (v/v), or about 60 volume percent to 70 volume percent (v/v), based on the total volume of the solution. Certain types of thickening agent or sweetening agent can also act as a solubilizing agent or a stabilizing agent, or both, or have other properties, when included as a component of a pharmaceutically acceptable carrier. For example, a sweetening agent such as glycerin can also act as a thickening agent. An oral liquid dosage form can also contain, in addition to a sweetening agent, a flavoring agent, for example, one or more of natural and artificial fruit, artificial banana, strawberry, and pineapple.

Exemplary thickening agents include acacia, alginic acid bentonite, carbomer, carboxymethylcellulose calcium or sodium, cetostearyl alcohol, methyl cellulose, ethylcellulose, glycerin, gelatin guar gum, hydroxyethyl cellulose, hydroxymethyl cellulose, hydroxypropyl cellulose, hydroxypropyl methyl cellulose (“HPMC”), any other suitable cellulose-based component, maltodextrin, polyvinyl alcohol, povidone, propylene carbonate, propylene glycol alginate, sodium alginate, sodium starch glycolate, starch tragacanth, and xanthan gum, or combinations thereof. A thickening agent or viscosity-enhancing agent can improve the mouth-feel of the liquid oral dosage form and/or to help coat the lining of the gastrointestinal tract.

In some instances, a thickening agent is present in an amount of about 0.1 volume percent to 20 volume percent (v/v), based on the total volume of the solution. In one example, glycerin can be present in an amount of about 1 volume percent to 10 volume percent (v/v), based on the total volume of the solution. Exemplary amounts of thickening agent can include from about 1 volume percent to 12 volume percent (v/v), or at an amount of about 4 volume percent to 10 volume percent (v/v), based on the total volume of the solution. An exemplary amount can include about 6 to 10 volume percent (v/v).

In some instances, an excipient includes cellulose ethers such as hydroxypropyl methylcellulose (e.g., Methocel K4M) or silicified microcrystalline cellulose; polyvinylacetate-based excipients such as, e.g., Kollidon S R, and polymers and copolymers based on methacrylates and methacrylic acid such as, e.g., Eudragit NE 30D; microcrystalline cellulose, sodium carboxymethyl cellulose, sodium starch glycolate, corn starch, colloidal silica, sodium laurel sulphate, magnesium stearate, Prosolve SMCC (HD90), croscarmellose sodium, Crospovidone NF, Avicel PH200 or combinations thereof.

In some instances, an excipient includes acesulfame potassium, glacial acetic acid, acetone, acetyltributyl citrate, acetyltriethyl citrate, adipic acid, albumin, aliphatic polyester, alitame, almond oil, alpha tocopherol, aluminum monostearate, aluminum oxide, aluminum phosphate adjuvant, ammonia, ammonium alginate, ammonium chloride, anthocyanidin, anthocyanin, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparaben, calcium acetate, calcium alginate, calcium chloride, calcium hydroxide, calcium lactate, calcium phosphate (tribasic), calcium silicate, canola oil, carbomer, carbon dioxide, carboxymethylcellulose calcium, carboxymethylcellulose sodium, carrageenan, casein, castor oil, podere cellulose, cellulose acetate, cellulose acetate phthalate, ceratonia, ceresin, cetostearyl alcohol, cetrimide, cetyl alcohol, cetylpyridinium chloride, chitosan, chlorhexidine, chlorobutanol, chlorocresol, chlorodifluoroethane, chlorofluorocarbon, chloroxylenol, cholesterol, chondrus, citric acid monohydrate, coconut oil, copovidone, corn oil, cottonseed oil, cresol, cyclomethicone, denatonium benzoate, dextrate, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, egg yolk, erythorbic acid, erythritol, ethyl acetate, ethyl lactate, ethyl maltol, ethyl oleate, ethyl vanillin, ethylene glycol stearate, ethylene vinyl acetate, ethylparaben, flavonoid, fructose, fumaric acid, glycerin, glyceryl monooleate, glyceryl palmitostearate, glycine, glycofurol, guar gum, hectorite, heptafluoropropane, hexetidine, hydrocarbon, hydrochloric acid, hydrophobic colloidal silica, hydroxyethyl cellulose, hydroxyethylmethyl cellulose, hydroxypropyl betadex, hydroxpropyl starch, hypromellose acetate succinate, imidurea, inulin, iron oxide, isomalt, isopropyl alcohol, isopropyl myristate, isopropyl palmitate, lactic acid, lactitol, lanolin, lanolin (hydrous), lanolin alcohol, lauric acid, lecithin, leucine, linoleic acid, magnesium aluminum silicate, magnesium carbonate, magnesium oxide, magnesium trisilicate, maleic acid, malic acid, maltitol, maltitol solution, maltodextrin, maltol, maltose, medium-chain triglyceride, meglumine, menthol, methionine, methylparaben, mineral oil, lanolin alcohol, monoethanolamine, monosodium glutamate, monothioglycerol, myristic acid, myristyl alcohol, neohesperidin dihydrochalcone, neotame, nitrogen, nitrous oxide, octyldodecanol, oleic acid, oleyl alcohol, olive oil, palmitic acid, paraffin, peanut oil, pectin, pentetic acid, petrolatum, petrolatum alcohol, phenol, phenoxyethanol, phenylethyl alcohol, phenylmercuric acetate, phenylmercuric borate, phenylmercuric nitrate, phospholipid, phosphoric acid, polacrilin potassium, poloxamer, polycarbophil, polydextrose, poly (dl-lactic acid), polyethylene oxide, poly(methyl vinylether/maleic anhydride), polyoxyethylene alkyl ether, polyoxyethylene sorbitan fatty acid ester, polyoxyethylene stearate, polyoxylglyceride, polyvinyl acetate phthalate, potassium alginate, potassium alum, potassium benzoate, potassium bicarbonate, potassium chloride, potassium citrate, potassium hydroxide, potassium metabisulfite, potassium sorbate, proanthocyanidin, propionic acid, propyl gallate, propylene carbonate, propylene glycol, propylene glycol alginate, propylparaben, propylparaben sodium, pyrrolidone, raffinose, saccharin, saccharin sodium, safflower oil, saponite, sesame oil, shellac, simethicone, sodium acetate, sodium ascorbate, sodium benzoate, sodium bicarbonate, sodium borate, sodium carbonate, sodium citrate dihydrate, sodium cyclamate, sodium formaldehyde sulfoxylate, sodium hyaluronate, sodium lactate, sodium metabisulfite, sodium phosphate (dibasic), sodium phosphate (monobasic), sodium propionate, sodium sulfite, sodium thiosulfate, sorbic acid, sorbitan fatty acid ester, soybean oil, stearyl alcohol, sucralose, sucrose octaacetate, sulfobutylether b-cyclodextrin, sulfur dioxide, sulfuric acid, sunflower oil, suppository bases (hard fat), tagatose, tartaric acid, tetrafluoroethane, thaumatin, thimerosal, thymol, trehalose, tributyl citrate, tricaprylin, triethanolamine, triolein, vanillin, hydrogenated vegetable oil, vitamin e polyethylene glycol succinate, water, wax (anionic emulsifying), wax (cetyl esters), wax (microcristalline), wax (nonionic emulsifying), wax (white), wax (yellow), xanthan gum, xylitol, zein, zinc acetate, zinc stearate, food red dye No. 2, food red dye No. 3, food yellow dye No. 4, food yellow dye No. 5, food blue dye No. 1, food blue dye No. 2, beta-carotene, chlorophyll, iron oxide red, titanium dioxide, gluconate, lactate, paraoxybenzoic acid ester, phenylethylalcohol, dehydroacetic acid, ethyl alcohol, trisaminomethane, sodium salicylate, ethanol, isopropanol, t-butanol, polypropylene glycol, hydroxymethylcellulose, acesulfame, sodium saccharate, glycyrrhizin dipotassium, acesulfame K, or ethylcellulose.

Examples of excipients include acacia, acesulfame potassium, acetic acid (glacial), acetone, acetyltributyl citrate, acetyltriethyl citrate, adipic acid, agar, albumin, alcohol, alginic acid, aliphatic polyesters, alitame, almond oil, alpha tocopherol, aluminum hydroxide adjuvant, aluminum monostearate, aluminum oxide, aluminum phosphate adjuvant, ammonia solution, ammonium alginate, ammonium chloride, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparaben, calcium acetate, calcium alginate, calcium carbonate, calcium chloride, calcium hydroxide, calcium lactate, calcium phosphate (dibasic anhydrous), calcium phosphate (dibasic dihydrate), calcium phosphate (tribasic), calcium silicate, calcium stearate, calcium sulfate, canola oil, carbomer, carbon dioxide, carboxymethylcellulose calcium, carboxymethylcellulose sodium, carrageenan, castor oil, castor oil (hydrogenated), cellulose (microcristalline), cellulose (microcrystalline and carboxymethylcellulose sodium), cellulose (podere), cellulose (silicified microcristalline), cellulose acetate, cellulose acetate phthalate, ceratonia, ceresin, cetostearyl alcohol, cetrimide, cetyl alcohol, cetylpyridinium chloride, chitosan, chlorhexidine, chlorobutanol, chlorocresol, chlorodifluoroethane (HCFC), chlorofluorocarbons (CFC), chloroxylenol, cholesterol, citric acid monohydrate, coconut oil, colloidal silicon dioxide, colorant agents, copovidone, corn oil, corn starch and pregelatinized starch, cottonseed oil, cresol, croscarmellose sodium (AC-Di-Sol), crospovidone, cyclodextrins, cyclomethicone, denatonium benzoate, dextrates, dextrin, dextrose, dibutyl phthalate, dibutyl sebacate, diethanolamine, diethyl phthalate, difluoroethane (HFC), dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol stearates, ethylene vinyl acetate, ethylparaben, fructose, fumaric acid, gelatin, glucose (liquid), glycerin, glyceryl behenate, glyceryl monooleate, glyceryl monostearate, glyceryl palmitostearate, glycine, glycofurol, guar gum, hectorite, heptafluoropropane (HFC), hexetidine, hydrocarbons (HC), hydrochloric acid, hydrophobic colloidal silica, hydroxyethyl cellulose, hydroxyethylmethyl cellulose, hydroxypropyl betadex, hydroxypropyl cellulose, hydroxypropyl cellulose (low-substituted), hydroxypropyl starch, hypromellose, hypromellose acetate succinate, hypromellose phthalate, imidurea, inulin, iron oxides, isomalt, isopropyl alcohol, isopropyl myristate, isopropyl palmitate, kaolin, lactic acid, lactitol, lactose (anhydrous), lactose (inhalation), lactose (monohydrate), lactose (monohydrate and corn starch), lactose (monohydrate and microcrystalline cellulose), lactose (monohydrate and povidone), lactose (monohydrate and powdered cellulose), lactose (spray-dried), lanolin, lanolin (hydrous), lanolin alcohols, lauric acid, lecithin, leucine, linoleic acid, macrogol 15 hydroxystearate, magnesium aluminum silicate, magnesium carbonate, magnesium oxide, magnesium silicate, magnesium stearate, magnesium trisilicate, maleic acid, malic acid, maltitol, maltitol solution, maltodextrin, maltol, maltose, mannitol, medium-chain triglycerides, meglumine, menthol, methionine, methylcellulose, methylparaben, mineral oil, mineral oil (light), mineral oil and lanolin alcohols, monoethanolamine, monosodium glutamate, monothioglycerol, myristic acid, myristyl alcohol, neohesperidin dihydrochalcone, neotame, nitrogen, nitrous oxide, octyldodecanol, oleic acid, oleyl alcohol, olive oil, palmitic acid, paraffin, peanut oil, pectin, pentetic acid, petrolatum, petrolatum and lanolin alcohols, phenol, phenoxyethanol, phenylethyl alcohol, phenylmercuric acetate, phenylmercuric borate, phenylmercuric nitrate, phospholipids, phosphoric acid, polacrilin potassium, poloxamer, polycarbophil, polydextrose, poly (dl-lactic acid), polyethylene glycol, polyethylene oxide, polymethacrylates, poly(methyl vinylether/maleic anhydride), polyoxyethylene alkyl ethers, polyoxyethylene castor oil derivatives, polyoxyethylene sorbitan fatty acid esters, polyoxyethylene stearates, polyoxylglycerides, polyvinyl acetate phthalate, polyvinyl alcohol, potassium alginate, potassium alum, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, propylparaben sodium, pyrrolidone, raffinose, saccharin, saccharin sodium, safflower oil, saponite, sesame oil, shellac, simethicone, sodium acetate, sodium alginate, sodium ascorbate, sodium benzoate, sodium bicarbonate, sodium borate, sodium carbonate, sodium chloride, sodium citrate dihydrate, sodium cyclamate, sodium formaldehyde sulfoxylate, sodium hyaluronate, sodium hydroxide, sodium lactate, sodium lauryl sulfate, sodium metabisulfite, sodium phosphate—dibasic, sodium phosphate (monobasic), sodium propionate, sodium starch glycolate, sodium stearyl fumarate, sodium sulfite, sodium thiosulfate, sorbic acid, sorbitan esters (sorbitan fatty acid esters), sorbitol, soybean oil, starch, starch (pregelatinized), starch (sterilizable maize), stearic acid, stearyl alcohol, sucralose, sucrose, sucrose octaacetate, sugar (compressibile), sugar (confectioner's), sugar spheres, sulfobutylether b-cyclodextrin, sulfur dioxide, sulfuric acid, sunflower oil, suppository bases (hard fat), tagatose, talc, tartaric acid, tetrafluoroethane (HFC), thaumatin, thimerosal, thymol, titanium dioxide, tragacanth, trehalose, triacetin, tributyl citrate, tricaprylin, triethanolamine, triethyl citrate, triolein, vanillin, vegetable oil (hydrogenated), vitamin e polyethylene glycol succinate, water, wax (anionic emulsifying), wax (carnauba), wax (cetyl esters), wax (microcristalline), wax (nonionic emulsifying), wax (white), wax (yellow), xanthan gum, xylitol, zein, zinc acetate, zinc stearate, or combinations thereof. Furthermore, dosage forms herein can comprise acceptable carriers or salts known in the art, such as those described in the Handbook of Pharmaceutical Excipients, American Pharmaceutical Association (1986), incorporated by reference herein in its entirety.

### Salts

In some instances, a pharmaceutical composition disclosed herein comprises a pharmaceutically active agent that can be in the form of its free base, its pharmaceutically acceptable salt, prodrug, analog, or complex. Exemplary pharmaceutically acceptable salts include metal salts, such as sodium salts, potassium salts, lithium salts; alkaline earth metals, such as calcium salts, magnesium salts; organic amine salts, such as triethylamine salts, pyridine salts, picoline salts, ethanolamine salts, triethanolamine salts, dicyclohexylamine salts, N,N′-dibenzylethylenediamine salts; inorganic acid salts such as hydrochloride salts, hydrobromide salts, sulfate salts, phosphate salts; organic acid salts such as formate salts, acetate salts, trifluoroacetate salts, maleate salts, tartrate salts; sulfonate salts such as methanesulfonate salts, benzenesulfonate salts, p-toluenesulfonate salts; and amino acid salts, such as arginate salts, asparginate salts, glutamate salts, or combinations thereof.

In some instances, a pharmaceutically acceptable salt includes bitartrate, bitartrate hydrate, hydrochloride, p-toluenesulfonate, phosphate, sulfate, trifluoroacetate, bitartrato hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, sulfate pentahydrate, or combinations thereof. In some instances, exemplary pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, flunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, or combinations thereof. In some instances, a pharmaceutically acceptable salt includes bitartrate, bitartrate hydrate, hydrochloride, p-toluenesulfonate, phosphate, sulfate, trifluoroacetate, or bitartrato hemipentahydrate.

### Effective Amount

An “effective amount” when used in connection with a pharmaceutical composition disclosed herein is an amount sufficient to produce a therapeutic result in a subject in need thereof. For example a therapeutic result can include, but is not limited to, treating or preventing pain, nausea, retching, vomiting, or constipation by a subject. An “effective amount” when used in connection with an opioid analgesic agent alone or in combination is an amount that is effective for treating or preventing pain, wherein the opioid analgesic agent is provided in combination with one or more pharmaceutically active agents disclosed herein. In some instances, the one or more pharmaceutically active agent is an antiemetic. An “effective amount” when used in connection with an antiemetic agent is an amount that is effective for preventing or reducing or eliminating one or more adverse effects associated with one or more pharmaceutically active agent disclosed herein. In some instances, an “effective amount” when used in connection with an antiemetic agent is an amount that is effective for increasing the subject's pain relief from that provided by an opioid analgesic and/or an non-opioid analgesic. In some instances, an “effective amount” when used in connection with an antiemetic agent is an amount that is effective for preventing or reducing or eliminating one or more adverse effects associated with one or more pharmaceutically active agent disclosed herein and for increasing the subject's pain relief from that provided by an opioid analgesic and/or an non-opioid analgesic.

An “effective amount” when used in connection with a pharmaceutical composition disclosed herein is an amount sufficient to produce a therapeutic result in a subject in need thereof. For example a therapeutic result can include, but is not limited to, treating or preventing pain, nausea, retching, vomiting, or constipation by a subject. An “effective amount” when used in connection with an opioid analgesic agent alone or in combination is an amount that is effective for treating or preventing pain, wherein the opioid analgesic agent is provided in combination with one or more pharmaceutically active agents disclosed herein. In some instances, the one or more pharmaceutically active agent is an antiemetic. An “effective amount” when used in connection with an antiemetic agent is an amount that is effective for preventing or reducing or eliminating one or more adverse effects associated with one or more pharmaceutically active agent disclosed herein. In some instances, an “effective amount” when used in connection with an antiemetic agent is an amount that is effective for increasing the subject's pain relief from that provided by an opioid analgesic and/or an non-opioid analgesic. In some instances, an “effective amount” when used in connection with an antiemetic agent is an amount that is effective for preventing or reducing or eliminating one or more adverse effects associated with one or more pharmaceutically active agent disclosed herein and for increasing the subject's pain relief from that provided by an opioid analgesic and/or an non-opioid analgesic. In some instances, an “effective amount” when used in connection with an antiemetic agent is an amount that is effective for preventing or reducing or eliminating one or more adverse effects associated with one or more pharmaceutically active agent disclosed herein and for decreasing subject's sleep disturbance from that provided by an opioid analgesic and/or an non-opioid analgesic.

In some instances, the one or more pharmaceutically active agent includes but is not limited to an opioid analgesic and/or a non-opioid analgesic. In further instances, such adverse effects which are reduced, prevented or eliminated include but are not limited to incidence of nausea, retching, vomiting, or constipation. Furthermore, an “effective amount” when used in connection with an antiemetic is an amount that is effective for preventing or reducing the incidence of nausea, retching, vomiting, or constipation, or preventing or reducing adverse effects associated with an opioid analgesic (e.g., opioid-induced nausea, vomiting, or constipation). In further instances, an “effective amount” of an antiemetic is an amount that is effective for preventing or reducing or eliminating nausea or vomiting such as opioid-induced nausea or vomiting (OINV). An “effective amount” when used in connection with a stimulant agent is an amount that is effective to increase alertness, or lessen soporific effects of an opioid agent, wherein the stimulant agent is present in a dosage formulation alone or in combination with one or more pharmaceutically active agent disclosed herein. In some instances, the one or more pharmaceutically active agent includes but is not limited to an antiemetic agent and a barbiturate. An “effective amount” when used in connection with a barbiturate agent is an amount that is effective for treating or preventing pain, producing a sedative effect, anesthetic effect or calming effect when provided alone or in combination with one or more pharmaceutically active agent disclosed herein. In some instances, the one or more pharmaceutically active agent includes but is not limited to an opioid analgesic, a non-opioid analgesic, an antiemetic or combination thereof. An “effective amount” when used in connection with an opioid antagonist agent is an amount that is effective for preventing or inhibiting abuse of a dosage form comprising an opioid analgesic agent, wherein the antagonist agent is provided in combination with one or more pharmaceutically active agent disclosed herein. In some instances, the one or more pharmaceutically active agent includes but is not limited to an opioid agent, a non-opioid analgesic, a stimulant, a barbiturate, or any combination thereof. An “effective amount” when used in connection with a laxative is an amount that is effective for preventing, reducing, or eliminating constipation (e.g., opioid-induced constipation). An “effective amount” when in used in connection with one or more of agents disclosed herein is the total amount of one or more of the agents that is useful for the treatment of pain.

### Pharmaceutical Compositions

In some instances, a pharmaceutically active agent disclosed herein is capable of use in a pharmaceutical composition disclosed herein. A pharmaceutically active agent, such as an opioid analgesic agent, a non-opioid analgesic agent, an antitussive agent, an antiemetic agent, a stimulant, or a barbituate, can be in the form of a pharmaceutically acceptable salt of any one of the foregoing, or any combination thereof. In some instances, a pharmaceutical composition comprises an analgesic agent (e.g., one analgesic or two, three or more analgesics) and an antiemetic agent (e.g., one, two or more of an antiemetic) that reduces or eliminates an adverse effect of an analgesic agent. In some instances, a pharmaceutical composition disclosed herein comprises one or more pharmaceutically active agents herein, or a pharmaceutically acceptable salt herein, or any combination thereof. In some instances, a pharmaceutical composition comprises an effective amount of an opioid analgesic agent, an effective amount of non-opioid analgesic agent, an effective amount of an agent that reduces or eliminates an adverse effect of an analgesic agent, or any combination thereof.

In some instances, a pharmaceutical composition comprises an antiemetic and about 70% to about 80% of the antiemetic dissolves in the stomach of a subject in need thereof after about 5 to about 10 minutes following oral administration. In some instances, about 100% of the antiemetic dissolves in the stomach of a subject about 40, about 50 or about 60 minutes following oral administration. In some instances, the antiemetic is promethazine or a pharmaceutically acceptable salt thereof. In another instance, the promethazine salt is promethazine hydrochloride (promethazine HCl). In some instances, a pharmaceutical composition comprises an opioid analgesic and about 30% to about 40% of the opioid analgesic dissolves in the stomach of a subject in need thereof after about 5 to about 10 minutes following oral administration. In some instances, about 100% of the opioid analgesic dissolves in the stomach of a subject in need thereof about 40, about 50 or about 60 minutes following oral administration. In some instances, the opioid analgesic is hydrocodone, oxycodone or a pharmaceutically acceptable salt of any one of the foregoing, or any combination thereof. In another instance, the hydrocodone salt is hydrocodone bitartrate; or the oxycodone salt is oxycodone HCl.

In some instances, a pharmaceutical composition disclosed herein is administered to a subject in need thereof at about every 4 to about 8 hours. In some instances, a pharmaceutical composition disclosed herein is administered to a subject in need thereof at about every 4 to about 6 hours as needed. In some instances, a pharmaceutical composition disclosed herein is administered to a subject in need thereof at about every 4 to about 8 hours. In some instances, a pharmaceutical composition disclosed herein is administered to a subject in need thereof at about every 6 to about 8 hours as needed. In some instances, a pharmaceutical composition disclosed herein is administered to a subject in need thereof at about every 4 hours, about every 5 hours, about every 6 hours, about every 7 hours, or about every 8 hours. In some instances, a pharmaceutical composition disclosed herein is administered once daily. In some instances, a pharmaceutical composition disclosed herein is administered not more than 2-6 times daily. In another instance, a pharmaceutical composition disclosed herein is administered not more than 4 times daily.

In some instances, an agent that reduces or eliminates an adverse effect is an antiemetic agent. In further instances, the adverse effect reduced or eliminated is a non-opioid analgesic. In some instances, an agent that reduces or eliminates an adverse effect of an opioid analgesic agent or a non-opioid analgesic agent includes but is not limited to promethazine, dolasetron, granisetron, ondansetron, tropisetron, palonosetron, domperidone, droperidol, haloperidol, chlorpromazine, prochloperazine, metoclopramide, alizapride, cyclizine, diphenhydramine, dimenhydrinate, meclizine, hydroxyzine, cannabis, dronabinol, nabilone, midazolam, lorazepam, hyoscine, dexamethasone, trimethobenzamide, emetrol, and propofol or a pharmaceutically acceptable salt of any one of the foregoing, or any combination thereof.

In some instances, a pharmaceutical composition disclosed herein comprises a non-opioid analgesic agent which is acetaminophen, ibuprofen, naproxen or flubiprofen, or a pharmaceutically acceptable salt of any one of the foregoing, or any combination thereof. In some instances, the agent is naproxen sodium or magnesium. In some instances, the opioid analgesic agent is hydrocodone or oxycodone, or a pharmaceutically acceptable salt thereof thiosemicarbazone, p-nitrophenylhydrazone, o-methyloxime, semicarbazone, or bis (methylcarbamate) derivative, (each of the foregoing being an opioid analgesic agent or derivative). In a further instance, the opioid analgesic agent is hydrocodone bitartrate or oxycodone hydrochloride.

In some instances, an opioid analgesic agent is tapentadol or a pharmaceutically acceptable salt thereof. In some instances, the opioid analgesic agent is tapentadol hydrochloride. In some instances, the opioid analgesic agent is tramadol or a pharmaceutically acceptable salt thereof. In some instances, the opioid analgesic agent is tramadol hydrochloride. In some instances, an opioid analgesic agent is a naturally occurring opiate, such as an alkaloid occurring in the opium poppy. In some instances, the naturally occurring opiate is morphine, codeine, narcotine, papaverine, narceine, thebaine, or a pharmaceutically acceptable salt of any one of the foregoing, or any combination thereof.

In some instances, a pharmaceutical composition comprises an effective amount of each of an opioid analgesic, a non-opioid analgesic and an antiemetic, wherein the pharmaceutical composition is capable of providing an effective plasma concentration of the antiemetic prior to an effective plasma concentration of the opioid and the non-opioid analgesic, post oral administration. For example, a pharmaceutical composition comprising an effective amount of each of an opioid analgesic, non-opioid analgesic, and an antiemetic—provides an effective plasma concentration of the latter antiemetic earlier than the effective plasma concentration of an analgesic. In some instances, a pharmaceutical composition comprises an effective amount of each of one or more pharmaceutically active agents disclosed herein. In some instances, the pharmaceutical composition is a bi-layer tablet comprising a controlled-release layer and an immediate-release layer.

In some instances, a pharmaceutical composition comprises an opioid analgesic and one or more excipients. In such instances, the opioid analgesic can comprise hydrocodone, oxycodone, propoxyphene, fentanyl, acetyldihydrocodeinone, diamorphine, codeine, pethidine, alfentanil, codeine, hydromorphone, levorphanol, meperidine, methadone, morphine sulfate, oxymorphone, remifentanil, sufentanil, tapentadol, tramadol, or a pharmaceutically acceptable salt of any one of the foregoing, or any combination thereof.

In some instances, a pharmaceutical composition comprises an antiemetic and one or more excipients. In such instances, the antiemetic can comprise aprepitant, dronabinol, perphenazine, palonosetron, trimethyobenzamide, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt of any one of the foregoing, or any combination thereof.

In some instances, about 70 to about 80% of a pharmaceutically active agent is capable of achieving dissolution from an immediate-release layer at about 5 to about 10 minutes following oral administration. In another instance, about 70 to about 80% of a pharmaceutically active agent is capable of achieving dissolution from the immediate-release layer at about 5 to about 10 minutes following contact with a dissolution fluid, such as the dissolution fluid described in Example 6 or as measured by any of the dissolution methods as described herein.

In some instances, about 100% of a pharmaceutically active agent is capable of achieving dissolution from the immediate-release layer at about 40 minutes following oral administration. In another instance, about 100% to of a pharmaceutically active agent is capable of achieving dissolution from the immediate-release layer at about 40 minutes following contact with a dissolution fluid, such as the dissolution fluid described in Example 6 or as measured by any of the dissolution methods as described herein.

In some instances, about 30 to about 40% of a pharmaceutically active agent is capable of achieving dissolution from the controlled-release layer at about 5 to about 10 minutes following oral administration. In another instance, about 30% to about 40% of a pharmaceutically active agent is capable of achieving dissolution from the controlled-release layer at about 5 to about 10 minutes following contact with a dissolution fluid, such as the dissolution fluid described in Example 6 or as measured by any of the dissolution methods as described herein.

In some instances, controlled-release includes dissolution of about 40% to about 50% of an active agent by 10 minutes after administration, or following contact with dissolution fluid, or as measured in an in vitro dissolution study. In one embodiment, controlled-release is dissolution of about 60% of an active agent by 20 minutes after administration, or following contact with dissolution fluid, or as measured in an in vitro dissolution study. In one embodiment, controlled-release is dissolution of about 70% to about 80% of an active agent by 30 minutes after administration, or following contact with dissolution fluid, or as measured in an in vitro dissolution study. In one embodiment, controlled-release is dissolution of about 80% to about 100% of an active agent by 60 minutes after administration, or following contact with dissolution fluid, or as measured in an in vitro dissolution study.

In some instances, about 90% of a pharmaceutically active agent is capable of achieving dissolution from the controlled-release layer at about 60 minutes following oral administration. In another instance, about 90% of a pharmaceutically active agent is capable of achieving dissolution from the controlled-release layer at about 60 minutes following contact with a dissolution fluid, such as the dissolution fluid described in Example 6 or as measured by any of the dissolution methods as described herein.

In some instances, from about 90 to about 100% of a pharmaceutically active agent is capable of achieving dissolution from the immediate-release layer at about 40, about 50 or about 60 minutes following oral administration. In yet another instance, from about 90 to about 100% of a pharmaceutically active agent is capable of achieving dissolution from the immediate-release layer at about 40, about 50 or about 60 minutes following contact with a dissolution fluid, such as the dissolution fluid described in Example 6 or as measured by any of the dissolution methods as described herein.

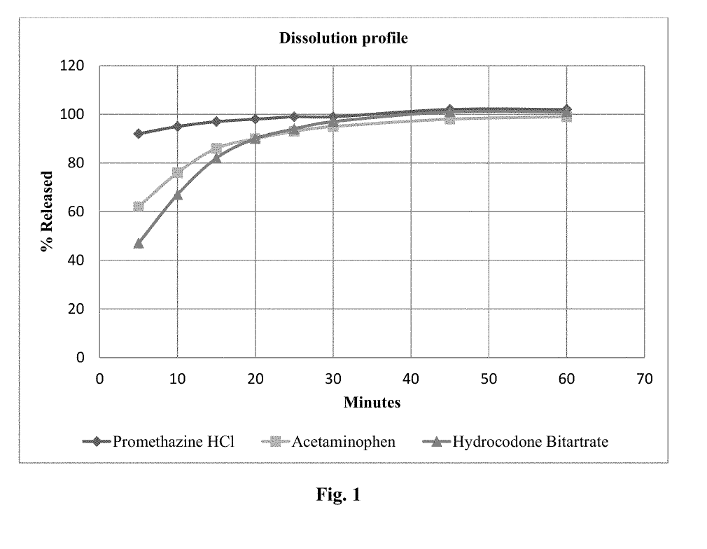

In some instances, immediate-release results in dissolution of an active agent within 1-20 minutes after entering the stomach and/or intestine. In some instances, dissolution is of all or less than the entire amount of the active agent. For example, dissolution of 100% of an active agent (for example, an antiemetic) can occur in the prescribed time. In some instances, dissolution of less than all of the active agent can occur in about 1 to about 20 minutes (e.g., dissolution of about 70%, about 75%, about 80%, about 85%, about 90%, about 91%, about 92%, about 93%, about 94%, about 95%, about 96%, about 97%, about 98%, about 99%, about 99.5% or 99.9% of an agent). In some instances, immediate-release occurs when there is dissolution of an agent within 1-20 minutes after oral administration. In another instance, immediate-release results in substantially complete dissolution within about 1 hour following oral administration to a subject in need thereof. In some instances, a pharmaceutical composition disclosed herein is capable of providing about 80% dissolution of an antiemetic in about 5 minutes (e.g., FIG. 1). In another instance, immediate-release results in complete or less than complete dissolution within about 1 hour following rectal administration to a subject in need thereof.

In some instances, immediate-release is through inhalation, such that dissolution occurs in a subject's lungs, as further described herein. Dissolution of less than all of an active includes but is not limited to dissolution of about 50%, 60%, 70%, 80%, 85%, 90%, 95%, 97%, 98%, 99%, 99.1%, 99.2%, 99.35, 99.4%, 99.5%, 99.6%, 99.7%, 99.8% or 99.99% of the active agent. Methods for measuring dissolution profiles are described herein (e.g., Example 6, infra).

In some instances, a pharmaceutical composition is in the form of any oral dosage form disclosed herein, including but not limited to a pill, tablet, or capsule. In some instances, the pharmaceutical composition is in the form of a bi-layer tablet having an immediate-release layer and a controlled-release layer, wherein one or more pharmaceutically active agents are present in the immediate-release layer and one or more pharmaceutically active agents are present in the controlled-release layer. In another instance, the immediate-release layer comprises one or more antiemetics, and the controlled-release layer comprises one or more pharmaceutically active agents disclosed herein, but which are not an antiemetic. In a further instance, an antiemetic is present in both the immediate-release and controlled-release layer. In another instance, the immediate-release layer comprises promethazine or a pharmaceutically acceptable salt thereof. In another instance, the promethazine salt is promethazine HCl. In another instance, the controlled-release layer comprises an opioid analgesic. In a further instance, the opioid analgesic is hydrocodone or oxycodone, or a pharmaceutically acceptable salt of any one of the foregoing, or any combination thereof. In some instances, the hydrocodone salt is hydrocodone bitartrate. In another instance, the oxycodone salt is oxycodone HCl. In a further instance, the controlled-release layer further comprises one or more non-opioid analgesic. In some instances, the non-opioid analgesic is acetaminophen or a pharmaceutically acceptable salt thereof. In some instances, the pharmaceutical composition is in a form that achieves a hardness of from about 5 to about 15 kiloponds and has a thickness of about 5, about 5.5, about 6, about 6.5, about 7, about 7.5, about 8, about 8.5, about 9, about 9.5 or 10 mm. In some instances, the pharmaceutical composition is in a form that achieves a hardness of from about 5 to about 30 kiloponds and has a thickness of about 5, about 5.5, about 6, about 6.5, about 7, about 7.5, about 8, about 8.5, about 9, about 9.5 or 10 mm. In some instances, the tablet has a hardness of about 12.5 kiloponds. In some instances, the tablet has a hardness of about 10, 11, 12, 13, 14, 15, 16, 17, 18, 19, 20, 21, 22, 23, 24, 25, 26, 27, 28, 29, or 30 kiloponds. In another instance, the tablet has a hardness of about 12.5 kiloponds. It would be understood that as to the kilopond and thickness measurements, increments of 0.1 decimal points are within the scope of the disclosure.

In some instances, a pharmaceutical composition is capable of providing an effective plasma concentration of an antiemetic in about 1 to about 20 minutes after administration to a subject in need thereof. In another instance, the antiemetic is promethazine or a pharmaceutically acceptable salt thereof. In a further instance, the salt is promethazine HCl. In some instances, a pharmaceutical composition comprises from about 1 to about 20% by weight of an antiemetic; from about 10 to about 80% by weight a non-opioid analgesic; and from about 1 to about 20% by weight of an opioid analgesic.

In some instances, a method is provided for reducing or eliminating an adverse effect of an analgesic agent, comprising administering to a subject in need thereof a pharmaceutical composition comprising an effective amount of each of an opioid analgesic agent, a non-opioid analgesic agent and an agent which reduces or eliminates an adverse effect of the analgesic agents. In some instances, a method is provided for treating or preventing pain, comprising administering to a subject in need thereof an effective amount of a pharmaceutical composition comprising an effective amount of each of an opioid analgesic, or a pharmaceutically acceptable salt thereof, a non-opioid analgesic, or a pharmaceutically acceptable salt thereof, and an agent which reduces an adverse effect associated with the opioid or non-opioid analgesic agent. In some instances, the agent that reduces an adverse effect is an antiemetic.

In some instances, an agent useful for reducing or eliminating an adverse effect associated with administration of an opioid or non-opioid analgesic agent, is promethazine, dolasetron, granisetron, ondansetron, tropisetron, palonosetron, domperidone, droperidol, haloperidol, chlorpromazine, prochloperazine, metoclopramide, alizapride, cyclizine, diphenhydramine, dimenhydrinate, meclizine, hydroxyzine, cannabis, dronabinol, nabilone, midazolam, lorazepam, hyoscine, dexamethasone, trimethobenzamide, emetrol, or propofol, or pharmaceutically acceptable salt of any one of the foregoing, or any combination thereof.

A pharmaceutical composition can be in any form disclosed herein, such as a multi-layer tablet (e.g., a bi-layer tablet or a two layer table, or a three layer tablet). In some instances, the multi-layer tablet is a bi-layer tablet that comprises: (a) an immediate-release layer that comprises an effective amount of an agent which reduces or eliminates an adverse effect of an opioid analgesic; and (b) a controlled-release layer that comprises an effective amount of each of an opioid analgesic agent and a non-opioid analgesic agent. In some instances, the multi-layer tablet is a two layer tablet that comprises: (a) an immediate-release layer that comprises an effective amount of an agent which reduces or eliminates an adverse effect of an opioid analgesic; and (b) a controlled-release layer that comprises an effective amount of each of an opioid analgesic agent and a non-opioid analgesic agent.

In some instances, an agent that reduces or eliminates an adverse effect associated with administration of an opioid or non-opioid analgesic agent is released in a subject in need thereof at a substantially faster rate than an opioid or non-opioid analgesic in a pharmaceutical composition disclosed herein. In some instances, a plasma concentration of the agent that reduces or eliminates an adverse effect of an opioid analgesic is achieved in about 1 to about 20 minutes following oral administration, as compared with a plasma concentration of an analgesic agent, which can be achieved in about 30 minutes to about 8 hours following oral administration. In some instances, the pharmaceutical compositions herein comprise an agent that reduces or eliminates an adverse effect associated with administration of an opioid analgesic or non-opioid analgesic, where the agent provides an effective plasma concentration in about 1 to about 20 minutes following oral administration.

In some instances, a pharmaceutical composition comprises an effective amount of an opioid analgesic agent, a non-opioid analgesic agent, and an agent that reduces or eliminates an adverse effect associated with administration of the opioid or non-opioid analgesic. An adverse effect of opioid or non-opioid analgesic agents includes but is not limited to nausea, retching, vomiting, constipation, other gastric upset, skin rash, sleep disturbance, an allergic reaction such as swelling, difficulty breathing, closing of throat, abdominal pain, unusual bleeding or bruising, sedation, CNS depression, or respiratory depression. In some instances, the adverse effect that is reduced or eliminated is nausea, retching, vomiting, constipation, sleep disturbance or any combination thereof. In some instances, an agent that reduces or eliminates an adverse effect associated with an opioid or a non-opioid analgesic is an antiemetic. In some instances, an opioid analgesic agent is, for example, hydrocodone, oxycodone propoxyphene, or fentanyl, or a pharmaceutically acceptable salt of any one of the foregoing, or any combination thereof; the non-opioid analgesic agent is, for example, acetaminophen, ibuprofen, ketaprofen, naproxen, or acetylsalicylic acid or a pharmaceutically acceptable salt of any one of the foregoing, or any combination thereof; and the agent useful for preventing and/or suppressing an adverse effect is, for example, an antiemetic such as promethazine or a pharmaceutically acceptable salt thereof. In some instances, the pharmaceutically acceptable salt of naproxen is naproxen sodium.

In some instances, an opioid analgesic agent, a non-opioid analgesic agent and an agent that reduces or eliminates an adverse effect are formulated in a bi-layer tablet. In some instances, an opioid analgesic agent, a non-opioid analgesic agent and an agent that reduces or eliminates an adverse effect are formulated in a two layer tablet. In some instances, the bi-layer tablet comprises an immediate-release layer and a controlled-release layer. In another instance, the immediate-release layer comprises one or more pharmaceutically active agent disclosed herein and the controlled-release layer comprises one or more pharmaceutically active agents disclosed herein. In some instances, the immediate-release layer comprises an antiemetic and the controlled-release layer comprises an opioid analgesic, a barbiturate, a stimulant, or any combination thereof.

In some instances, a pharmaceutical composition comprises an effective amount of each of an analgesic agent, an antitussive agent, and an agent that reduces or eliminates an adverse effect of the analgesic agent or the antitussive agent. In some instances, the antitussive is also an analgesic. In some instances, a pharmaceutical composition comprises acetaminophen, hydrocodone or oxycodone, or a pharmaceutically acceptable salt of any one of the foregoing, or any combination thereof; and an antitussive agent such as dolasetron, domperidone, meclizine, dronabinol, a benzodiazepine, an anticholinergic, hydrocodone or oxycodone, or a pharmaceutically acceptable salt of any one of the foregoing, or any combination thereof.

In some instances, an opioid analgesic agent is, for example, hydrocodone, or oxycodone or a pharmaceutically acceptable salt of any one of the foregoing, or any combination thereof; the non-opioid analgesic agent is, for example, acetaminophen, ibuprofen, ketoprofen, naproxen, lidocaine, or acetylsalicylic acid, or a pharmaceutically acceptable salt of any one of the foregoing, or any combination thereof; the antiemetic agent is, for example a 5-HT₃ receptor antagonist, a dopamine antagonist, an antihistamine, a cannabinoid, a benzodiazepine, an anticholinergic, wherein all or less than all of the total amount of the antiemetic agent is formulated for immediate-release.

Another instance is directed to a method for the treatment of pain, comprising administering an effective amount of each of an opioid analgesic agent, a non-opioid analgesic agent and an agent that reduces or eliminates an adverse effect of the opioid analgesic agent to a subject in need thereof.

In some instances, a method allows for use of an analgesic in populations at risk of adverse effects such as nausea, retching, vomiting, constipation, other gastric upsets, sleep disturbance, skin rashes, allergic reactions such as swelling, difficulty breathing, closing of throat, abdominal pain, unusual bleeding or bruising, skin rashes, sedation, CNS depression, or respiratory depression.

In some instances, a pharmaceutical composition disclosed herein comprises an effective amount of each of an opioid analgesic, an antiemetic, and an opioid antagonist, and is capable of providing protection from a metabolic consequence of vomiting, particularly severe vomiting, in a subject in need thereof particularly prone to adverse effects associated with an opioid analgesic. An example of metabolic consequence of vomiting is dehydration. In a further instance, the subject administered a pharmaceutical composition disclosed herein is about 55 years of age or older, about 60 years of age or older, about 65 years of age or older, or about 70 years of age or older. In some instances, the pharmaceutical composition administered to such a subject comprises an opioid analgesic and one or more antiemetic agent. In some instances, the pharmaceutical composition comprises oxycodone, promethazine, and naltrexone, or a pharmaceutically acceptable salt of any one of the foregoing, or any combination thereof.

In some instances, a dosage form comprising an antiemetic and an analgesic agent provides an effective plasma concentration of the antiemetic at a substantially faster release rate as compared with the release rate for the analgesic agent. In some instances, after administration to a subject, the antiemetic is released or an effective plasma concentration of an antiemetic is achieved before the analgesic agent is released or an effective plasma concentration of the analgesic agent is achieved. The analgesic agent can be an opioid analgesic or said non-opioid analgesic. In some instances, the dosage form provides an effective plasma concentration of the antiemetic at from about 1 to about 20 minutes after administration, such as about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, or 25 min following administration. In some instances, the dosage form provides an effective plasma concentration of the opioid analgesic or the non-opioid analgesic at from about 20 minutes to about 8 hours after administration, such as about 20 minutes, 30 minutes, 40 minutes, 50 minutes, 1 hr, 1.2 hrs, 1.4 hrs, 1.6 hrs, 1.8 hrs, 2 hrs, 2.2 hrs, 2.4 hrs, 2.6 hrs, 2.8 hrs, 3 hrs, 3.2 hrs, 3.4 hrs, 3.6 hrs, 3.8 hrs, 4 hrs, 5 hrs, 6 hrs, 7 hrs, or 8 hrs following administration.

In some instances, a pharmaceutical composition comprises an effective amount of each of an opioid analgesic, a non-opioid analgesic agent, an antihistamine, anti-psychotic, anti-anxiety agent, or other CNS depressant is administered a reduced dosage of one or lessen and adverse effect (e.g. CNS depression). In another instance, the dosage of one or more of pharmaceutically active agents is adjusted according to the severity of the pain and the response of the subject.

In some instances, a pharmaceutical composition comprises: hydrocodone, oxycodone, or a pharmaceutically acceptable salt of any one of the foregoing, or any combination thereof, in a dosage range of from about 1.0 to about 200 mg; acetaminophen or a pharmaceutically acceptable salt thereof in a dosage range of from about 200 to about 1000 mg; and, promethazine or a pharmaceutically acceptable salt thereof in a dosage range of from about 0.5 to about 100 mg. In some instances, a pharmaceutical composition comprises: oxycodone or a pharmaceutically acceptable salt thereof in a dosage range of from about 10 to about 80 mg; Naltrexone or a pharmaceutically acceptable salt thereof in a dosage range of from about 0.5 to about 0.75 mg; and, promethazine or a pharmaceutically acceptable salt thereof in a dosage range of from about 12.5 to about 50 mg. In some instances, a pharmaceutical composition comprises: oxycodone or a pharmaceutically acceptable salt thereof in a dosage range of from about 10 to about 80 mg; and promethazine or a pharmaceutically acceptable salt thereof in a dosage range of from about 12.5 to about 50 mg. Pharmaceutical composition disclosed herein can be formulated using conventional technologies to provide for a controlled release over a desired dosage interval, such as about 4 to 8 hours, e.g., about 4 hours, 5 hours, 6 hours, 7 hours, 8 hours, hours.

In another instance, the pharmaceutical compositions comprise about 7.5 mg of hydrocodone or a pharmaceutically acceptable salt thereof, about 325 mg of acetaminophen or a pharmaceutically acceptable salt thereof, and about 12.5 mg of promethazine or a pharmaceutically acceptable salt thereof. In another instance, the pharmaceutical compositions comprise about 5 mg of hydrocodone or a pharmaceutically acceptable salt thereof, about 325 mg of acetaminophen or a pharmaceutically acceptable salt thereof, and about 12.5 mg of promethazine or a pharmaceutically acceptable salt thereof. In another instance, the pharmaceutical compositions comprise about 10 mg of hydrocodone or a pharmaceutically acceptable salt thereof, about 325 mg of acetaminophen or a pharmaceutically acceptable salt thereof, and about 12.5 mg or 25 mg of promethazine or a pharmaceutically acceptable salt thereof. In some instances, a pharmaceutical composition comprises about 7.5 mg of oxycodone or a pharmaceutically acceptable salt thereof, about 325 mg of acetaminophen or a pharmaceutically acceptable salt thereof, and about 12.5 mg or 25 mg of promethazine or a pharmaceutically acceptable salt thereof.

In some instances, a pharmaceutical composition comprises an effective amount of hydrocodone or oxycodone or a pharmaceutically acceptable salt thereof; an effective amount of acetaminophen or a pharmaceutically acceptable salt thereof; and an effective amount of promethazine or a pharmaceutically acceptable salt thereof, combined in a single, oral pill, or tablet or lollipop, form having dosage levels that is safely doubled for combating severe pain.

In a one instance, all or less than the entire total amount of the promethazine or a pharmaceutically acceptable salt thereof is formulated for immediate-release into the subject's blood stream. In a further instance, all or less than the entire amount of the hydrocodone or oxycodone, or a pharmaceutically acceptable salt of any one of the foregoing, or any combination thereof is formulated for controlled-release into the subject's body. In some instances, an agent is formulated as a dosage form (e.g., tablet, capsule, gel, lollipop), parenteral, intraspinal infusion, inhalation, nasal spray, transdermal patch, iontophoresis transport, absorbing gel, liquid, liquid tannate, suppositories, injection, I.V. drip, other formulation, or any combination thereof to treat subjects. In some instances, an agent is formulated as single oral dosage form such as a tablet, capsule, cachet, soft gelatin capsule, hard gelatin capsule, extended release capsule, tannate tablet, oral disintegrating tablet, multi-layer tablet, effervescent tablet, bead, liquid, oral suspension, chewable lozenge, oral solution, lozenge, lollipop, oral syrup, sterile packaged powder including pharmaceutically-acceptable excipients, other oral dosage forms, or any combination thereof. In some instances, a pharmaceutical composition is a solid composition. In some instances, the pharmaceutical composition is a liquid composition. In some instances, the pharmaceutical composition is formulated as a patch. In another instance, a pharmaceutical composition disclosed herein comprises one or more further agents in immediate-release, controlled-release, other release formulations or patterns, or any combination thereof.

In some instances, a pharmaceutical composition disclosed herein comprises three active agents, such as a decongestant, an antitussive, an expectorant, a mucus-thinning drug, an analgesic or an antiemetic. In some instances one of the agents is an antitussive or a pharmaceutically acceptable salt thereof; the other agent is a decongestant such as, e.g., phenylephrine, pseudoephedrine, or a pharmaceutically acceptable salt thereof; and the other agent is an expectorant. One would recognize that an active agent can fit into more than one category (e.g., hydrocodone is an antitussive and opioid analgesic).

In some instances, a pharmaceutical composition disclosed herein is administered using one or more different dosage forms which are further described herein. For example, a pharmaceutical composition comprising multiple active agents can be administered in solid, semi-solid, micro-emulsion, gel, patch or liquid form. Such dosage forms are further described herein. Examples of such dosage forms are known, such as tablet forms disclosed in U.S. Pat. Nos. 3,048,526; 3,108,046; 4,786,505; 4,919,939; 4,950,484; gel forms disclosed in U.S. Pat. Nos. 4,904,479; 6,482,435; 6,572,871; 5,013,726; patches for delivery of pharmaceutical compositions such as those disclosed in U.S. Pat. Nos. 5,741,510; 4,624,665; 4,626,539; 4,834,978; 6,469,227; 5,919,479; 6,261,595; 6,303,142; 6,341,387; 6,465,006; 6,613,350; 6,780,426; 7,094,228; 6,756,053; capsule forms disclosed in U.S. Pat. Nos. 4,800,083; 4,532,126; 4,935,243; 6,258,380; liquid forms disclosed in U.S. Pat. Nos. 4,625,494; 4,478,822; 5,610,184; or I.V. forms disclosed in U.S. Pat. Nos. 4,871,353; 4,925,444; 5,484,406; each of which is incorporated herein by reference in its entirety.

In some instances, a pharmaceutical composition comprises an antiemetic in an amount capable of achieving a serum level Cₘₐₓ of from about 0.2 ng/mL to about 1 ng/mL at a Tₘₐₓ of from about 1 to about 6 hours following oral administration. In some instances, the antiemetic is promethazine or a pharmaceutically acceptable salt thereof. In another instance, the pharmaceutically acceptable salt is promethazine HCl. In a further instance, the pharmaceutical composition is a bi-layer tablet that has an immediate-release layer and a controlled-release layer. In yet a further instance, the controlled-release layer comprises an opioid analgesic agent or a non-opioid analgesic agent. In a further instance, the immediate-release layer comprises promethazine or a pharmaceutically acceptable salt thereof.

In some instances, a pharmaceutical composition comprises promethazine or a pharmaceutically acceptable salt thereof in an amount capable of achieving a serum level Cₘₐₓ of about 0.46 ng/mL at a Tₘₐₓ of about 2 to about 3 hours following oral administration. In some instances, the promethazine or a pharmaceutically acceptable salt is at a dose by weight in the pharmaceutical composition of about 10 to about 15 mg. In another instance, the promethazine or pharmaceutically acceptable salt is at a dose (by weight in the pharmaceutical composition) of about 12.5 mg. In a further instance, the pharmaceutical composition is in the form of a bi-layer tablet that has an immediate-release layer and a controlled-release layer. In some instances, the promethazine or a pharmaceutically acceptable salt is the only pharmaceutically active agent in the immediate-release layer of a bi-layer tablet herein. In some instances, the promethazine is promethazine HCl. In yet a further instance, the controlled-release layer comprises an opioid analgesic agent or a non-opioid analgesic agent. In some instances, the opioid analgesic is the only pharmaceutically active agent in the controlled-release layer of a bi-layer tablet, non-opioid analgesic is the only pharmaceutically active agent in the controlled-release layer of a bi-layer tablet or both the opioid analgesic and non-opioid analgesic are the only pharmaceutically active agents of the pharmaceutical composition.

An aspect of the disclosure provides a pharmaceutical composition, wherein the pharmaceutical composition can comprise an effective amount of i) one or more opioid analgesics wherein the one or more opioid analgesics are selected from the group consisting of: hydrocodone, oxycodone, oripavine, dihydromorphine, hydromorphinol, nicomorphine, dipropanoylmorphine, diacetyldihydromorphine, desomorphine, methyldesorphine, heterocodeine, benzylmorphine, dihydroheterocodeine, myrophine, pentamorphone, etorphine, acetyldihydrocodeine, nicocodeine, nicodicodeine, alphamethylfentanyl, carfentanil, parafluorofentanyl, thiofentanyl, anileridine, benzethidine, difenoxin, diphenoxylate, etoxeridine, furethidine, morpheridine, pheneridine, phenoperidine, piminodine, allylprodine, loperamide, dextropropoxyphene, dihydroetorphine, acetorphine, levophenacylmorphan, phenomorphan, drotebanol, dipipanone, normethadone, phenadoxone, dimepheptanol, levacetylmethadol, dextromoramide, diethylthiambutene, dimethylthiambutene, ethylmethylthiambutene, dextropropoxyphene, dimenoxadol, tilidine, ethoheptazine, proheptazine, piritramide, etonitazene, tapentadol, tramadol, a pharmaceutically acceptable salt of each of the foregoing, and any combination thereof, and ii) one or more antiemetics; and iii) a pharmaceutically acceptable carrier or vehicle.

In one aspect, the one or more antiemetics comprises promethazine, aprepitant, dronabinol, perphenazine, palonosetron, trimethyobenzamide, metoclopromide, domperidone, prochlorperazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt of any one of the foregoing, or any combination thereof.

In one aspect, the one or more antiemetics comprises promethazine or a pharmaceutically acceptable salt thereof. In some instances, the one or more opioid analgesics can comprise hydrocodone, oxycodone, or a pharmaceutically acceptable salt thereof and the one or more antiemetics can comprise promethazine or a pharmaceutically acceptable salt thereof and ondansetron or a pharmaceutically acceptable salt thereof.

In some instances, a pharmaceutical composition disclosed herein comprises from about 6.5 mg to about 8.5 mg of the hydrocodone, oxycodone, or a pharmaceutically acceptable salt of any one of the foregoing, or any combination thereof and further comprises from about 11 mg to about 14 mg of the promethazine or a pharmaceutically acceptable salt thereof.

In some instances, a pharmaceutical composition disclosed herein further comprises one or more non-opioid analgesics. In some instances, the one or more non-opioid analgesics comprises a non-steroidal anti-inflammatory agent, a cox-2 inhibitor, a local analgesic, an anti-depressant, an atypical analgesic, a psychotropic agent, an NMDA receptor, an alpha2-adrenoreceptor agonist, a synthetic drug having narcotic properties, or a pharmaceutically acceptable salt of any one of the foregoing, or any combination thereof. In some instances, the one or more non-opioid analgesics comprises acetaminophen, acetylsalicylic acid, amoxiprin, benorilate, choline magnesium salicylate, diflunisal, faislamine, methyl salicylate, magnesium salicylate, diclofenac, aceclofenac, acemetacin, bromfenac, etodolac, indometacin, nabumetone, sulindac, tolmetin, ibuprofen, carprofen, fenbuprofen, flubiprofen, ketaprofen, ketorolac, loxoprofen, naproxen, suprofen, mefenamic acid, meclofenamic acid, piroxicam, lomoxicam, meloxicam, tenoxicam, phenylbutazone, azapropazone, metamizole, oxyphenbutazone, sulfinprazone, or a pharmaceutically acceptable salt of any one of the foregoing, or any combination thereof. In some instances, the one or more non-opioid analgesics is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof. In one aspect of the disclosure, a pharmaceutical composition disclosed herein comprises two or more of the antiemetics. In some instances, the two or more of the antiemetics comprises promethazine or a pharmaceutically acceptable salt thereof and ondansetron or a pharmaceutically acceptable salt thereof. Another aspect of the disclosure provides for a pharmaceutical composition, wherein the pharmaceutical composition comprises a stimulant disclosed herein. In some instances, the stimulant comprises an amphetamine, a laxative, an agent that produces an anti-sedative effect, or a pharmaceutically acceptable salt of any one of the foregoing, or any combination thereof. Another aspect of the disclosure provides for a pharmaceutical composition, wherein the pharmaceutical composition comprises an opioid antagonist or an abuse deterrent agent herein. Another aspect of the disclosure provides for a pharmaceutical composition, wherein the pharmaceutical composition comprises one or more controlled-release dosage forms, one or more immediate-release forms, or any combination thereof.

In a further aspect, the pharmaceutical composition is formulated as a tablet, capsule, or lollipop. In some instances, the pharmaceutical composition formulated as the tablet is a bi-layer tablet, two layer tablet, a multi-layer tablet, a tannate tablet, an oral disintegrating tablet, an effervescent tablet, or any combination thereof. In some instances, the pharmaceutical composition formulated as the capsule is a soft gelatin capsule or a hard gelatin capsule. In some instances, the capsule can have micro drilled holes. In a further aspect, the tablet is a controlled-release layer and an immediate-release layer. In some instances, the tablet is a controlled-release layer comprising the one or more opioid analgesics and an immediate-release layer comprising the one or more antiemetics. In some instances, the one or more controlled-release dosage forms comprises an enteric coating, a particulate, a powder, a multi-layer, or any combination thereof. In some instances, the capsule is formulated with a controlled-release enteric coating. In some instances, the capsule is formulated with an immediate-release powder. In some instances, the tablet is formulated with a controlled-release enteric coating. In some instances, the capsule is formulated with one or more controlled-release particulates. In some instances, the particulate is a bead, a sphere, or a pellet. In some instances, the tablet or capsule comprises an inner dosage and an outer dosage, the latter being in the form of an envelope over the former. In some instances, the inner dosage and outer dosage components is separated by an enteric layer.

Another aspect of the disclosure provides for a pharmaceutical composition, wherein the pharmaceutical composition comprises one or more excipients herein. In some instances, the one or more excipients can comprise an antioxidant agent, a binder, a coating material, a colorant agent, a diluent, a disintegrant, a dispersant, an emulsifying agent, a flavoring agent, a glidant, a lubricant, a pH modifying agent, a plasticizer, a preservative agent, a solubilizing agent, a stabilizer, a surfactant, a sweetening agent, a thickening agent, a pharmaceutically inert material, or a pharmaceutically acceptable salt of any one of the foregoing, or any combination thereof.

Another aspect of the disclosure provides that when a pharmaceutical composition is administered to a mammal, one or more opioid analgesics have a Tmax that is about 1.4-2.0 hours, e.g., about: 1.4, 1.45, 1.5, 1.55, 1.6, 1.65, 1.7, 1.75, 1.8, 1.85, 1.9, 1.95, or 2 hours. In some instances, the Tmax is about 1.7 hours. Another aspect of the disclosure provides that when a pharmaceutical composition is administered to a mammal, one or more opioid analgesics have a Tmax that is about 5-60 minutes longer, e.g., about 10-30 minutes longer or about 5-10 minutes longer, than a corresponding standard-release opioid analgesic, e.g., in mean or median times. In some instances, when the pharmaceutical composition is administered to a mammal, the one or more opioid analgesics have a Tmax that is about: 5-10 minutes, 10-20 minutes, 20-30 minutes, 30-40 minutes, 40-50 minutes, or 50-60 minutes longer than a corresponding standard-release opioid analgesic, e.g., in mean or median times. In some instances, when the pharmaceutical composition is administered to a mammal, the one or more opioid analgesics have a Tmax that is about: 5, 10, 15, 20, 25, 30, 35, 40, 45, 50, 55, or 60 minutes longer than a corresponding standard-release opioid analgesic, e.g., in mean or median times. In some instances, when the pharmaceutical composition is administered to a mammal, the one or more opioid analgesics have a Tmax that is about 9-32 minutes longer than a corresponding standard-release opioid analgesic, e.g., in mean or median times.

Another aspect of the disclosure provides that when a pharmaceutical composition is administered to a mammal, one or more opioid analgesics have a Tmax that is about 30-60% longer, e.g., about 40-50% longer, than a corresponding standard-release opioid analgesic, e.g., in mean times. In some instances, when the pharmaceutical composition is administered to a mammal, the one or more opioid analgesics have a Tmax that is about: 30%, 35%, 40%, 41%, 42%, 43%, 44%, 45%, 46%, 47%, 48%, 49%, 50%, 55%, or 60% longer than a corresponding standard-release opioid analgesic, e.g., in mean times.

Another aspect of the disclosure provides that when a pharmaceutical composition is administered to a mammal, one or more opioid analgesics have a Tmax that is about 5-25% longer, e.g., about 5-15% longer, than a corresponding standard-release opioid analgesic, e.g., in median times. In some instances, when the pharmaceutical composition is administered to a mammal, the one or more opioid analgesics have a Tmax that is about: 5%, 6%, 7%, 8%, 9%, 10%, 11%, 12%, 13%, 14%, 15%, 16%, 17%, 18%, 19%, 20%, 21%, 22%, 23%, 24%, or 25% longer than a corresponding standard-release opioid analgesic, e.g., in median times. Another aspect of the disclosure provides that when a pharmaceutical composition is administered to a mammal, one or more antiemetics have a Tmax that is about 3.5 to 4.3 hours, e.g., about: 3.5, 3.6, 3.7, 3.75, 3.8, 3.85, 3.9, 3.95, 4, 4.1, 4.2, or 4.3 hours. In some instances, the Tmax is about 3.87 hours.

Another aspect of the disclosure provides that when a pharmaceutical composition is administered to a mammal, one or more antiemetics have a Tmax that is about 30-120 minutes shorter than a corresponding standard-release antiemetic, e.g., in median times. In some instances, when the pharmaceutical composition is administered to a mammal, the one or more antiemetics have a Tmax that is about 50-100 minutes or 60-90 minutes shorter, e.g., about: 30-40 minutes, 40-50 minutes, 50-60 minutes, 60-70 minutes, 70-80 minutes, 80-90 minutes, 90-100 minutes, 100-110 minutes, or 110-120 minutes shorter, than a corresponding standard-release antiemetic, e.g., in median times. In some instances, when the pharmaceutical composition is administered to a mammal, the one or more antiemetics have a Tmax that is about: 30, 35, 40, 45, 50, 55, 60, 65, 70, 75, 80, 85, 90, 95, 100, 105, 110, 115, or 120 minutes shorter than a corresponding standard-release antiemetic, e.g., in median times. In some instances, when the pharmaceutical composition is administered to a mammal, the one or more antiemetics have a Tmax that is about: 70, 71, 72, 73, 74, 75, 76, 77, 78, 79, or 80 minutes shorter than a corresponding standard-release antiemetic, e.g., in median times. In some instances, when the pharmaceutical composition is administered to a mammal, the one or more antiemetics have a Tmax that is about 75 minutes shorter than a corresponding standard-release antiemetic, e.g., in median times.

Another aspect of the disclosure provides that when a pharmaceutical composition is administered to a mammal, one or more antiemetics have a Tmax that is about 10-50% shorter, e.g., about 20-40% or 25-35% shorter, than a corresponding standard-release antiemetic, e.g., in median or mean times. In some instances, when the pharmaceutical composition is administered to a mammal, the one or more antiemetics have a Tmax that is about: 10-20%, 20-30%, 30%-40%, or 40%-50% shorter than a corresponding standard-release antiemetic, e.g., in median or mean times. In some instances, when the pharmaceutical composition is administered to a mammal, the one or more antiemetics have a Tmax that is about: 10%, 12%, 14%, 16%, 18%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, or 50% shorter than a corresponding standard-release antiemetic, e.g., in median or mean times.

Another aspect of the disclosure provides that when a pharmaceutical composition is administered to a mammal, one or more non-opioid analgesics have a Tmax that is about 0.9 to 1.1 hours, e.g., about: 0.9, 0.92, 0.94, 0.96, 0.98, 1, 1.02, 1.04, 1.06, 1.08, or 1.1 hours. In some instance, the Tmax is about 1.04 hours. Another aspect of the disclosure provides that when a pharmaceutical composition is administered to a mammal, one or more non-opioid analgesics have a Tmax that is about 5-30 minutes longer, e.g., about 10-25 minutes longer or about 10-15 minutes longer, than a corresponding standard-release non-opioid analgesic, e.g., in mean or median times. In some instances, when the pharmaceutical composition is administered to a mammal, the one or more non-opioid analgesics have a Tmax that is about: 5-10 minutes, 10-15 minutes, 15-20 minutes, 20-25 minutes, or 25-30 minutes longer than a corresponding standard-release non-opioid analgesic, e.g., in mean or median times. In some instances, when the pharmaceutical composition is administered to a mammal, the one or more non-opioid analgesics have a Tmax that is about: 5, 10, 15, 20, 25, or 30 minutes longer than a corresponding standard-release non-opioid analgesic, e.g., in mean or median times. In some instances, when the pharmaceutical composition is administered to a mammal, the one or more non-opioid analgesics have a Tmax that is about: 6, 7, 8, 9, 10, 11, 12, 13, 14, 15, 16, 17, 18, or 19 minutes longer than a corresponding standard-release non-opioid analgesic, e.g., in mean or median times. In some instances, when the pharmaceutical composition is administered to a mammal, the one or more non-opioid analgesics have a Tmax that is about 13 minutes longer than a corresponding standard-release non-opioid analgesic, e.g., in mean or median times.

Another aspect of the disclosure provides that when a pharmaceutical composition is administered to a mammal, one or more non-opioid analgesics have a Tmax that is about 10-50% longer, e.g., about 10-40% or 20-30% longer, than a corresponding standard-release non-opioid analgesic, e.g., in mean or median times. In some instances, when the pharmaceutical composition is administered to a mammal, the one or more non-opioid analgesics have a Tmax that is about: 10-20%, 20-30%, 30%-40%, or 40%-50% longer than a corresponding standard-release non-opioid analgesic, e.g., in mean or median times. In some instances, when the pharmaceutical composition is administered to a mammal, the one or more non-opioid analgesics have a Tmax that is about: 10%, 15%, 20%, 21%, 22%, 23%, 24%, 25%, 26%, 27%, 28%, 29%, 30%, 35%, 40%, 45%, or 50% longer than a corresponding standard-release non-opioid analgesic, e.g., in mean or median times.

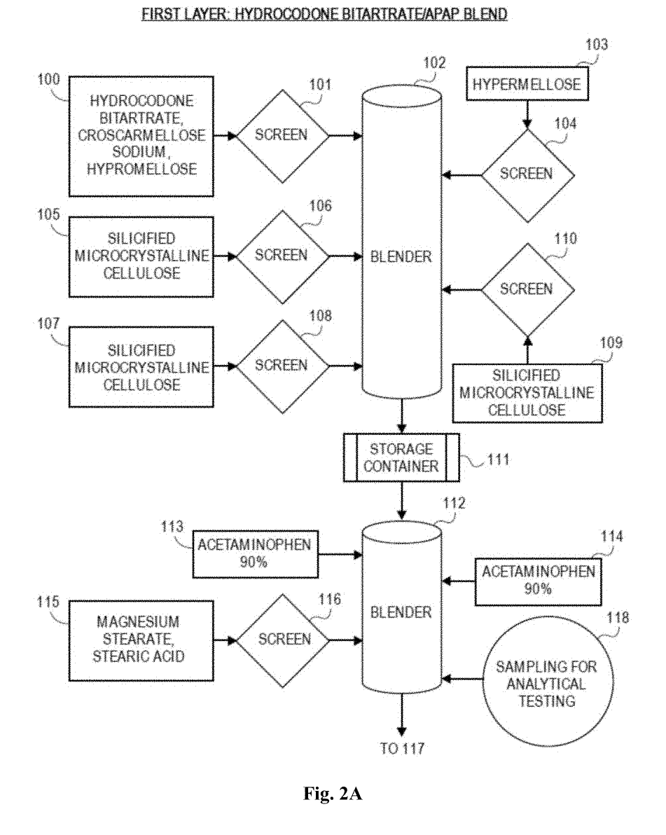

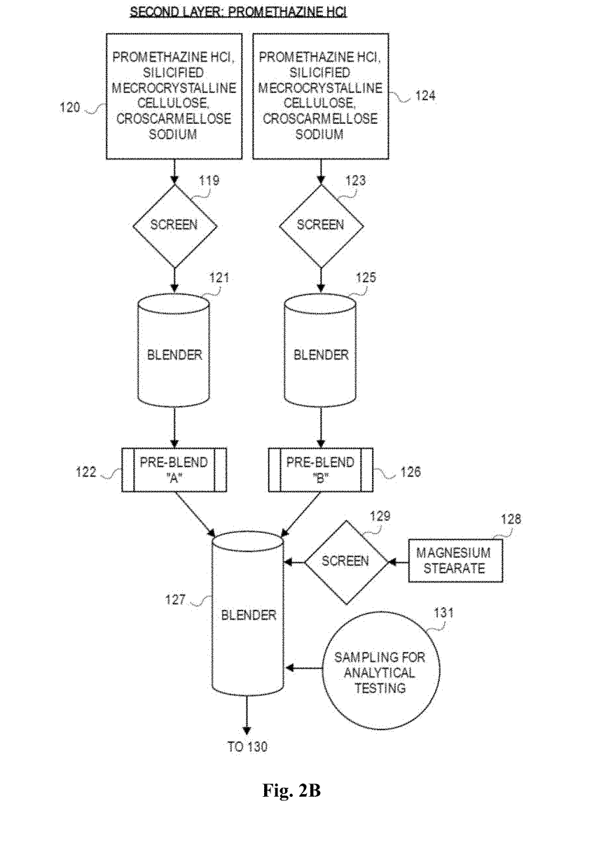

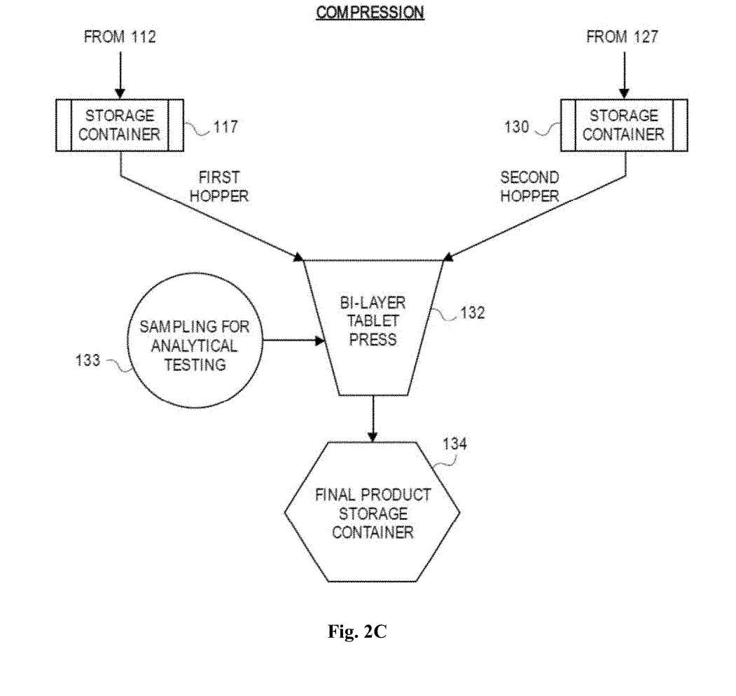

In some instances, tablets is formed by a manufacturing process, see FIG. 2A-2C. For example, a blend of hydrocodone bitartrate and acetaminophen is manufactured by passing hydrocodone bitartrate, croscarmellose sodium, and hypromellose (100) through a screen (101) and adding the contents to a clean blender (102). The contents are mixed in the blender. The hypromellose (103) is passed through a screen (104) and added to the blender (102). The contents of the blender are mixed. The silicified microcrystalline cellulose (105) is passed through a screen (106) and added to the blender (102). The contents of the blender were mixed. The silicified microcrystalline cellulose (107) is passed through a screen (108) and added to the blender (102). The contents of blender are mixed. The silicified microcrystalline cellulose (109) is passed through a screen (110) and added to the blender. The contents of the blender are mixed. The blended contents are discharged from the blender into an appropriately labeled container (111). A clean blender is setup (112). The hydrocodone bitartrate is added to the blend from the previous step (111) and half of the total acetaminophen (113) is also added to the clean blender and the contents are mixed. The second half of the total acetaminophen (114) is added to the blender and the contents are mixed. The magnesium stearate and stearic acid are passed (115) through a screen (116) and added to the blender with the hydrocodone bitartrate/acetaminophen blend. The contents of the blender are mixed. The blend is discharged into the appropriate container(s) (117). The accountable yield for the final blend (118) is calculated and recorded. A blend of promethazine HCl is manufactured by setting up a clean blender for the promethazine HCl blend. Half of the total specified amounts of promethazine HCl USP, cellulose microcrystalline-silicified, and croscarmellose sodium for the promethazine HCl (120) are screened (119). The contents of the blender are mixed (121). The pre-blend is discarded into the appropriate container (122) and set aside. The second half of the total specified amounts of promethazine HCl USP, cellulose microcrystalline-silicified, and croscarmellose sodium for the promethazine HCl blend (124) is screened (123). The contents of the blender are mixed (125). The second pre-blend is discharged into a separate appropriate container (126). The promethazine pre-blends from the separate containers (122 and 126) are added into a clean blender (127) and the contents are mixed. The magnesium stearate (128) is passed through a screen (129) and added to the blender (127) with the promethazine pre-blends. The contents of the blender are mixed. The blend is discarded into the appropriate storage container (130). The accountable yield for the final promethazine blend (131) is calculated and recorded. Tablet Compression is performed by transferring the first layer of hydrocodone/acetaminophen blend (117) into the first hopper. The press (132) is setup with its respective parameters appropriately. The second layer promethazine blend (130) is transferred into the second hopper. The press is started to begin producing the tablets to the specified parameters. During compressing, tablets are sampled at various times for in-process testing for weight, metal, microbiological contamination, thickness, hardness, and % friability. The accountable yield for the final blend (133) is calculated and recorded. At the end of the batch, the compressed tablets are deposited into an appropriate container (134) and stored.

In some instances, friability means the ability of a solid composition, such as a tablet, to be reduced to smaller pieces or particles or fibers with minimal force. In some embodiments, a mean loss of tablet mass for three determinations is not more than 1.0%. A friable tablet is one that is easily crumbled or pulverized. Friability can be measured using the methods and apparatus as described in the General Chapters and General Methods of the Harmonization section of the United States Pharmacopeia and the National Formulary (USP-NF), such as the General Chapter 1216 Tablet Friability section. In some instances, friability is measured in a friabilator (a rotating drum), see e.g., Example 4, and results are reported as a percent friability, i.e., percent tablet weight loss after rotation.

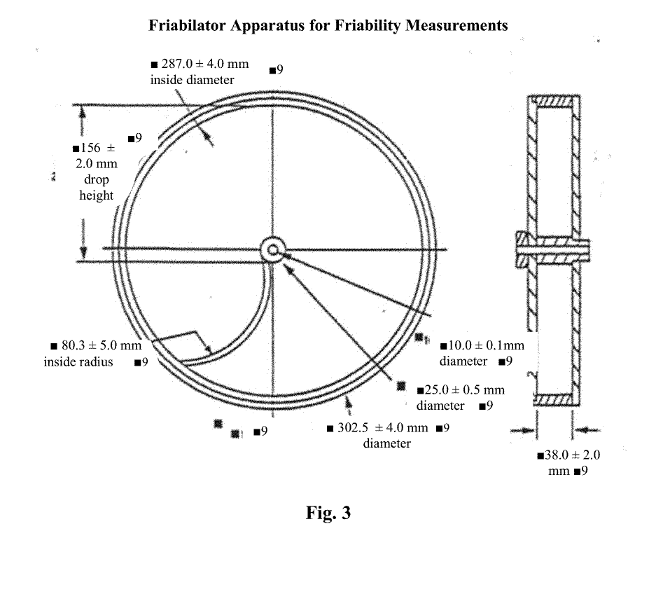

In some instances, friability measurement is calculated using a friabilator, with a horizontal axis that rotates at 25+/−1 rpm. The drum for the friabilator comprises a synthetic transparent polymer with an internal diameter between 283 and 291 mm and a depth between 36 and 40 mm. The drum comprises a curved projection with an inside radius between 75.5 and 85.5 mm, see FIG. 3. For example, tablets with a unit mass equal to or less than 650 mg, are measured using a representative sample of whole tablets corresponding to 6.5 g. If the tablet unit mass is greater than 650 mg, a representative sample of ten whole tablets is measured. Loose powder from the tablets is removed with the aid of air pressure or a soft brush. An initial tablet weight is recorded. The tablets are placed inside the friabilator drum, the drum is closed and rotated for 100 rotations for 4 minutes. Tablets are then removed from the drum. Any loose powder from all intact (non-broken, non-capped) tablets is removed. If any tablets are broken or capped, the number for each category is recorded. Pieces of broken or fractured tablets that do not pass through a 10-mesh screen are combined with the intact tablets and are accurately weighed and recorded as the final weight. The percent loss is calculated using the following equation:

$$Percent loss = \frac{( initial weight - final weight )}{initial weight} \times 100$$

In some instances, the pharmaceutical composition has a friability of 0.9% or less. In some instances, the pharmaceutical composition has a friability of 0.4% or less. In some instances, the pharmaceutical composition has a friability of 0.2% or less. In some instances, the pharmaceutical composition has a friability of about 0.1% to about 0.9%. In some instances, the pharmaceutical composition has a friability of about 0.05% to about 0.2%. In some instances, the pharmaceutical composition has a friability of about 0.05%. In some instances, the pharmaceutical composition has a friability of about 0.1%. In some instances, the pharmaceutical composition has a friability of about 0.15%. In some instances, the pharmaceutical composition has a friability of about 0.13%. In some instances, the pharmaceutical composition has a friability of about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, or about 0.95%. In some instances, the pharmaceutical composition has a friability of about 0.00, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or about 0.40%.

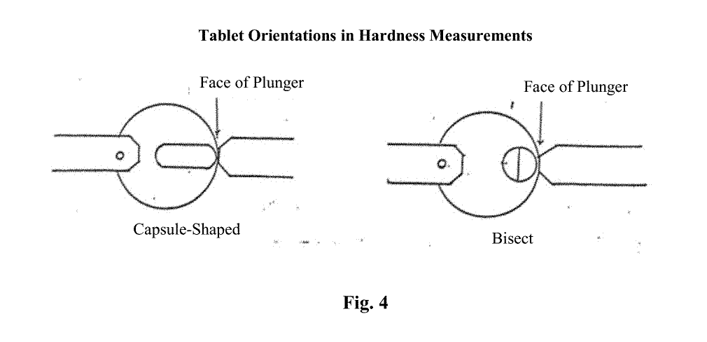

In some instances, tablet hardness measurements are calculated using a tablet hardness tester such as Key Model HT300 or Model HT500 or Pharma Test PTS/301. Using the limit knob on the left side of the housing, the plunger is adjusted according to the diameter of the tablet. The tablet is placed broadside down on the pedestal so that it was centered and was against the right side (stationary) plunger. Capsule-shaped tablets are placed with one end against the stationary plunger, breaking force applied end-to-end, see FIG. 4. When round or square tablets are scored, the tablet is positioned with the bisect parallel to the plunger contact surface.

In some instances, the pharmaceutical composition has a hardness of about 8 to about 22 kilopond (kp). In some instances, the pharmaceutical composition has a hardness of about 12-20 kp. In some instances, the pharmaceutical composition has a hardness of about 14-18 kp. In some instances, the pharmaceutical composition has a hardness of about 15-17 kp. In some instances, the pharmaceutical composition has a hardness of about 15.5 kp to about 16.5 kp. In some instances, the pharmaceutical composition has a hardness of about 16.5 kp. In some instances, the pharmaceutical composition has a hardness of about 8, 9, 10, 11, 12, 13, 14, 15, 16, 17, 18, 19, 20, 21, or about 22 kp. In some instances, the pharmaceutical composition has a hardness of about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, or about 22.2 kp.

In some instances, the pharmaceutical composition has a thickness of about 5 to about 8 mm. In some instances, the pharmaceutical composition has a thickness of about 6 to about 7 mm. In some instances, the pharmaceutical composition has a thickness of about 6.2 to about 6.8 mm. In some instances, the pharmaceutical composition has a thickness of about 6.0, 6.1, 6.2, 6.3, 6.4, 6.5, 6.6, 6.7, 6.8, 6.9, 7.0, 7.1, 7.2, 7.3, 7.4, or about 7.5 mm. In some instances, the pharmaceutical composition has a thickness of about 6.10, 6.11, 6.12, 6.13, 6.14, 6.15, 6.16, 6.17, 6.18, 6.19, 6.20, 6.21, 6.22, 6.23, 6.24, 6.25, 6.26, 6.27, 6.28, 6.29, 6.30, 6.31, 6.32, 6.33, 6.34, 6.35, 6.36, 6.37, 6.38, 6.39, 6.40, 6.41, 6.42, 6.43, 6.44, 6.45, 6.46, 6.47, 6.48, 6.49, 6.50, 6.51, 6.52, 6.53, 6.54, 6.55, 6.56, 6.57, 6.58, 6.59, 6.60, 6.61, 6.62, 6.63, 6.64, 6.65, 6.66, 6.67, 6.68, 6.69, 6.70, 6.71, 6.72, 6.73, 6.74, 6.75, 6.76, 6.77, 6.78, 6.79, 6.80, 6.81, 6.82, 6.83, 6.84, 6.85, 6.86, 6.87, 6.88, 6.89, 6.90, 6.91, 6.92, 6.93, 6.94, 6.95, 6.96, 6.97, 6.98, 6.99, 7.00, 7.01, 7.02, 7.03, 7.04, 7.05, 7.06, 7.07, 7.08, 7.09, 7.10, 7.11, 7.12, 7.13, 7.14, 7.15, 7.16, 7.17, 7.18, 7.19, or about 7.20 mm.

In some instances, weight measurements and calculations are conducted as follows. Approximately 100 tablets are weighed individually using an appropriate weighing device. The average weight and the relative standard deviation of the weights are calculated. For example, approximately 100 tablets are taken and weighed individually using a Mocon AB3 weigh balance. Pieces of tablets are not included. The printed results are inspected to ensure at least 90 individual weight results are recorded and that any rejected results are reasonable, (i.e., double or triple weights). Any weight result that is outside of a range (0.5 times the theoretical unit weight to 1.5 times the theoretical unit weight) is not included in the calculation. The software that analyzes the tablet weight data assigned T1 and T2 limits to tablets based on U.S. Pharmacopeial Convention weight variation criteria. Individual tablet weight results are flagged and counted as exceeding the T1 and T2 criteria as follows: if the mean tablet weight is 130 mg or less, T1 limits are ±10%, T2 limits are ±20%; if the mean tablet weight is between 130 and 325 mg, T1 limits are ±7.5%, T2 limits are ±15%; if the mean tablet weight is 325 mg or more, T1 limits are ±5.0%, T2 limits are ±10%. In some embodiments, if the items being weighed are capsules, individual gross weight results outside of the ±10% of the calculated average range are flagged and counted as exceeding T1. Any individual gross weight results outside of the ±15% of the calculated average range are flagged and counted as exceeding T2. If values exist outside this range, the actual empty capsule weight is subtracted from the mean weight to obtain a calculated net fill weight and a range of ±25% of the calculated net fill weight is determined. Tablets are flagged when individual results exceed the calculated average weight by the factor indicated.

### In Vitro Dissolution Methods

In vitro dissolution studies can be performed in accordance with guidelines from the United States Pharmacopeial Convention, the European Pharmacopoeia, and/or the Japanese Pharmacopoeia. The pharmaceutical compositions described herein can be tested for in vitro dissolution. In some instances, tablets can be tested for in vitro dissolution. In some instances, more than one of the same tablets can be tested for in vitro dissolution. In these cases, the more than one of the same tablets can be called a test batch. The size of the test batch can be at least about 10% of the largest batch planned. The size of the test batch can be 100,000 tablets. The size of the test batch can be less than 100,000 tablets. The size of the test batch can be more than 100,000 tablets.

In some instances, dissolution apparatus can be a USP Rotating Paddle Apparatus 2 with an automated sampling station (e.g., VK-8000 or equivalent). Dissolution fluid can be de-aerated 0.01 N HCl, maintained at 37.0+/−0.5° C. during dissolution procedure. The fluid can be prepared by diluting concentrated HCl in de-aerated water, and mixed. To measure peaks, a dual wavelength detector (e.g., Hitachi L-2420) can be used, or in some instances, two separate chromatographic systems can be used in order to measure the peaks at two different wavelengths.

In some instances, standard solution preparation: each ingredient is weighed in a volumetric flask, and diluted to volume with dissolution media. The resulting solution is mixed to form a stock solution. Different ingredients are similarly prepared to provide stock solutions (e.g., promethazine HCl, acetaminophen). Each aliquot of stock standard solutions is diluted with dissolution fluid and mixed to produce a final standard solution. For example, the concentration of hydrocodone bitartrate is about 0.0084 mg/mL, promethazine HCl is about 0.014 mg/mL, and acetaminophen is about 0.36 mg/mL.

In some instances, dissolution test solutions are prepared in 0.01 N HCl using the USP Rotating Paddle Apparatus at 50 WM. An aliquot of the dissolution solution is filtered and an aliquot is chromatographed on a 50-mm×4.6-mm (i.d.) Waters sunFire™ C₁₈, 3.5-μm particle size column using a gradient HPLC method. Mobile phase A consists of water/acetonitrile/TFA, 950/50/2 (v/v/v) and mobile phase B consisted of water/acetonitrile/TFA, 50/950/1.5 (v/v/v). The flow rate can be 2.0 mL/minute. For example, the amount of acetaminophen released can be determined at 300 nm by comparing the area obtained for the peak due to acetaminophen in the chromatogram of the dissolution test solution to that obtained for the corresponding peak in a chromatogram of a standard solution. The amount of hydrocodone bitartrate released can be determined at 230 nm by comparing the area obtained for the peak due to hydrocodone bitartrate in the chromatogram of the dissolution test solution to that obtained for the corresponding peak in a chromatogram of a standard solution. The amount of promethazine HCl released can be determined at 230 nm by comparing the area obtained for the peak due to promethazine HCl in the chromatogram of the dissolution test solution to that obtained for the corresponding peak in a chromatogram of a standard solution.

In some instances, paddle speed is 50 rpm; pull volume is 10 mL (no replacement); Pull points: 5, 10, 15, 20, 25, 30, 45 and 60 minutes. The amount of each component dissolved in the dissolution medium can be determined by HPLC. The method can use a high purity, bonded C18 stationary phase and a binary mobile phase consisting of an appropriate buffer and organic modifier.

In some instances, dissolution fluid is preheated to 37° C. and placed into each vessel. Tablets are weighed and placed in vessels respectively. At prescribed time intervals, an aliquot of the dissolution fluid can be drawn using the automated sampling station equipped with a 35 μm full flow filter connected to a sampling probe. Filtrate is allowed to cool to room temperature, to produce a final sample solution. Fluid withdrawn is not replaced. Samples are injected in HPLC for analysis after a baseline is established. The resolution between each peak is calculated, as well as the tailing factor. The mean and % RSD values for the acetaminophen peak areas at 300 nm can be measured; for example promethazine HCl and hydrocodone bitartrate at 230 nm. In some embodiments, the five replicate injections are not more than 2.0% RSD. In some embodiments, 50 μL aliquots of standard and sample solutions were subjected to liquid chromatography.

Calculation of the amount released per tablet can be determined using Equation I:

$$\underline{\frac{mg Released}{Tablet} = \frac{Au}{As} \times Cs \times Vn}$$

where:
Au=The peak area response obtained in the chromatogram of the dissolution test solution.
As=The peak area response obtained in the chromatogram of the standard solution.
Cs=The concentration of the standard solution.
Vn=The volume, in mL, of the dissolution solution at sampling time period n. It is calculated as follows:

Vn=[900−5(n−1)]

Calculation of the amount released as a percent of the label claim was determined using the following equation:

$$\% Released = \frac{mg \frac{Released}{Tablet}}{Label Claim} \times 100 \%$$

Calculation of the amount released at second and subsequent time periods in mg/tablet was determined using the following equation:

$$Wn = Un \underline{+ 5 {\sum }_{i = 1}^{n = 1} \frac{Ui}{Vi}}$$

where:
Wn=The mg released/tablet at time period n (corrected).
Un=The mg released/tablet at time n (uncorrected).
n=The current time period.
i=The time period index.
Ui=The mg released/tablet at time period i (uncorrected).
Vi=The volume, in mL, of the dissolution solution at sampling time period i. It is calculated as follows:

Vi=[900−5(i−1)]

In some instances, in vitro dissolution testing is collected from at least about 24 tablets. In some instances, in vitro dissolution testing can be collected from at least about: 10, 12, 16, 18, 20, 22, 24, 26, 28, 30, 32, 34, 36, 40, 50, or 100 tablets. In some instances, in vitro dissolution testing can be collected from more than about: 10, 12, 16, 18, 20, 22, 24, 26, 28, 30, 32, 34, 36, 40, 50, 100 tablets or more. In some instances, in vitro dissolution testing can be collected from less than about: 10, 12, 16, 18, 20, 22, 24, 26, 28, 30, 32, 34, 36, 40, 50, 100 tablets or less. Tablets can be from the same test batch. In some instances, in vitro dissolution testing can be done on equal numbers of test and reference tablets (e.g. 12 test tablets and 12 reference tablets).

Tablets can be added to a liquid, such as a dissolution medium, to initiate dissolution of the tablet and to permit sampling of the liquid to measure the amount of composition released from the tablet at one or more specific time points. Dissolution medium can be a solution. Dissolution medium can be a buffered solution. The buffered solution can have a pH of between about 4 and about 8. Dissolution medium can be an acid. The dissolution medium can be an acid between about 0.001N to about 0.1N, for example 0.1N hydrochloric acid. Dissolution medium can be deaerated. Dissolution medium can contain dissolved gases. Dissolution medium can be about pH 7.4. Dissolution medium can be about pH 7.0. Dissolution medium can be about pH 6.0. Dissolution medium can be 0.05 M pH 7.4 phosphate buffer. Dissolution medium can be deaerated water. Dissolution medium can be an acid. Dissolution medium can be a base. Dissolution medium can be 0.1 N hydrochloric acid. Dissolution medium can be water. Dissolution medium can be pH 6.0 phosphate buffer solution. Solutes such as surfactants can be added to the dissolution medium.

In vitro dissolution testing can be performed using an apparatus as described in the General Chapters and General Methods of the Harmonization section of the United States Pharmacopeia and the National Formulary (USP-NF), such as the General Methods 711 Dissolution Standards section. In some instances, a specific volume of dissolution medium can be placed into the vessel of the apparatus. A volume of about 500 mL dissolution medium can be added. A volume of about 900 mL dissolution medium can be added. A volume of about 1000 mL dissolution medium can be added. A volume of about: 200, 300, 400, 500, 600, 700, 750, 800, 900, 1000, 1100, 1200, 1300, 1400, 1500, 2000 mL dissolution medium can be added. The volume of dissolution medium can not be less than 3 times that need to form a saturated solution of the composition.

The apparatus can be a basket, for example, as described in the General Methods 711 Dissolution Standards of the USP-NF. The basket apparatus can be used for dissolution studies of capsules. The basket apparatus can be used for dissolution studies of controlled-release formulations. The apparatus can be a paddle, for example, as described in the General Methods 711 Dissolution Standards of the USP-NF. The paddle apparatus can be used for dissolution studies of solid formulations. The paddle apparatus can be used for dissolution studies of tablets. The apparatus can be a reciprocating cylinder, for example, as described in the General Methods 711 Dissolution Standards of the USP-NF. The reciprocating cylinder apparatus can be used for dissolution studies of bead-type controlled-release dosage forms. The apparatus can be a flow cell, for example, as described in the General Methods 711 Dissolution Standards of the USP-NF. The flow cell apparatus can be used for dissolution studies of controlled-release dosage forms. The flow cell apparatus can be used for dissolution studies of formulations with poor solubility. The apparatus can be a paddle over disk. The paddle over disk apparatus can be used for dissolution studies of transdermal dosage forms. The apparatus can be a cylinder. The cylinder apparatus can be used for dissolution studies of transdermal dosage forms. The apparatus can be a reciprocating disk. The reciprocating disk apparatus can be used for dissolution studies of non-disintegrating oral controlled-release dosage forms. A size 40-mesh screen can be used in the apparatus. The apparatus can be calibrated with the USP Salicyclic Acid and Prednisone Calibrator Tablets.

The apparatus can be assembled. The dissolution medium can be equilibrated to about 35+/−0.5 degrees Fahrenheit. The dissolution medium can be equilibrated to about internal body temperature. One test tablet or one reference tablet can be placed into the apparatus in the dissolution medium. The apparatus can be immediately set to operate. The apparatus can be set to operate. The apparatus can be set to operate at about 25 rotations per minute (rpm). The apparatus can be set to operate at about 50 rpm. The apparatus can be set to operate at about 100 rpm. In some instances, the apparatus can be set to operate at about: 10, 20, 25, 30, 35, 40, 45, 50, 55, 60, 65, 70, 75, 80, 85, 90, 95, 100, 105, 110, 115, 120, 125, 130, 135, 140, 145, 150, 200, or 300 rpm. Samples of dissolution medium can be withdrawn.

One or more samples of dissolution medium can be withdrawn at one or more specified times. Samples can be withdrawn following the addition of the tablet into the dissolution medium in the vessel of the apparatus. One or more samples can be collected at one or more times. In some instances, one or more samples can be collected at three different times. In some instances, the final time is chosen to show complete release of the composition. One or more samples can be collected at about: 5, 10, 15, 20, 30, 45, 60, 90, or 180 minutes. A volume of dissolution medium can be withdrawn from the vessel. The volume can be defined. The volume can be unknown. The volume of dissolution medium withdrawn can be the same for all times in a single in vitro dissolution study. The volume of dissolution medium can be withdrawn from the zone midway between the surface of the dissolution medium and the top of the rotating basket or blade and not less than 1 cm from the vessel wall. The volume of dissolution medium withdrawn from the vessel can be replaced with an equal volume of fresh dissolution medium. In this instance, the volume change can be incorporated into the dissolution calculation. The temperature of the dissolution medium in the vessels can be verified at one or more time points. In vitro dissolution testing can be repeated with one or more additional tablets. Withdraw of dissolution medium from the vessel can be automated.

In some instances, the amount of composition dissolved is at least about 75% in 15 minutes. In some instances, the amount of composition dissolved is at least about 80% in 20 minutes. In some instances, the amount of composition dissolved is at least about 85% in 30 minutes. In some instances, the amount of composition dissolved is at least about 80% in 30 minutes. In some instances, the amount of composition dissolved is at least about 75% in 60 minutes. In some instances, the amount of composition dissolved is at least about 80% in 15 minutes. In some instances, the amount of composition dissolved is at least about 80% in 45 minutes.

Percent dissolution refers to the weight percent of a pharmaceutical agent (for example, hydrocodone, acetaminophen, or promethazine) that is released from a tablet, see Example 6. For example, 0% dissolution of hydrocodone means no mass of hydrocodone is released from the tablet. In contrast, 100% dissolution of hydrocodone means the entire mass of hydrocodone is released from the tablet. Dissolution rate refers to the weight percent of a pharmaceutical agent released from a tablet in a given time interval (for example, 5 minutes or less, 10 minutes or less, or 15 minutes or less).

In some instances, an active agent of a pharmaceutical composition has a percent dissolution of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% in about 5 minutes or less. In some instances, an active agent of the pharmaceutical composition has a percent dissolution of about 30 to about 80% in about 5 minutes or less. In some instances, the pharmaceutical composition has a percent dissolution of hydrocodone of about 33 to about 72% in about 5 minutes or less. In some instances, the pharmaceutical composition has a percent dissolution of hydrocodone of about 35 to about 60% in about 5 minutes or less. In some instances, the percent dissolution of hydrocodone within about 5 minutes or less is about: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or about 72%. In some instances, the pharmaceutical composition has a percent dissolution of acetaminophen of about 55 to about 80% in about 5 minutes or less. In some instances, the percent dissolution of acetaminophen within about 5 minutes or less is about: 57, 58, 59, 60, 61, 62, 63, 64, 65, 66, 67, 68, 69, 70, 71, 72, 73, 74, 75, 76, 77, 78, 79, or about 80%. In some instances, the pharmaceutical composition has a percent dissolution of promethazine of about 65 to about 100% in about 5 minutes or less. In some instances, the pharmaceutical composition has a percent dissolution of promethazine of about 80 to about 100% in about 5 minutes or less. In some instances, the percent dissolution of promethazine within about 5 minutes or less is about: 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or about 98%.

In some instances, an active agent of a pharmaceutical composition has a percent dissolution of about 65 to about 100% in about 10 minutes or less. In some instances, the active agent of the pharmaceutical composition has a percent dissolution of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% in about 10 minutes or less. In some instances, the pharmaceutical composition has a percent dissolution of hydrocodone of about 65 to about 86% in about 10 minutes or less. In some instances, the percent dissolution of hydrocodone within about 10 minutes or less is about: 68, 69, 70, 71, 72, 73, 74, 75, 76, 77, 78, 79, 80, 81, 82, 83, 84, 85, 86, or about 87%. In some instances, the pharmaceutical composition has a percent dissolution of acetaminophen of about 65 to about 100% in about 10 minutes or less. In some instances, the percent dissolution of acetaminophen within about 10 minutes or less is about: 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100%. In some instances, the pharmaceutical composition has a percent dissolution of promethazine of about 78 to about 100% in about 10 minutes or less. In some instances, the percent dissolution of promethazine within about 10 minutes or less is about: 79, 80, 81, 82, 83, 84, 85, 86, 87, 88, 89, 90, 91, 92, 93, 94, 95, 96, 97, 98, 99, or about 100%.

In some instances, an active agent of a pharmaceutical composition has a percent dissolution of about 74 to about 100% in about 15 minutes or less. In some instances, the active agent of the pharmaceutical composition has a percent dissolution of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% in about 15 minutes or less. In some instances, the pharmaceutical composition has a percent dissolution of hydrocodone of about 78 to about 95% in about 15 minutes or less. In some instances, the percent dissolution of hydrocodone within about 15 minutes or less is about: 79, 80, 81, 82, 83, 84, 85, 86, 87, 88, 89, 90, 91, 92, 93, or about 94%. In some instances, the pharmaceutical composition has a percent dissolution of acetaminophen of about 75 to about 100% in about 15 minutes or less. In some instances, the percent dissolution of acetaminophen within about 15 minutes or less is about: 74, 75, 76, 77, 78, 79, 80, 81, 82, 83, 84, 85, 86, 87, 88, 89, 90, 91, 92, 93, 94, 95, 96, 97, 98, 99, or about 100%. In some instances, the pharmaceutical composition has a percent dissolution of promethazine of about 86 to about 100% in about 15 minutes or less. In some instances, the percent dissolution of promethazine within about 15 minutes or less is about: 86, 87, 88, 89, 90, 91, 92, 93, 94, 95, 96, 97, 98, 99, or about 100%.

In some instances, changing a process parameter alters a pharmaceutical composition. A non-limiting example of the process parameter can be a compression setting. In some instances, changing the compression setting alters the friability of the pharmaceutical composition. In some instances, changing the compression settings alters the hardness of the pharmaceutical composition. In some instances, changing the compression settings can alter the thickness of the pharmaceutical composition. In some instances, changing the compression settings alters the dissolution rate of a pharmaceutical agent within the pharmaceutical composition. In some instances, changing a single process parameter can alter the friability, hardness, thickness, dissolution rate, or combinations thereof.

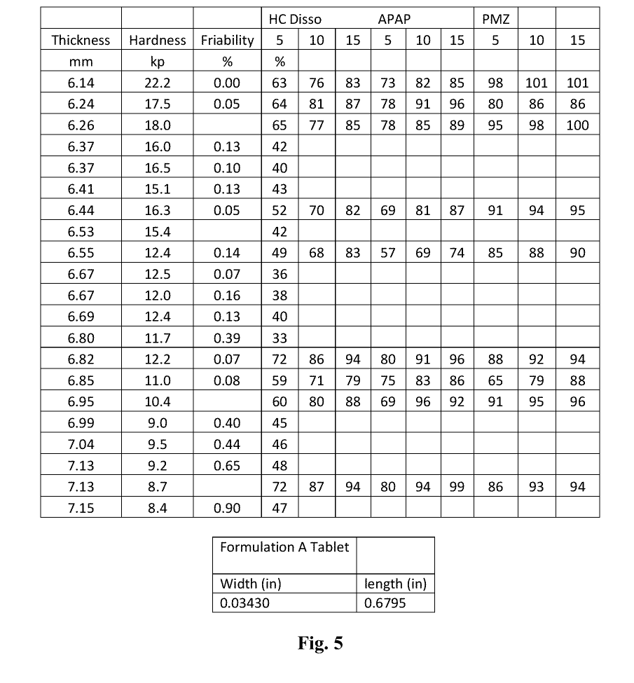

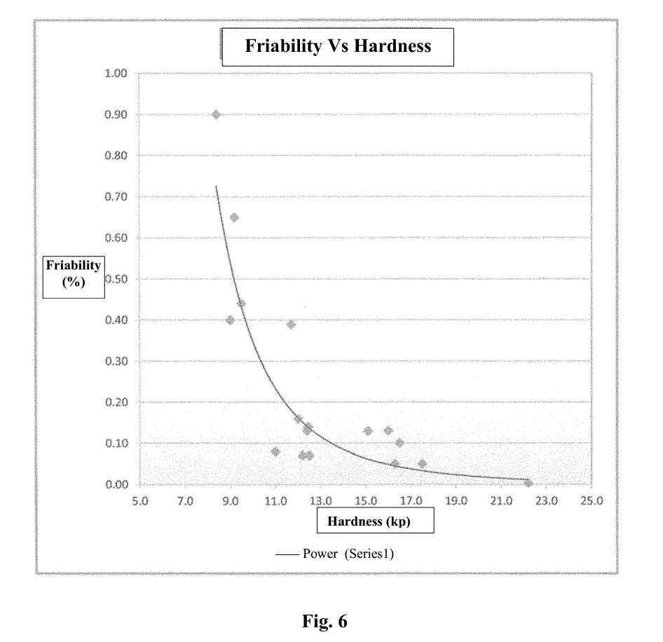

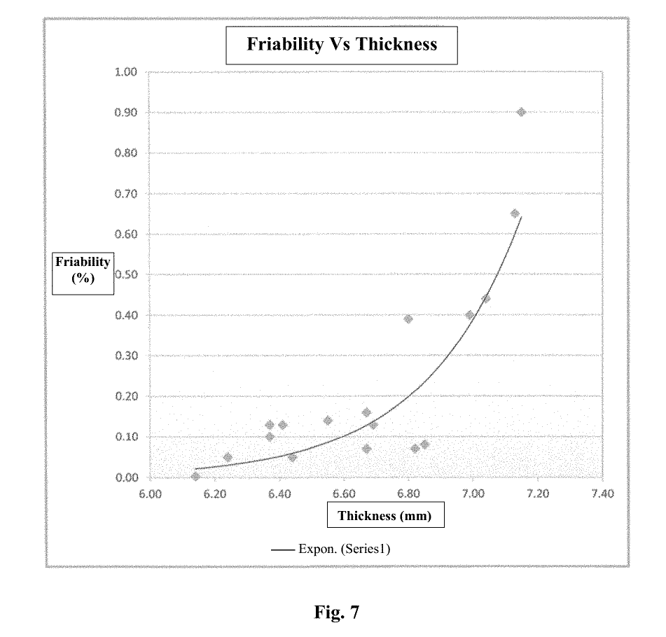

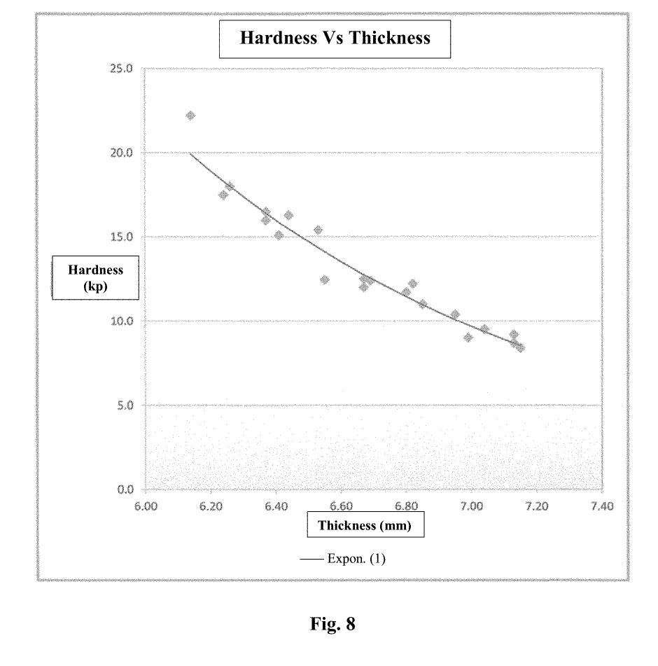

In some instances, friability of a pharmaceutical composition is correlated to hardness, FIG. 5 and FIG. 6. In some instances, friability of the pharmaceutical composition is correlated to thickness, FIG. 5 and FIG. 7. In some instances, an increase in friability correlates to a decrease in hardness. In some instances, an increase in friability correlates to an increase in thickness. In some instances, a decrease in friability correlates to an increase in hardness. In some instances, a decrease in friability correlates to a decrease in thickness. In some instances, hardness is correlated to thickness, FIG. 5 and FIG. 8. In some instances, an increase in hardness correlates to a decrease in thickness. In some instances, a decrease in hardness correlates to an increase in thickness. In some instances,

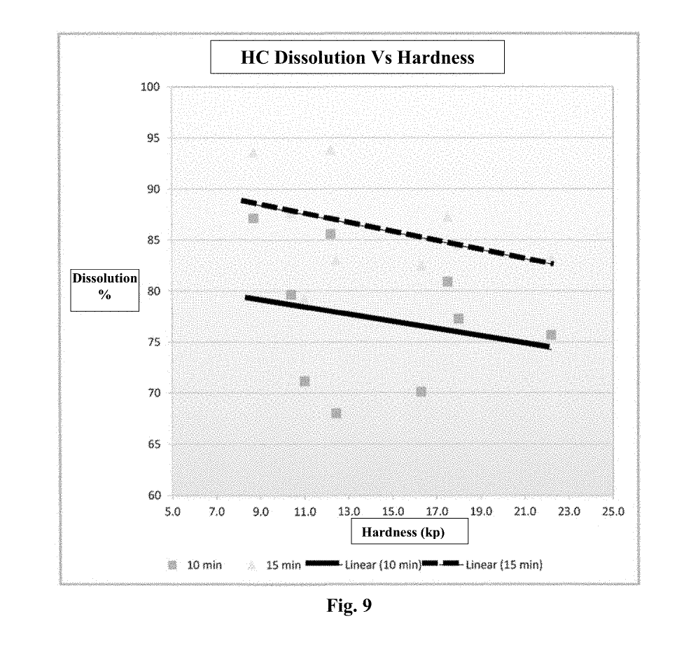

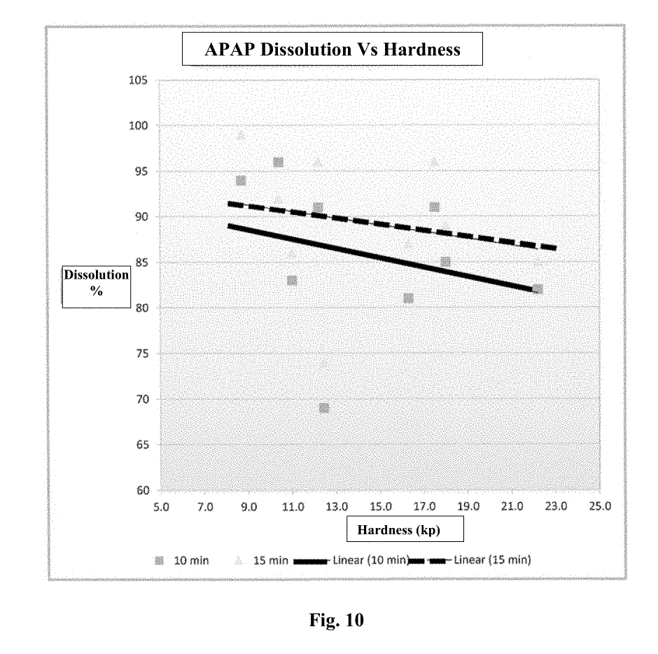

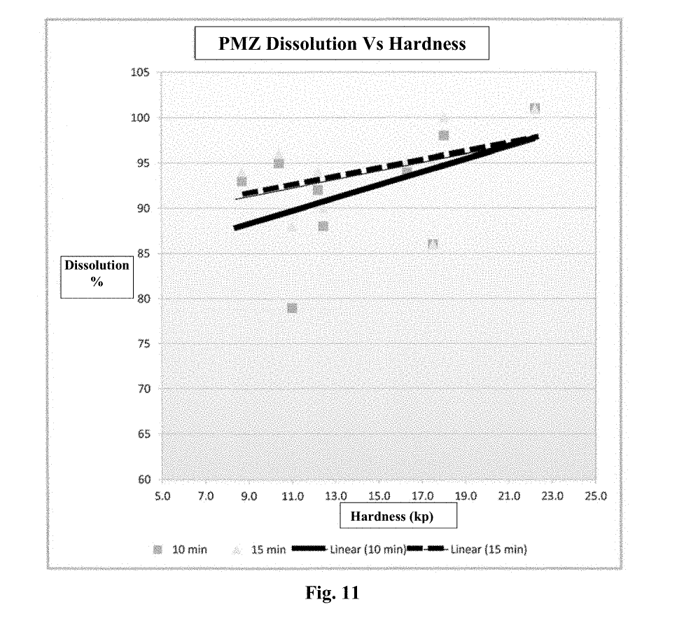

In some instances, hardness is correlated to a dissolution rate of a pharmaceutical agent, FIG. 5 and FIGS. 9-11. In some instances, an increase in hardness correlates to a decrease in dissolution rate of hydrocodone. In some instances, a decrease in hardness correlates to an increase in dissolution rate of hydrocodone. In some instances, an increase in hardness correlates to a decrease in dissolution rate of acetaminophen. In some instances, a decrease in hardness correlates to an increase in dissolution rate of acetaminophen. In some instances, an increase in hardness correlates to an increase in dissolution rate of promethazine. In some instances, a decrease in hardness correlates to a decrease in dissolution rate of promethazine.

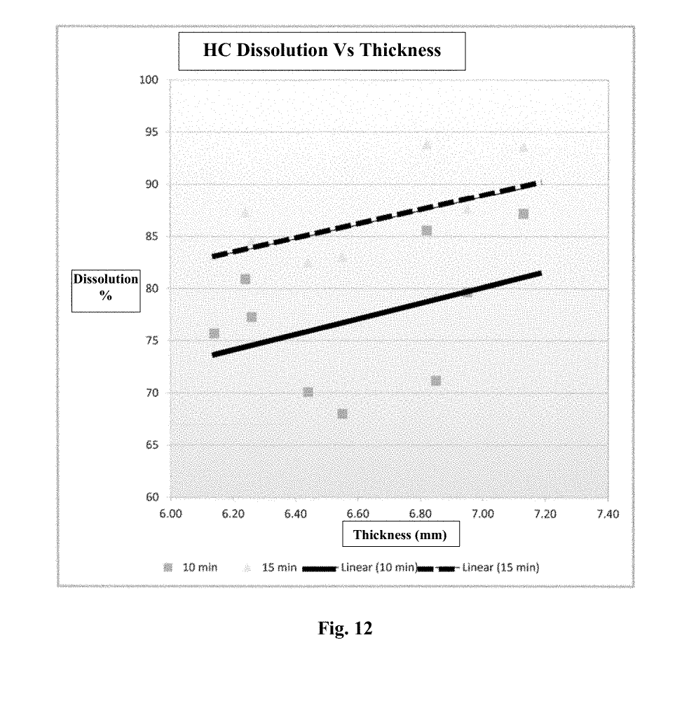

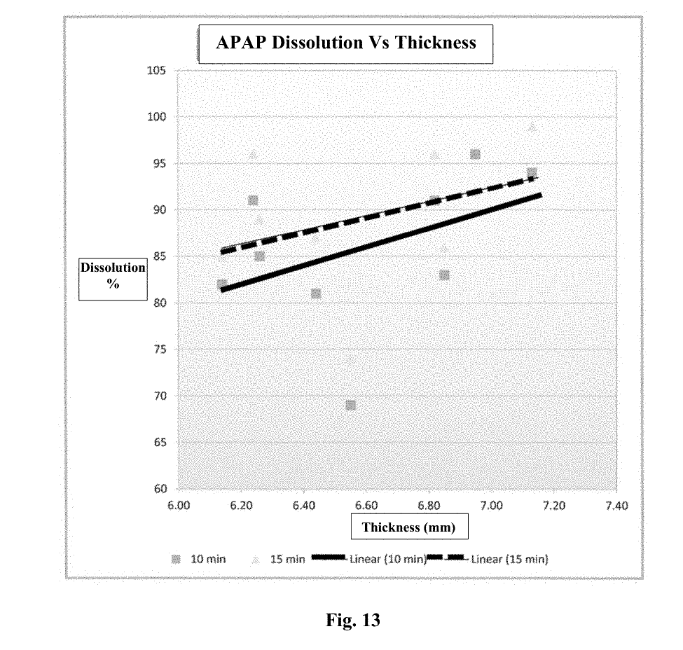

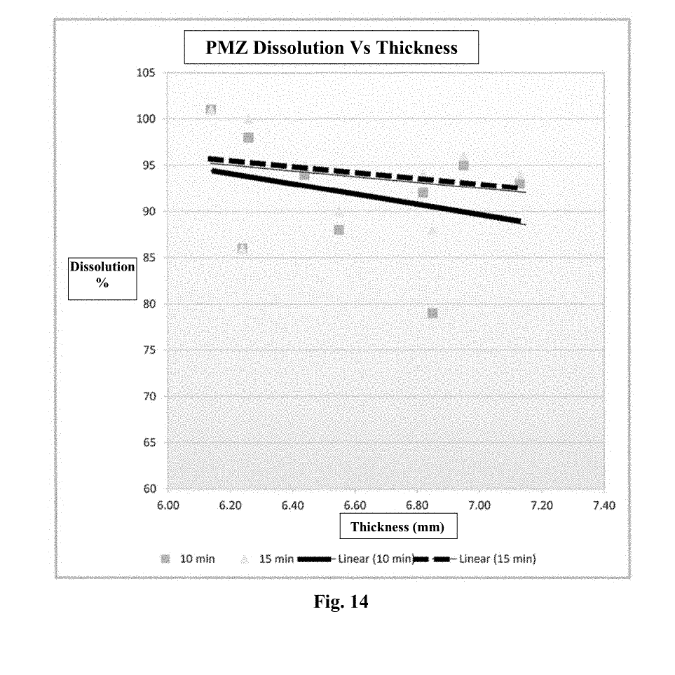

In some instances, thickness is correlated to a dissolution rate of a pharmaceutical agent, FIG. 5, and FIGS. 12-14. In some instances, an increase in thickness correlates to an increase in dissolution rate of hydrocodone. In some instances, a decrease in thickness correlates to a decrease in dissolution rate of hydrocodone. In some instances, an increase in thickness correlates to an increase in dissolution rate of acetaminophen. In some instances, a decrease in thickness correlates to a decrease in dissolution rate of acetaminophen. In some instances, an increase in thickness correlates to a decrease in dissolution rate of promethazine. In some instances, a decrease in thickness correlates to an increase in dissolution rate of promethazine.

In some instances, a pharmaceutical composition has a friability of about 0.1% to about 0.9%, a hardness of about 8 to about 22 kp, a dissolution of hydrocodone of about 33 to about 72% within about 5 minutes or less, a dissolution of acetaminophen of about 55 to about 80% within about 5 minutes or less, and a dissolution of promethazine of about 65 to about 100% within about 5 minutes or less.

In some instances, a pharmaceutical composition has a friability of about 0.1% to about 0.9%, a hardness of about 8 to about 22 kp, a dissolution of hydrocodone of about 65 to about 86% within about 10 minutes or less, a dissolution of acetaminophen of about 65 to about 100% within about 10 minutes or less, and a dissolution of promethazine of about 78 to about 100% within about 10 minutes or less.

In some instances, a pharmaceutical composition has a friability of about 0.1% to about 0.9%, a hardness of about 8 to about 22 kp, a dissolution of hydrocodone of about 78 to about 95% within about 15 minutes or less, a dissolution of acetaminophen of about 75 to about 100% within about 15 minutes or less, and a dissolution of promethazine of about 86 to about 100% within about 15 minutes or less. In some instances, a pharmaceutical composition has a friability of about 0.05% to about 0.2%, a hardness of about 12 to about 20 kp, a dissolution of hydrocodone of about 35 to about 60% within about 5 minutes or less, a dissolution of acetaminophen of about 55 to about 80% within about 5 minutes or less, and a dissolution of promethazine of about 80 to about 100% within about 5 minutes or less. In some instances, a pharmaceutical composition has a friability of about 0.05% to about 0.2%, a hardness of about 12 to about 20 kp, a dissolution of hydrocodone of about 65 to about 86% within about 10 minutes or less, a dissolution of acetaminophen of about 65 to about 100% within about 10 minutes or less, and a dissolution of promethazine of about 78 to about 100% within about 10 minutes or less. In some instances, the pharmaceutical composition has a friability of about 0.05% to about 0.2%, a hardness of about 12 to about 20 kp, a dissolution of hydrocodone of about 78 to about 95% within about 15 minutes or less, a dissolution of acetaminophen of about 75 to about 100% within about 15 minutes or less, and a dissolution of promethazine of about 86 to about 100% within about 15 minutes or less. In some instances, a pharmaceutical composition has a friability of about 0.05% to about 0.14%, a hardness of about 14 to about 18 kp, a dissolution of hydrocodone of about 40 to about 65% within about 5 minutes or less, a dissolution of acetaminophen of about 55 to about 80% within about 5 minutes or less, and a dissolution of promethazine of about 80 to about 100% within about 5 minutes or less. In some instances, a pharmaceutical composition has a friability of about 0.05% to about 0.14%, a hardness of about 15 to about 17 kp, a dissolution of hydrocodone of about 40 to about 52% within about 5 minutes or less, a dissolution of acetaminophen of about 55 to about 80% within about 5 minutes or less, and a dissolution of promethazine of about 80 to about 100% within about 5 minutes or less. In some instances, a pharmaceutical composition has a friability of about 0.05% to about 0.14%, a hardness of about 15.5 to about 16.5 kp, a dissolution of hydrocodone of about 40 to about 52% within about 5 minutes or less, a dissolution of acetaminophen of about 55 to about 80% within about 5 minutes or less, and a dissolution of promethazine of about 80 to about 100% within about 5 minutes or less. In some instances, a pharmaceutical composition has a hardness of about 16 kp, a friability of about 0.13%, a thickness of about 6.4 mm, and a hydrocodone dissolution rate of about 42% within about 5 minutes or less. In some instances, the pharmaceutical composition can have a hardness of about 16.5 kp, a friability of about 0.10%, a thickness of about 6.4 mm, and a hydrocodone dissolution rate of about 40% within about 5 minutes or less. In some instances, the pharmaceutical composition has a hardness of about 15.1 kp, a friability of about 0.13%, a thickness of about 6.4 mm, and a hydrocodone dissolution rate of about 43% within about 5 minutes or less. In some instances, the pharmaceutical composition can have a hardness of about 15.4 kp, a thickness of about 6.5 mm, and a hydrocodone dissolution rate of about 42% within about 5 minutes or less.

In some instances, a pharmaceutical composition has a hardness of about 16.3 kp, a friability of about 0.05%, a thickness of about 6.4 mm, a hydrocodone dissolution rate of about 52% within about 5 minutes or less, a hydrocodone dissolution rate of about 70% within 10 minutes or less, and a hydrocodone dissolution rate of about 82% within 15 minutes or less. In some instances, the pharmaceutical composition has a hardness of about 16.3 kp, a friability of about 0.05%, a thickness of about 6.4 mm, an acetaminophen dissolution rate of about 69% within about 5 minutes or less, an acetaminophen dissolution rate of about 81% within about 10 minutes or less, and an acetaminophen dissolution rate of about 87% within about 15 minutes or less. In some instances, the pharmaceutical composition has a hardness of about 16.3 kp, a friability of about 0.05%, a thickness of about 6.4 mm, a promethazine dissolution rate of about 91% within about 5 minutes or less, a promethazine dissolution rate of about 94% within about 10 minutes or less, and a promethazine dissolution rate of about 95% within about 15 minutes or less.

In some instances, a tablet is formed by a manufacturing process, see Example 2. One or more pharmaceutical agents, (for example, hydrocodone) are passed through individual screens and mixed in one or more blenders. Blended contents are then transferred to one or more hoppers that feed into a tablet press. The press produces tablets with specified parameters and at the end of the batch; the compressed tablets are deposited into a storage container.

In some instances, active agents within a tablet remain stable at ambient conditions for at least about one month, at least about 3 months, at least about 24 months, at least about 48 months or longer, depending on other components of the storage microenvironment, such as temperature, pressure, or humidity. In some cases, active agents remain stable at high temperature for at least about 6 months, at least about 9 months or longer. In some instances, high temperature can be about: 80, 81, 82, 83, 84, 85, 86, 87, 88, 89, 90, 91, 92, 93, 94, 95, 96, 97, 98, 99, 100, 101, 102, 103, or 104 degrees Fahrenheit. In some instances, high temperature can be more than 80, 81, 82, 83, 84, 85, 86, 87, 88, 89, 90, 91, 92, 93, 94, 95, 96, 97, 98, 99, 100, 101, 102, 103, 104 degrees Fahrenheit or more. In some instances, high temperature can be less than 80, 81, 82, 83, 84, 85, 86, 87, 88, 89, 90, 91, 92, 93, 94, 95, 96, 97, 98, 99, 100, 101, 102, 103, 104 degrees Fahrenheit or less.

In some instances, bioavailability (the rate and extent to which the active ingredient or active moiety in pharmaceutical compositions disclosed herein becomes available at the site of drug action) is documented using in vitro approaches. In some instances, such as for soluble, permeable, dissolving, or orally administered formulations, the rate and extent to which the active ingredient or active moiety in pharmaceutical compositions disclosed herein becomes available at the site of drug action can be documented using in vitro approaches. In some instances, an in vitro approach can be a dissolution study. Such in vivo studies can be performed in accordance with guidelines from the Food and Drug Administration, Office of Generic Drugs, Division of Bioequivalence.

Compositions described herein can be tested in vivo for the rate and extent to which the active ingredient or active moiety in pharmaceutical compositions disclosed herein becomes available at the site of drug action. In some instances, tablets can be tested in vivo for bioavailability. In some instances, more than one of the same tablets can be called a test batch. The size of the test batch can be at least about 10% of the largest batch planned. The size of the test batch can be 100,000 tablets. The size of the test batch can be less than 100,000 tablets. The size of the test batch can be more than 100,000 tablets. In some instances, the composition of a reference tablet does not differ from the test tablet by more than about 5% of the composition.

An in vivo bioavailability study can be a single-dose, randomized, fasting, two-period, two-treatment, two-sequence crossover study. An in vivo pharmacokinetics study can be in single-dose study. An in vivo bioavailability study can be a multi-dose study. An in vivo bioavailability study can be a randomized study. An in vivo bioequivalence study can be a non-randomized study. An in vivo bioavailability study can be a study in which all subjects fast prior to administering. An in vivo bioavailability study can compare equal doses of a test tablet and a reference tablet. An in vivo bioavailability study can be a crossover study. An in vivo bioavailability study can be a non-crossover study. A crossover study can be a two-period, two-treatment, two-sequence crossover study. A crossover study can include repeated measures in all subjects. An in vivo bioavailability study can be a longitudinal study. Subjects can receive different treatments in an in vivo bioavailability study. One treatment can be experimental. One treatment can be standard or placebo. Subjects can receive the same number of treatments. Subjects can receive a different number of treatments. Subjects can participate in the same number of periods. Subjects can participate in a different number of periods. In some instances, a crossover study can have four periods. In some instances, a crossover study can have two periods. An in vivo bioavailability study can include a washout period. In some cases, an in vivo bioavailability study does not include a washout period. A washout period can avoid carry-over effects when two sequential treatments are given close together in time to a single subject. At least a 1 week washout period can be observed between treatments. At least a 2 week washout period can be observed between treatments.

A dosing sequence can include type of dose (e.g. experimental or standard), amount of dose, frequency of treatment, or others. Subjects can be randomly assigned to one of one or more dosing sequences. Subjects can be randomly assigned to one of two possible dosing sequences. The proposed in vivo bioavailability study can be approved by an institutional review board. The in vivo bioavailability study can be performed in a clinical setting. The in vivo bioavailability study can be performed in a laboratory setting.

In some instances, at least about 24 subjects are enrolled in the in vivo bioavailability study. In some instances, at least about 10, 16, 18, 20, 22, 24, 26, 28, 30, 32, 34, 36, 40, 50, 100 subjects are enrolled in the study. Subjects can be healthy. Subjects can be non-smokers. Subjects can be smokers. Subjects can be between about 18 and about 50 years of age. Subjects can be less than 18 years of age. Subjects can be between about 11 and about 18 years of age. Subjects can be less than about 2 years of year. Subjects can be between about 2 and about 11 years of age. Subjects can be mammals. Subjects can be neonatal, infant, adolescent, or adult. Subjects can be pediatric subjects. Subjects can be adult subjects. Subjects can be within 10% ideal body weight. Subjects can be within 15% ideal body weight. Female subjects can not be pregnant. Subjects can not be selected to enroll in the study for any current or past medical condition that might significantly affect the pharmacokinetic or pharmacodynamics response. Written, informed consent can be obtained from a subject before enrollment into the study.

Subjects can fast for at least about 10 hours prior to administering. Subjects can fast for at least about: 8, 9, 12, 15, 16, 18, 24 hours prior to administering. Fasting can occur overnight. Fasting can not occur overnight. After completion of the fasting period, subjects can be administered one or more test tablets with 240 mL of water. After completion of the fasting period, subjects can be administered one or more reference tablets with 240 mL of water. In some cases, a subject can be administered one or more tablets with about: 100, 120, 140, 160, 180, 200, 220, 240, 260, 280, 300, 320, 340, 360, 380, 400 mL of water. In some cases, subjects can drink an additional 240 mL of water at about: 1, 2, 4, 6, 8, or 10 hours, or combinations thereof following administering of the one or more tablets and the initial 240 mL of water. Additional water can not be taken 1 hour before administering. Additional water can not be taken 1 hour after administering.

Subjects can fast for at least about 4 hours following administering. Subjects can fast for at least about 2, 3, 4, 5, 6, 8, 10, 12 hours following administering. Meals provided to subjects during the study can be standardized. Subjects can not consume alcohol for about 48 hours prior to administering. Subjects can not consume alcohol for about: 180, 96, 72, 48, 36, 24, 12, or 6 hours prior to administering. Subjects can not consume alcohol until after the last blood sample is collected for the study. Subjects can not consume additional medications at least about 2 weeks prior to administering. Subjects can not consume additional medications at least about: 52, 40, 30, 20, 10, 8, 7, 6, 5, 4, 3, 2 weeks prior to administering. Subjects can not consume additional medications at least about 7, 6, 5, 4, 3, 2 days prior to administering. Subjects can not consume additional medications until after the last blood sample is collected for the study.

Blood samples can be collected from subjects at one or more time points. Venous blood can be collected. Arterial blood can be collected. Blood can be collected from a peripheral intravenous line. Blood can be collected by heel or finger puncture. Collection of a blood sample from one or more subjects can occur at about: 10, 20 30, or 45 minutes after administering. Collection of a blood sample from one or more subjects can occur at about: 0, 0.17, 0.25, 0.33, 0.50, 0.67, 0.75, 1, 1.25, 1.33, 1.50, 1.67, 1.75, 2, 2.25, 2.5, 2.75, 3, 3.33, 3.5, 3.67, 4, 4.5, 5, 6, 7, 8, 10, 12, 14, 15, 16, 20, 24, 30, 36, 48, 72, 96, 120, 144, or 168 hours after administering. Collection of a blood sample from one or more subjects can occur at about: 8, 12, 15, 19, 22, 29, 33, 36, 43, 50, 57, 64, 70, 80, or 90 days after administering. Blood samples can be frozen immediately following collection. Blood samples can remain frozen until assayed. Blood samples can be analyzed as soon as they are collected. Plasma can be separated immediately following collection. Plasma can be frozen immediately following separation. Plasma can remain frozen until assayed.

### Multi-Layer Tablets

A pharmaceutical composition as described herein can be multi-layer tablets, such as bi-layer tablets or two layer tablets. In some instances, the bi-layer tablet comprises: (a) an immediate-release layer; and (b) a controlled-release layer. In some instances, the two layer tablet comprises: (a) an immediate-release layer; and (b) a controlled-release layer. In some instances, the immediate-release layer or the controlled-release layer comprises one or more pharmaceutically active agents. In some instances, a bi-layer tablet herein has a hardness of about 7, 7.5, 8, 8.5, 9, 9.5, 10, 10.5, 11, 11.5, 12, 12.5, 13, 13.5, 14, 14.5 or 15 kiloponds (kp). In some instances, the bi-layer tablet has a hardness of about 9.5 kp. In a further instance, a bi-layer tablet herein has a thickness of about 5, 5.5, 6, 6.5, 7, 7.5, 8, 8.5, 9, 9.5, or 10 mm. It can be understood that as to the kilopond and thickness measurements, increments of 0.1 decimal points are within the scope herein. In some instances, the tablet can be rectangular, tubular, oblong, circular, oval or in a capsule form.

In some instances, a bi-layer tablet herein provides an effective amount of one or more pharmaceutically active agents for about 4 to about 8 hours following oral administration, about 4-6 hours following oral administration, or about 6-8 hours following oral administration. In some instances, the one or more pharmaceutically active agents can be administered to a subject in about 4-hour, 5 hour, 6 hour, 7 hour or 8 hour dosing intervals. Therefore, a bi-layer tablet herein is capable of providing any of the one or more pharmaceutically active agents disclosed herein in the foregoing dosing intervals. In some instances, a pharmaceutical composition comprises promethazine or a pharmaceutically acceptable salt thereof and about 70 to about 80% of the promethazine or pharmaceutically acceptable salt thereof dissolves in the stomach of a subject after about 5 to about 10 minutes following oral administration. In some instances, the promethazine is promethazine HCl. In some instances, a pharmaceutical composition comprises hydrocodone or a pharmaceutically acceptable salt thereof and about 30 to about 60% of the hydrocodone or pharmaceutically acceptable salt thereof dissolves in the stomach of a subject after about 5 to about 10 minutes following oral administration. In some instances, the hydrocodone salt is hydrocodone bitartrate. In some instances, a pharmaceutical composition comprises acetaminophen or a pharmaceutically acceptable salt thereof and 50% to about 70% of the acetaminophen or pharmaceutically acceptable salt thereof dissolves in the stomach of a subject after about 5 to about 10 minutes following oral administration. In some instances, a pharmaceutical composition comprises promethazine or a pharmaceutically acceptable salt thereof, hydrocodone or a pharmaceutically acceptable salt thereof and acetaminophen or a pharmaceutically acceptable salt thereof, and at least 90% of the pharmaceutically active agents in the pharmaceutical composition dissolve in the stomach of a subject after about 45 minutes following oral administration. In some instances, the pharmaceutical composition is a bi-layer tablet comprising an immediate-release layer and a controlled-release layer.

In some instances, an immediate-release layer comprises promethazine or a pharmaceutically acceptable salt as the only pharmaceutically active agent. In another instance, the controlled-release layer comprises hydrocodone or a pharmaceutically acceptable salt and acetaminophen or a pharmaceutically acceptable salt as the only pharmaceutical ingredients. In some instances, the controlled-release layer comprises hydrocodone or a pharmaceutically acceptable salt as the only pharmaceutical ingredients.

In some instances, a controlled-release layer comprises an opioid analgesic or a non-opioid analgesic as the only pharmaceutically active agent. In another instance, the controlled-release layer comprises an opioid analgesic and a non-opioid analgesic as the only pharmaceutically active agents. In some instances, the immediate-release layer comprises an antiemetic or a stimulant as the only pharmaceutically active agent. In another instance, the immediate-release layer comprises an antiemetic and a stimulant as the only pharmaceutically active agents.

### Immediate-Release Layer

In some instances, an immediate-release layer is capable of releasing about 70 to about 80% of the one or more pharmaceutically active agent contained therein in the stomach of a subject in about 5 to about 10 minutes following oral administration. In some instances, the immediate-release layer is capable of releasing about 90 to about 100% of one or more pharmaceutically active agent contained therein in the stomach of a subject in about 40 minutes.

In some instances, about 80% to about 100% of an active agent in an immediate-release layer is released in about 5 to about 20 minutes as measured as measured by USP Apparatus 2 (Paddle Apparatus). In some instances, about 90% to about 100% of an active agent in an immediate-release layer is released in about 5 to about 20 minutes as measured as measured by USP Apparatus 2 (Paddle Apparatus). In some instances, about 80% to about 100% of an active agent in an immediate-release layer is released in about 5 to about 10 minutes as measured as measured by USP Apparatus 2 (Paddle Apparatus). In some instances, about 90% to about 100% of an active agent in an immediate-release layer is released in about 5 to about 10 minutes as measured as measured by USP Apparatus 2 (Paddle Apparatus).

In some instances, a one or more pharmaceutically active agent in the immediate-release layer is an antiemetic. In some instances, the antiemetic is promethazine or a pharmaceutically acceptable salt thereof. In another instance, the antiemetic is promethazine HCl. In some instances, an immediate-release layer comprises two or more agents, including an antiemetic and a stimulant.

In some instances, an immediate-release layer comprises one or more excipients, including but not limited to silicified microcrystalline cellulose (e.g., HD90), croscarmellose sodium (AC-Di-Sol), magnesium stearate.

In some instances, the total layer weight of an immediate-release layer is from about 100 to about 300 mg, such as about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg. In some instances, the total layer weight of an immediate-release layer is about 150 mg.

In some instances, the immediate-release layer comprises from about 75 to about 150 mg of silicified microcrystalline cellulose, from about 10 to about 20 mg croscarmellose sodium, from about 0.5 to 2 mg magnesium stearate. In yet a further instance, the immediate-release layer comprises from about 10 to about 15 mg promethazine, or a pharmaceutically acceptable salt thereof. In another instance, the immediate-release layer comprises about 12.5 mg promethazine or a pharmaceutically acceptable salt thereof. In another instance, the pharmaceutically acceptable salt is promethazine HCl. In some instances, an immediate-release layer comprise about 12.5 mg promethazine HCl, about 121.5 mg silicified microcrystalline cellulose, about 15 mg croscarmellose sodium, and about 1 mg magnesium stearate.

In some instances, a pharmaceutical composition comprising an effective amount of each of hydrocodone bitartrate, acetaminophen and promethazine HCl is capable of dissolving in the stomach of a subject so that an effective plasma concentration of each of pharmaceutically active ingredient is present in a subject in from about 5 to about 30 minutes. In some instances, a pharmaceutical composition comprising an effective amount of each of hydrocodone bitartrate and promethazine HCl is capable of dissolving in the stomach of a subject so that an effective plasma concentration of each of pharmaceutically active ingredient is present in a subject in from about 5 to about 30 minutes.

### Controlled-Release Layer

In some instances, a controlled-release layer is capable of releasing about 30 to about 40% of the one or more pharmaceutically active agent contained therein in the stomach of a subject in about 5 to about 10 minutes following oral administration. In another instance, the controlled-release layer is capable of releasing about 90% of the one or more pharmaceutically active agents are released in about 40 minutes after oral administration. In some instances, about 30% to about 90% of an active agent in a controlled-release layer is released in about 5 to about 20 minutes as measured by USP Apparatus 2 (Paddle Apparatus). In some instances, about 30% to about 80% of an active agent in a controlled-release layer is released in about 5 to about 20 minutes as measured by USP Apparatus 2 (Paddle Apparatus). In some instances, about 30% to about 70% of an active agent in a controlled-release layer is released in about 5 to about 20 minutes as measured by USP Apparatus 2 (Paddle Apparatus). In some instances, about 30% to about 60% of an active agent in a controlled-release layer is released in about 5 to about 20 minutes as measured by USP Apparatus 2 (Paddle Apparatus). In some instances, about 30% to about 90% of an active agent in a controlled-release layer is released in about 5 to about 10 minutes as measured by USP Apparatus 2 (Paddle Apparatus). In some instances, about 30% to about 80% of an active agent in a controlled-release layer is released in about 5 to about 10 minutes as measured by USP Apparatus 2 (Paddle Apparatus). In some instances, about 30% to about 70% of an active agent in a controlled-release layer is released in about 5 to about 70 minutes as measured by USP Apparatus 2 (Paddle Apparatus). In some instances, about 30% to about 60% of an active agent in a controlled-release layer is released in about 5 to about 10 minutes as measured by USP Apparatus 2 (Paddle Apparatus).

In some instances, a controlled-release layer comprises one or more excipients, including but not limited to silicified microcrystalline cellulose (e.g., HD90), croscarmellose sodium (AC-Di-Sol), or magnesium stearate.

In some instances, the total layer weight of a controlled-release layer is from about 100 to about 1000 mg, such as about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg; about: 350, 400, 450, 500, 550, 600, 650, 700, 750, 800, 850, 900, 950, or 1000 mg; or about: 500-600, 450-650, 400-700, 300-800, or 200-900 mg. In some instances, the total layer weight of a controlled-release layer is about 550 mg.

In some instances, a controlled-release layer comprises from about 75 mg to about 250 mg of silicified microcrystalline cellulose, from about 10 to about 40 mg hydroxyl methyl propyl cellulose, from about 0.5 to 5 mg magnesium stearate, and from about 0.5 to about 5 mg stearic acid. In some instances, a controlled-release layer comprises about 152 mg silicified microcrystalline cellulose, about 20 mg hydroxyl methyl propyl cellulose, about 2.75 mg magnesium stearate, about 2.75 stearic acid, about 7.5 mg hydrocodone or a pharmaceutically acceptable salt thereof, about 325 mg acetaminophen or a pharmaceutically acceptable salt thereof. In another instance, the controlled-release layer comprises about 152 mg silicified microcrystalline cellulose, about 20 mg hydroxyl methyl propyl cellulose, about 2.75 mg magnesium stearate, about 2.75 stearic acid, about 7.5 mg hydrocodone or a pharmaceutically acceptable salt thereof. In yet a further instance, the controlled-release layer comprises from about 5 to about 12.5 mg hydrocodone or a pharmaceutically acceptable salt thereof. In some instances, the controlled-release layer comprises about 7.5 mg hydrocodone or a pharmaceutically acceptable salt thereof. In some instances, the controlled-release layer comprises about 5 mg hydrocodone or a pharmaceutically acceptable salt thereof. In some instances, the controlled-release layer comprises about 10 mg hydrocodone or a pharmaceutically acceptable salt thereof. In another instance, the opioid analgesic is oxycodone or a pharmaceutically acceptable salt thereof. In some instances, the pharmaceutically acceptable salt is oxycodone HCl. In some instances, the amount of oxycodone present is about 5, 10, 15 mg or more. In some instances, the pharmaceutically acceptable salt for hydrocodone is hydrocodone bitartrate. In some instances, a controlled-release layer further comprises from about 290 mg to about 360 mg acetaminophen or a pharmaceutically acceptable salt thereof. In some instances, the controlled-release layer comprises about 325 mg acetaminophen or a pharmaceutically acceptable salt thereof. In some instances, an immediate-release layer comprises promethazine HCl and the controlled-release layer comprises hydrocodone bitartrate. In another instance, the controlled-release layer further comprises a non-opioid analgesic (e.g., acetaminophen).

In some instances, one or more pharmaceutically active agents of the controlled-release layer is an opioid analgesic. In some instances, the opioid analgesic is hydrocodone or oxycodone; or a pharmaceutically acceptable salt thereof. In some instances, the immediate-release layer is about 150 mg in total layer weight and the controlled-release layer is about 550 mg total weight.

In some instances, a controlled-release layer comprises about 325 mg acetaminophen, about 7.5 mg hydrocodone bitartrate, about 152 mg silicified microcrystalline cellulose, about 20 mg hydroxyl methyl propyl cellulose (HPMC), about 2.75 mg magnesium stearate, and about 2.75 mg stearic acid; and the immediate-release layer comprises about 12.5 mg promethazine HCl, about 121 mg silicified microcrystalline cellulose, about 15 mg croscarmellose sodium, and about 1 mg magnesium stearate. In some instances, the controlled-release layer comprises about 7.5 mg hydrocodone bitartrate, about 152 mg silicified microcrystalline cellulose, about 20 mg hydroxyl methyl propyl cellulose (HPMC), about 2.75 mg magnesium stearate, and about 2.75 mg stearic acid; and the immediate-release layer comprises about 12.5 mg promethazine HCl, about 121 mg silicified microcrystalline cellulose, about 15 mg croscarmellose sodium, and about 1 mg magnesium stearate.

In some instances, a bi-layer disclosed tablet herein comprises any combination of pharmaceutically active agents herein, wherein a controlled-release layer comprises one or more opioid analgesic agents, non-analgesic agents, barbiturates or stimulants, and an immediate-release layer comprises one or more stimulants. In some instances, a stimulant is present in the immediate-release layer, controlled-release layer or both layers; the immediate-release layer comprises one or more antiemetics; and the controlled-release layer comprises one or more non-opioid analgesics. In addition, either layer of the bi-layered tablet can comprise one or more anti-abuse agents disclosed herein. In some instances, a bi-layer tablet herein comprises a controlled-release layer comprising one or more analgesic agents as the only pharmaceutically active agents in the controlled-release layer. In another instance, a bi-layer tablet herein comprises an immediate-release layer comprising an antiemetic agent as the only pharmaceutically active agent in the immediate-release layer.

In some instances, a controlled-release layer further comprises one or more of: silicified microcrystalline cellulose, hydroxy methyl propyl cellulose, magnesium stearate, and stearic acid. In another instance, the immediate-release layer further comprises one or more of: silicified microcrystalline cellulose, croscarmellose sodium and magnesium stearate. In another instance, the tablet has a hardness of about 9.5 kilopond and thickness from about 6.9 to about 7.0 mm. In another instance, the hydrocodone salt is hydrocodone bitartrate. In another instance, the promethazine salt is promethazine HCL. In another instance, the controlled-release layer is an inner layer and wherein the immediate-release layer is an outer layer.

In some instances, for a pharmaceutical composition discloses herein, the opioid analgesic is oxycodone or pharmaceutically acceptable salt thereof and the one or more antiemetic is promethazine or a pharmaceutically acceptable salt thereof. In some instances, the effective amount is an amount effective for treating or preventing pain for a period of about 4-8 hours following administration to a subject (e.g., 4-6, 4-8, 6-8 hours). In some instances, a pharmaceutical composition disclosed herein is in the form of a tablet. In some cases, the tablet is bi-layer tablet. In some instances, the bi-layer tablet comprises an immediate-release layer and a controlled-release layer. In another instance, a two layer tablet comprises an immediate-release layer and a controlled-release layer. In another instance, the immediate-release layer comprises the promethazine or pharmaceutically acceptable salt thereof, and wherein the controlled-release layer comprises the oxycodone, or a pharmaceutically acceptable salt thereof. In another instance, about 70% of the promethazine or pharmaceutically acceptable salt thereof is capable of dissolving in a liquid solution in about 5 minutes after contact with the solution, and wherein about 30% of the oxycodone or pharmaceutically acceptable salt is capable of dissolving in a liquid solution in about 10 minutes after contact with the solution. In another instance, the controlled-release layer further comprises an antiemetic agent.

In some instances, the effective amount of hydrocodone or pharmaceutically acceptable salt thereof is an amount effective for treating or preventing pain for a period of about 4-8 hours following administration to a subject (e.g., 4-6, 4-8, 6-8 hours). In another instance, the controlled-release layer comprises about 7.5 mg of hydrocodone or a pharmaceutically acceptable salt thereof, about 360 mg of acetaminophen or a pharmaceutically acceptable salt thereof, about 152 mg of silicified microcrystalline cellulose, about 20 mg of hydroxy methyl propyl cellulose, about 2.7 mg of magnesium stearate, and about 2.7 mg of stearic acid; and the immediate-release layer comprises about 12.5 mg of promethazine or a pharmaceutically acceptable salt thereof, about 121.5 mg of silicified microcrystalline cellulose, about 15 mg of croscarmellose sodium and about 1 mg of magnesium stearate. In another instance, the controlled-release layer comprises about 7.5 mg of hydrocodone or a pharmaceutically acceptable salt thereof, about 152 mg of silicified microcrystalline cellulose, about 20 mg of hydroxy methyl propyl cellulose, about 2.7 mg of magnesium stearate, and about 2.7 mg of stearic acid; and the immediate-release layer comprises about 12.5 mg of promethazine or a pharmaceutically acceptable salt thereof, about 121.5 mg of silicified microcrystalline cellulose, about 15 mg of croscarmellose sodium and about 1 mg of magnesium stearate.

In some instances, a pharmaceutical composition comprises an effective amount of naltrexone or a pharmaceutically acceptable salt thereof. In another instance, the pharmaceutical composition is in the form of a bi-layer tablet. In another instance, the pharmaceutical composition is in the form of a two layer tablet. In another instance, the effective amount of the morphine or pharmaceutically acceptable salt thereof is an amount effective for treating or preventing pain for a period of about 4-8 hours following administration to a subject (e.g., 4-6, 4-8, 6-8 hours).

In some instances, a controlled-release layer comprises about 7.5 mg of hydrocodone or a pharmaceutically acceptable salt thereof, and about 360 mg of acetaminophen or a pharmaceutically acceptable salt thereof; and further wherein the immediate-release layer comprises about 12 mg of promethazine or a pharmaceutically acceptable salt thereof. In some instances, the controlled-release layer comprises about 7.5 mg of hydrocodone or a pharmaceutically acceptable salt thereof; and further wherein the immediate-release layer comprises about 12 mg of promethazine or a pharmaceutically acceptable salt thereof. In some instances, an effective amount is an amount effective for treating or preventing pain for a period of about 4-8 hours following administration to a subject (e.g., 4-6, 4-8, 6-8 hours) in need thereof. In some instances, an effective amount of the oxycodone or pharmaceutically acceptable salt thereof is an amount effective for treating or preventing pain for a period of about 4-8 hours following administration to a subject (e.g., 4-6, 4-8, 6-8 hours) in need thereof.

### Combination Formulations

Some instances of the disclosure are directed to pharmaceutical compositions comprising an effective amount of each of an analgesic and an active agent that is useful for reducing an adverse effect associated with the analgesic, such as one or more opioid analgesics, or one or more non-opioid analgesic. Such additional active agents include an antiemetic. In some instances, an analgesic is an opioid or non-opioid analgesic (e.g., hydrocodone or oxycodone, or a pharmaceutically acceptable salt thereof and acetaminophen or a pharmaceutically acceptable salt thereof). In a further instance, the active agent which reduces adverse effects of such analgesics is promethazine or a pharmaceutically acceptable salt thereof. In some instances, a pharmaceutical composition disclosed herein allows for higher dosages for an analgesic in a pharmaceutical composition, by reducing adverse effects associated with an opioid or non-opioid analgesic. For example, in a subject who could not otherwise tolerate a particular dosage of an opioid analgesic, it is believed that a pharmaceutical composition disclosed herein comprising an effective amount of each of an opioid analgesic, a non-opioid analgesic and promethazine or a pharmaceutically acceptable salt thereof, reduces an adverse effects (e.g. nausea, retching, vomiting, sleep disturbance, or constipation) associated with an opioid analgesic, thus allowing for increased dosages to be administered. Furthermore, is some instances, administration is through a single pharmaceutical composition.

In some instances, a pharmaceutical composition includes an opioid analgesic agent. In such instances, the opioid analgesic can comprise hydrocodone, oxycodone, acetyldihydrocodeinone, diamorphine, codeine, pethidine, alfentanil, codeine, fentanyl, hydromorphone, levorphanol, meperidine, methadone, morphine sulfate, oxymorphone, propoxyphene, remifentanil, sufentanil, tapentadol, tramadol, or a pharmaceutically acceptable salt of any one of the foregoing, or any combination thereof. In some instances, the opioid analgesic agent is hydrocodone, oxycodone, propoxyphene, or fentanyl or a pharmaceutically acceptable salt of any one of the foregoing, or any combination thereof.

In some instances, a dosage form comprises an opioid analgesic disclosed herein and one or more antiemetics disclosed. In some instances, the dosage form comprises hydrocodone, oxycodone, or a pharmaceutically acceptable salt thereof and promethazine or a pharmaceutically acceptable salt thereof. In some instance, a dosage form comprises hydrocodone or oxycodone or a pharmaceutically acceptable salt of any one of the foregoing, or any combination thereof and one or more antiemetics disclosed herein. In some instances, the dosage form comprises an effective amount of each of one opioid analgesic and one antiemetic. In some instances, the opioid analgesic is present in an amount of about: 1 to 30 mg; 1 to 25 mg; 1 to 20 mg; 1 to 15 mg; 1 to 10 mg; 2 to 8 mg; 3 to 7 mg; 4 to 6 mg; 5 to 15 mg; 7 to 13 mg; 8 to 12 mg; 9 to 11 mg; 5 to 10 mg; 6 to 9 mg; 7 to 8 mg; 1 mg; 2 mg; 3 mg; 4 mg; 5 mg; 5.5 mg, 6 mg; 6.5 mg; 7 mg; 7.5 mg; 8 mg; 8.5 mg; 9 mg; 9.5 mg; 10 mg; 11 mg; 12 mg; 13 mg; 14 mg; 15 mg; 16 mg; 17 mg; 18 mg; 19 mg; 20 mg; 25 mg; or 30 mg. In some instances, the antiemetic is present in an amount of about: 1 to 50 mg; 1 to 45 mg; 1 to 40 mg; 1 to 30 mg; 1 to 25 mg; 1 to 20 mg; 1 to 15 mg; 5 to 20 mg; 10 to 15 mg; 20 to 30 mg; 15 to 35 mg; 10 to 40 mg; 5 mg; 6 mg; 7 mg; 8 mg; 9 mg; 10 mg; 11 mg; 12 mg; 12.5 mg; 13 mg; 14 mg; 15 mg; 16 mg; 17 mg; 18 mg; 19 mg; 20 mg; 25 mg; 30 mg; 35 mg; 40 mg; 45 mg; or 50 mg. In some instances, the dosage form is a bi-layer tablet comprising a controlled-release layer and an immediate-release layer. In some instances, the controlled-release layer comprises the opioid analgesic, and the immediate-release layer comprises the antiemetic. In some instances, the dosage form comprising a controlled-release matrix and an immediate-release matrix. In some instances, the controlled-release matrix comprises the opioid analgesic, and the immediate-release matrix comprises the antiemetic. In some instances, the controlled-release matrix further comprises a non-opioid analgesic.

In some instances, a pharmaceutical composition disclosed herein comprises an opioid antagonist agent or abuse deterrent agent such as nalmefene, naloxone, niacin, naltrexone or a pharmaceutically acceptable salt of any one of the foregoing, or any combination thereof. The pharmaceutical composition can further comprise an antitussive such as codeine or dextromethorphan, dextrorphan, or a pharmaceutically acceptable salt of any one of the foregoing, or any combination thereof.

In some instances, a pharmaceutical composition comprises an opioid analgesic and an antiemetic further comprises one or more of an abuse deterrent agent, a barbiturate, a non-opioid analgesic, a laxative, a stimulant, or any combination thereof. In some instances, the pharmaceutical composition comprises an opioid analgesic, an antiemetic, and an abuse deterrent agent. In another instance, the pharmaceutical composition comprises an opioid analgesic, an antiemetic, and a non-opioid analgesic. In some instances, the pharmaceutical composition comprises an opioid analgesic, an antiemetic, and a non-opioid analgesic. In another instance, the pharmaceutical composition comprises an opioid analgesic, an antiemetic, and a laxative. In some instances, the pharmaceutical composition comprises an opioid analgesic, an antiemetic, and a stimulant. In some instances, the pharmaceutical composition comprises an opioid analgesic, an antiemetic, a laxative, and a stimulant. In some instances, the pharmaceutical composition comprises an opioid analgesic, an antiemetic, a non-opioid analgesic, and a laxative. In another instance, the pharmaceutical composition comprises an opioid analgesic, an antiemetic, a non-opioid analgesic, a laxative, and a stimulant.

In some instances, a pharmaceutical composition disclosed herein comprises an effective amount of each of an opioid analgesic agent and a non-opioid analgesic agent, where the opioid analgesic agent/non-opioid analgesic agent is codeine/acetaminophen, codeine/acetylsalicylic acid, codeine/naproxen, codeine/ibuprofen, hydrocodone/acetaminophen, hydrocodone/ibuprofen, hydrocodone/naproxen, hydrocodone/acetylsalicylic acid, oxycodone/acetaminophen, oxycodone/acetylsalicylic acid, oxycodone/naproxen, oxycodone/ibuprofen, propoxyphene/acetylsalicylic acid, propoxyphene/ibuprofen, propoxyphene/acetaminophen, or propoxyphene/naproxen, wherein the opioid analgesic agent or non-opioid analgesic agent can be in the form of a pharmaceutically acceptable salt thereof. In some instances, the hydrocodone salt is hydrocodone bitartrate, the oxycodone salt is oxycodone HCl, and the naproxen salt is naproxen Na or Mg.

In some instances, a pharmaceutical compositions disclosed herein can further comprise one or more of an opioid antagonist agent, abuse deterrent agent, a barbiturate agent, a stimulant agent, a laxative agent, an antiemetic agent, or any combination thereof. In some instances, a pharmaceutical composition comprises an effective amount of an opioid analgesic agent (such as hydrocodone or oxycodone or a pharmaceutically acceptable salt thereof), a non-opioid analgesic agent (such as acetaminophen or naproxen or a pharmaceutically acceptable salt thereof) and an active agent useful for reducing or eliminating adverse effects, such as an antiemetic (e.g., promethazine or a pharmaceutically acceptable salt thereof) or an antiemetic, as described herein. In some instances, the pharmaceutical composition is in the form of a tablet that comprises an immediate-release layer and a controlled-release layer. In some instances, the pharmaceutical composition is in the form of a bi-layer tablet that comprises an immediate-release layer and a controlled-release layer. In some instances, the pharmaceutical composition is in the form of a two layer tablet that comprises an immediate-release layer and a controlled-release layer. In a further instance, the immediate-release layer comprises one or more of an opioid agent, a non-opioid analgesic agent and an active agent useful for reducing or eliminating adverse effects. In a further instance, a controlled-release layer comprises an effective amount of one or more of an opioid agent, a non-opioid analgesic agent and an active agent useful for reducing or eliminating adverse effects associated with administration of an opioid analgesic agent or non-opioid analgesic agent. In some instances, a pharmaceutical composition further comprises an effective amount of an opioid antagonist agent or abuse deterrent agent. In a specific instance, the pharmaceutical composition comprises hydrocodone or oxycodone, or a pharmaceutically acceptable salt thereof, acetaminophen or a pharmaceutically acceptable salt thereof, or naproxen or a pharmaceutically acceptable salt thereof, and promethazine or a pharmaceutically acceptable salt thereof. In a specific instance, the pharmaceutical composition comprises hydrocodone or oxycodone, or a pharmaceutically acceptable salt thereof, and promethazine or a pharmaceutically acceptable salt thereof. In a specific instance, the pharmaceutical composition comprises hydrocodone or oxycodone, or a pharmaceutically acceptable salt thereof, acetaminophen or a pharmaceutically acceptable salt thereof, and promethazine or a pharmaceutically acceptable salt thereof. In a specific instance, the pharmaceutical composition comprises hydrocodone or oxycodone, or a pharmaceutically acceptable salt thereof, naproxen or a pharmaceutically acceptable salt thereof, and promethazine or a pharmaceutically acceptable salt thereof.

In some instances, an agent useful for preventing or alleviating an adverse effect associated with administration of an opioid analgesic or a non-opioid analgesic, a triptan, a barbiturate or a morphine narcotic, includes, for example, an antihistamine including a histamine agonist and an antagonist which is classified according to receptor subtype. The agent useful for preventing or suppressing an adverse effect can also include an H1, H2, H3, or H4 histamine antagonist.

In some instances, a pharmaceutical composition comprises two, three, four, five, six or more active agents. In some instances, at least one of the active agents is an antiemetic such as promethazine or a pharmaceutically acceptable salt thereof. As indicated herein, a pharmaceutical composition can comprise pharmaceutically active agents herein in any combinations. In some instances, a pharmaceutical composition comprises at least two analgesics; and one or more additional pharmaceutically active agents disclosed herein. In some instances, the pharmaceutical composition further comprises one antiemetic. In some instances, a pharmaceutical composition comprises a stimulant agent. In some instances, a pharmaceutical composition comprises a stimulant agent that provides an anti-sedative effect.

In some instances, a pharmaceutical composition comprised an effective amount of an opioid (such as hydrocodone, propoxyphene, fentanyl or oxycodone, or a pharmaceutically acceptable salt of any one of the foregoing, or any combination thereof) and a stimulant (such as modafinil or caffeine, or a pharmaceutically acceptable salt of any one of the foregoing, or any combination thereof). In some instances, a pharmaceutical composition comprises an antiemetic. In some instances, the antiemetic is promethazine or a pharmaceutically acceptable salt thereof. In yet another instance, the pharmaceutical composition further comprises a non-analgesic agent disclosed herein. In some instances, the non-opioid analgesic is acetaminophen or a pharmaceutically acceptable salt thereof, or naproxen or a pharmaceutically acceptable salt thereof.

In some instances, a pharmaceutical composition is in the form of a bi-layer tablet comprising an immediate-release layer and a controlled-release layer, wherein the immediate-release layer comprises and/or the chronic-release layer comprise a stimulant agent. In some instances, the controlled-release layer comprises an opioid agent. In some instances, the controlled-release layer further comprises an effective amount of a second or same stimulant agent as compared to the immediate-release layer. In some instances, the immediate-release layer and/or the controlled-release layer further comprises an antiemetic agent. In some instances, the immediate-release layer comprises an effective amount of one or more of an opioid agent, a stimulant agent and an antiemetic agent. In some instances, a controlled-release layer comprises an effective amount of one or more of an opioid agent, a stimulant agent, and an antiemetic agent. In some instances, the pharmaceutical composition further comprises an effective amount of an opioid antagonist agent or abuse deterrent agent.

In a specific instance, a pharmaceutical composition is provided that comprises hydrocodone or oxycodone, or a pharmaceutically acceptable salt of any one of the foregoing, or any combination thereof, modafinil or caffeine or a pharmaceutically acceptable salt of any one of the foregoing, or any combination thereof and promethazine or a pharmaceutically acceptable salt thereof. In a specific instance, a pharmaceutical composition is provided that comprises hydrocodone or oxycodone, or a pharmaceutically acceptable salt of any one of the foregoing, or any combination thereof, and modafinil or caffeine or a pharmaceutically acceptable salt of any one of the foregoing, or any combination thereof.

In another instance, a pharmaceutical composition is provided that comprises an effective amount of an opioid agent (such as hydrocodone, propoxyphene, fentanyl or oxycodone, or a pharmaceutically acceptable salt of any one of the foregoing, or any combination thereof); a non-opioid agent (such as acetaminophen or naproxen, or a pharmaceutically acceptable salt of any one of the foregoing, or any combination thereof); a barbiturate agent (such as butalbital, or a pharmaceutically acceptable salt thereof) and an antiemetic (such as promethazine, or a pharmaceutically acceptable salt thereof). In another instance, a pharmaceutical composition is provided that comprises an effective amount of an opioid agent (such as hydrocodone, propoxyphene, fentanyl or oxycodone, or a pharmaceutically acceptable salt of any one of the foregoing, or any combination thereof); a non-opioid agent (such as acetaminophen or naproxen, or a pharmaceutically acceptable salt of any one of the foregoing, or any combination thereof); and a barbiturate agent (such as butalbital, or a pharmaceutically acceptable salt thereof).

In a further instance, a pharmaceutical composition is in the form of a bi-layer tablet, wherein the pharmaceutical composition comprises an effective amount of each of an opioid agent, a non-opioid analgesic agent, a barbiturate agent and an antiemetic agent. In some instances, the bi-layer tablet comprises an immediate-release layer and a controlled-release layer. In some instances, the two layer tablet comprises an immediate-release layer and a controlled-release layer. In a further instance, the immediate-release layer comprises an effective amount of one or more of an opioid agent, a non-opioid analgesic agent, a barbiturate agent and an antiemetic agent. In another further instance, a controlled-release layer comprises an effective amount of one or more of an opioid agent, a barbiturate agent, a non-opioid analgesic agent, and an antiemetic agent. In some instances, a pharmaceutical composition further comprises an effective amount of an opioid antagonist agent or abuse deterrent agent. In a specific instance, a pharmaceutical composition comprises hydrocodone or oxycodone, or a pharmaceutically acceptable salt of any one of the foregoing, or any combination thereof, acetaminophen, or a pharmaceutically acceptable salt thereof, butalbital or a pharmaceutically acceptable salt thereof and promethazine or a pharmaceutically acceptable salt thereof. In a specific instance, a pharmaceutical composition comprises hydrocodone or oxycodone, or a pharmaceutically acceptable salt of any one of the foregoing, or any combination thereof, acetaminophen, or a pharmaceutically acceptable salt thereof, and butalbital or a pharmaceutically acceptable salt thereof.

In another instance, a pharmaceutical composition comprises an effective amount of each of an opioid agent (such as hydrocodone, propoxyphene, fentanyl or oxycodone or a pharmaceutically acceptable salt of any one of the foregoing, or any combination thereof); a barbiturate agent (such as butalbital or a pharmaceutically acceptable salt thereof); a stimulant agent (such as modafinil or caffeine or a pharmaceutically acceptable salt of any one of the foregoing, or any combination thereof); and a non-opioid agent (such as acetaminophen or naproxen or a pharmaceutically acceptable salt of any one of the foregoing, or any combination thereof). In another instance, a pharmaceutical composition comprises an effective amount of each of an opioid agent (such as hydrocodone, propoxyphene, fentanyl or oxycodone or a pharmaceutically acceptable salt of any one of the foregoing, or any combination thereof); a barbiturate agent (such as butalbital or a pharmaceutically acceptable salt thereof); and a stimulant agent (such as modafinil or caffeine or a pharmaceutically acceptable salt of any one of the foregoing, or any combination thereof). In some instances, the pharmaceutical composition further comprises an antiemetic (such as promethazine or a pharmaceutically acceptable salt thereof).

In some instances, a pharmaceutical composition is in the form of a bi-layer tablet, wherein the pharmaceutical composition comprises an effective amount of an opioid agent, a non-opioid analgesic agent, a barbiturate agent, a stimulant agent and an antiemetic agent. In some instances, such a pharmaceutical composition is in the form of a bi-layer tablet, wherein the pharmaceutical composition comprises an effective amount of an opioid agent, a non-opioid analgesic agent, a barbiturate agent, and a stimulant agent. In some instances, such a pharmaceutical composition is in the form of a two layer tablet, wherein the pharmaceutical composition comprises an effective amount of an opioid agent, a non-opioid analgesic agent, a barbiturate agent, a stimulant agent and an antiemetic agent. In some instances, such a pharmaceutical composition is in the form of a two layer tablet, wherein the pharmaceutical composition comprises an effective amount of an opioid agent, a non-opioid analgesic agent, a barbiturate agent, and a stimulant agent. In some instances, the bi-layer tablet comprises an immediate-release layer and a controlled-release layer. In some instances, the two layer tablet comprises an immediate-release layer and a controlled-release layer. In a further instance, the immediate-release layer comprises an effective amount of one or more of an opioid agent, a non-opioid analgesic agent, a barbiturate agent, a stimulant agent and an antiemetic agent. In another further instance, a controlled-release layer comprises an effective amount of one or more of an opioid agent, a non-opioid analgesic agent, a barbiturate agent, a stimulant agent and an antiemetic agent. In some instances, a pharmaceutical composition further comprises an effective amount of an opioid antagonist agent or abuse deterrent agent. In a specific instance, a pharmaceutical composition comprises hydrocodone, propoxyphene or oxycodone, or a pharmaceutically acceptable salt of any one of the foregoing, or any combination thereof; butalbital, naproxen, caffeine or a pharmaceutically acceptable salt of any one of the foregoing, or any combination thereof; and promethazine or a pharmaceutically acceptable salt thereof. In a specific instance, a pharmaceutical composition comprises hydrocodone, propoxyphene or oxycodone, or a pharmaceutically acceptable salt of any one of the foregoing, or any combination thereof; and butalbital, naproxen, caffeine or a pharmaceutically acceptable salt of any one of the foregoing, or any combination thereof.

In another instance, a pharmaceutical composition comprises an effective amount of an opioid agent (hydrocodone or oxycodone or a pharmaceutically acceptable salt of any one of the foregoing, or any combination thereof); and a barbiturate agent (such as butalbital or a pharmaceutically acceptable salt thereof). In some instances, a pharmaceutical composition further comprises an antiemetic (such as promethazine or a pharmaceutically acceptable salt thereof). In a further instance, the pharmaceutical composition is in the form of a bi-layer tablet, wherein the pharmaceutical composition comprises an effective amount of each of an opioid analgesic agent, a barbiturate agent, and an antiemetic agent. In a further instance, the pharmaceutical composition is in the form of a bi-layer tablet, wherein the pharmaceutical composition comprises an effective amount of each of an opioid analgesic agent, and a barbiturate agent. In some instances, the bi-layer tablet comprises an immediate-release layer and a controlled-release layer. In a further instance, the pharmaceutical composition is in the form of a two layer tablet, wherein the pharmaceutical composition comprises an effective amount of each of an opioid analgesic agent, a barbiturate agent, and an antiemetic agent. In a further instance, the pharmaceutical composition is in the form of a two layer tablet, wherein the pharmaceutical composition comprises an effective amount of each of an opioid analgesic agent, and a barbiturate agent. In some instances, the two layer tablet comprises an immediate-release layer and a controlled-release layer. In a further instance, the immediate-release layer comprises an effective amount of each of one or more of an opioid analgesic agent, a barbiturate agent, or an antiemetic agent. In another instance, a controlled-release layer comprises an effective amount of each of one or more of an opioid analgesic agent, a barbiturate agent, or an antiemetic agent. In some instances, the pharmaceutical composition further comprises an effective amount of an opioid antagonist agent or abuse deterrent agent. In a specific instance, a pharmaceutical composition comprises butalbital, hydrocodone or oxycodone, or a pharmaceutically acceptable salt of any one of the foregoing, or any combination thereof and promethazine or a pharmaceutically acceptable salt thereof. In a specific instance, a pharmaceutical composition comprises butalbital, hydrocodone or oxycodone, or a pharmaceutically acceptable salt of any one of the foregoing, or any combination thereof.

In another instance, a pharmaceutical composition comprises an effective amount of a non-opioid agent (such as acetaminophen, naproxen or ibuprofen or a pharmaceutically acceptable salt of any one of the foregoing, or any combination thereof); a barbiturate agent (such as butalbital or a pharmaceutically acceptable salt thereof); and an antiemetic (such as promethazine or a pharmaceutically acceptable salt thereof). In some instances, the pharmaceutical composition comprises about 50 mg butalbital or a pharmaceutically acceptable salt thereof, about 325 mg N-Acetyl-p-Aminophenol or a pharmaceutically acceptable salt thereof, and about 12.5 mg promethazine or a pharmaceutically acceptable salt thereof. In some instances, the promethazine salt is promethazine HCl.

In another instance, a pharmaceutical composition comprises an effective amount of each of a non-opioid agent (such as acetaminophen, naproxen or ibuprofen or a pharmaceutically acceptable salt of any one of the foregoing, or any combination thereof); a barbiturate agent (such as butalbital or a pharmaceutically acceptable salt thereof); and a stimulant agent (such as modafinil or caffeine or a pharmaceutically acceptable salt of any one of the foregoing, or any combination thereof). In some instances, the pharmaceutical composition further comprises an antiemetic (such as promethazine or a pharmaceutically acceptable salt thereof). In a further instance, an effective amount of a pharmaceutical composition is in the form of a bi-layer tablet, wherein the pharmaceutical composition comprises an effective amount of each of a non-opioid analgesic agent, a barbiturate agent, a stimulant agent and an antiemetic agent. In a further instance, an effective amount of a pharmaceutical composition is in the form of a bi-layer tablet, wherein the pharmaceutical composition comprises an effective amount of each of a non-opioid analgesic agent, a barbiturate agent, and a stimulant agent. In some instances, the bi-layer tablet comprises an immediate-release layer and a controlled-release layer. In a further instance, an effective amount of a pharmaceutical composition is in the form of a two layer tablet, wherein the pharmaceutical composition comprises an effective amount of each of a non-opioid analgesic agent, a barbiturate agent, a stimulant agent and an antiemetic agent. In a further instance, an effective amount of a pharmaceutical composition is in the form of a two layer tablet, wherein the pharmaceutical composition comprises an effective amount of each of a non-opioid analgesic agent, a barbiturate agent, and a stimulant agent. In some instances, the two layer tablet comprises an immediate-release layer and a controlled-release layer. In a further instance, the immediate-release layer comprises an effective amount of one or more of a non-opioid analgesic agent, a barbiturate agent, a stimulant agent or an antiemetic agent. In a further instance, a controlled-release layer comprises one or more of a non-opioid analgesic agent, a barbiturate agent, stimulant agent or an antiemetic agent. In a specific instance, a pharmaceutical composition comprises butalbital, naproxen, caffeine, or a pharmaceutically acceptable salt of any one of the foregoing, or any combination thereof and promethazine or a pharmaceutically acceptable salt thereof. In a specific instance, a pharmaceutical composition comprises butalbital, naproxen, caffeine, or a pharmaceutically acceptable salt of any one of the foregoing, or any combination thereof.

In another instance, a pharmaceutical composition comprises an effective amount of a barbiturate agent (such as butalbital or a pharmaceutically acceptable salt thereof) and a stimulant agent (such as modafinil or caffeine or a pharmaceutically acceptable salt of any one of the foregoing, or any combination thereof). In some instances, the pharmaceutical composition further comprises an antiemetic (such as promethazine or a pharmaceutically acceptable salt thereof). In another instance, a pharmaceutical composition is in the form of a bi-layer tablet, wherein the pharmaceutical composition comprises an effective amount of each of a barbiturate agent, a stimulant agent and an antiemetic agent. In another instance, a pharmaceutical composition is in the form of a bi-layer tablet, wherein the pharmaceutical composition comprises an effective amount of each of a barbiturate agent, and a stimulant agent. In some instances, the bi-layer tablet comprises an immediate-release layer and a controlled-release layer. In another instance, a pharmaceutical composition is in the form of a two layer tablet, wherein the pharmaceutical composition comprises an effective amount of each of a barbiturate agent, a stimulant agent and an antiemetic agent. In another instance, a pharmaceutical composition is in the form of a two layer tablet, wherein the pharmaceutical composition comprises an effective amount of each of a barbiturate agent, and a stimulant agent. In some instances, the two layer tablet comprises an immediate-release layer and a controlled-release layer. In a further instance, the immediate-release layer comprises an effective amount of each of one or more of a barbiturate agent, a stimulant agent or an antiemetic agent. In another instance, a controlled-release layer comprises an effective amount of each of one or more of a barbiturate agent, stimulant agent or an antiemetic agent. In a specific instance, a pharmaceutical composition comprises butalbital or a pharmaceutically acceptable salt thereof, caffeine or a pharmaceutically acceptable salt thereof and promethazine or a pharmaceutically acceptable salt thereof. In a specific instance, a pharmaceutical composition comprises butalbital or a pharmaceutically acceptable salt thereof, and caffeine or a pharmaceutically acceptable salt thereof.

In another instance, a pharmaceutical composition comprises an effective amount of a non-opioid agent (such as ibuprofen or naproxen or a pharmaceutically acceptable salt of any one of the foregoing, or any combination thereof) and a stimulant agent (such as modafinil or caffeine or a pharmaceutically acceptable salt of any one of the foregoing, or any combination thereof). In some instances, the pharmaceutical composition further comprises an antiemetic (such as promethazine or a pharmaceutically acceptable salt thereof). In some instances, the pharmaceutical composition is in the form of a bi-layer tablet, wherein the pharmaceutical composition comprises an effective amount of each of a non-opioid agent, a stimulant agent and an antiemetic agent. In some instances, the pharmaceutical composition is in the form of a bi-layer tablet, wherein the pharmaceutical composition comprises an effective amount of each of a non-opioid agent, and a stimulant agent. In some instances, the bi-layer tablet comprises an immediate-release layer and a controlled-release layer. In some instances, the pharmaceutical composition is in the form of a two layer tablet, wherein the pharmaceutical composition comprises an effective amount of each of a non-opioid agent, a stimulant agent and an antiemetic agent. In some instances, the pharmaceutical composition is in the form of a two layer tablet, wherein the pharmaceutical composition comprises an effective amount of each of a non-opioid agent, and a stimulant agent. In some instances, the two layer tablet comprises an immediate-release layer and a controlled-release layer. In a further instance, the immediate-release layer comprises an effective amount of each of one or more of a non-opioid agent, a stimulant agent or an antiemetic agent. In another further instance, the controlled-release layer comprises an effective amount of each of one or more of a non-opioid agent, stimulant agent or an antiemetic agent. In a specific instance, a pharmaceutical composition comprises naproxen or a pharmaceutically acceptable salt thereof and caffeine or a pharmaceutically acceptable salt thereof and promethazine or a pharmaceutically acceptable salt thereof. In a specific instance, a pharmaceutical composition comprises naproxen or a pharmaceutically acceptable salt thereof and caffeine or a pharmaceutically acceptable salt thereof.

In some instances, a pharmaceutical composition comprises one or more beta blockers, serotonin receptor agonists, vasoconstrictors, anti-platelet agents, anti-convulsants, ergots, or calcitonin-gene-related peptide (CGRP) receptor antagonists. In some instances, a pharmaceutical composition is administered to a subject in need thereof in a single dosage form which comprises one or more active agents and one or more beta blockers, serotonin receptor agonists, vasoconstrictors, anti-platelet agents, anti-convulsants, ergot alkaloids, and calcitonin-gene-related peptide (CGRP) receptor antagonists. In some instances, a single dosage form is a multilayered tablet which comprises one or more pharmaceutically active agents which includes one or more beta blockers, serotonin receptor agonists, vasoconstrictors, anti-platelet agents, anti-convulsants, ergot alkaloids, or calcitonin-gene-related peptide (CGRP) receptor antagonists. In some instances, a multilayer tablet comprises at least one immediate-release layer and at least one controlled-release layer. Pharmaceutical compositions herein can be administered using other dosage forms disclosed herein. In yet other instances, a pharmaceutical composition comprises one or more active agents disclosed herein are administered prior to, concurrent with, or after administration of one or more beta blockers, serotonin receptor agonists, vasoconstrictors, anti-platelet agents, anti-convulsants, ergot alkaloids, or calcitonin-gene-related peptide (CGRP) receptor antagonists. In some instances, the present methods for treating or preventing pain further comprise administering an effective amount of one or more beta blockers, serotonin receptor agonists, vasoconstrictors, anti-platelet agents, anti-convulsants, ergots, or CGRP receptor antagonists.

In some instances, a pharmaceutical composition can comprise one or more active agents comprise an opioid analgesic, an antiemetic, and a laxative. In some instances, the laxative is present in an amount effective to reduce or eliminate constipation. In some instances, the laxative is present in an amount effective to reduce or eliminate opioid induced constipation. The laxative can be a bulk-producing agent, a stool softener, a lubricant, a hydrating agent, a stimulant or irritant, a serotonin agonist, a chloride channel activator. In some instances, the pharmaceutical compositions further comprise a non-opioid analgesic, a barbiturate, an anti-abuse agent, a stimulant, or any combination thereof.

In some instances, a total weight of a composition herein (e.g., a tablet such as a bi-layer tablet) is from about 100 to about 1500 mg, such as about: 100, 200, 300, 350, 400, 450, 500, 550, 600, 650, 700, 750, 800, 850, 900, 950, 1000, 1100, 1200, 1300, 1400, or 1500 mg; or about: 650-750, 600-800, 500-900, or 400-1000 mg. In some instances, a total weight of a composition (e.g., a tablet such as a bi-layer tablet) is about 700 mg.

### Dosage

In some instances, a pharmaceutical composition disclosed herein is administered at a single dose or multiple doses. In some instances, the pharmaceutical composition is administered 2 to 6 times per day, or at least: 2, 3, 4, 5, or 6 times per day. In some instances, the pharmaceutical composition is administered about every 4 to 6 hours. In some instances, the pharmaceutical composition is administered about every 8 hours or less often.

In some instances, a pharmaceutical composition disclosed herein comprises multiple active agents at the same or different dosages. In some instances, the analgesic components can vary in dosages as further described herein, and the antiemetic dosage can be adjusted according to the particular analgesics used. In some instances, a pharmaceutical composition comprises an opioid analgesic agent that is present in a single dose from of about 0.05 mg to about 130 mg, including but not limited to about: 0.05 mg, 0.1 mg, 0.2 mg, 0.3 mg, 0.4 mg, 0.5 mg, 0.6 mg, 0.7 mg, 0.8 mg, 0.9 mg, 1.0 mg, 1.2 mg, 1.5 mg, 2.5 mg, 3.0 mg, 4.0 mg, 4.5 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, or 130 mg. In some instances, the opioid analgesic agent is hydrocodone, oxycodone, tapentadol, fentanyl or a pharmaceutically acceptable salt thereof. In another instance, the opioid analgesic agent is present in a bi-layer tablet that comprises an immediate-release and a controlled-release layer. In another instance, the opioid analgesic agent is present in a two layer tablet that comprises an immediate-release and a controlled-release layer.

In another instance, a pharmaceutical composition comprises a non-opioid analgesic that is present in a single dose from about 200 mg to about 1000 mg, including but not limited to about: 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. In some instances, the non-opioid analgesic agent is present in a bi-layer tablet that comprises an immediate-release and a controlled-release layer. In some instances, the non-opioid analgesic agent is present in a two layer tablet that comprises an immediate-release and a controlled-release layer.

In another instance, a pharmaceutical composition comprises an antiemetic agent (e.g., promethazine or a pharmaceutically acceptable salt thereof) that is present in a single dose from about 0.5 mg to about 200 mg, including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some instances, the antiemetic agent is present in a bi-layer tablet that comprises an immediate-release and a controlled-release layer. In some instances, the antiemetic agent is present in a two layer tablet that comprises an immediate-release and a controlled-release layer.

In some instances, a pharmaceutical composition disclosed herein comprises an opioid analgesic agent (such as hydrocodone), a pharmaceutically acceptable salt or its thiosemicarbazone, p-nitrophenylhydrazone, o-methyloxime, semicarbazone, or bis (methylcarbamate) (each of the foregoing being a hydrocodone agent or derivative); acetaminophen; and promethazine or salt thereof. In some cases, the opioid analgesic agent is present in a single dose from about 0.05 mg to about 130 mg, including but not limited to about: 0.05 mg, 0.1 mg, 0.2 mg, 0.3 mg, 0.4 mg, 0.5 mg, 0.6 mg, 0.7 mg, 0.8 mg, 0.9 mg, 1 mg, 1.2 mg, 1.5 mg, 2.5 mg, 3.0 mg, 4.0 mg, 4.8355 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, or 130 mg.

In some instances, a pharmaceutical composition disclosed herein comprises acetaminophen or a pharmaceutically acceptable salt thereof that is present in a single dose from about 200 mg to about 1000 mg, including but not limited to about: 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. In some cases, the promethazine or salt thereof is present in the pharmaceutical composition at a single dose between about 0.5 mg to about 200 mg, including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some instances, hydrocodone or a pharmaceutically acceptable salt thereof, acetaminophen or a pharmaceutically acceptable salt thereof, and promethazine or a pharmaceutically acceptable salt thereof are present in a bi-layer tablet that comprises an immediate-release and a controlled-release layer. In some instances, hydrocodone or a pharmaceutically acceptable salt thereof, acetaminophen or a pharmaceutically acceptable salt thereof, and promethazine or a pharmaceutically acceptable salt thereof are present in a two layer tablet that comprises an immediate-release and a controlled-release layer. In some instances, the immediate-release layer comprises promethazine or a pharmaceutically acceptable salt thereof and the controlled-release layer comprises hydrocodone or a pharmaceutically acceptable salt thereof and acetaminophen or a pharmaceutically acceptable salt thereof.

In some instances, a pharmaceutical composition comprises an opioid analgesic, an antiemetic, without a non-opioid analgesic. In some instances, the opioid analgesic is hydrocodone or a pharmaceutically acceptable salt thereof and the antiemetic is promethazine or a pharmaceutically acceptable salt thereof. In some instances, the pharmaceutical composition comprises about 5 mg of hydrocodone or a pharmaceutically acceptable salt thereof and about 12.5 mg of promethazine or a pharmaceutically acceptable salt thereof. In some instances, the pharmaceutical composition comprises about 5 mg of hydrocodone bitartrate and about 12.5 mg of promethazine hydrochloride. In some instances, the pharmaceutical composition comprises about 7.5 mg of hydrocodone or a pharmaceutically acceptable salt thereof and about 12.5 mg of promethazine or a pharmaceutically acceptable salt thereof. In some instances, the pharmaceutical composition comprises about 7.5 mg of hydrocodone bitartrate and about 12.5 mg of promethazine hydrochloride. In some instances, the pharmaceutical composition comprises about 10 mg of hydrocodone or a pharmaceutically acceptable salt thereof and about 12.5 mg of promethazine or a pharmaceutically acceptable salt thereof. In some instances, the pharmaceutical composition comprises about 10 mg of hydrocodone bitartrate and about 12.5 mg of promethazine hydrochloride.

In some instances, a pharmaceutical composition comprises an opioid analgesic, an antiemetic, and a non-opioid analgesic. In some instances the opioid analgesic is hydrocodone or a pharmaceutically acceptable salt thereof, the antiemetic is promethazine or a pharmaceutically acceptable salt thereof, and the non-opioid analgesic is acetaminophen or a pharmaceutically acceptable salt thereof. In some instances, the pharmaceutical composition comprises about 5 mg of hydrocodone or a pharmaceutically acceptable salt thereof, about 12.5 mg of promethazine or a pharmaceutically acceptable salt thereof, and about 325 mg of acetaminophen or a pharmaceutically acceptable salt thereof. In some instances, the pharmaceutical composition comprises about 5 mg of hydrocodone bitartrate, about 12.5 mg of promethazine hydrochloride, and about 325 mg of acetaminophen. In some instances, the pharmaceutical composition comprises about 7.5 mg of hydrocodone or a pharmaceutically acceptable salt thereof, about 12.5 mg of promethazine or a pharmaceutically acceptable salt thereof, and about 325 mg of acetaminophen or a pharmaceutically acceptable salt thereof. In some instances, the pharmaceutical composition comprises about 7.5 mg of hydrocodone bitartrate, about 12.5 mg of promethazine hydrochloride, and about 325 mg of acetaminophen. In some instances, the pharmaceutical composition comprises about 10 mg of hydrocodone or a pharmaceutically acceptable salt thereof, about 12.5 mg of promethazine or a pharmaceutically acceptable salt thereof, and about 325 mg of acetaminophen or a pharmaceutically acceptable salt thereof. In some instances, the pharmaceutical composition comprises about 10 mg of hydrocodone bitartrate, about 12.5 mg of promethazine hydrochloride, and about 325 mg of acetaminophen.

In some instances, a pharmaceutical composition comprises an opioid analgesic and a non-opioid analgesic. In some instances the opioid analgesic is hydrocodone or a pharmaceutically acceptable salt thereof and the non-opioid analgesic is acetaminophen or a pharmaceutically acceptable salt thereof. In some instances, the pharmaceutical composition comprises about 300 mg acetaminophen or a pharmaceutically acceptable salt thereof and about 10 mg hydrocodone or a pharmaceutically acceptable salt thereof. In some instances, the pharmaceutical composition comprises about 300 mg acetaminophen and about 10 mg hydrocodone bitartrate. In some instances, the pharmaceutical composition comprises about 325 mg acetaminophen or a pharmaceutically acceptable salt thereof and about 7.5 mg hydrocodone or a pharmaceutically acceptable salt thereof. In some instances, the pharmaceutical composition comprises about 325 mg acetaminophen and about 7.5 mg hydrocodone bitartrate. In some instances, the pharmaceutical composition comprises about 325 mg acetaminophen or a pharmaceutically acceptable salt thereof and about 10 mg hydrocodone or a pharmaceutically acceptable salt thereof. In some instances, the pharmaceutical composition comprises about 325 mg acetaminophen and about 10 mg hydrocodone bitartrate. In some instances, the pharmaceutical composition comprises about 325 mg acetaminophen or a pharmaceutically acceptable salt thereof and about 5 mg hydrocodone or a pharmaceutically acceptable salt thereof. In some instances, the pharmaceutical composition comprises about 325 mg acetaminophen and about 5 mg hydrocodone bitartrate. In some instances, the pharmaceutical composition comprises about 300 mg acetaminophen or a pharmaceutically acceptable salt thereof and about 5 mg hydrocodone or a pharmaceutically acceptable salt thereof. In some instances, the pharmaceutical composition comprises about 300 mg acetaminophen and about 5 mg hydrocodone bitartrate. In some instances, the pharmaceutical composition comprises about 300 mg acetaminophen or a pharmaceutically acceptable salt thereof and about 7.5 mg hydrocodone or a pharmaceutically acceptable salt thereof. In some instances, the pharmaceutical composition comprises about 300 mg acetaminophen and about 7.5 mg hydrocodone bitartrate. In some instances, the pharmaceutical composition comprises about 325 mg acetaminophen or a pharmaceutically acceptable salt thereof and about 2.5 mg hydrocodone or a pharmaceutically acceptable salt thereof. In some instances, the pharmaceutical composition comprises about 325 mg acetaminophen and about 2.5 mg hydrocodone bitartrate.

In some instances, a pharmaceutical composition comprises an opioid analgesic and a non-opioid analgesic. In some instances, the opioid analgesic is hydrocodone or a pharmaceutically acceptable salt thereof and the non-opioid analgesic is ibuprofen or a pharmaceutically acceptable salt thereof. In some instances, the pharmaceutical composition comprises about 200 mg ibuprofen or a pharmaceutically acceptable salt thereof and about 5 mg hydrocodone or a pharmaceutically acceptable salt thereof. In some instances, the pharmaceutical composition comprises about 200 mg ibuprofen and about 5 mg hydrocodone bitartrate. In some instances, the pharmaceutical composition comprises about 200 mg ibuprofen or a pharmaceutically acceptable salt thereof and about 7.5 mg hydrocodone or a pharmaceutically acceptable salt thereof. In some instances, the pharmaceutical composition comprises about 200 mg ibuprofen and about 7.5 mg hydrocodone bitartrate. In some instances, the pharmaceutical composition comprises about 200 mg ibuprofen or a pharmaceutically acceptable salt thereof and about 10 mg hydrocodone or a pharmaceutically acceptable salt thereof. In some instances, the pharmaceutical composition comprises about 200 mg ibuprofen and about 10 mg hydrocodone bitartrate. In some instances, the pharmaceutical composition comprises about 200 mg ibuprofen or a pharmaceutically acceptable salt thereof and about 2.5 mg hydrocodone or a pharmaceutically acceptable salt thereof. In some instances, the pharmaceutical composition comprises about 200 mg ibuprofen and about 2.5 mg hydrocodone bitartrate.

In some instances, a pharmaceutical composition comprises an opioid analgesic. In some instances, the opioid analgesic is hydrocodone or a pharmaceutically acceptable salt thereof. In some instances, the pharmaceutical composition comprises about 10 mg hydrocodone or a pharmaceutically acceptable salt thereof. In some instances, the pharmaceutical composition comprises about 10 mg hydrocodone bitartrate. In some instances, the pharmaceutical composition comprises about 15 mg hydrocodone or a pharmaceutically acceptable salt thereof. In some instances, the pharmaceutical composition comprises about 15 mg hydrocodone bitartrate. In some instances, the pharmaceutical composition comprises about 20 mg hydrocodone or a pharmaceutically acceptable salt thereof. In some instances, the pharmaceutical composition comprises about 20 mg hydrocodone bitartrate. In some instances, the pharmaceutical composition comprises about 30 mg hydrocodone or a pharmaceutically acceptable salt thereof. In some instances, the pharmaceutical composition comprises about 30 mg hydrocodone bitartrate. In some instances, the pharmaceutical composition comprises about 40 mg hydrocodone or a pharmaceutically acceptable salt thereof. In some instances, the pharmaceutical composition comprises about 40 mg hydrocodone bitartrate. In some instances, the pharmaceutical composition comprises about 50 mg hydrocodone or a pharmaceutically acceptable salt thereof. In some instances, the pharmaceutical composition comprises about 50 mg hydrocodone bitartrate. In some instances, the pharmaceutical composition comprises about 60 mg hydrocodone or a pharmaceutically acceptable salt thereof. In some instances, the pharmaceutical composition comprises about 60 mg hydrocodone bitartrate. In some instances, the pharmaceutical composition comprises about 80 mg hydrocodone or a pharmaceutically acceptable salt thereof. In some instances, the pharmaceutical composition comprises about 80 mg hydrocodone bitartrate. In some instances, the pharmaceutical composition comprises about 100 mg hydrocodone or a pharmaceutically acceptable salt thereof. In some instances, the pharmaceutical composition comprises about 100 mg hydrocodone bitartrate. In some instances, the pharmaceutical composition comprises about 120 mg hydrocodone or a pharmaceutically acceptable salt thereof. In some instances, the pharmaceutical composition comprises about 120 mg hydrocodone bitartrate.

In some instances, a pharmaceutical composition comprises an opioid analgesic and a non-opioid analgesic. In some instances, the opioid analgesic is oxycodone or a pharmaceutically acceptable salt and the non-opioid analgesic is acetaminophen or a pharmaceutically acceptable salt. In some instances, the pharmaceutical composition comprises about 325 mg acetaminophen or a pharmaceutically acceptable salt thereof and about 5 mg oxycodone or a pharmaceutically acceptable salt thereof. In some instances, the pharmaceutical composition comprises about 325 mg acetaminophen and about 5 mg oxycodone hydrochloride. In some instances, the pharmaceutical composition comprises about 325 mg acetaminophen or a pharmaceutically acceptable salt thereof and about 7.5 mg oxycodone or a pharmaceutically acceptable salt thereof. In some instances, the pharmaceutical composition comprises about 325 mg acetaminophen and about 7.5 mg oxycodone hydrochloride. In some instances, the pharmaceutical composition comprises about 325 mg acetaminophen or a pharmaceutically acceptable salt thereof and about 10 mg oxycodone or a pharmaceutically acceptable salt thereof. In some instances, the pharmaceutical composition comprises about 325 mg acetaminophen and about 10 mg oxycodone hydrochloride. In some instances, the pharmaceutical composition comprises about 325 mg acetaminophen or a pharmaceutically acceptable salt thereof and about 2.5 mg oxycodone or a pharmaceutically acceptable salt thereof. In some instances, the pharmaceutical composition comprises about 325 mg acetaminophen and about 2.5 mg oxycodone hydrochloride. In some instances, the pharmaceutical composition comprises about 300 mg acetaminophen or a pharmaceutically acceptable salt thereof and about 10 mg oxycodone or a pharmaceutically acceptable salt thereof. In some instances, the pharmaceutical composition comprises about 300 mg acetaminophen and about 10 mg oxycodone hydrochloride. In some instances, the pharmaceutical composition comprises about 300 mg acetaminophen or a pharmaceutically acceptable salt thereof and about 2.5 mg oxycodone or a pharmaceutically acceptable salt thereof. In some instances, the pharmaceutical composition comprises about 300 mg acetaminophen and about 2.5 mg oxycodone hydrochloride. In some instances, the pharmaceutical composition comprises about 300 mg acetaminophen or a pharmaceutically acceptable salt thereof and about 5 mg oxycodone or a pharmaceutically acceptable salt thereof. In some instances, the pharmaceutical composition comprises about 300 mg acetaminophen and about 5 mg oxycodone hydrochloride. In some instances, the pharmaceutical composition comprises about 300 mg acetaminophen or a pharmaceutically acceptable salt thereof and about 7.5 mg oxycodone or a pharmaceutically acceptable salt thereof. In some instances, the pharmaceutical composition comprises about 300 mg acetaminophen and about 7.5 mg oxycodone hydrochloride.

In some instances, a pharmaceutical composition comprises an opioid analgesic and a non-opioid analgesic. In some instances, the opioid analgesic is oxycodone or a pharmaceutically acceptable salt and the non-opioid analgesic is acetylsalicylic acid or a pharmaceutically acceptable salt. In some instances, the pharmaceutical composition comprises about 325 mg acetylsalicylic acid or a pharmaceutically acceptable salt thereof and about 4.8355 mg oxycodone or a pharmaceutically acceptable salt thereof. In some instances, the pharmaceutical composition comprises about 325 mg acetylsalicylic acid and about 4.8355 mg oxycodone hydrochloride. In some instances, the opioid analgesic is oxycodone or a pharmaceutically acceptable salt and the non-opioid analgesic is ibuprofen or a pharmaceutically acceptable salt. In some instances, the pharmaceutical composition comprises about 400 mg ibuprofen or a pharmaceutically acceptable salt thereof and about 5 mg oxycodone or a pharmaceutically acceptable salt thereof. In some instances, the pharmaceutical composition comprises about 400 mg ibuprofen and about 5 mg oxycodone hydrochloride.

In some instances, a pharmaceutical composition comprises an opioid analgesic. In some instances, the opioid analgesic is oxycodone or a pharmaceutically acceptable salt. In some instances, the pharmaceutical composition comprises about 5 mg oxycodone or a pharmaceutically acceptable salt thereof. In some instances, the pharmaceutical composition comprises about 5 mg oxycodone hydrochloride. In some instances, the pharmaceutical composition comprises about 7.5 mg oxycodone or a pharmaceutically acceptable salt thereof. In some instances, the pharmaceutical composition comprises about 7.5 mg oxycodone hydrochloride. In some instances, the pharmaceutical composition comprises about 10 mg oxycodone or a pharmaceutically acceptable salt thereof. In some instances, the pharmaceutical composition comprises about 10 mg oxycodone hydrochloride. In some instances, the pharmaceutical composition comprises about 15 mg oxycodone or a pharmaceutically acceptable salt thereof. In some instances, the pharmaceutical composition comprises about 15 mg oxycodone hydrochloride. In some instances, the pharmaceutical composition comprises about 20 mg oxycodone or a pharmaceutically acceptable salt thereof. In some instances, the pharmaceutical composition comprises about 20 mg oxycodone hydrochloride. In some instances, the pharmaceutical composition comprises about 30 mg oxycodone or a pharmaceutically acceptable salt thereof. In some instances, the pharmaceutical composition comprises about 30 mg oxycodone hydrochloride. In some instances, the pharmaceutical composition comprises about 40 mg oxycodone or a pharmaceutically acceptable salt thereof. In some instances, the pharmaceutical composition comprises about 40 mg oxycodone hydrochloride. In some instances, the pharmaceutical composition comprises about 60 mg oxycodone or a pharmaceutically acceptable salt thereof. In some instances, the pharmaceutical composition comprises about 60 mg oxycodone hydrochloride. In some instances, the pharmaceutical composition comprises about 80 mg oxycodone or a pharmaceutically acceptable salt thereof. In some instances, the pharmaceutical composition comprises about 80 mg oxycodone hydrochloride. In some instances, the pharmaceutical composition comprises about 100 mg oxycodone or a pharmaceutically acceptable salt thereof. In some instances, the pharmaceutical composition comprises about 100 mg oxycodone hydrochloride.

In some instances, a pharmaceutical composition comprises an antiemetic. In some instances, the antiemetic is promethazine or a pharmaceutically acceptable salt thereof. In some instances, the pharmaceutical composition comprises about 25 mg promethazine or a pharmaceutically acceptable salt thereof. In some instances, the pharmaceutical composition comprises about 25 mg promethazine hydrochloride. In some instances, the pharmaceutical composition comprises about 50 mg promethazine or a pharmaceutically acceptable salt thereof. In some instances, the pharmaceutical composition comprises about 50 mg promethazine hydrochloride. In some instances, the pharmaceutical composition comprises about 12.5 mg promethazine or a pharmaceutically acceptable salt thereof. In some instances, the pharmaceutical composition comprises about 12.5 mg promethazine hydrochloride. In some instances, the pharmaceutical composition comprises about 6.25 mg promethazine or a pharmaceutically acceptable salt thereof. In some instances, the pharmaceutical composition comprises about 6.25 mg promethazine hydrochloride.

In some instances, a pharmaceutical composition comprises an opioid analgesic. In some instances, the opioid analgesic is tapentadol or a pharmaceutically acceptable salt thereof, such as tapentadol hydrochloride. In some instances, the pharmaceutical composition can comprise about 50 mg tapentadol or a pharmaceutically acceptable salt thereof, such as tapentadol hydrochloride. In some instances, the pharmaceutical composition can comprise about 75 mg tapentadol or a pharmaceutically acceptable salt thereof, such as tapentadol hydrochloride. In some instances, the pharmaceutical composition can comprise about 100 mg tapentadol or a pharmaceutically acceptable salt thereof, such as tapentadol hydrochloride. In some instances, the pharmaceutical composition can comprise about 150 mg tapentadol or a pharmaceutically acceptable salt thereof, such as tapentadol hydrochloride. In some instances, the pharmaceutical composition can comprise about 200 mg tapentadol or a pharmaceutically acceptable salt thereof, such as tapentadol hydrochloride. In some instances, the pharmaceutical composition can comprise about 250 mg tapentadol or a pharmaceutically acceptable salt thereof, such as tapentadol hydrochloride.

In some instances, a pharmaceutical composition comprises an opioid analgesic, a non-opioid analgesic, and an antiemetic. In some instances, the opioid analgesic is tramadol or a pharmaceutically acceptable salt thereof, such as tramadol hydrochloride. In some instances, the non-opioid analgesic is acetaminophen or a pharmaceutically acceptable salt thereof. In some instances, the antiemetic is promethazine or a pharmaceutically acceptable salt thereof. In some instances, the pharmaceutical composition can comprise about 37.5 mg tramadol or a pharmaceutically acceptable salt thereof, such as tramadol hydrochloride, about 325 mg of acetaminophen or a pharmaceutically acceptable salt thereof, and about 12.5 mg of promethazine or a pharmaceutically acceptable salt thereof. In some instances, the pharmaceutical composition can comprise about 37.5 mg tramadol or a pharmaceutically acceptable salt thereof, such as tramadol hydrochloride, and about 12.5 mg of promethazine or a pharmaceutically acceptable salt thereof. In some instances, the pharmaceutical composition can comprise about 37.5 mg tramadol or a pharmaceutically acceptable salt thereof, such as tramadol hydrochloride, and about 325 mg of acetaminophen or a pharmaceutically acceptable salt thereof.

In some instances, a pharmaceutical composition comprises about 50 mg tramadol or a pharmaceutically acceptable salt thereof, such as tramadol hydrochloride, about 325 mg of acetaminophen or a pharmaceutically acceptable salt thereof, and about 12.5 mg of promethazine or a pharmaceutically acceptable salt thereof. In some instances, the pharmaceutical composition can comprise about 50 mg tramadol or a pharmaceutically acceptable salt thereof, such as tramadol hydrochloride, and about 12.5 mg of promethazine or a pharmaceutically acceptable salt thereof. In some instances, the pharmaceutical composition can comprise about 50 mg tramadol or a pharmaceutically acceptable salt thereof, such as tramadol hydrochloride, and about 325 mg of acetaminophen or a pharmaceutically acceptable salt thereof.

In some instances, a pharmaceutical composition comprises about 100 mg tramadol or a pharmaceutically acceptable salt thereof, such as tramadol hydrochloride, about 325 mg of acetaminophen or a pharmaceutically acceptable salt thereof, and about 12.5 mg of promethazine or a pharmaceutically acceptable salt thereof. In some instances, the pharmaceutical composition can comprise about 100 mg tramadol or a pharmaceutically acceptable salt thereof, such as tramadol hydrochloride, and about 12.5 mg of promethazine or a pharmaceutically acceptable salt thereof. In some instances, the pharmaceutical composition can comprise about 100 mg tramadol or a pharmaceutically acceptable salt thereof, such as tramadol hydrochloride, and about 325 mg of acetaminophen or a pharmaceutically acceptable salt thereof.

In some instances, a pharmaceutical composition comprises about 200 mg tramadol or a pharmaceutically acceptable salt thereof, such as tramadol hydrochloride, about 325 mg of acetaminophen or a pharmaceutically acceptable salt thereof, and about 12.5 mg of promethazine or a pharmaceutically acceptable salt thereof. In some instances, the pharmaceutical composition can comprise about 200 mg tramadol or a pharmaceutically acceptable salt thereof, such as tramadol hydrochloride, and about 12.5 mg of promethazine or a pharmaceutically acceptable salt thereof. In some instances, the pharmaceutical composition can comprise about 200 mg tramadol or a pharmaceutically acceptable salt thereof, such as tramadol hydrochloride, and about 325 mg of acetaminophen or a pharmaceutically acceptable salt thereof.

In some instances, a pharmaceutical composition comprises about 300 mg tramadol or a pharmaceutically acceptable salt thereof, such as tramadol hydrochloride, about 325 mg of acetaminophen or a pharmaceutically acceptable salt thereof, and about 12.5 mg of promethazine or a pharmaceutically acceptable salt thereof. In some instances, the pharmaceutical composition can comprise about 300 mg tramadol or a pharmaceutically acceptable salt thereof, such as tramadol hydrochloride, and about 12.5 mg of promethazine or a pharmaceutically acceptable salt thereof. In some instances, the pharmaceutical composition can comprise about 300 mg tramadol or a pharmaceutically acceptable salt thereof, such as tramadol hydrochloride, and about 325 mg of acetaminophen or a pharmaceutically acceptable salt thereof.

In some instances, a pharmaceutical composition disclosed herein comprises an opioid analgesic agent (such as hydrocodone, oxycodone, tapentadol or a pharmaceutically acceptable salt of any one of the foregoing, or any combination thereof), acetaminophen or a pharmaceutically acceptable salt thereof and promethazine or a pharmaceutically acceptable salt thereof, wherein the pharmaceutical composition comprises the respective agents, opioid analgesic agent: acetaminophen or a salt thereof: promethazine or a pharmaceutically acceptable salt thereof in a ratio by weight of about (1 to 2):(40 to 45):(1 to 2), such as about: 1:40:1, 1:40:1.1, 1:40:1.2, 1:40:1.3, 1:40:1.4, 1:40:1.5, 1:40:1.6, 1:40:1.7, 1:40:1.8, 1:40:1.9, 1:40:2, 1.1:40:1, 1.2:40:1, 1.3:40:1, 1.4:40:1, 1.5:40:1, 1.6:40:1, 1.7:40:1, 1.8:40:1, 1.9:40:1, 2:40:1, 1:41:1, 1:41:1.1, 1:41:1.2, 1:41:1.3, 1:41:1.4, 1:41:1.5, 1:41:1.6, 1:41:1.7, 1:41:1.8, 1:41:1.9, 1:41:2, 1.1:41:1, 1.2:41:1, 1.3:41:1, 1.4:41:1, 1.5:41:1, 1.6:41:1, 1.7:41:1, 1.8:41:1, 1.9:41:1, 2:41:1, 1:42:1, 1:42:1.1, 1:42:1.2, 1:42:1.3, 1:42:1.4, 1:42:1.5, 1:42:1.6, 1:42:1.7, 1:42:1.8, 1:42:1.9, 1:42:2, 1.1:42:1, 1.2:42:1, 1.3:42:1, 1.4:42:1, 1.5:42:1, 1.6:42:1, 1.7:42:1, 1.8:42:1, 1.9:42:1, 2:42:1, 1:43:1, 1:43:1.1, 1:43:1.2, 1:43:1.3, 1:43:1.4, 1:43:1.5, 1:43:1.6, 1:43:1.7, 1:43:1.8, 1:43:1.9, 1:43:2, 1.1:43:1, 1.2:43:1, 1.3:43:1, 1.4:43:1, 1.5:43:1, 1.6:43:1, 1.7:43:1, 1.8:43:1, 1.9:43:1, 2:43:1, 1:43.1:1, 1:43.1:1.1, 1:43.1:1.2, 1:43.1:1.3, 1:43.1:1.4, 1:43.1:1.5, 1:43.1:1.6, 1:43.1:1.7, 1:43.1:1.8, 1:43.1:1.9, 1:43.1:2, 1.1:43.1:1, 1.2:43.1:1, 1.3:43.1:1, 1.4:43.1:1, 1.5:43.1:1, 1.6:43.1:1, 1.7:43.1:1, 1.8:43.1:1, 1.9:43.1:1, 2:43.1:1, 1:43.2:1, 1:43.2:1.1, 1:43.2:1.2, 1:43.2:1.3, 1:43.2:1.4, 1:43.2:1.5, 1:43.2:1.6, 1:43.2:1.7, 1:43.2:1.8, 1:43.2:1.9, 1:43.2:2, 1.1:43.2:1, 1.2:43.2:1, 1.3:43.2:1, 1.4:43.2:1, 1.5:43.2:1, 1.6:43.2:1, 1.7:43.2:1, 1.8:43.2:1, 1.9:43.2:1, 2:43.2:1, 1:43.3:1, 1:43.3:1.1, 1:43.3:1.2, 1:43.3:1.3, 1:43.3:1.4, 1:43.3:1.5, 1:43.3:1.6, 1:43.3:1.7, 1:43.3:1.8, 1:43.3:1.9, 1:43.3:2, 1.1:43.3:1, 1.2:43.3:1, 1.3:43.3:1, 1.4:43.3:1, 1.5:43.3:1, 1.6:43.3:1, 1.7:43.3:1, 1.8:43.3:1, 1.9:43.3:1, 2:43.3:1, 1:43.4:1, 1:43.4:1.1, 1:43.4:1.2, 1:43.4:1.3, 1:43.4:1.4, 1:43.4:1.5, 1:43.4:1.6, 1:43.4:1.7, 1:43.4:1.8, 1:43.4:1.9, 1:43.4:2, 1.1:43.4:1, 1.2:43.4:1, 1.3:43.4:1, 1.4:43.4:1, 1.5:43.4:1, 1.6:43.4:1, 1.7:43.4:1, 1.8:43.4:1, 1.9:43.4:1, 2:43.4:1, 1:43.5:1, 1:43.5:1.1, 1:43.5:1.2, 1:43.5:1.3, 1:43.5:1.4, 1:43.5:1.5, 1:43.5:1.6, 1:43.5:1.7, 1:43.5:1.8, 1:43.5:1.9, 1:43.5:2, 1.1:43.5:1, 1.2:43.5:1, 1.3:43.5:1, 1.4:43.5:1, 1.5:43.5:1, 1.6:43.5:1, 1.7:43.5:1, 1.8:43.5:1, 1.9:43.5:1, 2:43.5:1, 1:43.6:1, 1:43.6:1.1, 1:43.6:1.2, 1:43.6:1.3, 1:43.6:1.4, 1:43.6:1.5, 1:43.6:1.6, 1:43.6:1.7, 1:43.6:1.8, 1:43.6:1.9, 1:43.6:2, 1.1:43.6:1, 1.2:43.6:1, 1.3:43.6:1, 1.4:43.6:1, 1.5:43.6:1, 1.6:43.6:1, 1.7:43.6:1, 1.8:43.6:1, 1.9:43.6:1, 2:43.6:1, 1:43.7:1, 1:43.7:1.1, 1:43.7:1.2, 1:43.7:1.3, 1:43.7:1.4, 1:43.7:1.5, 1:43.7:1.6, 1:43.7:1.7, 1:43.7:1.8, 1:43.7:1.9, 1:43.7:2, 1.1:43.7:1, 1.2:43.7:1, 1.3:43.7:1, 1.4:43.7:1, 1.5:43.7:1, 1.6:43.7:1, 1.7:43.7:1, 1.8:43.7:1, 1.9:43.7:1, 2:43.7:1, 1:43.8:1, 1:43.8:1.1, 1:43.8:1.2, 1:43.8:1.3, 1:43.8:1.4, 1:43.8:1.5, 1:43.8:1.6, 1:43.8:1.7, 1:43.8:1.8, 1:43.8:1.9, 1:43.8:2, 1.1:43.8:1, 1.2:43.8:1, 1.3:43.8:1, 1.4:43.8:1, 1.5:43.8:1, 1.6:43.8:1, 1.7:43.8:1, 1.8:43.8:1, 1.9:43.8:1, 2:43.8:1, 1:43.9:1, 1:43.9:1.1, 1:43.9:1.2, 1:43.9:1.3, 1:43.9:1.4, 1:43.9:1.5, 1:43.9:1.6, 1:43.9:1.7, 1:43.9:1.8, 1:43.9:1.9, 1:43.9:2, 1.1:43.9:1, 1.2:43.9:1, 1.3:43.9:1, 1.4:43.9:1, 1.5:43.9:1, 1.6:43.9:1, 1.7:43.9:1, 1.8:43.9:1, 1.9:43.9:1, 2:43.9:1, 1:44:1, 1:44:1.1, 1:44:1.2, 1:44:1.3, 1:44:1.4, 1:44:1.5, 1:44:1.6, 1:44:1.7, 1:44:1.8, 1:44:1.9, 1:44:2, 1.1:44:1, 1.2:44:1, 1.3:44:1, 1.4:44:1, 1.5:44:1, 1.6:44:1, 1.7:44:1, 1.8:44:1, 1.9:44:1, 2:44:1, 1:45:1, 1:45:1.1, 1:45:1.2, 1:45:1.3, 1:45:1.4, 1:45:1.5, 1:45:1.6, 1:45:1.7, 1:45:1.8, 1:45:1.9, 1:45:2, 1.1:45:1, 1.2:45:1, 1.3:45:1, 1.4:45:1, 1.5:45:1, 1.6:45:1, 1.7:45:1, 1.8:45:1, 1.9:45:1, or 2:45:1. In some instances, the ratio of amounts for each active agent is about (1): (43.33):(1.67) for hydrocodone or a salt thereof: acetaminophen or a salt thereof: promethazine or a pharmaceutically acceptable salt thereof, respectively. In some instances, a pharmaceutically acceptable salt of hydrocodone, acetaminophen or promethazine is provided. In some instances, an opioid analgesic agent (such as hydrocodone or oxycodone or a salt thereof), acetaminophen or a salt thereof; and promethazine or a salt thereof are present in a bi-layer tablet that comprises an immediate-release and a controlled-release layer. In some instances, an opioid analgesic agent (such as hydrocodone or oxycodone or a salt thereof), acetaminophen or a salt thereof; and promethazine or a salt thereof are present in a two layer tablet that comprises an immediate-release and a controlled-release layer.

In some instances, a pharmaceutical composition comprises oxycodone, a pharmaceutically acceptable salt or its thiosemicarbazone, p-nitrophenylhydrazone, o-methyloxime, semicarbazone, or bis (methylcarbamate) (each of the foregoing being a hydrocodone agent or derivative); acetaminophen or a salt thereof; and promethazine or a salt thereof. Furthermore, the oxycodone or a salt thereof is present in a range of about 1 mg to about 200 mg, including but not limited to about: 1.0 mg, 1.5 mg, 2.5 mg, 3.0 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg or 20 mg, 30 mg, 40 mg, 50 mg, 70 mg, 100 mg, 130 mg, 160, 190 mg, 200 mg. Furthermore, the acetaminophen or a salt thereof is in a range of between about 200 mg to about 1000 mg, including but not limited to 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. The pharmaceutical compositions can further comprise between about 0.5 mg to about 200 mg of an antiemetic (e.g., promethazine or a salt thereof), including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some instances, oxycodone or a salt thereof, acetaminophen or a salt thereof and promethazine or a salt thereof are present in a bi-layer tablet that comprises an immediate-release and a controlled-release layer. In some instances, oxycodone or a salt thereof, acetaminophen or a salt thereof and promethazine or a salt thereof are present in a two layer tablet that comprises an immediate-release and a controlled-release layer.

In some instances, a pharmaceutical composition comprises promethazine or a salt thereof in an amount of 12.5 mg. In some instances, the pharmaceutical compositions herein comprise oxycodone or a salt thereof, acetaminophen or a salt thereof and promethazine or a salt thereof, wherein the pharmaceutical composition comprises the agents in a weight ratio of about (1 to 2):(40 to 45):(1 to 2), respectively. In some instances, a pharmaceutically acceptable salt of oxycodone, acetaminophen or promethazine is provided. In some instances, the weight ratio of amounts for each active agent is about (1):(43.33):(1.67) for oxycodone or a salt thereof, acetaminophen or a salt thereof and promethazine or a salt thereof, respectively. In some instances, the pharmaceutical compositions herein comprise an antiemetic (e.g., promethazine or a salt thereof) at a lower dosage than that which the antiemetic is administered alone. In some instances, the antiemetic is provided in the pharmaceutical composition at a dosage to prevent sedation, which can be observed with relatively higher dosages of promethazine or a salt thereof. Thus in some instances, promethazine is provided at about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. For example 12.5 mg or 25 mg of promethazine or a salt thereof per dose. Therefore, an antiemetic (e.g., promethazine or a salt thereof) can be provided at a dosage that is effective for reducing adverse effects associated with the opioid analgesic or non-opioid analgesic, but is at a relative low enough dosage (e.g., given the subject's weight) to prevent sedation associated with the antiemetic. Examples of adverse effects associated with the opioid analgesic or non-opioid analgesic include acute liver toxicity, allergic reactions, such as swelling, difficulty breathing, closing of throat, abdominal pain, nausea, retching, sleep disturbance, vomiting, constipation, unusual bleeding or bruising. In some instances, oxycodone or a salt thereof, acetaminophen or a salt thereof; and promethazine or a salt thereof are present in a bi-layer tablet that comprises an immediate-release and a controlled-release layer. In some instances, oxycodone or a salt thereof, acetaminophen or a salt thereof; and promethazine or a salt thereof are present in a two layer tablet that comprises an immediate-release and a controlled-release layer.

In some instances, a pharmaceutical composition disclosed herein comprises about 6-8 mg of hydrocodone or a salt thereof (such as about 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, or 8.0 mg,), about 310-330 mg of acetaminophen (such as about 310 mg, 315 mg, 320 mg, or 325 mg), and about 5-13 mg of promethazine or a salt thereof (such as about 10 mg, 10.5 mg, 11.0 mg, 11.5 mg, 12.0 mg, 12.5 mg, 13.0, mg, 13.5 mg, 14.0 mg, 14.5 mg, or 15 mg). In some instances, a pharmaceutically acceptable salt of hydrocodone, acetaminophen or promethazine is provided. The hydrocodone and the acetaminophen can be formulated using conventional technologies to provide for an extended time release over a desired dosage interval. All or some of the promethazine can be formulated for immediate-release to help abate common adverse effects associated with the hydrocodone and acetaminophen including nausea, retching, vomiting, constipation, other gastric upsets, sleep disturbance, skin rashes, allergic reactions such as swelling, difficulty breathing, closing of throat, abdominal pain, unusual bleeding or bruising, sedation, CNS depression, or respiratory depression. In some instances, hydrocodone, acetaminophen; and promethazine are present in a bi-layer tablet that comprises an immediate-release and a controlled-release layer. In some instances, hydrocodone, acetaminophen; and promethazine are present in a two layer tablet that comprises an immediate-release and a controlled-release layer.

In some instances, a pharmaceutical composition disclosed herein comprises from 1% to 20% by weight of an antiemetic (such as about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%); from 10% to 80% by weight a non-opioid analgesic (such as about: 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%); and from 1% to 20% by weight of an opioid analgesic (such as about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%). In some instances, an opioid analgesic agent, a non-opioid analgesic and an antiemetic are present in a bi-layer tablet that comprises an immediate-release and a controlled-release layer. In some instances, an opioid analgesic agent, a non-opioid analgesic and an antiemetic are present in a two layer tablet that comprises an immediate-release and a controlled-release layer.

In some instances, a pharmaceutical composition disclosed herein comprise about 6-8 mg of oxycodone HCL (such as about 7.5 mg), about 310-330 mg of acetaminophen (such as about 325 mg), and about 6-15 mg of promethazine HCL (such as about 12.5 mg). The oxycodone HCL and the acetaminophen can be formulated using conventional technologies to provide for an extended time release over a desired dosage interval. All or some of the promethazine can be formulated for immediate-release. In some instances, the pharmaceutical composition is in the form of a bi-layer tablet comprising an immediate-release layer comprising promethazine HCl and a controlled-release layer and a controlled-release layer comprising acetaminophen and oxycodone or a salt thereof. In some instances, the pharmaceutical composition is in the form of a two layer tablet comprising an immediate-release layer comprising promethazine HCl and a controlled-release layer and a controlled-release layer comprising acetaminophen and oxycodone or a salt thereof.

In some instances, administration of a pharmaceutical composition disclosed herein that comprises an antiemetic agent (such as promethazine or a salt thereof) can produce an outcome in a subject, such as reduced, abated or eliminated adverse effects associated with the administration of an opioid agent or non-opioid agent, such as oxycodone HCL, hydrocodone bitartrate and acetaminophen. Reduced, abated or eliminated adverse effects include but are not limited to including nausea, retching, vomiting, constipation, other gastric upsets, skin rashes, sleep disturbance, allergic reactions such as swelling, difficulty breathing, closing of throat, abdominal pain, unusual bleeding or bruising, sedation, CNS depression, or respiratory depression or any combination thereof.

In some instances, dosages and concentrations of active agents in a pharmaceutical composition can be varied as desired, as further described herein. Depending on the subject and/or condition being treated and on the administration route, the active agent in a pharmaceutical composition can generally be administered in dosages of 0.01 mg to 500 mg per kg body weight per day, e.g. about 20 mg/day for an average person. The dosage can be adjusted based on the mode of administration. A typical dosage can be one administration daily or multiple administrations daily. In some instances, for a controlled-release dosage form the unit dose can be designed for administration over a defined period of time. In some instances, dosage for one or a combination of agents can be from about 0.01 to 5 mg, 1 to 10 mg, 5 to 20 mg, 10 to 50 mg, 20 to 100 mg, 50 to 150 mg, 100 to 250 mg, 150 to 300 mg, 250 to 500 mg, 300 to 600 mg or 500 to 1000 mg V/kg body weight. Dose levels can vary as a function of the specific compound, the severity of the symptoms and the susceptibility of the subject to adverse effects. In another instance, a pharmaceutical composition comprises multiple active agents at the same or different dosages, where the pharmaceutical composition comprises an effective amount of: an opioid analgesic; an antiemetic; and a stimulant. In some instances, the pharmaceutical composition can further comprise a barbiturate or a non-opioid active agent, or both. The dosage can be adjusted according to the particular actives selected.

In some instances, a pharmaceutical composition comprises an effective amount of: an opioid analgesic; an antiemetic; and a stimulant. In this instance, the antiemetic (e.g., promethazine or a salt thereof), that is present at about 0.5 mg to about 60 mg, including but not limited to a dose of about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg. In some instances, the antiemetic is promethazine or a salt thereof. In other instances, the antiemetic is one described herein above. As described herein, in some instances, the antiemetic is a component of an immediate-release formulation. For example, in a further instance, the immediate-release is in a capsule, a tablet, a transdermal means, or achieved through injection, intramuscular administration or other means disclosed herein. In some instances, an opioid analgesic agent, a stimulant and an antiemetic are present in a bi-layer tablet that comprises an immediate-release and a controlled-release layer. In some instances, an opioid analgesic agent, a stimulant and an antiemetic are present in a two layer tablet that comprises an immediate-release and a controlled-release layer. In some instances, the stimulant and an antiemetic are present in the immediate-release layer and the opioid analgesic agent is present in the controlled-release layer. In another instance, an opioid analgesic agent, a non-opioid analgesic, a stimulant and an antiemetic are present in a bi-layer tablet that comprises an immediate-release and a controlled-release layer. In another instance, an opioid analgesic agent, a non-opioid analgesic, a stimulant and an antiemetic are present in a two layer tablet that comprises an immediate-release and a controlled-release layer. In some instances, the stimulant and an antiemetic are present in the immediate-release layer and the opioid analgesic agent and a non-opioid analgesic are present in the controlled-release layer.

In some instances, a pharmaceutical composition disclosed herein comprises: an effective amount of an opioid analgesic agent; an antiemetic agent; and a stimulant agent or a non-opioid agent, or both. In some instances, each agent is present at a dose of about: 0.5 mg to about 20 mg, 5 mg to 30 mg, 10 mg to 100 mg, including but not limited to about: 0.5 mg, 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 25 mg, 30 mg, 35 mg, 40 mg, 45 mg, or 50 mg. In some instances, an opioid analgesic agent, a stimulant and an antiemetic are present in a bi-layer tablet that comprises an immediate-release and a controlled-release layer. In some instances, an opioid analgesic agent, a stimulant and an antiemetic are present in a two layer tablet that comprises an immediate-release and a controlled-release layer. In some instances, the stimulant and an antiemetic are present in the immediate-release layer and the opioid analgesic agent is present in the controlled-release layer.

In some instances, a pharmaceutical composition can comprise: an effective amount of an opioid analgesic, a stimulant and an antiemetic. In some instances, the pharmaceutical composition comprising: an effective amount of an opioid analgesic, and a stimulant. In some instances, the pharmaceutical composition comprises a stimulant at a dose of about: 1 mg to 350 mg, 5 mg to 25 mg, 10 mg to 50 mg, 25 to 100 mg, 50 to 150 mg, 100 mg to 250 mg, 75 mg to 350 mg, including but not limited to about: 1.0 mg, 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, or 350 mg. In some instances, an opioid analgesic agent, a stimulant and an antiemetic are present in a bi-layer tablet that comprises an immediate-release and a controlled-release layer. In some instances, an opioid analgesic agent, a stimulant and an antiemetic are present in a two layer tablet that comprises an immediate-release and a controlled-release layer. In some instances, the stimulant and an antiemetic are present in the immediate-release layer and the opioid analgesic agent is present in the controlled-release layer.

In some instances, a pharmaceutical composition disclosed herein comprises: an opioid analgesic, a stimulant, and an antiemetic, wherein the relative ratio by weight of each of an opioid:a stimulant:an antiemetic is about (1 to 2):(40 to 45):(1 to 2), such as about: 1:40:1, 1:40:1.1, 1:40:1.2, 1:40:1.3, 1:40:1.4, 1:40:1.5, 1:40:1.6, 1:40:1.7, 1:40:1.8, 1:40:1.9, 1:40:2, 1.1:40:1, 1.2:40:1, 1.3:40:1, 1.4:40:1, 1.5:40:1, 1.6:40:1, 1.7:40:1, 1.8:40:1, 1.9:40:1, 2:40:1, 1:41:1, 1:41:1.1, 1:41:1.2, 1:41:1.3, 1:41:1.4, 1:41:1.5, 1:41:1.6, 1:41:1.7, 1:41:1.8, 1:41:1.9, 1:41:2, 1.1:41:1, 1.2:41:1, 1.3:41:1, 1.4:41:1, 1.5:41:1, 1.6:41:1, 1.7:41:1, 1.8:41:1, 1.9:41:1, 2:41:1, 1:42:1, 1:42:1.1, 1:42:1.2, 1:42:1.3, 1:42:1.4, 1:42:1.5, 1:42:1.6, 1:42:1.7, 1:42:1.8, 1:42:1.9, 1:42:2, 1.1:42:1, 1.2:42:1, 1.3:42:1, 1.4:42:1, 1.5:42:1, 1.6:42:1, 1.7:42:1, 1.8:42:1, 1.9:42:1, 2:42:1, 1:43:1, 1:43:1.1, 1:43:1.2, 1:43:1.3, 1:43:1.4, 1:43:1.5, 1:43:1.6, 1:43:1.7, 1:43:1.8, 1:43:1.9, 1:43:2, 1.1:43:1, 1.2:43:1, 1.3:43:1, 1.4:43:1, 1.5:43:1, 1.6:43:1, 1.7:43:1, 1.8:43:1, 1.9:43:1, 2:43:1, 1:43.1:1, 1:43.1:1.1, 1:43.1:1.2, 1:43.1:1.3, 1:43.1:1.4, 1:43.1:1.5, 1:43.1:1.6, 1:43.6:1, 1:43.6:1.1, 1:43.6:1.2, 1:43.6:1.3, 1:43.6:1.4, 1:43.6:1.5, 1:43.6:1.6, 1:43.6:1.7, 1:43.6:1.8, 1:43.6:1.9, 1:43.6:2, 1.1:43.6:1, 1.2:43.6:1, 1.3:43.6:1, 1.4:43.6:1, 1.5:43.6:1, 1.6:43.6:1, 1.7:43.6:1, 1.8:43.6:1, 1.9:43.6:1, 2:43.6:1, 1:43.7:1, 1:43.7:1.1, 1:43.7:1.2, 1:43.7:1.3, 1:43.7:1.4, 1:43.7:1.5, 1:43.7:1.6, 1:43.7:1.7, 1:43.7:1.8, 1:43.7:1.9, 1:43.7:2, 1.1:43.7:1, 1.2:43.7:1, 1.3:43.7:1, 1.4:43.7:1, 1.5:43.7:1, 1.6:43.7:1, 1.7:43.7:1, 1.8:43.7:1, 1.9:43.7:1, 2:43.7:1, 1:43.8:1, 1:43.8:1.1, 1:43.8:1.2, 1:43.8:1.3, 1:43.8:1.4, 1:43.8:1.5, 1:43.8:1.6, 1:43.8:1.7, 1:43.8:1.8, 1:43.8:1.9, 1:43.8:2, 1.1:43.8:1, 1.2:43.8:1, 1.3:43.8:1, 1.4:43.8:1, 1.5:43.8:1, 1.6:43.8:1, 1.7:43.8:1, 1.8:43.8:1, 1.9:43.8:1, 2:43.8:1, 1:43.9:1, 1:43.9:1.1, 1:43.9:1.2, 1:43.9:1.3, 1:43.9:1.4, 1:43.9:1.5, 1:43.9:1.6, 1:43.9:1.7, 1:43.9:1.8, 1:43.9:1.9, 1:43.9:2, 1.1:43.9:1, 1.2:43.9:1, 1.3:43.9:1, 1.4:43.9:1, 1.5:43.9:1, 1.6:43.9:1, 1.7:43.9:1, 1.8:43.9:1, 1.9:43.9:1, 2:43.9:1, 1:44:1, 1:44:1.1, 1:44:1.2, 1:44:1.3, 1:44:1.4, 1:44:1.5, 1:44:1.6, 1:44:1.7, 1:44:1.8, 1:44:1.9, 1:44:2, 1.1:44:1, 1.2:44:1, 1.3:44:1, 1.4:44:1, 1.5:44:1, 1.6:44:1, 1.7:44:1, 1.8:44:1, 1.9:44:1, 2:44:1, 1:45:1, 1:45:1.1, 1:45:1.2, 1:45:1.3, 1:45:1.4, 1:45:1.5, 1:45:1.6, 1:45:1.7, 1:45:1.8, 1:45:1.9, 1:45:2, 1.1:45:1, 1.2:45:1, 1.3:45:1, 1.4:45:1, 1.5:45:1, 1.6:45:1, 1.7:45:1, 1.8:45:1, 1.9:45:1, or 2:45:1. In some instances, an opioid analgesic agent, a stimulant and an antiemetic are present in a bi-layer tablet that comprises an immediate-release and a controlled-release layer. In some instances, an opioid analgesic agent, a stimulant and an antiemetic are present in a two layer tablet that comprises an immediate-release and a controlled-release layer. In some instances, the stimulant and an antiemetic are present in the immediate-release layer and the opioid analgesic agent is present in the controlled-release layer.

In some instances, a pharmaceutical composition disclosed herein has an effective amount of an opioid (such as hydrocodone, fentanyl or oxycodone or a salt thereof); a non-opioid (such as acetaminophen or naproxen salt thereof); and a barbiturate (such as butalbital or a salt thereof). In some instances, the pharmaceutical compositions further comprise an antiemetic (such as promethazine or a salt thereof). In some instances, the pharmaceutical composition further comprises a stimulant agent. In some instances, the barbiturate is present at a dose of about: 1 mg to 350 mg, 5 mg to 25 mg, 10 mg to 50 mg, 25 to 100 mg, 50 to 150 mg, 100 mg to 250 mg, 75 mg to 350 mg, including but not limited to about: 1.0 mg, 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, or 350 mg.

In some instances, a pharmaceutical composition comprises an effective amount of an opioid (such as hydrocodone, fentanyl or oxycodone or a salt thereof); a non-opioid agent (such as acetaminophen or naproxen or a salt thereof); and a barbiturate (such as butalbital or a salt thereof). In some instances, the opioid agent (such as hydrocodone, oxycodone, tapentadol or a salt thereof) is present in a range of about 1 mg to about 200 mg, including but not limited to about: 1.0 mg, 1.5 mg, 2.5 mg, 3.0 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg or 20 mg, 30 mg, 40 mg, 50 mg, 70 mg, 100 mg, 130 mg, 160, 190 mg, 200 mg. Furthermore, the non-opioid agent (such as acetaminophen or naproxen or a salt thereof) is present in a range of between about 200 mg to about 1000 mg, including but not limited to about: 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. Additionally, the barbiturate (e.g., butalbital or a salt thereof) is present at a dose between about 0.5 mg to about 200 mg, including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some instances, an opioid analgesic agent, a non-opioid agent, and a barbiturate agent are present in a bi-layer tablet that comprises an immediate-release and a controlled-release layer. In a further instance, the bi-layer tablet comprises an antiemetic agent, such as an antiemetic. In some instances, an opioid analgesic agent, a non-opioid agent, and a barbiturate agent are present in a two layer tablet that comprises an immediate-release and a controlled-release layer. In a further instance, the two layer tablet comprises an antiemetic agent, such as an antiemetic. In some instances, the antiemetic is present in the immediate-release layer and the opioid analgesic agent, non-opioid agent, and barbiturate agent are present in the controlled-release layer.

In some instances, a pharmaceutical composition disclosed herein comprises an effective amount of a barbiturate agent (such as butalbital or a salt thereof); a non-opioid agent (such as acetaminophen or naproxen or a salt thereof); and a stimulant agent (such as caffeine or a salt thereof). In some instances, the barbiturate agent (such as butalbital or a salt thereof); is present in a range of about 0.5 mg to about 200 mg, including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. Furthermore, the non-opioid agent (such as acetaminophen or naproxen or a salt thereof) is present in a range of between about 200 mg to about 1000 mg, including but not limited to about: 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. Additionally, the stimulant agent (e.g., caffeine) is present at a dose from about 0.5 mg to about 200 mg including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some instances, a stimulant agent, a non-opioid agent, and a barbiturate agent are present in a bi-layer tablet that comprises an immediate-release and a controlled-release layer. In some instances, a stimulant agent, a non-opioid agent, and a barbiturate agent are present in a two layer tablet that comprises an immediate-release and a controlled-release layer. In some instances, the stimulant is present in the immediate-release layer and the non-opioid analgesic agent and barbiturate are present in the controlled-release layer. In a further instance, the bi-layer tablet comprises an antiemetic agent, such as an antihistamine (e.g., promethazine). In some instances, the two layer tablet comprises an antiemetic agent, such as an antihistamine (e.g., promethazine). In some instances, the stimulant and an antihistamine are present in the immediate-release layer and the non-opioid analgesic agent and barbiturate are present in the controlled-release layer.

In some instances, a pharmaceutical composition provided herein can comprise an effective amount of a barbiturate and a stimulant. In some instances, the pharmaceutical composition comprises a stimulant at a dose of about 1 mg to about 350 mg (such as about: 5 mg to 25 mg, 10 mg to 50 mg, 25 to 100 mg, 50 to 150 mg, 100 mg to 250 mg, 75 mg to 350 mg) including but not limited to about: 1.0 mg, 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, or 350 mg. Additionally, the barbiturate agent (such as butalbital or a salt thereof); is present in a range of about 0.5 mg to about 200 mg, including but not limited to about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some instances, a barbiturate agent, and a stimulant are present in a bi-layer tablet that comprises an immediate-release and a controlled-release layer. In a further instance, the bi-layer tablet further comprises an antiemetic agent, such as an antihistamine (e.g. promethazine or a salt thereof). In some instances, a barbiturate agent, and a stimulant are present in a two layer tablet that comprises an immediate-release and a controlled-release layer. In a further instance, the two layer tablet further comprises an antiemetic agent, such as an antihistamine (e.g. promethazine or a salt thereof). In some instances, the stimulant and an antihistamine are present in the immediate-release layer and the barbiturate agent is present in the controlled-release layer.

In some instances, a pharmaceutical composition comprises an effective amount of a non-opioid agent (such as naproxen or ibuprofen or a salt thereof) and a stimulant (such as caffeine or a salt thereof). In some instances, the non-opioid agent (such as naproxen or ibuprofen or a salt thereof) is present in a range of between about 200 mg to about 1000 mg, including but not limited to about: 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. In these instances, a pharmaceutical composition comprises a stimulant at a dose of about 1 mg to about 350 mg, (such as about: 5 mg to 25 mg, 10 mg to 50 mg, 25 to 100 mg, 50 to 150 mg, 100 mg to 250 mg, or 75 mg to 350 mg), including but not limited to about: 1.0 mg, 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, or 350 mg. In some instances, a non-opioid agent and a stimulant are formulated as a bi-layer tablet that comprises an immediate-release and a controlled-release layer. In one example naproxen and caffeine are formulated in a bi-layer tablet. In some instances, a non-opioid agent and a stimulant are formulated as a two layer tablet that comprises an immediate-release and a controlled-release layer. In one example naproxen and caffeine are formulated in a two layer tablet. In some instances, the caffeine is present in the immediate-release layer and naproxen is present in the controlled-release layer.

In some instances, a pharmaceutical composition disclosed herein comprises an effective amount of propoxyphene or a salt thereof and a non-opioid agent (such as naproxen or a salt thereof). In some instances, the pharmaceutical composition further comprises an antiemetic (such as promethazine or a salt thereof). In some instances, the pharmaceutical compositions further comprise a stimulant agent. In some instances, the propoxyphene or salt thereof is present in a range of about 1.0 mg to about 100 mg, including but not limited to about: 11.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, or 100 mg. Furthermore, the non-opioid agent is in a range of about 200 mg to about 1000 mg, including but not limited to about: 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. In some instances, propoxyphene or a salt thereof and naproxen (such as naproxen sodium or naproxen magnesium) are present in a bi-layer tablet. In some instances, propoxyphene or a salt thereof and naproxen (such as naproxen sodium or naproxen magnesium) are present in a two layer tablet. In a further instance, the pharmaceutical composition comprises an antiemetic (e.g., promethazine or a salt thereof). In some instances, the antihistamine is present in the immediate-release layer and propoxyphene and naproxen are present in the controlled-release layer.

In some instances, a pharmaceutical composition described herein comprises an effective amount of an antiemetic (e.g., promethazine or a salt thereof), that is present in the range of at about 0.5 mg to about 60 mg, including but not limited to a dose of about: 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg. In some instances, the antiemetic is promethazine or a salt thereof. In other instances, the antiemetic is another described herein above. As described herein, in some instances, the antiemetic is a component of an immediate-release formulation. For example, in a further instance, the immediate-release is in a lollipop, a capsule, a tablet, a transdermal means, through injection, intramuscular administration or other means disclosed herein.

In some instances, any of the pharmaceutical compositions disclosed herein can comprise one or more laxatives. In some instances, a pharmaceutical composition comprises an opioid analgesic, an antiemetic, and a laxative. The laxative can in an amount effective to reduce or eliminate constipation, such as opioid-induced constipation. The effective amount can depend upon the laxative and/or route of administration. In some instances, a single dose of a pharmaceutical composition disclosed herein comprises from about 1 mg to about 1000 mg of a laxative. For example, the pharmaceutical composition can comprise about: 11000 mg, 1-750 mg, 1-500 mg, 1-250 mg, 1-150 mg, 1-100 mg, 1-75 mg, 1-50 mg, 1-25 mg, 1-10 mg, 10-1000 mg, 10-750 mg, 10-500 mg, 10-250 mg, 10-150 mg, 10-100 mg, 10-75 mg, 10-50 mg, 10-25 mg, 25-1000 mg, 25-750 mg, 25-500 mg, 25-250 mg, 25-150 mg, 25-100 mg, 25-75 mg, 2550 mg, 50-1000 mg, 50-750 mg, 50-500 mg, 50-250 mg, 50-150 mg, 50-100 mg, 50-75 mg, 75-1000 mg, 75-750 mg, 75-500 mg, 75-250 mg, 75-150 mg, 75-100 mg, 100-1000 mg, 100-750 mg, 100500 mg, 100-250 mg, 100-150 mg, 150-1000 mg, 150-750 mg, 150-500 mg, 150-250 mg, 250-1000 mg, 250-750 mg, 250-500 mg, 500-1000 mg, 500-750 mg, 750-1000 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg of a laxative. The laxative can be a bulk-producing agent (e.g., polycarbophil calcium, methyl cellulose, a soluble dietary fibre, an insoluble dietary fibre), a stool softener (e.g., dioctyl calcium sulfosuccinate, dioctyl sodium sulfosuccinate, or dioctyl potassium sulfosuccinate), a lubricant (e.g., mineral oil), a hydrating agent (e.g., sodium chloride, sodium bicarbonate, potassium chloride, sodium sulfate, sodium phosphate, potassium sodium tartrate, magnesium citrate, magnesium hydroxide, magnesium sulfate, sorbitol, lactulose, polyethylene glycol), a stimulant or irritant (e.g., dantron, emodine, aloe emodin, a senna glycoside, bisacodyl, phenolphthalein), a serotonin agonist (e.g., tegaserod, cisapride, prucalopride), or a chloride channel activator (e.g., lubiprostone).

In some instances, a single dose of a pharmaceutical composition disclosed herein from about 0.1 g to about 20 g of a bulk-producing agent such as polycarbophil calcium, methyl cellulose, a soluble dietary fibre, an insoluble dietary fibre, or any combination thereof. For example, the single dose can comprise about: 0.1-20 g, 0.1-15 g, 0.1-10 g, 0.1-7.5 g, 0.1-5 g, 0.1-2 g, 0.1-1 g, 0.1-0.5 g, 0.5-20 g, 0.5-15 g, 0.5-10 g, 0.5-7.5 g, 0.5-5 g, 0.5-2 g, 0.5-1 g, 1-20 g, 1-15 g, 1-10 g, 1-7.5 g, 1-5 g, 1-2 g, 220 g, 2-15 g, 2-10 g, 2-7.5 g, 2-5 g, 5-20 g, 5-15 g, 5-10 g, 5-7.5 g, 7.5-20 g, 7.5-15 g, 7.5-10 g, 1020 g, 10-15 g, 15-20 g, 0.1 g, 0.2 g, 0.3 g, 0.4 g, 0.5 g, 0.6 g, 0.7 g, 0.8 g, 0.9 g, 1 g, 1.1 g, 1.2 g, 1.3 g, 1.4 g, 1.5 g, 1.6 g, 1.7 g, 1.8 g, 1.9 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, 10 g, 11 g, 12 g, 13 g, 14 g, 15 g, 16 g, 17 g, 18 g, 19 g, or 20 g of a bulk-producing agent.

In some instances, a single dose of a pharmaceutical composition disclosed herein comprises from about 1 mg to about 1000 mg of a stool softener such as dioctyl calcium sulfosuccinate, dioctyl sodium sulfosuccinate, or dioctyl potassium sulfosuccinate. For example, the pharmaceutical composition can comprise about: 1-1000 mg, 1-750 mg, 1-500 mg, 1-250 mg, 1-150 mg, 1-100 mg, 1-75 mg, 1-50 mg, 1-25 mg, 1-10 mg, 10-1000 mg, 10-750 mg, 10-500 mg, 10-250 mg, 10-150 mg, 10-100 mg, 1075 mg, 10-50 mg, 10-25 mg, 25-1000 mg, 25-750 mg, 25-500 mg, 25-250 mg, 25-150 mg, 25-100 mg, 25-75 mg, 25-50 mg, 50-1000 mg, 50-750 mg, 50-500 mg, 50-250 mg, 50-150 mg, 50-100 mg, 50-75 mg, 75-1000 mg, 75-750 mg, 75-500 mg, 75-250 mg, 75-150 mg, 75-100 mg, 100-1000 mg, 100-750 mg, 100-500 mg, 100-250 mg, 100-150 mg, 150-1000 mg, 150-750 mg, 150-500 mg, 150250 mg, 250-1000 mg, 250-750 mg, 250-500 mg, 500-1000 mg, 500-750 mg, 750-1000 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg of a stool softener such as dioctyl calcium sulfosuccinate, dioctyl sodium sulfosuccinate, or dioctyl potassium sulfosuccinate. In some instances, the single dose of the pharmaceutical composition comprises from about 15 mg to about 500 mg of a stool softener such as dioctyl calcium sulfosuccinate, dioctyl sodium sulfosuccinate, or dioctyl potassium sulfosuccinate. In another instance, the single dose of the pharmaceutical composition comprises from about 15 mg to about 125 mg of a stool softener such as dioctyl calcium sulfosuccinate, dioctyl sodium sulfosuccinate, or dioctyl potassium sulfosuccinate. In another instance, the single dose of the pharmaceutical composition comprises from about 15 mg to about 500 mg of a stool softener such as dioctyl calcium sulfosuccinate, dioctyl sodium sulfosuccinate, or dioctyl potassium sulfosuccinate.

In some instances, a single dose of a pharmaceutical composition disclosed herein comprises from about 1 mg to about 100 mg of a stimulant or irritant such as an anthracenedione, a triphenylmethane, or castor oil. Suitable anthracenediones include dantron (1,8-dihydroxyanthraquinone), emodine (6-methyl-1,3,8-trihydroxyanthraquinone), aloe emodin (1,8-Dihydroxy-3-(hydroxymethyl)-9,10-anthracenedione), and senna glycosides. Suitable triphenylmethanes include bisacodyl [4,4′-(pyridin-2-ylmethylene)bis(4,1-phenylene) diacetate] and phenolphthalein. For example, the pharmaceutical composition can comprise about: 1-100 mg, 1-75 mg, 1-50 mg, 125 mg, 1-15 mg, 1-10 mg, 1-7.5 mg, 1-5 mg, 1-2.5 mg, 2.5-100 mg, 2.5-75 mg, 2.5-50 mg, 2.5-25 mg, 2.5-15 mg, 2.5-10 mg, 2.5-7.5 mg, 2.5-5 mg, 5-100 mg, 5-75 mg, 5-50 mg, 5-25 mg, 5-15 mg, 5-10 mg, 5-7.5 mg, 7.5-100 mg, 7.5-75 mg, 7.5-50 mg, 7.5-25 mg, 7.5-15 mg, 7.5-10 mg, 10-100 mg, 10-75 mg, 10-50 mg, 10-25 mg, 10-15 mg, 15-100 mg, 15-75 mg, 15-50 mg, 15-25 mg, 25-100 mg, 25-75 mg, 25-50 mg, 50-100 mg, 50-75 mg, 75-100 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, or 100 mg of the stimulant or irritant. In some instances, the single dose of the pharmaceutical composition comprises from about 1 mg to about 50 mg of the stimulant or irritant. In another instance, the single dose of the pharmaceutical composition comprises from about 5 mg to about 15 mg of the stimulant or irritant.

In some instances, a single dose of a pharmaceutical composition disclosed herein comprises from about 1 g to about 50 g of a saline laxative such as sodium chloride, sodium bicarbonate, potassium chloride, sodium sulfate, sodium phosphate, potassium sodium tartrate, magnesium citrate, magnesium hydroxide, magnesium sulfate, or any combination thereof. For example, the single dose of the pharmaceutical composition can comprise about: 1-50 g, 1-30 g, 1-25 g, 1-20 g, 1-15 g, 1-10 g, 1-5 g, 5-50 g, 530 g, 5-25 g, 5-20 g, 5-15 g, 5-10 g, 10-50 g, 10-30 g, 10-25 g, 10-20 g, 10-15 g, 15-50 g, 15-30 g, 15-25 g, 15-20 g, 20-50 g, 20-30 g, 20-25 g, 25-50 g, 25-30 g, 30-50 g, 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, or 50 g of the saline laxative. In some instances, the pharmaceutical composition comprises about 10 g of the saline laxative. In another instance, the pharmaceutical composition comprises about 20 g of the saline laxative. In another instance, the pharmaceutical composition comprises about 30 g of the saline laxative.

In some instances, a single dose of a pharmaceutical composition disclosed herein comprises from about 1 g to about 50 g of a hyperosmotic agent such as sorbitol, lactulose, polyethylene glycol or glycerin. For example, the single dose of the pharmaceutical composition can comprise about: 1-50 g, 1-30 g, 1-25 g, 1-20 g, 1-15 g, 1-10 g, 1-5 g, 5-50 g, 5-30 g, 5-25 g, 5-20 g, 5-15 g, 5-10 g, 10-50 g, 10-30 g, 1025 g, 10-20 g, 10-15 g, 15-50 g, 15-30 g, 15-25 g, 15-20 g, 20-50 g, 20-30 g, 20-25 g, 25-50 g, 25-30 g, 30-50 g, 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, or 50 g of the hyperosmotic agent. In some instances, the single dose of the pharmaceutical composition comprises about 5 g of the hyperosmotic agent. In another instance, the single dose of the pharmaceutical composition comprises about 10 g of the hyperosmotic agent. In another instance, the single dose of the pharmaceutical composition comprises about 15 g of the hyperosmotic agent. In another instance, the single dose of the pharmaceutical composition comprises about 20 g of the hyperosmotic agent. In another instance, the single dose of the pharmaceutical composition comprises about 30 g of the hyperosmotic agent.

In some instances, a single dose of a pharmaceutical composition disclosed herein comprises from about 1 μg to about 100 μg of a chloride channel activator such as lubiprostone. For example, the single dose of the pharmaceutical composition can comprise about: 1-100 μg, 1-75 μg, 1-50 μg, 1-25 μg, 1-15 μg, 110 μg, 1-7.5 μg, 1-5 μg, 1-2.5 μg, 2.5-100 μg, 2.5-75 μg, 2.5-50 μg, 2.5-25 μg, 2.5- 15 μg, 2.5-10 μg, 2.5-7.5 μg, 2.5-5 μg, 5-100 μg, 5-75 μg, 5-50 μg, 5-25 μg, 5-15 μg, 5-10 μg, 5-7.5 μg, 7.5-100 μg, 7.5-75 μg, 7.5-50 μg, 7.5-25 μg, 7.5-15 μg, 7.5-10 μg, 10-100 μg, 10-75 μg, 10-50 μg, 10-25 μg, 10-15 μg, 15-100 μg, 15-75 μg, 15-50 μg, 15-25 μg, 25-100 μg, 25-75 μg, 25-50 μg, 50-100 μg, 50-75 μg, 75-100 μg, 1 μg, 1.5 μg, 2 μg, 2.5 μg, 3 μg, 3.5 μg, 4 μg, 4.5 μg, 5 μg, 5.5 μg, 6 μg, 6.5 μg, 7 μg, 7.5 μg, 8 μg, 8.5 μg, 9 μg, 9.5 μg, 10 μg, 10.5 μg, 11 μg, 11.5 μg, 12 μg, 12.5 μg, 13 μg, 13.5 μg, 14 μg, 14.5 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 55 μg, 60 μg, 65 μg, 70 μg, 75 μg, 80 μg, 85 μg, 90 μg, 95 μg, or 100 μg of the chloride channel activator. In some instances, the single dose of the pharmaceutical composition comprises from about 5 μg to about 50 μg of the chloride channel activator. In another instance, the single dose of the pharmaceutical composition comprises from about 20 μg to about 30 μg of the chloride channel activator.

In some instances, a single dose of a pharmaceutical composition disclosed herein comprises from about 1 mg to about 25 mg of a serotonin agonist such as tegaserod, cisapride, or prucalopride. For example, the single dose of the pharmaceutical composition can comprise about: 1-25 mg, 1-20 mg, 1-15 mg, 1-10 mg, 1-5 mg, 5-25 mg, 5-20 mg, 5-15 mg, 5-10 mg, 10-25 mg, 10-20 mg, 10-15 mg, 15-25 mg, 15-20 mg, 20-25 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg of the serotonin agonist. In some instances, the single dose of the pharmaceutical composition comprises from about 1 mg to about 10 mg of the serotonin agonist. In another instance, the single dose of the pharmaceutical composition comprises about 6 mg of the serotonin agonist.

### Dosage Forms

### Oral Dosage Forms

Some instances relate to methods and pharmaceutical compositions formulated for oral delivery to a subject in need. In some instances, a pharmaceutical composition is formulated so as to deliver one or more pharmaceutically active agents to a subject through a mucosa layer in the mouth or esophagus. In another instance, the pharmaceutical composition is formulated to deliver one or more pharmaceutically active agents to a subject through a mucosa layer in the stomach and/or intestines.

In some instances, a pharmaceutical composition is provided in controlled-release dosage forms (such as immediate-release, controlled-release or both), which comprise an effective amount of an opioid analgesic (such as oxycodone or hydrocodone or a salt thereof), a non-opioid analgesic (such as acetaminophen, naproxen or ibuprofen or a salt thereof) and an antiemetic (such as promethazine or a salt thereof); and one or more release controlling excipients as described herein. In some instances, the opioid analgesic is formulated for controlled release. In some instances, the non-opioid analgesic is formulated for controlled release. In some instances, the antiemetic is formulated for immediate release. Suitable controlled-release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions can also comprise non-release controlling excipients.

In some instances, a pharmaceutical composition is provided in enteric coated dosage forms. The pharmaceutical compositions can also comprise non-release controlling excipients. In another instance, pharmaceutical compositions are provided in effervescent dosage forms. The pharmaceutical compositions can also comprise non-release controlling excipients. In some instances, a pharmaceutical composition is provided in a dosage form that has at least one component that can facilitate the immediate-release of an active agent, and at least one component that can facilitate the controlled-release of an active agent. In some instances, the dosage form is capable of giving a discontinuous release of the compound in the form of at least two consecutive pulses separated in time from 0.1 up to 8 hours. The pharmaceutical compositions can comprise one or more release controlling and non-release controlling excipients, such as those excipients suitable for a disruptable semi-permeable membrane and as swellable substances.

In some instances, a pharmaceutical composition is provided in a dosage form for oral administration to a subject in need thereof, which comprises one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer. In some instances, a pharmaceutical composition is in the form of enteric-coated granules, as controlled-release capsules for oral administration. The pharmaceutical compositions can further comprise cellulose disodium hydrogen phosphate, hydroxypropyl cellulose, hypromellose, lactose, mannitol, and sodium lauryl sulfate. In some instances, a pharmaceutical composition is in the form of enteric-coated pellets, as controlled-release capsules for oral administration. The pharmaceutical compositions can further comprise glyceryl monostearate 40-50, hydroxypropyl cellulose, hypromellose, magnesium stearate, methacrylic acid copolymer type C, polysorbate 80, sugar spheres, talc, and triethyl citrate. In some instances, a pharmaceutical composition is enteric-coated controlled-release tablets for oral administration. The pharmaceutical compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide. In some instances, a pharmaceutical composition comprises calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In some instances, a pharmaceutical composition provided herein is in a unit-dosage form or multiple-dosage form. Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another instance, the multiple dosage forms comprise different pharmaceutically active agents. For example, a multiple dosage form can be provided which comprises a first dosage element comprising an immediate-release form of an antiemetic (such as in a liquid form) and a second dosage element comprising an opioid and/or non opioid analgesic, which can be in a controlled-release or immediate-release form. In this example, a pair of dosage elements can make a single unit dosage.

In some instances, a kit is provided comprising multiple unit dosages, wherein each unit comprises a first dosage element comprising an immediate-release form of an antiemetic (such as in a liquid form) and a second dosage element comprising an opioid or non-opioid analgesic or both, which can be in a controlled-release form or an immediate-release form. In another instance, the kit further comprises a set of instructions. In yet a further instance, the antiemetic is promethazine or a pharmaceutically acceptable salt thereof, the opioid analgesic is oxycodone or hydrocodone or pharmaceutically acceptable salt thereof, the non-opioid analgesic is acetaminophen or a pharmaceutically acceptable salt thereof.

In some instances, a pharmaceutical composition disclosed herein is formulated in various dosage forms for oral, parenteral, and topical administration. The pharmaceutical compositions can also be formulated as an immediate-, controlled-release dosage forms. The pharmaceutical compositions can also be formulated as gastric retention dosage forms. These dosage forms can be prepared according to known methods and techniques. In some instances, a pharmaceutical composition disclosed herein is in one or more dosage form. For example, a pharmaceutical composition can be administered in a solid or liquid-form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be coated or scored, having indicia inscribed thereon and can be so formulated as to cause immediate or controlled release of the opioid analgesics (such as oxycodone or hydrocodone) and/or the non-opioid analgesics (such as acetaminophen) and or the antiemetic (such as promethazine). Furthermore, dosage forms herein can comprise acceptable carriers or salts known in the art, such as those described in the Handbook of Pharmaceutical Excipients, American Pharmaceutical Association (1986), incorporated by reference herein in its entirety.

In some instances, one or more pharmaceutically active agents are mixed with a pharmaceutical excipient to form a solid preformulation pharmaceutical composition comprising a homogeneous mixture of compounds described herein. When referring to these pharmaceutical compositions as “homogeneous”, it is meant that the agents are dispersed evenly throughout the pharmaceutical composition so that the pharmaceutical composition can be subdivided into unit dosage forms such as tablets or capsules. This solid preformulation pharmaceutical composition can then be subdivided into unit dosage forms of the type described above comprising from, for example, about 1.0 to about 15 mg of an opioid analgesic, such as hydrocodone or oxycodone or a pharmaceutically acceptable salt of any one of the foregoing, or any combination thereof.

In some instances, a pharmaceutical composition can be formulated, in the instance of capsules or tablets, to be swallowed whole, for example with water. The inclusion of the side-effect-reducing agent such as an antiemetic to abate common symptoms of nausea or vomiting are believed beneficial in that promethazine or a salt thereof, or the like can eliminate or minimize the amount of discomfort. Adverse effects reduced or eliminated include but are not limited to nausea, retching, vomiting, other gastric upsets, sleep disturbance, constipation, skin rashes, allergic reactions such as swelling, difficulty breathing, closing of throat, abdominal pain, unusual bleeding or bruising, CNS suppression and respiratory suppression.

Frequently, subjects taking opioids have adverse effects including vomiting that can occur shortly after taking a first or subsequent dose. As a consequence, a portion of the opioid dose is subsequently lost, making it difficult to accurately gauge replacement dosages for the subject, and for subjects outside of a hospital or clinic environment, there might not be any alternative form of pain medication readily available. As a consequence, subjects experiencing gastric discomfort such as vomiting can lack the beneficial effects of the opioid analgesic and experience the additional discomfort and enhanced pain associated with vomiting. This problem is solved by also administering promethazine or a salt thereof, which reduces side-effects.

A dosage form described herein can be manufactured using processes that are well known to those of skill in the art. For example, for the manufacture of tablets (including but not limited to single layer, bi-layer, two layer, coated, of multi-layer tablets) or capsules, the agents can be dispersed uniformly in one or more excipients, for example, using high shear granulation, low shear granulation, fluid bed granulation, or by blending for direct compression.

A controlled-release formulation can comprise one or more combinations of excipients that slow the release of the agents by coating or temporarily bonding or decreasing their solubility of the active agents. In some instances, the opioid analgesic or non-opioid agents (e.g., hydrocodone or oxycodone or a salt thereof, and acetaminophen or a salt thereof) are formulated for controlled-release while the promethazine or a salt thereof is formulated for immediate-release. In another instance, the opioid analgesic or non-opioid agents (e.g., hydrocodone or oxycodone or a salt thereof, and acetaminophen or a salt thereof) are formulated for controlled-release while the promethazine or a salt thereof is formulated for immediate-release. In another instance, all agents are formulated for controlled-release.

An immediate-release formulation can comprise one or more combination of excipients that allow for a rapid release of a pharmaceutically active agent (such as from 1 minute to 1 hour after administration, or following contact with dissolution fluid, or as measured in an in vitro dissolution study), such as an antiemetic. In some instances, an immediate-release excipient can be microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, Avicel PH200, and combinations of such excipients.

In some instances, pharmaceutical carriers or vehicles suitable for administration of the compounds provided herein include all such carriers known to those skilled in the art to be suitable for the particular mode of administration. In addition, the pharmaceutical compositions can one or more components that do not impair the desired action, or with components that supplement the desired action, or have another action. As noted above, the pharmaceutical compositions can comprise additional (e.g., a fourth, fifth, sixth, etc.) additional active agents.

In some instances, a pharmaceutical composition comprised three or more pharmaceutically active agents wherein at least one active agent is formulated in an immediate-release form. In this instance, the immediate-release form can be included in an amount that is effective to shorten the time to its maximum concentration in the blood. By way of example, certain immediate-release pharmaceutical preparations are taught in United States Patent Publication US 2005/0147710A1 entitled, “Powder Compaction and Enrobing” which is incorporated herein in its entirety by reference.

In some instances, a component of an immediate-release form or layer is a component that reduces abates or eliminates and/or suppresses an adverse effect associated with one or more opioid analgesics. For example, the immediate-release active can be an antiemetic, which reduces, abates or eliminates an adverse effect associated with opioid and/or non-opioid analgesics described herein. In a further instance, all or less than the entire amount of the antiemetic agent is formulated in immediate-release form, as described herein.

A variety of methods and materials can be used to bring about immediate-release. For instance, placement of the agent along an exterior of a tablet (e.g., coating the exterior or formulating the outer layer with the agent) and/or combined with forming a tablet by compressing the powder using low compaction can produce immediate-release of the agent from the pharmaceutical composition.

In some embodiments, an effective amount of promethazine or a salt thereof in an immediate-release form is coated onto a substrate. For example, where the controlled release of one or more analgesics from a formulation is due to a controlled-release coating, an immediate-release layer comprising promethazine or a salt thereof can overcoat the controlled-release coating. In another example, an immediate-release layer can be coated onto the surface of a substrate wherein an opioid agent, a non-opioid agent, a barbiturate, or a stimulant is incorporated in a controlled-release matrix. Where a plurality of controlled-release substrates (e.g., multiparticulate systems including pellets, spheres, beads and the like) are incorporated into a hard gelatin capsule, a side-effect-reducing compound can be incorporated into the gelatin capsule via inclusion of an amount of immediate-release promethazine or a salt thereof, as a powder or granulate within the capsule. In some instances, the gelatin capsule itself can be coated with an immediate-release layer of promethazine. One skilled in the art recognizes still other alternative means of incorporating an immediate-release side-effect-reducing compound into the unit dose. By including an effective amount of immediate-release side-effect-reducing compound in the unit dose, the experience of adverse effects including nausea, retching, vomiting, constipation, other gastric upsets, sleep disturbance, skin rashes, allergic reactions such as swelling, difficulty breathing, closing of throat, abdominal pain, unusual bleeding or bruising, skin rashes, sedation, CNS depression, or respiratory depression in subjects can be significantly reduced.

In some instances, a pharmaceutical composition comprises three or more active agents wherein at least one active agent is in controlled-release form. The controlled-release form can be in an amount that is effective to protect the agent from rapid elimination from the body. Certain preparations relating to the controlled-release of a pharmaceutical are taught in United States Patent Publication US 2005/0147710A1 entitled, “Powder Compaction and Enrobing” which is incorporated herein in its entirety by reference. Examples of time release coated beads are disclosed in U.S. Application Publication No. 20080131517.

In a further instance, at least one pharmaceutically active agent in a controlled-release form is an opioid analgesic agent. In some instances, pharmaceutical compositions comprise one or more carriers that protect the agents against rapid elimination from the body, such as time-release formulations or coatings. Such carriers include controlled-release formulations, including, for example, microencapsulated delivery systems. The active agents can be included in the pharmaceutically acceptable carrier in amounts sufficient to treat a subject's pain, with reduced adverse effects.

In some instances, a pharmaceutical composition is in an oral-dosage form and comprises a matrix that includes, for example, a controlled-release material and an opioid or non-opioid analgesic described herein. In certain instances, the matrix is compressible into a tablet and can be overcoated with a coating that can control the release of the opioid or non-opioid analgesic from the pharmaceutical composition. In this instance, blood levels of analgesics are maintained within a therapeutic range over an extended period of time. In certain alternate instances, the matrix is encapsulated. In some instances, a pharmaceutical composition is in an oral-dosage form and comprises a matrix that includes, for example, an immediate-release material and an antiemetic described herein.

Tablets or capsules containing a pharmaceutical composition described herein can be coated or otherwise compounded to provide a dosage form affording the advantage of prolonged action. For example, the tablet or capsule can contain an inner dosage and an outer dosage component, the latter being in the form of an envelope over the former. The two components can be separated by an enteric layer that serves to resist disintegration in the stomach and permit the inner component to pass intact into the duodenum or to be controlled in release. For controlled release, the capsule can also have micro drilled holes.

A coating comprising a side-effect-reducing compound, in immediate-release form, can be added to the outside of a controlled-release tablet core to produce a final dosage form. Such a coating can be prepared by administering a compound like promethazine with polyvinylpyrrolidone (PVP) 29/32 or hydroxypropyl methylcellulose (HPMC) and water/isopropyl alcohol and triethyl acetate. Such an immediate-release coating can be spray coated onto the tablet cores. The immediate-release coating can also be applied using a press-coating process with a blend consisting of 80% by weight promethazine and 20% by weight of lactose and hydroxypropyl methylcellulose type 2910. Press-coating techniques are known in the art and are described in U.S. Pat. No. 6,372,254, which is herein incorporated by reference in its entirety.

The immediate-release or controlled-release dosage forms described herein can also take the form of a bi-layered or a two layer tablet, which comprises a first layer and a second layer. The first layer comprises a first drug that is an analgesic, or antiemetic. The second layer comprises a second drug that is an analgesic, or antiemetic. The second drug is different from the first drug.

In a further instance of a bi-layered tablet, one layer is an immediate-release layer and the other layer is a controlled-release layer. In one example a bi-layered is formulated using the methods disclosed in U.S. Pat. No. 4,820,522, which is herein incorporated by reference in its entirety. In some instances of the bi-layered tablet described herein, both layers can comprise an opioid analgesic, a non-opioid analgesic and a compound to reduce or suppress adverse effects. In a further instance of the bi-layered tablet described herein, the immediate-release layer comprises promethazine or a salt thereof and the controlled-release layer comprises hydrocodone or oxycodone or a pharmaceutically acceptable salt of any one of the foregoing, or any combination thereof. In some instances, the immediate or controlled-release layer can further comprise acetaminophen or naproxen or a salt thereof. In some instances of the multi-layered tablet, the second drug can have a plasma half-life that differs from the plasma half-life of the first drug by at least about 5 hours.

In another instance, an effective amount of an antiemetic agent in an immediate-release form can be coated onto a substrate. For example, where the one or more opioid analgesics and one or more stimulant are components of a controlled-release formulation, an immediate-release layer comprising the antiemetic agent can overcoat the controlled-release formulation.

In another instance, an immediate-release layer can be coated onto the surface of a substrate having a controlled-release matrix. Where a plurality of controlled-release substrates comprising an effective unit dose of a pharmaceutically active agent (e.g., multiparticulate systems including pellets, spheres, beads and the like) are incorporated into a hard gelatin capsule, another agent can be incorporated into the gelatin capsule via inclusion of an amount of immediate-release agent as a powder or granulate within the capsule. In some instances, the gelatin capsule itself can be coated with an immediate-release layer. One skilled in the art recognizes still other alternative means of incorporating the immediate-release side-effect-reducing compound into the unit dose. Therefore, in some instances, by including an effective amount of an antiemetic agent in the unit dose, the subject is prepared for the eventual and subsequent release of one or more opioid analgesic in the controlled-release layer, where the antiemetic agent can reduce or prevent adverse effects associated with an opioid agent including but not limited to nausea, retching, vomiting, constipation, other gastric upsets, skin rashes, sleep disturbance, allergic reactions such as swelling, difficulty breathing, closing of throat, abdominal pain, unusual bleeding or bruising, skin rashes, sedation, CNS depression, or respiratory depression in subjects can be significantly reduced. In some instances, a stimulant is included in the unit dose.

An immediate-release or controlled-release dosage form described herein can also take the form of a bi-layered tablet, which can comprise an immediate-release layer and a controlled-release layer. The immediate-release or controlled-release dosage forms described herein can also take the form of a two layer tablet, which can comprise an immediate-release layer and a controlled-release layer. In some instances the immediate-release layer comprises an antiemetic agent, a stimulant and a non-opioid analgesic. In some instances the immediate-release layer comprises an antiemetic agent and a stimulant. In some instances the immediate-release layer comprises an antiemetic agent and a non-opioid analgesic. In some instances, the first layer can comprise one, two, three or more active agents. The controlled-release layer can comprise an opioid analgesic or non-opioid analgesic or stimulant. Such classes of active agents are described herein above.

An immediate-release or controlled-release dosage form described herein can also take the form of pharmaceutical particles manufactured by a variety of methods, including but not limited to high-pressure homogenization, wet or dry ball milling, or small particle precipitation (nano spray). Other methods to make a suitable powder formulation are the preparation of a solution of active ingredients and excipients, followed by precipitation, filtration, and pulverization, or followed by removal of the solvent by freeze-drying, followed by pulverization of the powder to the desired particle size.

In some instances, particles disclosed herein have a final size of 3-1000 μM, such as at most 3, 4, 5, 6, 7, 8, 9, 10, 20, 30, 40, 50, 60, 70, 80, 90, 100, 150, 200, 250, 300, 3500, 400, 4500, 500, 550, 600, 650, 700, 750, 800, 850, 900, 950, 1000 μM. In some instances, the pharmaceutical particles have a final size of 10-500 μM. In some instances, the pharmaceutical particles have a final size of 50-600 μM. In some instances, the pharmaceutical particles have a final size of 100-800 μM. These dosage forms can include immediate-release particles in combination with controlled-release particles in a ratio sufficient useful for delivering the desired dosages of active agents. In an alternative instance, a dosage unit can be divided into or exclusively included into both immediate-release and controlled-release particles.

In some instances, a dosage form herein can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water and/or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as one or more pharmaceutically active agents coated with a solvent protective or enteric coating. The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as, for example, sodium bicarbonate, sodium carbonate, potassium bicarbonate and potassium carbonate, magnesium carbonate and the like. Reactants which evolve oxygen or other gasses and which are safe for human consumption are also included. In some instances, citric acid and sodium bicarbonate is used.

In some instances, a dosage form disclosed herein can be in a candy form (e.g., matrix), such as a lollipop or lozenge. In some instances, one or more pharmaceutically active agents is dispersed within a candy matrix. In some instances, the candy matrix comprises one or more sugars (such as dextrose or sucrose). In another instance, the candy matrix is a sugar-free matrix. The choice of a particular candy matrix is subject to wide variation. Conventional sweeteners such as sucrose can be utilized, or sugar alcohols suitable for use with diabetic subjects, such as sorbitol or mannitol might be employed. Other sweeteners, such as aspartame, can also be easily incorporated into a pharmaceutical composition in accordance with pharmaceutical compositions described herein. The candy base can be very soft and fast dissolving, or can be hard and slower dissolving. Various forms can have advantages in different situations.

A containing candy mass comprising at least one pharmaceutically active agent can be orally administered to a subject in need thereof so that the agent can be released into the subject's mouth as the candy mass dissolves. The drug rapidly enters the subject bloodstream, and importantly, the blood in the veins draining from the mouth and the pharyngeal and esophageal areas passes through a substantial portion of the body (so that the drug can be absorbed) before the blood passes through the liver (where the drug can be inactivated). A subject in need thereof can include a human adult or child in pain, such as a child in sickle cell crisis, a child undergoing bone marrow transplant or a lumbar puncture procedure, a child with cancer (e.g., metastatic cancer, leukemia or lymphoma).

In some instances, candy matrix (lollipop or lozenge) comprises a pharmaceutical composition that lacks a stimulant. In some other instances, the candy matrix (lollipop or lozenge) comprises a pharmaceutical composition that comprises a stimulant. In these instances, the pharmaceutical composition provides an anti-sedative effect in addition to providing pain relief to a subject in need thereof.

In some instances a candy mass is prepared that comprises one or more layers which can comprise different pharmaceutically active agents and or rates of dissolution. In some instances, a multilayer candy mass (such as a lollipop) comprises an outer layer with a concentration of one or more pharmaceutically active agents differing from that of one or more inner layers. Such a drug delivery system has a variety of applications. By way of example, it can be desirable to quickly get a predetermined dose of a first pharmaceutically active agent into the bloodstream to obtain a desired effect and then use a different inner layer to deliver one or more other agents.

The choices of matrix and the concentration of the drug in the matrix can be important factors with respect to the rate of drug uptake. A matrix that dissolves quickly can deliver drug into the subject's mouth for absorption more quickly than a matrix that is slow to dissolve. Similarly, a candy matrix that contains one or more pharmaceutically active agents in a high concentration can release more of the one or more pharmaceutically active agents in a given period of time than a candy having a low concentration. In some instances, a candy matrix such as one disclosed in U.S. Pat. No. 4,671,953 or US Application 2004/0213828 (which is herein incorporated by reference in their entirety) is used to deliver the pharmaceutically active agents disclosed herein.

An immediate-release or controlled release dosage form described herein can also take the form of pharmaceutical particles manufactured by a variety of methods, including but not limited to high-pressure homogenization, wet or dry ball milling, or small particle precipitation (e.g., nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In some instances, the pharmaceutical particles have a final size of 3-1000 uM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 uM. In another instance, the pharmaceutical particles have a final size of 10-500 uM. In another instance, the pharmaceutical particles have a final size of 50-600 uM. In another instance, the pharmaceutical particles have a final size of 100-800 uM. These dosage forms can include immediate-release particles in combination with controlled-release particles in a ratio sufficient useful for delivering the desired dosages of active agents. For example, the immediate-release particles within a single tablet can comprise about 12.5 mg of promethazine or a salt thereof, and the controlled-release particles within a single tablet can comprise about 7.5 mg of hydrocodone or oxycodone or a salt thereof, and about 325 mg of acetaminophen or a salt thereof. For example, the immediate-release particles within a single tablet can comprise about 12.5 mg of promethazine or a salt thereof, and the controlled-release particles within a single tablet can comprise about 5 mg of hydrocodone or oxycodone or a salt thereof, and about 325 mg of acetaminophen or a salt thereof. For example, the immediate-release particles within a single tablet can comprise about 12.5 mg of promethazine or a salt thereof, and the controlled-release particles within a single tablet can comprise about 10 mg of hydrocodone or oxycodone or a salt thereof, and about 325 mg of acetaminophen or a salt thereof.

In some instances, an agent disclosed herein is released from a multi-layered tablet that comprises at least a first layer, a second layer and a third layer. The layers containing a pharmaceutically active agent can be separated by one or more layers of inert materials. In some instances, the layers containing a pharmaceutically active agent can have similar rates of release, e.g., all are immediate-release or all are controlled-release. In some instances, the layers have different rates of release. In such a case, at least one layer can be an immediate-release layer and at least one layer can be a controlled-release layer. In some instances, the multilayer tablet comprises at least three layers, each of which contains a different agent, such as: layer one contains promethazine or a salt thereof; layer two comprises hydrocodone or oxycodone or a salt thereof; and layer three comprises acetaminophen or a salt thereof. In this instance, the promethazine layer can be immediate-release, while the other two layers can be controlled-release.

In some instances, any of the pharmaceutical compositions disclosed herein can be formulated in a liquid dosing form. The liquid dosing form can be for oral administration, intravenous injection, intramuscular injection, or for topical administration (e.g., as a cream or gel). An orally administered liquid dosing form can be beneficial for subjects that have dysphagia or difficulty swallowing. A single dose of an orally administered liquid dosing form can be from 1 mL to about 500 mL in volume, or more. For example, the single dose of an orally administered liquid dosing form can be about 1-500 mL, 1-250 mL, 1-100 mL, 1-50 mL, 1-30 mL, 1-20 mL, 1-15 mL, 1-10 mL, 1-5 mL, 12.5 mL, 2.5-50 mL, 2.5-30 mL, 2.5-20 mL, 2.5-15 mL, 2.5-10 mL, 2.5-5 mL, 5-50 mL, 5-30 mL, 520 mL, 5-15 mL, 5-10 mL, 10-50 mL, 10-30 mL, 10-20 mL, 10-15 mL, 15-50 mL, 15-30 mL, 15-20 mL, 20-50 mL, 20-30 mL, 30-50 mL, 1 mL, 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 30 mL, 35 mL, 40 mL, 45 mL, 50 mL, 60 mL, 70 mL, 80 mL, 90 mL, 100 mL, 110 mL, 120 mL, 130 mL, 140 mL, 150 mL, 160 mL, 170 mL, 180 mL, 190 mL, 200 mL, 250 mL, 300 mL, 350 mL, 400 mL, 450 mL, or 500 mL. The liquid dosing form can comprise one or more of an opioid analgesic, a non-opioid analgesic, an antiemetic, a laxative, a stimulant, a barbiturate, an abuse-deterrent agent, or other active ingredient(s). In some instances, the liquid dosing form comprises an opioid analgesic such as hydrocodone, a non-opioid analgesic such as acetaminophen, and an antiemetic such as promethazine.

In some instances, any of the pharmaceutical compositions disclosed herein can be formulated in a liquid dosing form. The liquid dosing form can be for oral administration, intravenous injection, intramuscular injection, or for topical administration (e.g., as a cream or gel). An orally administered liquid dosing form can be beneficial for subjects that have dysphagia or difficulty swallowing. The liquid dosage form can include one or more pharmaceutically acceptable carriers or excipients.

Through selection and combination of the pharmaceutically acceptable carriers and excipients liquid dosage forms of any of the compositions disclosed herein can be provided that exhibit improved or more desired performance with respect to drug concentration, dissolution, dispersion, stability, safety, emulsification, efficacy, flavor, patient compliance, bioavailability, and/or other pharmacokinetic, chemical and/or physical properties. In some instances, an effective amount of one or more active ingredients (e.g., opioid analgesic, non-opioid analgesic, antiemetic, laxative, barbiturate, stimulant, etc.) can be dissolved to generate a substantially stable, or stable, solution with one or more of the pharmaceutically acceptable carriers or excipients as described herein.

In some instances, an oral liquid dosage form is a controlled-release oral liquid dosage form. The controlled-release oral liquid dosage form can provide for controlled or sustained release of one or more active ingredients (e.g., opioid analgesic, non-opioid analgesic, antiemetic, laxative, barbiturate, stimulant, etc.) from a gel, matrix, capsule, or resin material, or any combination of controlled or sustained release technology that can be suspended or dissolved in a liquid formulation.

The controlled-release oral liquid dosage form can comprise one or more excipients such as xanthan gum, sodium alginate, complex coacervate pairs such as gelatin or other polymers and carrageenan, and thermal gelling methycellulose formulations. Such excipients can influence the dissolution and/or diffusion rate of a suspended active ingredient so as to modify the absorption characteristics of the active ingredient as compared to a conventional oral liquid dosage form. The controlled-release oral liquid dosage form can be administered in a normally liquid formulation and only subsequently form a semi-solid or gel-like persistent matrix in the environment of the stomach.

In some instances, a controlled-release oral liquid dosage form comprises an aqueous, partially aqueous or non-aqueous solution or suspension of xanthan gum, sodium alginate, or sodium alginate LV (low viscosity, calcium depleted), gelatin and carrageenan, methylcellulose, or any combination thereof. In some instances, the controlled-release oral liquid dosage form comprises xanthan gum (e.g., Kelco SS-4749 and other commercially available types) in an amount of from about 0.3 to about 3.0 percent by weight. In another instance, the controlled-release oral liquid dosage form comprises xanthan gum in an amount of about 1.0 percent by weight. In some instances, the controlled-release oral liquid dosage form comprises sodium alginate in an amount of from about 0.5 to about 3.0 weight percent, or from about 0.3 to about 1.5 percent by weight of each gelatin and carrageenan. In some instances, each carrageenan of the iota type and gelatin type B is present at levels of at least about 0.5 percent by weight. In another instance, the controlled-release oral liquid dosage form comprises at least about 1 weight percent of sodium alginate. In another instance, the controlled-release oral liquid dosage form comprises methylcellulose (e.g., Type A15C, Dow Chemical Co.) in an amount of from about 1.0 to about 3.0 weight percent. In another instance, the controlled-release oral liquid dosage form comprises methylcellulose (e.g., Type A15C, Dow Chemical Co.) in an amount of about 2.0 weight percent.

The controlled-release oral liquid dosage form can comprise other excipients such as, for example, locust bean gum, salts such as NaCl, sugars such as sorbitol, Na₃ PO₄, CaCO₃, Ca₂HPO₄ and the like. The controlled-release oral liquid dosage form can comprise carbonate compounds such as calcium carbonate. The calcium carbonate can “float” the gelatinous matrix in a predetermined region of the stomach so that it is contacted with the most appropriate pH environment for a prolonged time period due to delayed gastric emptying.

The controlled-release oral liquid dosage form can include aqueous solutions or suspensions, partially aqueous solutions or suspensions such as, for example, high or low molecular weight glycerin, alcohols and glycols or even non-aqueous solutions or suspensions such as, for example, high or low molecular weight glycerin, alcohols and glycols.

Further information on liquid formulations can be found in U.S. Pat. No. 4,717,713; U.S. Pat. No. 4,788,055; and US 2007/0286875, each of which is hereby incorporated by reference in its entirety.

### Transdermal Dosage Forms

Another instance relates to a method of use and a system for a transdermal delivery of one or more pharmaceutically active agents into a subject. In some instances, a portion of the skin of a subject is sealed with a thin, film layer of a base material to occlude the skin and transport a desired dosage of at least one pharmaceutically active agent across the a layer, which can be from a rate-controlling system in contact with the thin layer. The rate-controlling system can be a thin rate-controlling membrane interposed between one or more agents and the thin layer. In another instance, a reservoir delivers at least one pharmaceutically active agent to the layer for delivery into a subject. In some instances, the pharmaceutically active agents to be delivered are: an opioid analgesic, a non-opioid analgesic and an antiemetic; or pharmaceutically acceptable salts, solvates, or prodrugs thereof; one or more pharmaceutically acceptable excipients or carriers.

In some instances, the rate-controlling system or reservoir comprises at least one pharmaceutically active agent to be delivered, is dispersed in a base material and contained within a container system. In some instances, at least one pharmaceutically active agent is dissolved in the base material. In another instance, at least one pharmaceutically active agent is uniformly dispersed in the base material. In another instance, the rate-controlling system or reservoir comprises microparticles of at least one pharmaceutically active agent to be delivered suspended in a base material and contained within a container system. In some instances, the base material is a viscous material. The container system can comprise a macroporous, non-rate-controlling face membrane with an impervious backing to form a pool or patch-like system of desired face membrane area with the face of the membrane placed over and in contact with the thin, occluding, viscous layer on the skin. The thin viscous layer can be coated or placed on the skin repeatedly, and the patch system placed on top of the thin, viscous layer or the viscous layer formed in situ by exudation through the membrane face when the patch or pool system is placed in position on the skin. In some instances, the patch or pool container system generally is retained in a transdermal position by the use of a peripheral adhesive layer about the patch or pool. In some instances, the face or transport area of the membrane is covered prior to use by a removable cover such as a peelable strip of impervious sheet material. In another instance, microcapsules containing a drug for delivery can be suspended in a viscous base material, and the pharmaceutical composition then spread as a layer over the skin of the user with or without a covering material. In some instances, the pharmaceutical compositions are administered to a subject via a transdermal patch. In some instances, transdermal patches (such as those disclosed in U.S. Pat. Nos. 4,906,463; 4,588,580; 4,685,911, 4,626,539, 4,834,978 and 5,635,204 d) can be used for the practice methods and compositions described herein, which are herein incorporated by reference in their entirety.

### Suppository Dosage Forms

In some instances, pharmaceutical compositions disclosed herein are in the form of a suppository. In some instances, the suppository is useful for vaginal or rectal administration. In some instances the suppository is effervescent. In some instances, the suppository base material contains hydrophobic or hydrophilic media, each of which can melt at body temperature. In some instances, the suppository base material used can be cocoa butter or similar material. In another instance, the suppository base material can be a moist polymer is then mixed with the one or more pharmaceutically active agents and compressed into the desired form. In some instances, at least one pharmaceutically active agent is dissolved in the suppository base material. In another instance, at least one pharmaceutically active agent is uniformly dispersed in the suppository base material. In another instance, the suppository base material comprises microparticles of at least one pharmaceutically active agent to be delivered suspended in the suppository base material. In some instances (such as vaginal suppositories), the suppository is effervescent. In some instances, the effervescing properties are imparted for the purpose of enhancing the rapid disintegration properties of the suppository. In other instances, U.S. Pat. Nos. 4,265,875 and 4,853,211 disclose useful suppositories which can be used for the practice of methods and compositions described herein, which are herein incorporated by reference in their entirety.

### Abuse Safeguard Dosage Forms

### Adverse-Effect Agents

In some instances, a pharmaceutical composition safeguards against abuse of the opioid analgesic agent. For example, a pharmaceutical composition disclosed herein can further comprise an effective amount of an adverse-effect agent or antagonist agent that reduces or eliminates one or more of: (1) the capacity of the opioid analgesic agent to produce the kind of physical dependence in which withdrawal causes sufficient distress to bring about drug-seeking behavior; (2) the ability to suppress withdrawal symptoms caused by withdrawal from the opioid analgesic agent; and (3) the induction of euphoria. Useful adverse-effect agents include, but are not limited to, opioid antagonists. In some instances, an antidote of the opioid analgesic can be included as an adverse-effect agent to reduce the potential for an overdose. The phrase “adverse-effect agent” is also meant to encompass all pharmaceutically acceptable salts of the adverse-effect agent. Examples of adverse-effect agents can include opioid antagonists. In such instances, exemplary opioid antagonists can include naloxone, naltrexone, nalmefene, cyclazacine, levallorphan, or a salt thereof, and mixtures thereof. In certain instances, the opioid antagonist can be naloxone, naltrexone or a pharmaceutically acceptable salt of any one of the foregoing, or any combination thereof.

In some instances, an opioid agent and the opioid antagonist is present in a ratio of opioid antagonist to opioid agent (analgesic) which is analgesically effective when the combination is administered orally, but which is aversive in a physically dependent subject. In this manner, the combination product (antagonist/agonist) could in essence be therapeutic to one population (subjects in pain), while being unacceptable (aversive) in a different population (e.g., physically dependent subjects) when orally administered at the same dose or at a higher dose than the usually prescribed dosage, e.g., about 2-3 times the usually prescribed dose of the opioid analgesic. Thus, the oral dosage form would have less potential for parenteral as well as oral abuse. In some instances, where the opioid is hydrocodone or oxycodone or a salt thereof and the antagonist is naltrexone or a salt thereof, the ratio of naltrexone or a salt thereof to hydrocodone or a salt thereof is from about 0.02-0.35:1 by weight, and in some instances from about 0.05-0.2:1 by weight. In some instances, the ratio of naltrexone or a salt thereof is in an amount from about 0.5 to about 4 mg per 15 mg of hydrocodone or a salt thereof. In another instance, the ratio of naltrexone or a salt thereof is in an amount from about 0.75 mg to about 3 mg per 15 mg hydrocodone or a salt thereof. In another example where the opioid antagonist is naltrexone or a salt thereof and the opioid agent is hydromorphone or a salt thereof, the ratio of naltrexone or a salt thereof to hydromorphone or a salt thereof can be from about 0.14:1 to about 1.19:1, or from about 0.222:1 to about 0.889:1. In another example where the opioid antagonist is naltrexone or a salt thereof and the opioid agent is oxycodone or a salt thereof, the ratio of naltrexone or a salt thereof to oxycodone or a salt thereof is about 0.03:1 to about 0.3:1, or from about 0.056:1 to about 0.222:1. In some instances, the opioid is hydrocodone, hydromorphone, oxycodone, fentanyl, or a pharmaceutically acceptable salt of any one of the foregoing, or any combination thereof.

In some instances, an opioid antagonist is administered in an amount (i) which does not cause a reduction in the level of analgesia elicited from the dosage form upon oral administration to a non-therapeutic level and (ii) which provides at least a mildly negative, “aversive” experience in physically dependent subjects (e.g., precipitated abstinence syndrome) when the subjects attempt to take at least twice the usually prescribed dose at a time (and often 2-3 times that dose or more), as compared to a comparable dose of the opioid without the opioid antagonist present. In certain instances, an amount of naltrexone or a salt thereof is included in the oral dosage form and is less positively reinforcing (e.g., less “liked”) to a non-physically dependent opioid user than a comparable oral dosage form without the antagonist included. In some instances, the composition provides effective analgesia when orally administered. In some instances, the oral dosage form can be administered on a twice-a-day or a once-a-day basis. In some instances, the user can be an addict. In some instances, the user can be physically dependent on the opioid. In some instances, the user can be a recreational user.

In some instances, a pharmaceutical composition is formulated as a controlled oral formulation in any suitable tablet, coated tablet or multiparticulate formulation known to those skilled in the art. In some instances, the controlled-release dosage form includes a carrier which is incorporated into a matrix or can be applied as a controlled-release coating. In some instances, in which an opioid analgesic is hydrocodone (or a pharmaceutically acceptable salt thereof), controlled release oral dosage forms can include analgesic doses from about 4 mg to about 60 mg of hydrocodone or a salt thereof per dosage unit. In a controlled-release oral dosage forms where hydromorphone or a salt thereof is the therapeutically active opioid, it can be included in an amount from about 2 mg to about 64 mg hydromorphone hydrochloride. In yet another instance, the opioid analgesic is oxycodone and the controlled-release oral dosage forms include from about 2.5 mg to about 800 mg oxycodone HCl. In some instances, a dosage form can contain molar equivalent amounts of other salts of the opioids useful in pharmaceutical compositions described herein. In some instances, U.S. Pat. Nos. 6,228,863; 6,475,494; 7,201,920; and U.S. Pat. Nos. 7,172,767, 7,201,920 disclose useful opioid agent/opioid antagonist formulations which can be used for the methods and compositions described herein, which are herein incorporated by reference in their entirety.

In some instances, one or more non-opioid analgesic agents, in addition to the opioid antagonist, can be included in the dosage form. Such non-opioid drugs can provide additional analgesia, and include, for example, acetylsalicylic acid; acetaminophen; non-steroidal anti-inflammatory drugs (“NSAIDS”), e.g., ibuprofen, naproxen, ketoprofen, etc.; N-methyl-D-aspartate (NMDA) receptor antagonists, e.g., a morphinan such as dextromethorphan or dextrorphan, or ketamine; cycooxygenase-II inhibitors (“COX-II inhibitors”); and/or glycine receptor antagonists.

### Abuse Deterrent Agents

In some instances, a pharmaceutical composition can comprise an opioid analgesic safeguards against abuse by further comprising one or more abuse deterrent agents. The choice of which abuse deterrent agent to include in a pharmaceutical composition can be varied depending on the route of administration and intended method of treatment. For example, different abuse deterrent agents can be used in conjunction with same pharmaceutically active agents depending on if they are formulated as an oral dosage form or a transdermal dosage form. Similarly, pharmaceutical compositions intended to treat a cancer associated pain in a subject can comprise a different abuse deterrent agent than a pharmaceutical composition intended to treat headache associated pain in a subject.

In some instances, an abuse deterrent agent is formulated as a gel-forming agent, and can comprise one or more mucous membrane irritants or nasal passageway tissue irritants. In another instance, the pharmaceutical compositions described herein include a pharmaceutical composition comprising an analgesic, one or more gel-forming agents and one or more emetics as described herein. In another instance, the pharmaceutical compositions comprise an opioid analgesic, one or more mucous membrane irritants or nasal passageway tissue irritants and one or more emetics as described herein. In one particular instance, the pharmaceutical compositions comprise an analgesic, one or more gel-forming agents, one or more mucous membrane irritants and/or nasal passageway tissue irritants, and one or more emetics.

Suitable gel-forming agents include compounds that, upon contact with a solvent (e.g., water), absorb the solvent and swell, thereby forming a viscous or semi-viscous substance that significantly reduces and/or minimizes the amount of free solvent which can contain an amount of solublized drug, and which can be drawn into a syringe. The gel can also reduce the overall amount of drug extractable with the solvent by entrapping the drug in a gel matrix. In some instances, typical gel-forming agents include pharmaceutically acceptable polymers, typically hydrophilic polymers, such as hydrogels.

In some instances, a polymer herein exhibits a high degree of viscosity upon contact with a suitable solvent. The high viscosity can enhance the formation of highly viscous gels when attempts are made by an abuser to crush and dissolve the contents of a dosage form in an aqueous vehicle and inject it intravenously. In some instances, a polymeric material described herein provides viscosity to the dosage form when it is tampered. In such instances, when an abuser crushes and dissolves the dosage form in a solvent (e.g., water or saline), a viscous or semi-viscous gel is formed. The increase in the viscosity of the solution discourages the abuser from injecting the gel intravenously or intramuscularly by preventing the abuser from transferring sufficient amounts of the solution to a syringe to cause a desired “high” once injected. Suitable polymers include one or more pharmaceutically acceptable polymers selected from any pharmaceutical polymer that can undergo an increase in viscosity upon contact with a solvent. Polymers can include polyethylene oxide, polyvinyl alcohol, hydroxypropyl methyl cellulose and carbomers.

In some instances, a pharmaceutical composition comprises an abuse deterrent agent that is a mucous membrane irritant or nasal passageway tissue irritant, or both. These irritants are designed to deter abuse via the improper administration of a dosage form comprising an opioid (e.g., crushing and snorting). In some instances, suitable mucous membrane irritants or nasal passageway tissue irritants include compounds that are generally considered pharmaceutically inert, yet can induce irritation. Such compounds include, but are not limited to surfactants. In some instances, suitable surfactants include sodium lauryl sulfate, poloxamer, sorbitan monoesters and glyceryl monooleates. Other suitable compounds are believed to be within the knowledge of a practitioner skilled in the relevant art, and can be found in the Handbook of Pharmaceutical Excipients, 4th Ed. (2003), the entire content of which is hereby incorporated by reference.

In some instances, an irritant is present in amount of from 1 to 10 percent by weight on a solid basis, such as from about 1 to 5 percent by weight on a solid basis. In another instance, the amount of irritant can be present in an amount from 1 to 3 percent by weight. In some instances, an irritant can deter abuse of a dosage form when a potential abuser tampers with a dosage form described herein. Specifically, in such instances, when an abuser crushes the dosage form, the irritant is exposed. The irritant discourages inhalation of the crushed dosage form by inducing pain and/or irritation of the abuser's mucous membrane and/or nasal passageway tissue. In some instances, the irritant discourages inhalation (e.g., via snorting through the nose) by inducing pain and/or irritation of the abuser's nasal passageway tissue. In some instances, a pharmaceutical composition described herein can comprise one or more mucous membrane irritants that cause irritation of mucous membranes located anywhere on or in the body, including membranes of the mouth, eyes and intestinal tract. Such pharmaceutical compositions can deter abuse via oral, intra-ocular or rectal or vaginal routes.

In some instances, a pharmaceutical composition comprises an abuse deterrent agent that is an emetic or emesis inducing agent. In some instances, the emetic can be a pharmaceutically acceptable inert excipient that only induces emesis after a certain threshold amount is ingested. In another instance, the emetic can be a pharmaceutically active emetic. In some instances, an amount of emetic present in a pharmaceutical composition described herein can be tied directly to the amount of drug in the pharmaceutical composition. Thus, by controlling the quantity of the emetic compound in the pharmaceutical composition, emesis can be avoided if normal prescription directions are followed. However, if an overdosage occurs by ingesting more than a prescribed quantity of a drug in a pharmaceutical composition described herein, the amount of ingested emetic can exceed the threshold amount necessary to induce emesis.

In some instances, a threshold amount of emetic for inducing emesis is reached when the normal prescription directions are inappropriately increased by factors of 2, 3, 4, 5, 6, 7, or 8 times, or more. Thus, in some instances, the amount of emetic present in a pharmaceutical composition described herein is an amount such that the amount of emetic ingested does not exceed the threshold amount necessary for inducing emesis until a subject ingests 2, 3, 4, 5, 6, 7, or 8 or more times the amount of drug normally prescribed. In some instances, emesis can preclude death or serious illness in the subject. In some instances, an emetic is zinc sulfate. Zinc sulfate is an excipient, which can induce emesis when more than about 0.6 to 2.0 gm is ingested, typically more than about 0.6 gm, or about 5 to 25 percent by weight on a solid basis, more typically about 5 to 10 percent by weight.

In some instances, a pharmaceutical composition described herein can be easily designed to induce emesis if a prescribed dosage is exceeded and/or if prescription directions are not followed for dosage forms containing a pharmaceutical composition described herein. Typically, suitable instances include less than about 0.6 to 2.0 gm of zinc sulfate. For example, a dosage form can induce emesis only after a pre-determined number of dosage forms are ingested (such as 4, 5, 6 or more), in this instance the amount of zinc sulfate in each dosage form should not exceed about 0.19 gm. Thus, if three dosage forms are ingested, the amount of emetic can be 0.57 gm, which is less than a typical threshold amount of the particular emetic. However, if a fourth dosage form having 0.19 gm. of zinc sulfate is ingested, the amount of emetic exceeds the threshold amount, and emesis is induced.

In some instances, a pharmaceutical composition comprises an effective amount of an abuse deterrent agent that induces flushing, (i.e. redness of the skin, including redness of the skin of one or more of the face, neck, chest, back and trunk and legs) and/or itching and/or discomfort and/or temporary pain (a flushing/pain inducing agent or flushing inducing agent), and/or generalized pruritis, and/or intense warmth, and/or chills when administered at or in excess of a threshold amount. With respect to flushing, discomfort and pain inducing agents, a threshold amount is an amount below which one or more adverse effects is absent or below which a subject can experience a beneficial effect. In some instances, a flushing agent or itching agent or pain-inducing agent is a drug. In certain instances, the drug is obtainable “over the counter” and in certain instances, the “over the counter” drug is a vitamin. In yet another instance, the vitamin is niacin. Another instance includes vitamin. Accordingly, In some instances an amount of flushing, itching, or pain inducing agent present in a pharmaceutical composition described herein can be tied directly to the amount of drug in the pharmaceutical composition. Thus, by controlling the quantity of the flushing, itching, or pain inducing agent in the pharmaceutical composition, flushing, itching, or pain can be avoided if normal prescription directions are followed. However, if an overdosage occurs by ingesting more than a prescribed quantity of a drug in a pharmaceutical composition described herein (e.g., by ingesting more than the prescribed dose), the total amount of flushing, itching, or pain inducing agent can, in certain instances, exceed the threshold amount necessary to induce flushing, itching, or pain thereby inducing flushing, itching, or pain.

In some instances, a pharmaceutical composition or method described herein includes about 10 mg to about 500 mg of the flushing, itching, or pain inducing agent. In yet another instance, a pharmaceutical composition comprises about 15 mg to about 150 mg of a flushing, itching, or pain agent. In another instance, a pharmaceutical composition comprises 15, 30, 45, 60, 75, 90 or 105 mg of a flushing, itching, or pain inducing agent. In some instances, pharmaceutical compositions and methods described herein includes a flushing, itching, or pain inducing agent in an amount of about 1% to 25%, typically about 3% to 15%, more typically about 1%, 3%, 6%, 9%, 12%, 15% or 20% by weight, including or excluding the weight of any analgesic and/or other drug susceptible to abuse.

In some instances of dosage forms having a controlled-release layer or formulation, an amount of flushing inducing agent (and in other instances, the amount of any abuse deterrent component or opioid antagonist described herein), can exceed the threshold amount present in an immediate-release form. This can be because in controlled-release formulations, the amount of drug which is susceptible to abuse is typically higher than in an immediate-release formulation and the flushing inducing agent (or other abuse deterrent component) becomes bioavailable at a slower rate than the immediate-release form. Thus, the amount of abuse deterrent component which is bioavailable typically also remains below the amount sufficient to cause an abuse deterrent effect. However, if the dosage form is tampered with (e.g., ground, chewed or crushed), a large portion of the abuse deterrent component becomes immediately bioavailable, thus inducing one or more abuse deterrent effects.

Examples of abuse deterrent agents that can be used in pharmaceutical compositions described herein are disclosed in US Patent Application Nos: US20060177380A1; US20060110327A1; and US20070231268A1, which are herein incorporated by reference in its entirety.

### Abuse Deterrence Via Chemical Modification of Active Agents

In some instances, a pharmaceutical composition can comprise an opioid agent that is conjugated to a chemical moiety. The chemical moiety can be any chemical substance that can be attached to the opioid agent in a manner that renders it pharmacologically inactive. Analgesics and stimulants produce their pharmacological effects through binding to specific receptors or uptake proteins. The attachment of certain chemical moieties can therefore prevent the active substance from binding its receptor(s) or recognition site on its uptake protein. Further, without being bound by theory, the covalent modification is believed to prevent the pharmacological effect by preventing the drug from crossing the blood-brain barrier. The attachment of the chemical moiety to the opioid agent can also prevent or substantially delay the absorption of the compound, particularly when the compound is delivered by routes other than oral administration.

In some instances, a chemical moiety is attached to an opioid agent in a manner in which it is not readily released by conditions found in the mouth (saliva), the intranasal cavity, the surface of the lungs, or in the serum. Extreme acid conditions encountered in the stomach are not present elsewhere in humans. Therefore, any acid dependent release mechanism can occur only after oral administration. Although, degradative enzymes are present in the aforementioned environments, they are not generally present in the high concentrations found in the intestinal tract. Thus, release of the opioid agent by enzymatic cleavage cannot occur rapidly when the novel compounds are administered by routes other than oral delivery.

In another instance, an opioid agent is attached to a polymer of serine (or other amino acid containing a hydroxyl side chain e.g. threonine, tyrosine) via side chain hydroxyl groups. In some instances, attachment is to a polymer of glutamic acid through the carboxyl group of the delta carbon of glutamic acid. The resulting ester (carbonate) linkages can be hydrolysed by lipases (esterases) encountered in the small intestine. Esterases are not present at high levels in saliva or on the mucosal surfaces of the nasal cavity, lungs, or oral cavity. Thus, opioid agents attached to polyglutamic acid by this method would not be rapidly released by saliva or when delivered intranasally or by inhalation.

In another instance, an opioid agent is attached to an oligopeptide, which can consist of between one and five amino acids. In a further instance, the amino acids are a heterogeneous mixture of the twenty naturally occurring amino acids. Hydrophilic amino acids can tend to prevent passive absorption of the analgesic peptide conjugate through nasal membranes. In some instances, hydrophilic amino acids can be included in the oligopeptide. In another instance, lipophilic amino acids can be attached closer to the analgesic for optimum stability. Both lipophilic and hydrophilic properties (i.e., amphiphilic) can be satisfied with between three and five amino acids. In a further instance, the oligopeptide that is attached to the analgesic can be an amphiphilic tripeptide.

Amphiphilic amino acids/oligopeptides can contain (i) hydrophobic amino acids, located in positions next to the active agent to provide increased stability; (ii) amino acid sequences designed to be cleaved by intestinal enzymes (e.g. pepsin, trypsin, chymotrypsin, elastase, carboxypeptidases A and B, etc.) provide for increased bioavailability; (iii) peptides longer than three amino acids for increased stability, increased anti-abuse e.g. less membrane permeability, and potentially more efficient intestinal digestion e.g. major intestinal enzymes target proteins and polypeptides, (iv) or mixtures thereof. In some instances, the carrier portion of the conjugate is designed for intestinal cleavage. In another instance, cleavage specificity is directed to pepsin and/or chymotrypsin. Examples of carriers include XXXAA or XXAAA, where X is selected from any amino acid, except Arg, Lys, His, Pro, and Met and A is selected from Tyr, Phe, Tm, or Leu. Examples of other carriers are selected from XXXPheLeu wherein X is Glu; XXXPheLeu wherein X is Gly; XXPheLeuLeu wherein X is Glu; and XXPheLeuLeu wherein X is Gly. In another instance, cleavage specificity is directed to trypsin. Examples of more carriers include XXXAA or XXAAA wherein X is any amino acid except Pro and Cys and A is Arg or Lys. Examples of yet more carriers are selected from XXXArgLeu wherein X is Glu; XXXArgLeu wherein X is Gly; XXArgLeuLeu wherein X is Gly; XXXArgLeuLeu wherein X is Gly. Examples of chemical modifications to opioid agents that can be used in pharmaceutical compositions described herein are disclosed in US Patent Application No: 20050080012, which is herein incorporated by reference in its entirety.

In some instances, one or more adverse-effect-reducing active agents in addition to the opioid antagonist agent or abuse deterrent component is included in the dosage form. Adverse-effect-reducing active agents include but are not limited to promethazine, dolasetron, granisetron, ondansetron, tropisetron, palonosetron, domperidone, droperidol, haloperidol, chlorpromazine, prochloperazine, metoclopramide, alizapride, cyclizine, diphenhydramine, dimenhydrinate, meclizine, hydroxyzine, cannabis, dronabinol, nabilone, midazolam, lorazepam, hyoscine, dexamethasone, trimethobenzamide, emetrol and propofol.

### Administration

Described herein are methods for preventing an adverse effect such as nausea, retching, vomiting, constipation, other gastric upsets, sleep disturbance, skin rashes, itching, allergic reactions such as swelling, difficulty breathing, closing of throat, abdominal pain, unusual bleeding or bruising, skin rashes, sedation, CNS depression, or respiratory depression in a subject receiving, or in need of, opioid analgesic therapy. The prevention of an adverse effect can be accomplished by the administration of an effective amount of promethazine or other antiemetic along with the chosen analgesic agent or agents. In some instances, provided herein are methods for treating pain, comprising administering to a subject in need thereof an effective amount of an opioid analgesic agent, a non-opioid analgesic agent, an agent that reduces side effects of the opioid analgesic agent and a stimulant agent. In some instances, provided herein are methods for treating pain, comprising administering to a subject in need thereof an effective amount of an opioid analgesic agent, a non-opioid analgesic agent, an agent that reduces side effects of the opioid analgesic agent. In some instances, the non-opioid analgesic agent is acetaminophen. In another instance, the agent that reduces an adverse effect is promethazine. In another instance, provided herein are methods for treating pain, comprising administering to a subject in need thereof an effective amount of an opioid analgesic agent, a non-opioid analgesic agent, a barbiturate agent, an agent that reduces side effects of the opioid analgesic agent and a stimulant agent. In another instance, provided herein are methods for treating pain, comprising administering to a subject in need thereof an effective amount of an opioid analgesic agent, a non-opioid analgesic agent, a barbiturate agent, an agent that reduces side effects of the opioid analgesic agent. In another instance, provided herein are methods for treating pain, comprising administering to a subject in need thereof an effective amount of an opioid analgesic agent, a barbiturate agent, an agent that reduces side effects of the opioid analgesic agent and a stimulant agent. In another instance, provided herein are methods for treating pain, comprising administering to a subject in need thereof an effective amount of an opioid analgesic agent, a barbiturate agent, an agent that reduces side effects of the opioid analgesic agent. In another instance, provided herein are methods for treating pain, comprising administering to a subject in need thereof an effective amount of a non-opioid analgesic agent, a barbiturate agent, an agent that reduces side effects of the opioid analgesic agent and a stimulant agent. In another instance, provided herein are methods for treating pain, comprising administering to a subject in need thereof an effective amount of a non-opioid analgesic agent, a barbiturate agent, an agent that reduces side effects of the opioid analgesic agent. In another instance, provided herein are methods for treating pain, comprising administering to a subject in need thereof an effective amount of an opioid analgesic agent, an agent that reduces side effects of the opioid analgesic agent and a stimulant agent. In another instance, provided herein are methods for treating pain, comprising administering to a subject in need thereof an effective amount of an opioid analgesic agent, an agent that reduces side effects of the opioid analgesic agent.

An administration herein can continue for a relatively short period of time in the instance of an acute condition requiring opioid therapy or for a long period of time in the instance of conditions requiring chronic use of opioid analgesics. The dosing of analgesics can be dependent upon the condition being treated, the subject's individual perception of pain, the use of the opioid as a prophylactic to prevent the onset of pain by administering on a set time schedule or on an as needed basis in response to perceived pain. The choice of selecting a dosage of a pharmaceutical composition that contains suitable amount of promethazine can be dependent upon the extent and severity of the adverse effects including nausea, retching, vomiting, constipation, other gastric upsets, sleep disturbance, skin rashes, allergic reactions such as swelling, difficulty breathing, closing of throat, abdominal pain, unusual bleeding or bruising, skin rashes, sedation, CNS depression, or respiratory depression in a subject, upon the sensitivity to side-effect-reducing compounds such as promethazine in a subject, upon the likelihood of subject losing medication by vomiting, and/or on an as needed basis in response to perceived adverse effects. The dosage can be assessed by a prescribing professional evaluating the subject, the condition treated, the analgesic to be used, diet and the expected duration of therapy.

Also provided herein is a method for treating a subject suffering from or susceptible to pain, comprising administering to a subject in need thereof an effective amount of a pharmaceutical composition comprising an effective amount of a first component which is a non-opioid analgesic, or a pharmaceutically acceptable salt thereof, an effective amount of a second component which is a non-opioid analgesic, or a pharmaceutically acceptable salt thereof and an effective amount of a third component which is an antiemetic.

In some instances, a method for treating a subject is provided comprises administering an effective amount of a pharmaceutical composition comprising: an effective amount of a first pharmaceutically active agent which is an opioid analgesic, or a pharmaceutically acceptable salt thereof an effective amount of a second pharmaceutically active agent which is a non-opioid analgesic, or a pharmaceutically acceptable salt thereof and an effective amount of a third pharmaceutically active agent which is an antiemetic. In some instances, the at least one adverse effect is nausea, vomiting, retching, constipation, other gastric upsets, sleep disturbance, skin rashes, allergic reactions such as swelling, difficulty breathing, closing of throat, itching, abdominal pain, unusual bleeding or bruising, sedation, CNS depression, or respiratory depression. In some instances, the non-opioid analgesic is acetaminophen or analogue thereof. In some instances, the antiemetic is promethazine. In some instances, the opioid analgesic is hydrocodone. In another instance, the opioid analgesic is oxycodone. In another instance, provided herein are methods for preventing or ameliorating an adverse effect associated with administration of an analgesic, comprising administering to a subject in need thereof an effective amount of an opioid analgesic agent, a non-opioid analgesic agent, an agent that reduces side effects of the opioid analgesic agent and a stimulant agent. In another instance, provided herein are methods for preventing or ameliorating an adverse effect associated with administration of an analgesic, comprising administering to a subject in need thereof an effective amount of an opioid analgesic agent, a non-opioid analgesic agent, an agent that reduces side effects of the opioid analgesic agent. In some instances, the non-opioid analgesic agent is acetaminophen. In another instance, the agent that reduces an adverse effect is promethazine. In another instance, provided herein are methods for preventing or ameliorating an adverse effect associated with administration of an analgesic, comprising administering to a subject in need thereof an effective amount of an opioid analgesic agent, a non-opioid analgesic agent, a barbiturate agent, an agent that reduces side effects of the opioid analgesic agent and a stimulant agent. In another instance, provided herein are methods for preventing or ameliorating an adverse effect associated with administration of an analgesic, comprising administering to a subject in need thereof an effective amount of an opioid analgesic agent, a non-opioid analgesic agent, a barbiturate agent, an agent that reduces side effects of the opioid analgesic agent. In another instance, provided herein are methods for preventing or ameliorating an adverse effect associated with administration of an analgesic, comprising administering to a subject in need thereof an effective amount of an opioid analgesic agent, a barbiturate agent, an agent that reduces side effects of the opioid analgesic agent and a stimulant agent. In another instance, provided herein are methods for preventing or ameliorating an adverse effect associated with administration of an analgesic, comprising administering to a subject in need thereof an effective amount of an opioid analgesic agent, a barbiturate agent, an agent that reduces side effects of the opioid analgesic agent. In another instance, provided herein are methods for preventing or ameliorating an adverse effect associated with administration of an analgesic, comprising administering to a subject in need thereof an effective amount of an a non-opioid analgesic agent, a barbiturate agent, an agent that reduces side effects of the opioid analgesic agent and a stimulant agent. In another instance, provided herein are methods for preventing or ameliorating an adverse effect associated with administration of an analgesic, comprising administering to a subject in need thereof an effective amount of an a non-opioid analgesic agent, a barbiturate agent, an agent that reduces side effects of the opioid analgesic agent. In another instance, provided herein are methods for preventing or ameliorating an adverse effect associated with administration of an analgesic, comprising administering to a subject in need thereof an effective amount of an opioid analgesic agent, an agent that reduces side effects of the opioid analgesic agent and a stimulant agent. In another instance, provided herein are methods for preventing or ameliorating an adverse effect associated with administration of an analgesic, comprising administering to a subject in need thereof an effective amount of an opioid analgesic agent, an agent that reduces side effects of the opioid analgesic agent.

Also provided herein is are methods for preventing an adverse effect such as nausea, retching, vomiting, constipation, sleep disturbance, and a skin rash in a subject receiving or in need of opioid therapy by the administration of an effective amount of acetaminophen or analogue thereof and promethazine with the opioid analgesic agent. In some instances, the opioid analgesic is hydrocodone. In another instance, the opioid analgesic is oxycodone. In some instances, administration of a pharmaceutical composition comprising a non-opioid analgesic and an antiemetic enhances the reduction or elimination of adverse effects associated with an opioid analgesic. For example, addition of promethazine and acetaminophen/ibuprofen reduces or eliminates an adverse effect associated with an opioid analgesic in a synergistic manner.

In some instances, administration of a pharmaceutical composition disclosed herein results in treatment of a subject in need thereof which includes elimination or reduction of an adverse effect associated with analgesics (e.g., opioids) and enhance the beneficial uses of such analgesics. Such an adverse effect can otherwise render administration of certain analgesics intolerable, due to for example vomiting, nausea, retching, constipation, sleep disturbance, and skin rashes. Therefore, some instances of the methods herein are directed to target populations of subjects that are susceptible to such an adverse effect(s), thus allowing such subjects to benefit from the pain-alleviating effects of analgesic-based pain relief, administration of which would otherwise be intolerable.

For example, by reducing the risk of vomiting, the risk of subjects losing the analgesics (and losing the pain-relieving beneficial effects of analgesics) by vomiting is minimized. Furthermore, administration can be adjusted to provide the dose of side-effect-reducing compound to match the subject's analgesic ingestion without separate intervention by the health care professionals. Adding one or more additional active agents, such as promethazine, to the present pharmaceutical compositions is believed to result in a pharmaceutical composition having reduced potential for abuse and diversion.

### Routes of Administration

In some instances, active agents disclosed herein are formulated to be administered through oral dosage forms (e.g., tablets, capsules, gels, lollipops), inhalations, nasal sprays, patches, absorbing gels, liquids, liquid tannates, suppositories, injections, I.V. drips, other delivery methods, or any combination thereof to treat subjects in need thereof. Administration can be performed in a variety of ways, including, but not limited to orally, subcutaneously, intravenously, intranasally, intraoptically, transdermally, topically (e.g., gels, salves, lotions, creams, etc.), intraperitoneally, intramuscularly, intrapulmonary (e.g., AERx® inhalable technology commercially available from Aradigm, or Inhance, pulmonary delivery system commercially available from Inhale Therapeutics), vaginally, parenterally, rectally, or intraocularly.

To prepare a pharmaceutical composition disclosed herein, an effective amount of active agents can be mixed with a suitable pharmaceutically acceptable carrier. Upon mixing of the compounds, the resulting pharmaceutical composition can be a solid, a half-solid, a semi-solid, a solution, suspension, or an emulsion. Such pharmaceutical compositions can be prepared according to methods known to those skilled in the art. The forms of the resulting pharmaceutical compositions can depend upon a variety of factors, including the intended mode of administration and the solubility of the compounds in the selected carrier or vehicle. The effective concentration of analgesics is sufficient for lessening or alleviating pain. In some instances, the components of the present pharmaceutical compositions are at least one opioid analgesic agent (e.g., hydrocodone/oxycodone), one non-opioid analgesic agent (e.g., acetaminophen), and one antiemetic agent (e.g., promethazine). In other instances, administration comprises administration of an antiemetic (e.g., promethazine) separately, prior to, or during administration of the analgesic formulations described herein (e.g., which comprises hydrocodone and acetaminophen). In another instance, the components of the present pharmaceutical compositions are at least one opioid analgesic agent, a non-opioid analgesic agent, an agent that reduces side effects of the opioid analgesic agent and a stimulant agent. In another instance, the components of the present pharmaceutical compositions are at least one opioid analgesic agent, a non-opioid analgesic agent, a barbiturate agent, an agent that reduces side effects of the opioid analgesic agent and a stimulant agent. In another instance, the components of the present pharmaceutical compositions are at least one opioid analgesic agent, a non-opioid analgesic agent, a barbiturate agent, an agent that reduces side effects of the opioid analgesic agent. In another instance, the components of the present pharmaceutical compositions are at least one opioid analgesic agent, a barbiturate agent, an agent that reduces side effects of the opioid analgesic agent and a stimulant agent. In another instance, the components of the present pharmaceutical compositions are at least one opioid analgesic agent, a barbiturate agent, an agent that reduces side effects of the opioid analgesic agent. In another instance, the components of the present pharmaceutical compositions are at least one non-opioid analgesic agent, a barbiturate agent, an agent that reduces side effects of the opioid analgesic agent and a stimulant agent. In another instance, the components of the present pharmaceutical compositions are at least one non-opioid analgesic agent, a barbiturate agent, an agent that reduces side effects of the opioid analgesic agent. In another instance, components of the present pharmaceutical compositions are at least one opioid analgesic agent, an agent that reduces side effects of the opioid analgesic agent and a stimulant agent. In another instance, components of the present pharmaceutical compositions are at least one opioid analgesic agent, an agent that reduces side effects of the opioid analgesic agent.

In some instances, an agent described herein is administered by the nasal inhalation route using conventional nebulizers or by oxygen aerosolization to provide convenient pain relief with reduced adverse effects. The agents can be suspended or dissolved in a pharmacologically acceptable inhalation carrier. Examples of such carriers are distilled water, water/ethanol mixtures, and physiological saline solution. Conventional additives including sodium chloride, glucose, citric acid and the like can be employed in these dosage forms to stabilize or to provide isotonic media. In some instances, the pharmaceutical compositions suitable for nasal inhalation by oxygen aerosolization administration comprise hydrocodone or oxycodone, acetaminophen, and promethazine. In another instance, the pharmaceutical compositions suitable for nasal inhalation by oxygen aerosolization administration comprise hydrocodone or oxycodone, and promethazine. In other instances, an antiemetic (e.g., promethazine) can be administered separately, prior to, or during administration of the pharmaceutical compositions described herein (e.g., those comprising hydrocodone and acetaminophen).

In some instances, an agent described herein can also be administered as a self-propelled dosage unit in an aerosol form suitable for inhalation therapy. Suitable means for employing the aerosol inhalation therapy technique are described, for example, in U.S. Pat. No. 6,913,768 to Couch et al., a reference which is incorporated herein by reference in its entirety. The agent can be suspended in an inert propellant such as a mixture of dichlorodifluoromethane and dichlorotetrafluoroethane, together with a co-solvent such as ethanol, together with flavoring materials and stabilizers. In some instances, the agents useful for a self-propelled dosage unit in aerosol form administration are hydrocodone or oxycodone, acetaminophen, and promethazine. In another instance, the agents useful for a self-propelled dosage unit in aerosol form administration are hydrocodone or oxycodone, and promethazine. In a further instance, the dosage unit can further comprise an agent such as a bronchodilator (e.g., albuterol).

In some instances, an agent described herein can also be administered as nasal spray/drop pharmaceutical compositions, which can conveniently and safely be applied to subjects in need thereof to effectively treat pain with reduced adverse effects. The pharmaceutical compositions can further comprise a water soluble polymer such as polyvinylpyrrolidone, together with other medications and together with bioadhesive material. In some instances, the components of a pharmaceutical composition for nasal spray or drop administration are hydrocodone or oxycodone agent, acetaminophen, and promethazine, or a pharmaceutically acceptable salt of any one of the foregoing, or any combination thereof. In another instance, the components of a pharmaceutical composition for nasal spray or drop administration are hydrocodone or oxycodone agent, and promethazine, or a pharmaceutically acceptable salt of any one of the foregoing, or any combination thereof.

In some instances, a pharmaceutical composition described herein can also be administered topically to the skin of a subject in need thereof. The agents can be mixed with a pharmaceutically acceptable carrier or a base which is suitable for topical application to skin to form a dermatological pharmaceutical composition. Suitable examples of carriers or bases include but not limited to: water, glycols, alcohols, lotions, creams, gels, emulsions, and sprays. A dermatological pharmaceutical composition comprising an analgesic agent can be integrated into a topical dressing, medicated tape, dermal patch absorbing gel and cleansing tissues. In some instances, the dermatological pharmaceutical composition comprises hydrocodone or oxycodone, acetaminophen, and promethazine. In another instance, the dermatological pharmaceutical composition comprises hydrocodone or oxycodone, and promethazine.

The pharmaceutical compositions described herein can also be in liquid or liquid tannate form. The liquid formulations can comprise, for example, an agent in water-in-solution and/or suspension form; and a vehicle comprising polyethoxylated castor oil, alcohol and/or a polyoxyethylated sorbitan mono-oleate with or without flavoring. Each dosage form comprises an effective amount of an active agent and can comprise pharmaceutically inert agents, such as conventional excipients, vehicles, fillers, binders, disintegrants, pH adjusting substances, buffer, solvents, solubilizing agents, sweeteners, colorant agents and any other inactive agents that can be included in pharmaceutical dosage forms for oral administration. Examples of such vehicles and additives can be found in Remington's Pharmaceutical Sciences, 17th edition (1985). Therefore, In some instances a liquid pharmaceutical composition disclosed herein comprises an opioid analgesic (e.g., hydrocodone or oxycodone), a non-opioid analgesic (e.g., acetaminophen) and an antiemetic (e.g., promethazine). In another instance, a liquid pharmaceutical composition disclosed herein comprises at least one opioid analgesic agent, a non-opioid analgesic agent, an agent that reduces side effects of the opioid analgesic agent and a stimulant agent. In another instance, a liquid pharmaceutical composition disclosed herein comprises at least one opioid analgesic agent, a non-opioid analgesic agent, a barbiturate agent, an agent that reduces side effects of the opioid analgesic agent and a stimulant agent. In another instance, a liquid pharmaceutical composition disclosed herein comprises at least one opioid analgesic agent, a non-opioid analgesic agent, a barbiturate agent, an agent that reduces side effects of the opioid analgesic agent. In another instance, a liquid pharmaceutical composition disclosed herein comprises at least one opioid analgesic agent, a barbiturate agent, an agent that reduces side effects of the opioid analgesic agent and a stimulant agent. In another instance, a liquid pharmaceutical composition disclosed herein comprises at least one opioid analgesic agent, a barbiturate agent, an agent that reduces side effects of the opioid analgesic agent. In another instance, a liquid pharmaceutical composition disclosed herein comprises at least one non-opioid analgesic agent, a barbiturate agent, an agent that reduces side effects of the opioid analgesic agent and a stimulant agent. In another instance, a liquid pharmaceutical composition disclosed herein comprises at least one non-opioid analgesic agent, a barbiturate agent, an agent that reduces side effects of the opioid analgesic agent. In another instance, a liquid pharmaceutical composition disclosed herein comprises at least one opioid analgesic agent, an agent that reduces side effects of the opioid analgesic agent and a stimulant agent. In another instance, a liquid pharmaceutical composition disclosed herein comprises at least one opioid analgesic agent, an agent that reduces side effects of the opioid analgesic agent.

In some cases, the pharmaceutical compositions described herein are administered in a suppository form, comprising an outer layer containing the pharmaceutical composition in a suppository base. The suppository base can, for example, be any conventional suppository base material such as glycogelatin, polyethylene glycol, fractionated palm kernel oil, or one or more natural, synthetic or semi synthetic hard fats such as cocoa butter. Therefore, In some instances, the base material is mixed with an opioid analgesic (e.g., hydrocodone/oxycodone), a non-opioid analgesic (e.g., acetaminophen) and an antiemetic (e.g., promethazine).

In some cases, the pharmaceutical compositions described herein are administered in injection-ready stable liquids for injection or I.V. drip. For example, saline or other injection-ready liquid can be mixed with an opioid analgesic (e.g., hydrocodone or oxycodone), a non-opioid analgesic (e.g., acetaminophen) and an antiemetic (e.g., promethazine). In some instances, a pharmaceutical composition disclosed herein is administered by a subject administered injection. For example a subject can administer the pharmaceutical composition via a hand-held injection device such as a pen type injector. In one example, a subject can use a device or component disclosed in U.S. Pat. Nos. 6,146,361; 5,536,249; or 5,954,700 (which are herein incorporated by reference in their entirety) to administer a pharmaceutical composition disclosed herein.

### Applications

One aspect of the disclosure provides a method of treating or preventing pain in a subject, wherein the method comprises administering to a subject in need thereof a pharmaceutical composition disclosed herein. In particular the disclosure provides a method of providing increased pain relief to a subject with a pharmaceutical composition disclosed herein. In some instance, a method is provided for reducing pain (e.g., severe pain or moderate to severe pain) in a subject with a pharmaceutical composition disclosed herein. In some instances, the pharmaceutical composition comprises an effective amount of each of an opioid analgesic and an antiemetic, as disclosed herein. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In some instances, the pharmaceutical composition is in the form of a tablet. In some instances, the pharmaceutical composition is in the form of a tablet comprising two layers. In some instances, the pharmaceutical composition is in the form of a bi-layer tablet that comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the reduction in pain is at least 10%, at least 20%, at least 30%, at least 40%, at least 50%, at least 60%, at least 70%, at least 80%, at least 90%, or at least 95%. In some instances, the reduction in pain is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 55%, about 60%, about 65%, about 70%, about 75%, about 80%, about 85%, about 90%, or about 95%. In some instances, the reduction in pain comprises reduction in severity, duration, and/or occurrence of pain. In some instances, the reduction in pain is over 4 hours or more, over 6 hours or more, over 12 hours or more, over 24 hours or more, over 48 hours or more, over 72 hours or more, over 96 hours or more, or over 120 hours or more following administration of the pharmaceutical composition. In some instances, the reduction in pain is over 24 hours or more following administration of the pharmaceutical composition. In some instances, the reduction in pain is over 48 hours following administration of the pharmaceutical composition. In some instances, the reduction in pain is over 120 hours or more following administration of the pharmaceutical composition. In some instance, the reduction in pain is that compared to a pharmaceutical composition which does not comprise an antiemetic. In some instance, the reduction in pain is that compared to placebo. Example levels of pain prevented or reduced with compositions disclosed herein include moderate pain, severe pain and moderate to severe pain. In some instances, the method reduces the intensity or/and frequency of pain. Another aspect of the disclosure provides a method of reducing or eliminating an adverse effect associated with administration of an opioid analgesic to a subject, wherein the method comprises administering to a subject in need thereof a pharmaceutical composition disclosed herein. In some instances, the adverse effect can comprise nausea, retching, vomiting, constipation, gastric upset, sleep disturbance, skin rash, swelling, difficulty breathing, closing of throat, itching, unusual bleeding or bruising, abdominal pain, sedation, CNS depression, respiratory depression, or any combination thereof. In some instances, the method reduces or prevents an adverse effect, e.g., reduces or prevents the incidence of nausea or/and vomiting.

One aspect of the disclosure provides a method of providing pain relief and treating, reducing, or preventing agonizing pain in a subject in need thereof, comprising administering to the subject a pharmaceutical composition disclosed herein, wherein administration of the pharmaceutical composition provides for treatment, reduction or prevention of agonizing pain in the subject. One aspect of the disclosure provides a method of providing pain relief and treating, reducing, or preventing retching in a subject in need thereof, comprising comprises administering to the subject a pharmaceutical composition disclosed herein, wherein administration of the pharmaceutical composition provides for treatment, reduction or prevention of retching in the subject. One aspect of the disclosure provides a method of providing pain relief and treating, reducing, or preventing sleep disturbance in a subject in need thereof, comprising comprises administering to the subject a pharmaceutical composition disclosed herein, wherein administration of the pharmaceutical composition provides for treatment, reduction or prevention of sleep disturbance in the subject.

Another aspect of the disclosure provides a method of treating or preventing pain in a post-operative subject without a supplemental analgesic, comprising administering to the subject a pharmaceutical composition disclosed herein. In some instances, the supplemental analgesic is not needed after a period of time post the operation, e.g., after about 6-192 hours. In some instances, the period of time is about: 6, 12, 24, 48, 72, 96, 120, 144, 168, or 192 hours, e.g., 24 hours. Another aspect of the disclosure provides a method of treating or preventing nausea or vomiting in a post-operative (post-operative nausea and vomiting or PONV) subject without a supplemental antiemetic, comprising administering to the subject a pharmaceutical composition disclosed herein. In some instances, a pharmaceutical composition is provided wherein the composition comprises an effective amount of each of an opioid analgesic and an antiemetic, as disclosed herein. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In some instances, the pharmaceutical composition is in the form of a tablet. In some instances, the pharmaceutical composition is in the form of a tablet comprising two layers. In some instances, the pharmaceutical composition is in the form of a bi-layer tablet. In some cases, the bi-layer tablet comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the reduction in PONV is at least 50%, at least 60%, at least 70%, or at least 80%. In some instances, the reduction in PONV is about 51%, about 52%, about 53%, about 54%, about 55%, about 56%, about 57%, about 58%, about 59%, about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%. In some instances, the reduction in PONV comprises reduction in severity and/or occurrence of nausea or vomiting. In some instances, the reduction in PONV is over 4 hours or more, over 6 hours or more, over 12 hours or more, over 24 hours or more, over 48 hours or more, over 72 hours or more, over 96 hours or more, or over 120 hours or more following administration of the pharmaceutical composition. In some instances, the reduction in PONV is over 24 hours or more following administration of the pharmaceutical composition. In some instances, the reduction in PONV is over 48 hours following administration of the pharmaceutical composition. In some instances, the reduction in PONV is over 120 hours or more following administration of the pharmaceutical composition. In some instances, the reduction in PONV is compared to a pharmaceutical composition which does not comprise an antiemetic. In some instances, the supplemental antiemetic is not needed after a period of time post the operation, e.g., about 6-192 hours. In some instances, the period of time is about: 6, 12, 24, 48, 72, 96, 120, 144, 168, or 192 hours, e.g., about 24 hours. Another aspect of the disclosure provides a method of treating or preventing pain in a post-operative subject, comprising administering to the subject a pharmaceutical composition disclosed herein. In some instances, the method reduces the intensity or/and frequency of pain.

In some instances, pain is measured or quantified. In some instances, pain is measured by a Categorical Pain Intensity Scale (PI-CAT), wherein 0=no pain, 1=mild pain, 2=moderate pain, and 3=severe pain. Pain can be quantified as the time-weighted sum of pain intensity differences over a specific time interval (such as 24 hours), comparing a pharmaceutical composition to a placebo, called summed pain intensity differences (SPID_{24 hours}). In some instances, pain is measured by a Visual Analog Pain Intensity Scale (PI-VAS), where 0=no pain and 100=severe pain. In some instances, moderate pain intensity can be 50-70 on a 100 PI-VAS. In some instances, severe pain intensity can be 70-100 on a 100 PI-VAS. In some instances, pain is measured by a Total Pain Relief (REL). REL can be measured on a categorical relief scale (How much pain relief do you have now?): 0=No Relief, 1=Slight Relief, 2=Mild Relief, 3=Moderate Relief, 4=Considerable Relief, 5=Almost Complete Relief, 6=Complete Relief. In some instances, pain is measured by a Qualities of Dental Pain Index (QDPI) or Qualities of Pain Index (QPI). In some instances, QDPI or QPI can be measured on a pain scale of 0 (not at all) to 10 (very much) on each of 15 words (11 sensory: Aching, Throbbing, Hot, Heavy, Pulling, Sharp, Radiating, Pressing, Swollen, Tight, and Stabbing, Stinging; 2 evaluative: aching and hurting; and 2 affective qualities of pain: annoying and agonizing). In some instances, the affective quality of pain is measured is agnozing. In some instances, pain can be measured or quantified as time to first use of rescue medication.

In some aspects, a method is provided for reducing the affective, sensory, and evaluative qualities of pain in a subject, comprising administering to the subject a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In some instances, the pharmaceutical composition is in the form of a tablet. In some instances, the pharmaceutical composition is in the form of a tablet comprising two layers. In some instances, the pharmaceutical composition is in the form of a bi-layer tablet. In some instances, the pharmaceutical composition is in the form of a bi-layer tablet that comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the reduction in the affective, sensory, and evaluative qualities of pain is at least 10%, at least 20%, at least 30%, at least 40%, or at least 50%. In some instances, the reduction in the affective, sensory, and evaluative qualities of pain is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some instances, the reduction in the affective, sensory, and evaluative qualities of pain is over 4 hours or more, over 6 hours or more, over 12 hours or more, over 24 hours or more, over 48 hours or more, over 72 hours or more, over 96 hours or more, or over 120 hours or more following administration of the pharmaceutical composition.

In some instances, the reduction in the affective, sensory, and evaluative qualities of pain is over 24 hours or more following administration of a pharmaceutical composition described herein. In some instances, the reduction in the affective, sensory, and evaluative qualities of pain is over 48 hours following administration of the pharmaceutical composition. In some instances, the reduction in the affective, sensory, and evaluative qualities of pain is over 120 hours or more following administration of the pharmaceutical composition. In some instance, the reduction in the affective, sensory, and evaluative qualities of pain is compared to a pharmaceutical composition which does not comprise an antiemetic.

In some aspects, a method is provided for reducing or preventing OINV in a subject, comprising administering to the subject a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In some instances, the pharmaceutical composition is in the form of a tablet. In some instances, the pharmaceutical composition is in the form of a tablet comprising two layers. In some instances, the pharmaceutical composition is in the form of a bi-layer tablet. In some instances, the pharmaceutical composition is in the form of a bi-layer tablet that comprises an immediate-release layer and a controlled-release layer. In some instances, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the reduction in OINV is at least 30%, at least 40%, at least 50%, at least 60%, at least 70%, or at least 80%. In some instances, the reduction in OINV is about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%. In some instances, the reduction in OINV comprises reduction in severity and/or occurrence of nausea or vomiting. In some instances, the reduction in OINV is over 4 hours or more, over 6 hours or more, over 12 hours or more, over 24 hours or more, over 48 hours or more, over 72 hours or more, over 96 hours or more, or over 120 hours or more following administration of the pharmaceutical composition. In some instances, the reduction in OINV is over 24 hours or more following administration of the pharmaceutical composition. In some instances, the reduction in OINV is over 48 hours following administration of the pharmaceutical composition. In some instances, the reduction in OINV is over 120 hours or more following administration of the pharmaceutical composition. In some instance, the reduction in OINV is compared to a pharmaceutical composition which does not comprise an antiemetic.

In some instances, reducing or preventing OINV is reducing intensity of nausea. In some instances, reducing or preventing OINV is reducing the frequency of vomiting. In some instances, reducing or preventing OINV is reducing the relative risk of vomiting. In some instances, reducing or preventing OINV is reducing or preventing the incidence of nausea. In some instances, reducing or preventing OINV is reducing or preventing the severity of nausea. In some instances, reducing or preventing OINV is reducing the intensity of retching or vomiting. In some instances, reducing or preventing OINV is reducing the occurrence of retching or vomiting. In some instances, reducing or preventing OINV is reducing the intensity of retching. In some instances, reducing or preventing OINV is reducing the occurrence of retching. In some instances, reducing or preventing OINV is the reduced need or no need for rescue medication.

In some aspects, methods are provides for reducing or eliminating the need for rescue medication, comprising administering to the subject a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof.

In some instances, the pharmaceutical composition is in the form of a tablet. In some instances, the pharmaceutical composition is in the form of a tablet comprising two layers. In some instances, the pharmaceutical composition is in the form of a bi-layer tablet. In some instances, the pharmaceutical composition is in the form of a tablet that comprises an immediate-release layer and a controlled-release layer. In some instances, the pharmaceutical composition is in the form of a bi-layer tablet that comprises an immediate-release layer and a controlled-release layer. In some instances, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the rescue medication need is reduced by at least 50%, at least 60%, at least 70%, or at least 80%. In some instances, the rescue medication need is reduced by about 50%, about 51%, about 52%, about 53%, about 54%, about 55%, about 56%, about 57%, about 58%, about 59%, about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%. In some instances, the reduction or elimination of the need for rescue medication is over 4 hours or more, over 6 hours or more, over 12 hours or more, over 24 hours or more, over 48 hours or more, over 72 hours or more, over 96 hours or more, or over 120 hours or more following administration of the pharmaceutical composition. In some instances, the reduction or elimination of the need for rescue medication is over 24 hours or more following administration of the pharmaceutical composition. In some instances, the reduction or elimination of the need for rescue medication is over 48 hours following administration of the pharmaceutical composition. In some instances, the reduction or elimination of the need for rescue medication is over 120 hours or more following administration of the pharmaceutical composition. In some instance, the reduction or elimination of the need for rescue medication is compared to a pharmaceutical composition which does not comprise an antiemetic.

In some instances, the rescue medication is a supplemental antiemetic. In some instances, the reduction or elimination of the need for rescue medication is a reduction in the number of doses of a supplemental antiemetic administered. In some instances, the reduction or elimination of the need for rescue medication is a reduction in the number of doses of a supplemental oral antiemetic administered. In some instances, the reduction or elimination of the need for rescue medication is a reduction in the number of doses of a supplemental parenteral antiemetic administered.

In some aspects, a method is provided for eliminating the likelihood of nausea, vomiting, or the use of a rescue medication, comprising administering to the subject a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In some instances, the pharmaceutical composition is in the form of a tablet that comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the pharmaceutical composition is in the form of a tablet. In some instances, the pharmaceutical composition is in the form of a tablet comprising two layers. In some instances, the pharmaceutical composition is in the form of a bi-layer tablet. In some instances, the pharmaceutical composition is in the form of a bi-layer tablet that comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In some instances, the elimination of the likelihood of nausea, vomiting, or the use of a rescue medication is at least at least 50%, at least 60%, at least 70%, at least 80%, at least 90%, or at least 100%. In some instances, the elimination of the likelihood of nausea, vomiting, or the use of a rescue medication is about 50%, about 51%, about 52%, about 53%, about 54%, about 55%, about 56%, about 57%, about 58%, about 59%, about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 66%, about 87%, about 88%, about 89%, about 90%, about 91%, about 92%, about 93%, about 94%, about 95%, about 96%, about 97%, about 98%, about 99%, or about 100%. In some instances, the elimination of the likelihood of nausea, vomiting, or the use of a rescue medication is over 4 hours or more, over 6 hours or more, over 12 hours or more, over 24 hours or more, over 48 hours or more, over 72 hours or more, over 96 hours or more, or over 120 hours or more following administration of the pharmaceutical composition. In some instances, the elimination of the likelihood of nausea, vomiting, or the use of a rescue medication is over 24 hours or more following administration of the pharmaceutical composition. In some instances, the elimination of the likelihood of nausea, vomiting, or the use of a rescue medication is over 48 hours following administration of the pharmaceutical composition. In some instances, the elimination of the likelihood of nausea, vomiting, or the use of a rescue medication is over 120 hours or more following administration of the pharmaceutical composition. In some instance, the elimination of the likelihood of nausea, vomiting, or the use of a rescue medication is compared to a pharmaceutical composition which does not comprise an antiemetic.

In some instances, nausea is measured or quantified. Nausea can be measured using the Nausea Intensity Scale (NIS). In some instances, nausea can be measured on a scale from 0 to 10, where 0=no nausea and 10=severe nausea. In some instances, nausea intensity can be measured as peak nausea. In some instances, nausea intensity can be measured as summed nausea severity. In some instances, nausea can be measured or quantified using the Stomach Scale (StomS). In some instances, nausea measurements can be based on the worst stomach feeling with a time interval (such as 1 hour), where 0 is normal stomach and 10 is vomiting. In some instances, nausea is measured or quantified using the Visual Analog Scale (VAS). In some instances, nausea can be measured or quantified using a 100 mm nausea VAS. In some instances, nausea can be measured or quantified as time to first use of rescue medication.

In some instances, retching is measured or quantified. Retching (also known as dry heaving) is the reverse movement (peristalsis) of the stomach and esophagus without vomiting. Vomiting (the expulsion of gastric contents) is often preceded by retching, but retching and vomiting can occur separately and involve different sets of muscles Retching can be measured using the Retching/Vomiting Index (RVI). In some instances, retching can be measured on a scale from 0 to 7, where 0=no other stomach symptoms, 1=retching, 2=retching more than once, 3=vomiting, 4=retching and vomiting, 5=retching more than once and vomiting, 6=vomiting more than once, and 7=retching and vomiting more than once. In some instances, retching can be measured or quantified as time to first use of rescue medication.

In some instances, vomiting is measured or quantified. In some instances, vomiting can be measured using the Vomiting Frequency Scale (VFS). In some instances, vomiting can be measured based on the question ‘how often did one vomit over a time interval (such as one hour)?’, where 0=not at all, 1=one time, 2=two times, 3=three or more times. In some instances, vomiting can be measured or quantified using the Vomiting Intensity Scale (VIS). In some instances, vomiting is measured as the time of the first episode of vomiting or retching. In some instances, vomiting can be measured as the time to delivery of a rescue medication, such as an anti-nausea or anti-vomiting medication. In some instances, vomiting is measured or quantified using the Stomach Scale (StomS). In some instances, vomiting measurements can be based on the worst stomach feeling with a time interval (such as 1 hour), where 0 is normal stomach and 10 is vomiting.

In some instances, a reduction or prevention of OINV is measured by soliciting feedback from a subject. Soliciting feedback from a subject can occur in a clinical setting or can occur in a residential setting. Soliciting feedback from a subject can occur in the form of a self-assessment, a written questionnaire, a verbal questionnaire solicited by a physician or other health professional to the subject, an online questionnaire, or a questionnaire on a personal electronic device. Soliciting feedback from a subject can occur before administration. Soliciting feedback from a subject can occur after administration such as every hour, such as every 2 hours, such as every 4 hours, such as every 8 hours, such as every 12 hours, such as every 24 hours, or longer. In some instances, feedback can be solicited before the subject goes to sleep.

In some instances, the one or more opioid-related side effects is measured or quantified. In some instances, one or more opioid-related adverse effects is measured or quantified using the Opioid Symptom Scale (OSS). The OSS can be a 4-point categorical scale. In some instances, one or more opioid-related adverse effects can be measured or quantified by soliciting feedback from the subject. In some instances, one or more opioid-related adverse effects can be measured or quantified using the Opioid-Related Symptom Distress Scale (ORSDS). In some instances, the one or more opioid-related adverse effects can be measured or quantified using a 4-point categorical scale, wherein the 4-points can be categorized as frequency of the adverse effect such as 1=rarely, 2=occasionally, 3=frequently, 4=almost constantly. In some instances, the categorical scale can evaluate one or more dimensions of the adverse effect such as “how often”, “how severe”, and “how bothersome”. In some instances, the categorical scale evaluates one or more adverse effects, such as 12 adverse effects, such as nausea, retching, vomiting, constipation, difficulty passing urine, difficulty concentrating, drowsiness, feeling lightheaded or dizzy, feeling confused, fatigue, itchiness, dry mouth, headache, or others. In some instances, the categorical scale, such as ORSDS, can be designed to evaluate the level of distress associated with the adverse effects of commonly used opiates.

### Treatment or Prevention of Photophobia

In some instances, provided herein are methods for treating or preventing photophobia, comprising administering to a subject in need thereof a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In a further instance, the pharmaceutical composition is in the form of a bi-layer tablet that comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In a further instance, the photophobia is associated with a migraine headache.

### Treatment or Prevention of Phonophobia

In some instances, provided herein are methods for treating or preventing phonophobia, comprising administering to a subject in need thereof a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In a further instance, the pharmaceutical composition is in the form of a bi-layer tablet that comprises an immediate-release layer and a controlled-release layer. In another instance, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In a further instance, the phonophobia can be sonophobia, such as the fear of loud sounds. In a further instance, the phonophobia can be a fear of voices. In a further instance, the phonophobia can be ligyrophobia, such as the dear of devices that can emit loud sounds.

### Treatment or Prevention of Sleep Disturbance

In some aspects, provided herein are methods for treating or preventing sleep disturbance, comprising administering to a subject in need thereof a pharmaceutical composition disclosed herein. In some instances, the pharmaceutical composition comprises an effective amount of each of an opioid analgesic and an antiemetic, as disclosed herein above. In some instances, the antiemetic is promethazine or a pharmaceutically acceptable salt thereof and the opioid analgesic is hydrocodone, oxycodone or a pharmaceutically acceptable salt of any one of the foregoing, or any combination thereof. In some instances, the pharmaceutical composition is in the form of a tablet. In some instances, the pharmaceutical composition is in the form of a bi-layer tablet that comprises an immediate-release layer and a controlled-release layer. In some instances, the immediate-release layer comprises promethazine or a pharmaceutically acceptable salt thereof, and the controlled-release layer comprises hydrocodone, oxycodone or a pharmaceutically acceptable salt of any one of the foregoing, or any combination thereof. In a further instance, the sleep disturbance is an opioid-induced sleep disturbance. In some instances, the sleep disturbance is indicated by waking up at night to complain of nausea, retching, vomit, or use an antiemetic (on Day 1, Day 2 and both days). In some instances, the sleep disturbance is assessed from the NIS (nausea intensity scale), RVI (retching/vomiting index) and NVSM (nausea and vomiting supplemental medications). In some instances, the reduction in sleep disturbance is at least 50%, at least 60%, at least 70%, or at least 80%. In some instances, the reduction in sleep disturbance is about 50%, about 51%, about 52%, about 53%, about 54%, about 55%, about 56%, about 57%, about 58%, about 59%, about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%. In some instances, the reduction in sleep disturbance is over 4 hours or more, over 6 hours or more, over 12 hours or more, over 24 hours or more, over 48 hours or more, over 72 hours or more, over 96 hours or more, or over 120 hours or more following administration of the pharmaceutical composition. In some instances, the reduction in sleep disturbance is over 24 hours or more following administration of the pharmaceutical composition. In some instances, the reduction in sleep disturbance is over 48 hours following administration of the pharmaceutical composition. In some instances, the reduction in sleep disturbance is over 120 hours or more following administration of the pharmaceutical composition. In some instance, the reduction in sleep disturbance is that compared to a pharmaceutical composition which does not comprise an antiemetic. In some instance, the reduction in sleep disturbance is that compared to placebo.

### Combination of Drug Agents

In some instances, a pharmaceutical composition comprises two or more of agents selected from an opioid agent, a non-opioid agent, an antiemetic agent, a barbiturate agent, a stimulant agent, an opioid antagonist agent, an abuse deterrent agent, or any combination thereof. In some embodiments, the pharmaceutical composition comprises an opioid agent and a an antiemetic agent, and optionally one or more agents selected from a non-opioid agent, an antiemetic agent, a barbiturate agent, a stimulant agent, an opioid antagonist agent, an abuse deterrent agent, or any combination thereof. In some embodiments, the pharmaceutical composition comprises an opioid agent, a non-opioid agent and an antiemetic agent, and optionally one or more agents selected from an antiemetic agent, a barbiturate agent, a stimulant agent, an opioid antagonist agent, an abuse deterrent agent, or any combination thereof.

In some instances, a pharmaceutical composition is formulated in a variety of dosage forms (e.g., tablets, capsules, geld, lollipops), parenteral, intraspinal infusion, inhalations, nasal sprays, transdermal patches, iontophoresis transport, absorbing gels, liquids, liquid tannates, suppositories, injections, I.V. drips, other delivery methods, or any combination thereof to treat subjects in need thereof. In some instances, a dosage form comprising an effective amount of promethazine or a pharmaceutically acceptable salt thereof can be orally administered to a subject in need thereof having a tendency to exhibit one or more adverse effect of opioid administration, such as gastric upset, nausea, retching, vomiting, sleep disturbance constipation, skin rash, sedation, CNS depression, or respiratory depression in response to opioid administration.

In some instances, each agent disclosed herein is present in a pharmaceutical composition as its pharmaceutically acceptable salt. In some instances, a pharmaceutical composition comprises hydrocodone, and the hydrocodone is in the form of hydrocodone bitartrate. In some instances, a pharmaceutical composition comprises oxycodone, and the oxycodone is in the form of oxycodone hydrochloride. In some instances, a pharmaceutical composition comprises ibuprofen, and the ibuprofen is in the form of ibuprofen sodium. In some instances, a pharmaceutical composition comprises naproxen, and the naproxen is in the form of naproxen sodium. In some instances, a pharmaceutical composition comprises promethazine, and the promethazine is in the form of promethazine hydrochloride. In some instances, a pharmaceutical composition comprises naltrexone, and the naltrexone is in the form of naltrexone hydrochloride. In some instances, a pharmaceutical composition is in the form of a bi-layer tablet comprising an immediate-release layer and a controlled-release layer. In some embodiments, the controlled-release layer comprises one or more of hydrocodone, oxycodone, propoxyphene, ibuprofen, acetaminophen, or naproxen, or a pharmaceutically acceptable salt thereof. In some embodiments, the immediate-release layer comprises promethazine or a pharmaceutically acceptable salt thereof.

In some instances, a pharmaceutical composition disclosed herein comprises a combination of agents as listed in Table 1. In some instances, pharmaceutical compositions of Table 1 can be formulated in a variety of dosage forms (e.g., tablets, capsules, geld, lollipops), parenteral, intraspinal infusion, inhalations, nasal sprays, transdermal patches, iontophoresis transport, absorbing gels, liquids, liquid tannates, suppositories, injections, I.V. drips, other delivery methods, or any combination thereof to treat subjects. In some instances, each agent disclosed in Table 1 can be present in a pharmaceutical composition as its pharmaceutically acceptable salt. In some instances, ibuprofen of a pharmaceutical composition of Table 1 is in the form of ibuprofen sodium. In some instances, naproxen of a pharmaceutical composition of Table 1 is in the form of naproxen sodium. In some instances, promethazine of a pharmaceutical composition of Table 1 is in the form of promethazine hydrochloride. In some instances, naltrexone of a pharmaceutical composition of Table 1 is in the form of naltrexone hydrochloride. In some instances, a dosage form comprising an effective amount of promethazine or a pharmaceutically acceptable salt thereof is orally administered to a subject having a tendency to exhibit one or more adverse effect of opioid administration, such as gastric upset, nausea, retching, vomiting, sleep disturbance, constipation, skin rash, sedation, CNS depression, or respiratory depression in response to opioid administration. In some instances, one or more of pharmaceutical compositions of Table 1 are in the form of a bi-layer tablet comprising an immediate-release layer and a controlled-release layer.

TABLE 1
Multi-Drug Compositions

| | | | | | | Opioid | Abuse |
|---|---|---|---|---|---|---|---|
| Cmp. | Opioid | Non-opioid | Antiemetic | Barbiturate | Stimulant | antagonist | det. |
| # | agent | agent | agent | agent | agent | agent | agent |
| 1 | Tramadol | Acetaminophen | — | — | — | — | — |
| 2 | Tramadol | — | Promethazine | — | — | — | — |
| 3 | Tramadol | — | — | Butalbital | — | — | — |
| 4 | Tramadol | — | — | — | Modafinil | — | — |
| 5 | Tramadol | — | — | — | Caffeine | — | — |
| 6 | Tramadol | — | — | — | — | Naltrexone | — |
| 7 | Tramadol | — | — | — | — | — | Niacin |
| 8 | Tramadol | Acetaminophen | Promethazine | — | — | — | — |
| 9 | Tramadol | Acetaminophen | — | Butalbital | — | — | — |
| 10 | Tramadol | Acetaminophen | — | — | Modafinil | — | — |
| 11 | Tramadol | Acetaminophen | — | — | Caffeine | — | — |
| 12 | Tramadol | Acetaminophen | — | — | — | Naltrexone | — |
| 13 | Tramadol | Acetaminophen | — | — | — | — | Niacin |
| 14 | Tramadol | — | Promethazine | Butalbital | — | — | — |
| 15 | Tramadol | — | Promethazine | — | Modafinil | — | — |
| 16 | Tramadol | — | Promethazine | — | Caffeine | — | — |
| 17 | Tramadol | — | Promethazine | — | — | Naltrexone | — |
| 18 | Tramadol | — | Promethazine | — | — | — | Niacin |
| 19 | Tramadol | Acetaminophen | Promethazine | Butalbital | — | — | — |
| 20 | Tramadol | Acetaminophen | Promethazine | — | Modafinil | — | — |
| 21 | Tramadol | Acetaminophen | Promethazine | — | Caffeine | — | — |
| 22 | Tramadol | Acetaminophen | Promethazine | — | — | Naltrexone | — |
| 23 | Tramadol | Acetaminophen | Promethazine | — | — | — | Niacin |
| 24 | Tramadol | Acetaminophen | — | Butalbital | Modafinil | — | — |
| 25 | Tramadol | Acetaminophen | — | Butalbital | Caffeine | — | — |
| 26 | Tramadol | Acetaminophen | — | Butalbital | — | Naltrexone | — |
| 27 | Tramadol | Acetaminophen | — | Butalbital | — | — | Niacin |
| 28 | Tramadol | Acetaminophen | — | — | Modafinil | Naltrexone | — |
| 29 | Tramadol | Acetaminophen | — | — | Modafinil | — | Niacin |
| 30 | Tramadol | Acetaminophen | — | — | Caffeine | Naltrexone | — |
| 31 | Tramadol | Acetaminophen | — | — | Caffeine | — | Niacin |
| 32 | Tramadol | Acetaminophen | — | — | — | Naltrexone | Niacin |
| 33 | Tramadol | — | Promethazine | Butalbital | Modafinil | — | — |
| 34 | Tramadol | — | Promethazine | Butalbital | Caffeine | — | — |
| 35 | Tramadol | — | Promethazine | Butalbital | — | Naltrexone | — |
| 36 | Tramadol | — | Promethazine | Butalbital | — | — | Niacin |
| 37 | Tramadol | — | Promethazine | — | Modafinil | Naltrexone | — |
| 38 | Tramadol | — | Promethazine | — | Caffeine | Naltrexone | — |
| 39 | Tramadol | — | Promethazine | — | Modafinil | — | Niacin |
| 40 | Tramadol | — | Promethazine | — | Caffeine | — | Niacin |
| 41 | Tramadol | — | Promethazine | — | — | Naltrexone | Niacin |
| 42 | Tramadol | Acetaminophen | Promethazine | Butalbital | Modafinil | — | — |
| 43 | Tramadol | Acetaminophen | Promethazine | Butalbital | Caffeine | — | — |
| 44 | Tramadol | Acetaminophen | Promethazine | Butalbital | — | Naltrexone | — |
| 45 | Tramadol | Acetaminophen | Promethazine | Butalbital | — | — | Niacin |
| 46 | Tramadol | Acetaminophen | Promethazine | — | Modafinil | Naltrexone | — |
| 47 | Tramadol | Acetaminophen | Promethazine | — | Caffeine | Naltrexone | — |
| 48 | Tramadol | Acetaminophen | Promethazine | — | Modafinil | — | Niacin |
| 49 | Tramadol | Acetaminophen | Promethazine | — | Caffeine | — | Niacin |
| 50 | Tramadol | Acetaminophen | Promethazine | — | — | Naltrexone | Niacin |
| 51 | Tramadol | Acetaminophen | — | Butalbital | Modafinil | Naltrexone | — |
| 52 | Tramadol | Acetaminophen | — | Butalbital | Caffeine | Naltrexone | — |
| 53 | Tramadol | Acetaminophen | — | Butalbital | Modafinil | — | Niacin |
| 54 | Tramadol | Acetaminophen | — | Butalbital | Caffeine | — | Niacin |
| 55 | Tramadol | Acetaminophen | — | Butalbital | — | Naltrexone | Niacin |
| 56 | Tramadol | Acetaminophen | — | — | Modafinil | Naltrexone | Niacin |
| 57 | Tramadol | Acetaminophen | — | — | Caffeine | Naltrexone | Niacin |
| 58 | Tramadol | — | Promethazine | Butalbital | Modafinil | Naltrexone | — |
| 59 | Tramadol | — | Promethazine | Butalbital | Caffeine | Naltrexone | — |
| 60 | Tramadol | — | Promethazine | Butalbital | Modafinil | — | Niacin |
| 61 | Tramadol | — | Promethazine | Butalbital | Caffeine | — | Niacin |
| 62 | Tramadol | — | Promethazine | Butalbital | — | Naltrexone | Niacin |
| 63 | Tramadol | Acetaminophen | Promethazine | Butalbital | Modafinil | Naltrexone | — |
| 64 | Tramadol | Acetaminophen | Promethazine | Butalbital | Caffeine | Naltrexone | — |
| 65 | Tramadol | Acetaminophen | Promethazine | Butalbital | Modafinil | — | Niacin |
| 66 | Tramadol | Acetaminophen | Promethazine | Butalbital | Caffeine | — | Niacin |
| 67 | Tramadol | — | Promethazine | Butalbital | Modafinil | Naltrexone | Niacin |
| 68 | Tramadol | — | Promethazine | Butalbital | Caffeine | Naltrexone | Niacin |
| 69 | Tramadol | Acetaminophen | — | Butalbital | Modafinil | Naltrexone | Niacin |
| 70 | Tramadol | Acetaminophen | — | Butalbital | Caffeine | Naltrexone | Niacin |
| 71 | Tramadol | Acetaminophen | Promethazine | — | Modafinil | Naltrexone | Niacin |
| 72 | Tramadol | Acetaminophen | Promethazine | — | Caffeine | Naltrexone | Niacin |
| 73 | Tramadol | Acetaminophen | Promethazine | Butalbital | — | Naltrexone | Niacin |
| 74 | Tramadol | Acetaminophen | Promethazine | Butalbital | Modafinil | Naltrexone | Niacin |
| 75 | Tramadol | Acetaminophen | Promethazine | Butalbital | Caffeine | Naltrexone | Niacin |
| 76 | Tramadol | — | Promethazine | — | — | — | — |
| 77 | Tramadol | Naproxen | Promethazine | — | — | — | — |
| 78 | Tramadol | — | Promethazine | Butalbital | — | — | — |
| 79 | Tramadol | — | Promethazine | — | Modafinil | — | — |
| 80 | Tramadol | — | Promethazine | — | Caffeine | — | — |
| 81 | Tramadol | — | Promethazine | — | — | Naltrexone | — |
| 82 | Tramadol | — | Promethazine | — | — | — | Niacin |
| 83 | Tramadol | Naproxen | Promethazine | Butalbital | — | — | — |
| 84 | Tramadol | Naproxen | Promethazine | — | Modafinil | — | — |
| 85 | Tramadol | Naproxen | Promethazine | — | Caffeine | — | — |
| 86 | Tramadol | Naproxen | Promethazine | — | — | Naltrexone | — |
| 87 | Tramadol | Naproxen | Promethazine | — | — | — | Niacin |
| 88 | Tramadol | — | Promethazine | Butalbital | Modafinil | — | — |
| 89 | Tramadol | — | Promethazine | Butalbital | Caffeine | — | — |
| 90 | Tramadol | — | Promethazine | Butalbital | — | Naltrexone | — |
| 91 | Tramadol | — | Promethazine | Butalbital | — | — | Niacin |
| 92 | Tramadol | — | Promethazine | — | Modafinil | Naltrexone | — |
| 93 | Tramadol | — | Promethazine | — | Caffeine | Naltrexone | — |
| 94 | Tramadol | — | Promethazine | — | Modafinil | — | Niacin |
| 95 | Tramadol | — | Promethazine | — | Caffeine | — | Niacin |
| 96 | Tramadol | — | Promethazine | — | — | Naltrexone | Niacin |
| 97 | Tramadol | Naproxen | Promethazine | Butalbital | Modafinil | — | — |
| 98 | Tramadol | Naproxen | Promethazine | Butalbital | Caffeine | — | — |
| 99 | Tramadol | Naproxen | Promethazine | Butalbital | — | Naltrexone | — |
| 100 | Tramadol | Naproxen | Promethazine | Butalbital | — | — | Niacin |
| 101 | Tramadol | Naproxen | Promethazine | — | Modafinil | Naltrexone | — |
| 102 | Tramadol | Naproxen | Promethazine | — | Caffeine | Naltrexone | — |
| 103 | Tramadol | Naproxen | Promethazine | — | Modafinil | — | Niacin |
| 104 | Tramadol | Naproxen | Promethazine | — | Caffeine | — | Niacin |
| 105 | Tramadol | Naproxen | Promethazine | — | — | Naltrexone | Niacin |
| 106 | Tramadol | — | Promethazine | Butalbital | Modafinil | Naltrexone | — |
| 107 | Tramadol | — | Promethazine | Butalbital | Caffeine | Naltrexone | — |
| 108 | Tramadol | — | Promethazine | Butalbital | Modafinil | — | Niacin |
| 109 | Tramadol | — | Promethazine | Butalbital | Caffeine | — | Niacin |
| 110 | Tramadol | — | Promethazine | Butalbital | — | Naltrexone | Niacin |
| 111 | Tramadol | Naproxen | Promethazine | Butalbital | Modafinil | Naltrexone | — |
| 112 | Tramadol | Naproxen | Promethazine | Butalbital | Caffeine | Naltrexone | — |
| 113 | Tramadol | Naproxen | Promethazine | Butalbital | Modafinil | — | Niacin |
| 114 | Tramadol | Naproxen | Promethazine | Butalbital | Caffeine | — | Niacin |
| 115 | Tramadol | — | Promethazine | Butalbital | Modafinil | Naltrexone | Niacin |
| 116 | Tramadol | — | Promethazine | Butalbital | Caffeine | Naltrexone | Niacin |
| 117 | Tramadol | Naproxen | Promethazine | — | Modafinil | Naltrexone | Niacin |
| 118 | Tramadol | Naproxen | Promethazine | — | Caffeine | Naltrexone | Niacin |
| 119 | Tramadol | Naproxen | Promethazine | Butalbital | — | Naltrexone | Niacin |
| 120 | Tramadol | Naproxen | Promethazine | Butalbital | Modafinil | Naltrexone | Niacin |
| 121 | Tramadol | Naproxen | Promethazine | Butalbital | Caffeine | Naltrexone | Niacin |
| 122 | Tramadol | — | Promethazine | — | — | — | — |
| 123 | Tramadol | Ibuprofen | Promethazine | — | — | — | — |
| 124 | Tramadol | — | Promethazine | Butalbital | — | — | — |
| 125 | Tramadol | — | Promethazine | — | Modafinil | — | — |
| 126 | Tramadol | — | Promethazine | — | Caffeine | — | — |
| 127 | Tramadol | — | Promethazine | — | — | Naltrexone | — |
| 128 | Tramadol | — | Promethazine | — | — | — | Niacin |
| 129 | Tramadol | Ibuprofen | Promethazine | Butalbital | — | — | — |
| 130 | Tramadol | Ibuprofen | Promethazine | — | Modafinil | — | — |
| 131 | Tramadol | Ibuprofen | Promethazine | — | Caffeine | — | — |
| 132 | Tramadol | Ibuprofen | Promethazine | — | — | Naltrexone | — |
| 133 | Tramadol | Ibuprofen | Promethazine | — | — | — | Niacin |
| 134 | Tramadol | — | Promethazine | Butalbital | Modafinil | — | — |
| 135 | Tramadol | — | Promethazine | Butalbital | Caffeine | — | — |
| 136 | Tramadol | — | Promethazine | Butalbital | — | Naltrexone | — |
| 137 | Tramadol | — | Promethazine | Butalbital | — | — | Niacin |
| 138 | Tramadol | — | Promethazine | — | Modafinil | Naltrexone | — |
| 139 | Tramadol | — | Promethazine | — | Caffeine | Naltrexone | — |
| 140 | Tramadol | — | Promethazine | — | Modafinil | — | Niacin |
| 141 | Tramadol | — | Promethazine | — | Caffeine | — | Niacin |
| 142 | Tramadol | — | Promethazine | — | — | Naltrexone | Niacin |
| 143 | Tramadol | Ibuprofen | Promethazine | Butalbital | Modafinil | — | — |
| 144 | Tramadol | Ibuprofen | Promethazine | Butalbital | Caffeine | — | — |
| 145 | Tramadol | Ibuprofen | Promethazine | Butalbital | — | Naltrexone | — |
| 146 | Tramadol | Ibuprofen | Promethazine | Butalbital | — | — | Niacin |
| 147 | Tramadol | Ibuprofen | Promethazine | — | Modafinil | Naltrexone | — |
| 148 | Tramadol | Ibuprofen | Promethazine | — | Caffeine | Naltrexone | — |
| 149 | Tramadol | Ibuprofen | Promethazine | — | Modafinil | — | Niacin |
| 150 | Tramadol | Ibuprofen | Promethazine | — | Caffeine | — | Niacin |
| 151 | Tramadol | Ibuprofen | Promethazine | — | — | Naltrexone | Niacin |
| 152 | Tramadol | — | Promethazine | Butalbital | Modafinil | Naltrexone | — |
| 153 | Tramadol | — | Promethazine | Butalbital | Caffeine | Naltrexone | — |
| 154 | Tramadol | — | Promethazine | Butalbital | Modafinil | — | Niacin |
| 155 | Tramadol | — | Promethazine | Butalbital | Caffeine | — | Niacin |
| 156 | Tramadol | — | Promethazine | Butalbital | — | Naltrexone | Niacin |
| 157 | Tramadol | Ibuprofen | Promethazine | Butalbital | Modafinil | Naltrexone | — |
| 158 | Tramadol | Ibuprofen | Promethazine | Butalbital | Caffeine | Naltrexone | — |
| 159 | Tramadol | Ibuprofen | Promethazine | Butalbital | Modafinil | — | Niacin |
| 160 | Tramadol | Ibuprofen | Promethazine | Butalbital | Caffeine | — | Niacin |
| 161 | Tramadol | — | Promethazine | Butalbital | Modafinil | Naltrexone | Niacin |
| 162 | Tramadol | — | Promethazine | Butalbital | Caffeine | Naltrexone | Niacin |
| 163 | Tramadol | Ibuprofen | Promethazine | — | Modafinil | Naltrexone | Niacin |
| 164 | Tramadol | Ibuprofen | Promethazine | — | Caffeine | Naltrexone | Niacin |
| 165 | Tramadol | Ibuprofen | Promethazine | Butalbital | — | Naltrexone | Niacin |
| 166 | Tramadol | Ibuprofen | Promethazine | Butalbital | Modafinil | Naltrexone | Niacin |
| 167 | Tramadol | Ibuprofen | Promethazine | Butalbital | Caffeine | Naltrexone | Niacin |
| 168 | Tapentadol | Acetaminophen | — | — | — | — | — |
| 169 | Tapentadol | — | Promethazine | — | — | — | — |
| 170 | Tapentadol | — | — | Butalbital | — | — | — |
| 171 | Tapentadol | — | — | — | Modafinil | — | — |
| 172 | Tapentadol | — | — | — | Caffeine | — | — |
| 173 | Tapentadol | — | — | — | — | Naltrexone | — |
| 174 | Tapentadol | — | — | — | — | — | Niacin |
| 175 | Tapentadol | Acetaminophen | Promethazine | — | — | — | — |
| 176 | Tapentadol | Acetaminophen | — | Butalbital | — | — | — |
| 177 | Tapentadol | Acetaminophen | — | — | Modafinil | — | — |
| 178 | Tapentadol | Acetaminophen | — | — | Caffeine | — | — |
| 179 | Tapentadol | Acetaminophen | — | — | — | Naltrexone | — |
| 180 | Tapentadol | Acetaminophen | — | — | — | — | Niacin |
| 181 | Tapentadol | — | Promethazine | Butalbital | — | — | — |
| 182 | Tapentadol | — | Promethazine | — | Modafinil | — | — |
| 183 | Tapentadol | — | Promethazine | — | Caffeine | — | — |
| 184 | Tapentadol | — | Promethazine | — | — | Naltrexone | — |
| 185 | Tapentadol | — | Promethazine | — | — | — | Niacin |
| 186 | Tapentadol | Acetaminophen | Promethazine | Butalbital | — | — | — |
| 187 | Tapentadol | Acetaminophen | Promethazine | — | Modafinil | — | — |
| 188 | Tapentadol | Acetaminophen | Promethazine | — | Caffeine | — | — |
| 189 | Tapentadol | Acetaminophen | Promethazine | — | — | Naltrexone | — |
| 190 | Tapentadol | Acetaminophen | Promethazine | — | — | — | Niacin |
| 191 | Tapentadol | Acetaminophen | — | Butalbital | Modafinil | — | — |
| 192 | Tapentadol | Acetaminophen | — | Butalbital | Caffeine | — | — |
| 193 | Tapentadol | Acetaminophen | — | Butalbital | — | Naltrexone | — |
| 194 | Tapentadol | Acetaminophen | — | Butalbital | — | — | Niacin |
| 195 | Tapentadol | Acetaminophen | — | — | Modafinil | Naltrexone | — |
| 196 | Tapentadol | Acetaminophen | — | — | Modafinil | — | Niacin |
| 197 | Tapentadol | Acetaminophen | — | — | Caffeine | Naltrexone | — |
| 198 | Tapentadol | Acetaminophen | — | — | Caffeine | — | Niacin |
| 199 | Tapentadol | Acetaminophen | — | — | — | Naltrexone | Niacin |
| 200 | Tapentadol | — | Promethazine | Butalbital | Modafinil | — | — |
| 201 | Tapentadol | — | Promethazine | Butalbital | Caffeine | — | — |
| 202 | Tapentadol | — | Promethazine | Butalbital | — | Naltrexone | — |
| 203 | Tapentadol | — | Promethazine | Butalbital | — | — | Niacin |
| 204 | Tapentadol | — | Promethazine | — | Modafinil | Naltrexone | — |
| 205 | Tapentadol | — | Promethazine | — | Caffeine | Naltrexone | — |
| 206 | Tapentadol | — | Promethazine | — | Modafinil | — | Niacin |
| 207 | Tapentadol | — | Promethazine | — | Caffeine | — | Niacin |
| 208 | Tapentadol | — | Promethazine | — | — | Naltrexone | Niacin |
| 209 | Tapentadol | Acetaminophen | Promethazine | Butalbital | Modafinil | — | — |
| 210 | Tapentadol | Acetaminophen | Promethazine | Butalbital | Caffeine | — | — |
| 211 | Tapentadol | Acetaminophen | Promethazine | Butalbital | — | Naltrexone | — |
| 212 | Tapentadol | Acetaminophen | Promethazine | Butalbital | — | — | Niacin |
| 213 | Tapentadol | Acetaminophen | Promethazine | — | Modafinil | Naltrexone | — |
| 214 | Tapentadol | Acetaminophen | Promethazine | — | Caffeine | Naltrexone | — |
| 215 | Tapentadol | Acetaminophen | Promethazine | — | Modafinil | — | Niacin |
| 216 | Tapentadol | Acetaminophen | Promethazine | — | Caffeine | — | Niacin |
| 217 | Tapentadol | Acetaminophen | Promethazine | — | — | Naltrexone | Niacin |
| 218 | Tapentadol | Acetaminophen | — | Butalbital | Modafinil | Naltrexone | — |
| 219 | Tapentadol | Acetaminophen | — | Butalbital | Caffeine | Naltrexone | — |
| 220 | Tapentadol | Acetaminophen | — | Butalbital | Modafinil | — | Niacin |
| 221 | Tapentadol | Acetaminophen | — | Butalbital | Caffeine | — | Niacin |
| 222 | Tapentadol | Acetaminophen | — | Butalbital | — | Naltrexone | Niacin |
| 223 | Tapentadol | Acetaminophen | — | — | Modafinil | Naltrexone | Niacin |
| 224 | Tapentadol | Acetaminophen | — | — | Caffeine | Naltrexone | Niacin |
| 225 | Tapentadol | — | Promethazine | Butalbital | Modafinil | Naltrexone | — |
| 226 | Tapentadol | — | Promethazine | Butalbital | Caffeine | Naltrexone | — |
| 227 | Tapentadol | — | Promethazine | Butalbital | Modafinil | — | Niacin |
| 228 | Tapentadol | — | Promethazine | Butalbital | Caffeine | — | Niacin |
| 229 | Tapentadol | — | Promethazine | Butalbital | — | Naltrexone | Niacin |
| 230 | Tapentadol | Acetaminophen | Promethazine | Butalbital | Modafinil | Naltrexone | — |
| 231 | Tapentadol | Acetaminophen | Promethazine | Butalbital | Caffeine | Naltrexone | — |
| 232 | Tapentadol | Acetaminophen | Promethazine | Butalbital | Modafinil | — | Niacin |
| 233 | Tapentadol | Acetaminophen | Promethazine | Butalbital | Caffeine | — | Niacin |
| 234 | Tapentadol | — | Promethazine | Butalbital | Modafinil | Naltrexone | Niacin |
| 235 | Tapentadol | — | Promethazine | Butalbital | Caffeine | Naltrexone | Niacin |
| 236 | Tapentadol | Acetaminophen | — | Butalbital | Modafinil | Naltrexone | Niacin |
| 237 | Tapentadol | Acetaminophen | — | Butalbital | Caffeine | Naltrexone | Niacin |
| 238 | Tapentadol | Acetaminophen | Promethazine | — | Modafinil | Naltrexone | Niacin |
| 239 | Tapentadol | Acetaminophen | Promethazine | — | Caffeine | Naltrexone | Niacin |
| 240 | Tapentadol | Acetaminophen | Promethazine | Butalbital | — | Naltrexone | Niacin |
| 241 | Tapentadol | Acetaminophen | Promethazine | Butalbital | Modafinil | Naltrexone | Niacin |
| 242 | Tapentadol | Acetaminophen | Promethazine | Butalbital | Caffeine | Naltrexone | Niacin |
| 243 | Tapentadol | — | Promethazine | — | — | — | — |
| 244 | Tapentadol | Naproxen | Promethazine | — | — | — | — |
| 245 | Tapentadol | — | Promethazine | Butalbital | — | — | — |
| 246 | Tapentadol | — | Promethazine | — | Modafinil | — | — |
| 247 | Tapentadol | — | Promethazine | — | Caffeine | — | — |
| 248 | Tapentadol | — | Promethazine | — | — | Naltrexone | — |
| 249 | Tapentadol | — | Promethazine | — | — | — | Niacin |
| 250 | Tapentadol | Naproxen | Promethazine | Butalbital | — | — | — |
| 251 | Tapentadol | Naproxen | Promethazine | — | Modafinil | — | — |
| 252 | Tapentadol | Naproxen | Promethazine | — | Caffeine | — | — |
| 253 | Tapentadol | Naproxen | Promethazine | — | — | Naltrexone | — |
| 254 | Tapentadol | Naproxen | Promethazine | — | — | — | Niacin |
| 255 | Tapentadol | — | Promethazine | Butalbital | Modafinil | — | — |
| 256 | Tapentadol | — | Promethazine | Butalbital | Caffeine | — | — |
| 257 | Tapentadol | — | Promethazine | Butalbital | — | Naltrexone | — |
| 258 | Tapentadol | — | Promethazine | Butalbital | — | — | Niacin |
| 259 | Tapentadol | — | Promethazine | — | Modafinil | Naltrexone | — |
| 260 | Tapentadol | — | Promethazine | — | Caffeine | Naltrexone | — |
| 261 | Tapentadol | — | Promethazine | — | Modafinil | — | Niacin |
| 262 | Tapentadol | — | Promethazine | — | Caffeine | — | Niacin |
| 263 | Tapentadol | — | Promethazine | — | — | Naltrexone | Niacin |
| 264 | Tapentadol | Naproxen | Promethazine | Butalbital | Modafinil | — | — |
| 265 | Tapentadol | Naproxen | Promethazine | Butalbital | Caffeine | — | — |
| 266 | Tapentadol | Naproxen | Promethazine | Butalbital | — | Naltrexone | — |
| 267 | Tapentadol | Naproxen | Promethazine | Butalbital | — | — | Niacin |
| 268 | Tapentadol | Naproxen | Promethazine | — | Modafinil | Naltrexone | — |
| 269 | Tapentadol | Naproxen | Promethazine | — | Caffeine | Naltrexone | — |
| 270 | Tapentadol | Naproxen | Promethazine | — | Modafinil | — | Niacin |
| 271 | Tapentadol | Naproxen | Promethazine | — | Caffeine | — | Niacin |
| 272 | Tapentadol | Naproxen | Promethazine | — | — | Naltrexone | Niacin |
| 273 | Tapentadol | — | Promethazine | Butalbital | Modafinil | Naltrexone | — |
| 274 | Tapentadol | — | Promethazine | Butalbital | Caffeine | Naltrexone | — |
| 275 | Tapentadol | — | Promethazine | Butalbital | Modafinil | — | Niacin |
| 276 | Tapentadol | — | Promethazine | Butalbital | Caffeine | — | Niacin |
| 277 | Tapentadol | — | Promethazine | Butalbital | — | Naltrexone | Niacin |
| 278 | Tapentadol | Naproxen | Promethazine | Butalbital | Modafinil | Naltrexone | — |
| 279 | Tapentadol | Naproxen | Promethazine | Butalbital | Caffeine | Naltrexone | — |
| 280 | Tapentadol | Naproxen | Promethazine | Butalbital | Modafinil | — | Niacin |
| 281 | Tapentadol | Naproxen | Promethazine | Butalbital | Caffeine | — | Niacin |
| 282 | Tapentadol | — | Promethazine | Butalbital | Modafinil | Naltrexone | Niacin |
| 283 | Tapentadol | — | Promethazine | Butalbital | Caffeine | Naltrexone | Niacin |
| 284 | Tapentadol | Naproxen | Promethazine | — | Modafinil | Naltrexone | Niacin |
| 285 | Tapentadol | Naproxen | Promethazine | — | Caffeine | Naltrexone | Niacin |
| 286 | Tapentadol | Naproxen | Promethazine | Butalbital | — | Naltrexone | Niacin |
| 287 | Tapentadol | Naproxen | Promethazine | Butalbital | Modafinil | Naltrexone | Niacin |
| 288 | Tapentadol | Naproxen | Promethazine | Butalbital | Caffeine | Naltrexone | Niacin |
| 289 | Tapentadol | — | Promethazine | — | — | — | — |
| 290 | Tapentadol | Ibuprofen | Promethazine | — | — | — | — |
| 291 | Tapentadol | — | Promethazine | Butalbital | — | — | — |
| 292 | Tapentadol | — | Promethazine | — | Modafinil | — | — |
| 293 | Tapentadol | — | Promethazine | — | Caffeine | — | — |
| 294 | Tapentadol | — | Promethazine | — | — | Naltrexone | — |
| 295 | Tapentadol | — | Promethazine | — | — | — | Niacin |
| 296 | Tapentadol | Ibuprofen | Promethazine | Butalbital | — | — | — |
| 297 | Tapentadol | Ibuprofen | Promethazine | — | Modafinil | — | — |
| 298 | Tapentadol | Ibuprofen | Promethazine | — | Caffeine | — | — |
| 299 | Tapentadol | Ibuprofen | Promethazine | — | — | Naltrexone | — |
| 300 | Tapentadol | Ibuprofen | Promethazine | — | — | — | Niacin |
| 301 | Tapentadol | — | Promethazine | Butalbital | Modafinil | — | — |
| 302 | Tapentadol | — | Promethazine | Butalbital | Caffeine | — | — |
| 303 | Tapentadol | — | Promethazine | Butalbital | — | Naltrexone | — |
| 304 | Tapentadol | — | Promethazine | Butalbital | — | — | Niacin |
| 305 | Tapentadol | — | Promethazine | — | Modafinil | Naltrexone | — |
| 306 | Tapentadol | — | Promethazine | — | Caffeine | Naltrexone | — |
| 307 | Tapentadol | — | Promethazine | — | Modafinil | — | Niacin |
| 308 | Tapentadol | — | Promethazine | — | Caffeine | — | Niacin |
| 309 | Tapentadol | — | Promethazine | — | — | Naltrexone | Niacin |
| 310 | Tapentadol | Ibuprofen | Promethazine | Butalbital | Modafinil | — | — |
| 311 | Tapentadol | Ibuprofen | Promethazine | Butalbital | Caffeine | — | — |
| 312 | Tapentadol | Ibuprofen | Promethazine | Butalbital | — | Naltrexone | — |
| 313 | Tapentadol | Ibuprofen | Promethazine | Butalbital | — | — | Niacin |
| 314 | Tapentadol | Ibuprofen | Promethazine | — | Modafinil | Naltrexone | — |
| 315 | Tapentadol | Ibuprofen | Promethazine | — | Caffeine | Naltrexone | — |
| 316 | Tapentadol | Ibuprofen | Promethazine | — | Modafinil | — | Niacin |
| 317 | Tapentadol | Ibuprofen | Promethazine | — | Caffeine | — | Niacin |
| 318 | Tapentadol | Ibuprofen | Promethazine | — | — | Naltrexone | Niacin |
| 319 | Tapentadol | — | Promethazine | Butalbital | Modafinil | Naltrexone | — |
| 320 | Tapentadol | — | Promethazine | Butalbital | Caffeine | Naltrexone | — |
| 321 | Tapentadol | — | Promethazine | Butalbital | Modafinil | — | Niacin |
| 322 | Tapentadol | — | Promethazine | Butalbital | Caffeine | — | Niacin |
| 323 | Tapentadol | — | Promethazine | Butalbital | — | Naltrexone | Niacin |
| 324 | Tapentadol | Ibuprofen | Promethazine | Butalbital | Modafinil | Naltrexone | — |
| 325 | Tapentadol | Ibuprofen | Promethazine | Butalbital | Caffeine | Naltrexone | — |
| 326 | Tapentadol | Ibuprofen | Promethazine | Butalbital | Modafinil | — | Niacin |
| 327 | Tapentadol | Ibuprofen | Promethazine | Butalbital | Caffeine | — | Niacin |
| 328 | Tapentadol | — | Promethazine | Butalbital | Modafinil | Naltrexone | Niacin |
| 329 | Tapentadol | — | Promethazine | Butalbital | Caffeine | Naltrexone | Niacin |
| 330 | Tapentadol | Ibuprofen | Promethazine | — | Modafinil | Naltrexone | Niacin |
| 331 | Tapentadol | Ibuprofen | Promethazine | — | Caffeine | Naltrexone | Niacin |
| 332 | Tapentadol | Ibuprofen | Promethazine | Butalbital | — | Naltrexone | Niacin |
| 333 | Tapentadol | Ibuprofen | Promethazine | Butalbital | Modafinil | Naltrexone | Niacin |
| 334 | Tapentadol | Ibuprofen | Promethazine | Butalbital | Caffeine | Naltrexone | Niacin |

Cmp. # is an abbreviation for Composition Number.
Abuse det. agent is an abbreviation for Abuse deterrent agent.

### EXAMPLES

### Example 1. Formulations of Bi-Layer Tablets

Bi-layer tablets containing acetaminophen, hydrocodone and promethazine and excipients in the amounts shown below in Table were prepared and tested.

TABLE 2
Formulation A (Total weight 700 mg)

| | (mg/tablet) |
|---|---|
| Layer 1 Ingredients (550 mg in total) | |
| Acetaminophen 90% (Compap L) USP | 361.1 |
| Hydrocodone Bitartrate USP C-II | 8.3* |
| Microcrystalline Cellulose - Silicified NF | 149.6** |
| Hypromellose USP | 15.5 |
| Croscarmellose Sodium NF | 10 |
| Magnesium Stearate NF | 2.75 |
| Stearic Acid (Vegetable Grade) NF | 2.75 |
| Layer 2 Ingredients (150 mg in total) | |
| Promethazine HCl USP | 12.5 |
| Microcrystalline Cellulose - Silicified NF | 121.5 |
| Croscarmellose Sodium NF | 15.3 |
| Magnesium Stearate NF | 1 |

*Potency adjusted for assay result and water content. The hydrocodone bitartrate anhydrous content is 7.5 mg/tablet.
**Microcrystalline Cellulose adjusted to maintain layer weight of 550 mg.

### Example 2. One of Manufacturing Process Approaches

Tablets were formed by a basic manufacturing process, see FIG. 2A-2C. For example, a blend of hydrocodone bitartrate and acetaminophen was manufactured by passing hydrocodone bitartrate, croscarmellose sodium, and hypromellose (100) through a screen (101) and adding the contents to a clean blender (102). The contents were mixed in the blender. The hypromellose (103) was passed through a screen (104) and added to the blender (102). The contents of the blender were mixed. The silicified microcrystalline cellulose (105) was passed through a screen (106) and added to the blender (102). The contents of the blender were mixed. The silicified microcrystalline cellulose (107) was passed through a screen (108) and added to the blender (102). The contents of blender were mixed. The silicified microcrystalline cellulose (109) was passed through a screen (110) and added to the blender. The contents of the blender were mixed. The blended contents were discharged from the blender into an appropriately labeled container (111). A clean blender was setup (112). The hydrocodone bitartrate was added to the blend from the previous step (111) and half of the total acetaminophen (113) was also added to the clean blender and the contents were mixed. The second half of the total acetaminophen (114) was added to the blender and the contents were mixed. The magnesium stearate and stearic acid were passed (115) through a screen (116) and added to the blender with the hydrocodone bitartrate/acetaminophen blend. The contents of the blender were mixed. The blend was discharged into the appropriate container(s) (117). The accountable yield for the final blend (118) was calculated and recorded.

A blend of promethazine HCl was manufactured by setting up a clean blender for the promethazine HCl blend. Half of the total specified amounts of promethazine HCl USP, cellulose microcrystalline-silicified, and croscarmellose sodium for the promethazine HCl (120) were screened (119). The contents of the blender were mixed (121). The pre-blend was discarded into the appropriate container (122) and set aside. The second half of the total specified amounts of promethazine HCl USP, cellulose microcrystalline-silicified, and croscarmellose sodium for the promethazine HCl blend (124) were screened (123). The contents of the blender were mixed (125). The second pre-blend was discharged into a separate appropriate container (126). The promethazine pre-blends from the separate containers (122 and 126) were added into a clean blender (127) and the contents were mixed. The magnesium stearate (128) was passed through a screen (129) and added to the blender (127) with the promethazine pre-blends. The contents of the blender were mixed. The blend was discarded into the appropriate storage container (130). The accountable yield for the final promethazine blend (131) was calculated and recorded.

Tablet Compression was performed by transferring the first layer of hydrocodone/acetaminophen blend (117) into the first hopper. The press (132) was setup with its respective parameters appropriately. The second layer promethazine blend (130) was transferred into the second hopper. The press was started to begin producing the tablets to the specified parameters. During compressing, tablets were sampled at various times for in-process testing for weight, metal, microbiological contamination, thickness, hardness, and % friability. The accountable yield for the final blend (133) was calculated and recorded. At the end of the batch, the compressed tablets were deposited into an appropriate container (134) and stored.

### Example 3. Hardness Measurement

Tablet hardness measurements were calculated using a tablet hardness tester such as Key Model HT300 or Model HT500 or Pharma Test PTS/301. Using the limit knob on the left side of the housing, the plunger was adjusted according to the diameter of the tablet. The tablet was placed broadside down on the pedestal so that it was centered and was against the right side (stationary) plunger. Capsule-shaped tablets were placed with one end against the stationary plunger, breaking force applied end-to-end, see FIG. 4. When round or square tablets were scored, the tablet was positioned with the bisect parallel to the plunger contact surface. A total of 5 tablets representative of the batch were tested and the individual and average hardness results were reported.

### Example 4. Friability Measurement

Friability measurements were calculated using a friabilator, with a horizontal axis that rotates at 25+/−1 rpm. The drum for the friabilator comprised a synthetic transparent polymer with an internal diameter between 283 and 291 mm and a depth between 36 and 40 mm. The drum comprised a curved projection with an inside radius between 75.5 and 85.5 mm, see FIG. 3. For example, tablets with a unit mass equal to or less than 650 mg, were measured using a representative sample of whole tablets corresponding to 6.5 g. If the tablet unit mass was greater than 650 mg, a representative sample of ten whole tablets was measured. Loose powder from the tablets was removed with the aid of air pressure or a soft brush. An initial tablet weight was recorded. The tablets were placed inside the friabilator drum, the drum was closed and rotated for 100 rotations for 4 minutes. Tablets were then removed from the drum. Any loose powder from all intact (non-broken, non-capped) tablets was removed. If any tablets had been broken or capped, the number for each category was recorded. Pieces of broken or fractured tablets that did not pass through a 10-mesh screen were combined with the intact tablets and were accurately weighed and recorded as the final weight. The percent loss was calculated as follows:

$$Percent loss = \frac{( initial weight - final weight )}{initial weight} \times 100$$

### Example 5. Weight Variation Measurement

Approximately 100 tablets were weighed individually using an appropriate weighing device. The average weight and the relative standard deviation of the weights were calculated. For example, approximately 100 tablets were taken and weighed individually using a Mocon AB3 weigh balance. Pieces of tablets were not included. The printed results were inspected to ensure at least 90 individual weight results were recorded and that any rejected results were reasonable, (i.e., double or triple weights). Any weight result that was outside of a range (0.5 times the theoretical unit weight to 1.5 times the theoretical unit weight) was not included in the calculation. The software that analyzes the tablet weight data assigned T1 and T2 limits to tablets based on U.S. Pharmacopeial Convention weight variation criteria. Individual tablet weight results were flagged and counted as exceeding the T1 and T2 criteria as follows: if the mean tablet weight is 130 mg or less, T1 limits are ±10%, T2 limits are ±20%; if the mean tablet weight is between 130 and 325 mg, T1 limits are ±7.5%, T2 limits are ±15%; if the mean tablet weight is 325 mg or more, T1 limits are ±5.0%, T2 limits are ±10%.

For example, if the items being weighed were capsules, individual gross weight results outside of the ±10% of the calculated average range were flagged and counted as exceeding T1. Any individual gross weight results outside of the ±15% of the calculated average range were flagged and counted as exceeding T2. If values exist outside this range, the actual empty capsule weight was subtracted from the mean weight to obtain a calculated net fill weight and a range of ±25% of the calculated net fill weight was determined. Tablets were flagged when individual results exceed the calculated average weight by the factor indicated.

### Example 6. Dissolution Measurements

Dissolution apparatus was a USP Rotating Paddle Apparatus 2 with an automated sampling station (e.g., VK-8000 or equivalent). Dissolution fluid was 900 mL of de-aerated 0.01 N HCl, maintained at 37.0+/−0.5° C. during dissolution procedure. The fluid was prepared by diluting 5 mL of concentrated HCl in 6000 mL of de-aerated water, and mixed. To measure peaks, a dual wavelength detector (e.g., Hitachi L-2420) was used, or in some instances, two separate chromatographic systems can be used in order to measure the peaks at two different wavelengths.

Standard Solution Preparation: Each ingredient was weighed (e.g., 21 mg of hydrocodone bitartrate) into a 50 mL volumetric flask, and diluted to volume with dissolution media. The resulting solution was mixed to form a stock solution. Different ingredients were similarly prepared to provide stock solutions (e.g., promethazine HCl, acetaminophen). 2 mL each of stock standard solutions were diluted with dissolution fluid and mixed to produce a final standard solution. For example, the concentration of hydrocodone bitartrate was about 0.0084 mg/mL, promethazine HCl was about 0.014 mg/mL, and acetaminophen was about 0.36 mg/mL.

Dissolution test solutions were prepared in 900 mL of 0.01 N HCl using the USP Rotating Paddle Apparatus at 50 WM. An aliquot of the dissolution solution was filtered and a 50-μL aliquot was chromatographed on a 50-mm×4.6-mm (i.d.) Waters sunFire™ C₁₈, 3.5-μm particle size column using a gradient HPLC method. Mobile phase A consisted of water/acetonitrile/TFA, 950/50/2 (v/v/v) and mobile phase B consisted of water/acetonitrile/TFA, 50/950/1.5 (v/v/v). The flow rate was 2.0 mL/minute. For example, the amount of acetaminophen released was determined at 300 nm by comparing the area obtained for the peak due to acetaminophen in the chromatogram of the dissolution test solution to that obtained for the corresponding peak in a chromatogram of a standard solution. The amount of hydrocodone bitartrate released was determined at 230 nm by comparing the area obtained for the peak due to hydrocodone bitartrate in the chromatogram of the dissolution test solution to that obtained for the corresponding peak in a chromatogram of a standard solution. The amount of promethazine HCl released was determined at 230 nm by comparing the area obtained for the peak due to promethazine HCl in the chromatogram of the dissolution test solution to that obtained for the corresponding peak in a chromatogram of a standard solution.

Paddle speed was 50 rpm; pull volume was 10 mL (no replacement); Pull points: 5, 10, 15, 20, 25, 30, 45 and 60 minutes. The amount of each component dissolved in the dissolution medium was determined by HPLC. The method can use a high purity, bonded C18 stationary phase and a binary mobile phase consisting of an appropriate buffer and organic modifier.

900 mL of dissolution fluid preheated to 37° C. was placed into each vessel. Tablets were weighed and placed in vessels respectively. At prescribed time intervals, 5 mL aliquot of the dissolution fluid was drawn using the automated sampling station equipped with a 35 μm full flow filter connected to a sampling probe. Filtrate was allowed to cool to room temperature, to produce a final sample solution. Fluid withdrawn was not replaced. Samples were injected in HPLC for analysis after a baseline was established. Peak area responses were measured for each component: acetaminophen peak eluted at about 1.5 minutes; hydrocodone bitartrate eluted at about 3.3 minutes and promethazine HCl eluted at about 4.8 minutes. The resolution between each peak was calculated, as well as the tailing factor. The mean and % RSD values for the acetaminophen peak areas at 300 nm were measured; promethazine HCl and hydrocodone bitartrate at 230 nm. The five replicate injections were not more than 2.0% RSD. 50 μL aliquots of standard and sample solutions were subjected to liquid chromatography.

Calculation of the amount released per tablet was determined using Equation I:

$$\underline{\frac{mg Released}{Tablet} = \frac{Au}{As} \times Cs \times Vn}$$

where:
Au=The peak area response obtained in the chromatogram of the dissolution test solution.
As=The peak area response obtained in the chromatogram of the standard solution.
Cs=The concentration of the standard solution.
Vn=The volume, in mL, of the dissolution solution at sampling time period n. It is calculated as follows:

Vn=[900−5(n−1)]

Calculation of the amount released as a percent of the label claim was determined using the following equation:

$$\% Released = \frac{mg \frac{Released}{Tablet}}{Label Claim} \times 100 \%$$

Calculation of the amount released at second and subsequent time periods in mg/tablet was determined using the following equation:

$$Wn = Un \underline{+ 5 {\sum }_{i = 1}^{n = 1} \frac{Ui}{Vi}}$$

where:
Wn=The mg released/tablet at time period n (corrected).
Un=The mg released/tablet at time n (uncorrected).
n=The current time period.
i=The time period index.
Ui=The mg released/tablet at time period i (uncorrected).
Vi=The volume, in mL, of the dissolution solution at sampling time period i. It is calculated as follows:

Vi=[900−5(i−1)]

The dissolution profile results for the bi-layer tablet Formulation A are provided in Table 3. The formulation was designed to release the promethazine immediately with the acetaminophen and hydrocodone bitartrate to be released slightly slower. FIG. 1 provides a graphical representation of the dissolution profile results for Formulation A.

TABLE 3
Dissolution rates of Formulation A

| Minutes | 5 | 10 | 15 | 20 | 25 | 30 | 45 | 60 |
|---|---|---|---|---|---|---|---|---|
| Promethazine HCL | 92 | 95 | 97 | 98 | 99 | 99 | 102 | 102 |
| (PMZ) % | | | | | | | | |
| Acetaminophen | 62 | 76 | 86 | 90 | 93 | 95 | 98 | 99 |
| (APAP) % | | | | | | | | |
| Hydrocodone | 47 | 67 | 82 | 90 | 94 | 97 | 101 | 101 |
| Bitartrate (HC) % | | | | | | | | |

### Example 7. Pharmacokinetic Studies of Formulation A

A single-dose, open-label, randomized, four-period crossover study was designed to compare the relative bioavailability of hydrocodone, acetaminophen, and promethazine in Formulation A to hydrocodone in Vicoprofen, (hydrocodone 7.5 mg/ibuprofen 200 mg) manufactured by Halo Pharmaceuticals, Inc. for Abbott Laboratories, promethazine 12.5 mg manufactured by Zydus Pharmaceuticals Inc., and acetaminophen in Ultracet (tramadol HCl 37.5 mg/acetaminophen 325 mg) manufactured by Janssen Pharmaceuticals Inc., under fasted and fed conditions.

This was a single-dose, open-label, randomized, four-period, four-treatment crossover study. Twenty (20) healthy subjects were enrolled. Subjects were randomly assigned to a treatment sequence and received four separate single-dose administrations of study medication, one treatment per period, according to the randomization schedule. Dosing days were separated by a washout period of at least 14 days. Subjects received each of the treatments listed below during the four treatment periods:

Treatment A: Test Formulation (Fasted Subjects)
Formulation A (hydrocodone 7.5 mg/acetaminophen 325 mg/promethazine 12.5 mg) tablet
Dose=1×7.5 mg/325 mg. 12.5 g

Treatment B: Test Formulation (Fed Subjects)
Formulation A (hydrocodone 7.5 mg/acetaminophen 325 mg/promethazine 12.5 mg) tablet
Dose=1×7.5 mg/325 mg. 12.5 g

Treatment C: Comparator Product (Fasted Subjects)
Vicoprofen (hydrocodone 7.5 mg/ibuprofen 200 mg) tablet
Dose=1×7.5 mg/200 mg
Halo Pharmaceuticals, Inc. for Abbott Laboratories
AND
Ultracet (tramadol 37.5 mg/acetaminophen 325 mg) tablet
Dose=1×325 mg
Janssen Pharmaceuticals Inc.
AND
Promethazine 12.5 mg tablet
Dose=1×12.5 mg
Zydus Pharmaceuticals Inc.

Treatment D: Comparator Product (Fed Subjects)
Vicoprofen (hydrocodone 7.5 mg/ibuprofen 200 mg) tablet
Dose=1×7.5 mg/200 mg
Halo Pharmaceuticals, Inc. for Abbott Laboratories
AND
Ultracet (tramadol 37.5 mg/acetaminophen 325 mg) tablet
Dose=1×325 mg
Janssen Pharmaceuticals Inc.
AND
Promethazine 12.5 mg tablet
Dose=1×12.5 mg
Zydus Pharmaceuticals Inc.

Clinical procedure summary. During each study period, 10 mL blood samples were obtained prior to each dosing and following each dose at selected times through 48 hours post-dose. A total of 64 pharmacokinetic blood samples were to be collected from each subject, 16 samples in each study period. In addition, blood was drawn and urine was collected for clinical laboratory testing at screening and study exit. In each study period, subjects were admitted to the study unit in the evening prior to the scheduled dose. Subjects were confined to the research center during each study period until completion of the 24-hour blood collection and other study procedures. Subjects returned to the study unit for outpatient pharmacokinetic blood samples at 48 hours. Procedures for Collecting Samples for Pharmacokinetic Analysis Blood samples (1×10 mL) were collected in Vacutainer tubes containing K₂-EDTA as a preservative at pre-dose (0) and at 0.25, 0.5, 0.75, 1, 1.5, 2, 2.5, 3, 3.5, 4, 6, 8, 12, 24, and 48 hours after dosing.

Bioanalytical summary. Plasma samples were analyzed for hydrocodone, acetaminophen, and promethazine by Worldwide Clinical Trials (WCT) using validated LC-MS-MS procedures. The methods were validated for ranges of 0.100 to 50.0 ng/mL for hydrocodone, based on the analysis of 0.250 mL of human EDTA plasma, 0.0250 to 15.0 μg/mL for acetaminophen, based on the analysis of 0.200 mL of human EDTA plasma, and 0.0500 to 20.0 ng/mL for promethazine, based on the analysis of 0.200 mL of human EDTA plasma. Data were stored in Watson Laboratory Information Management System (LIMS; Version 7.2.0.03, Thermo Fisher Scientific).

Pharmacokinetic Analysis. Concentration-time data were transferred from Watson Laboratory Information Management System directly to Phoenix™ WinNonlin® (Version 6.3, Pharsight Corporation) using the Custom Query Builder option for analysis. Data were analyzed by noncompartmental methods in WinNonlin. Concentration-time data that were below the limit of quantification (BLQ) were treated as zero in the data summarization and descriptive statistics. In the pharmacokinetic analysis, BLQ concentrations were treated as zero from time-zero up to the time at which the first quantifiable concentration was observed; embedded and/or terminal BLQ concentrations were treated as “missing”. Full precision concentration data (not rounded to three significant figures) and actual sample times were used for all pharmacokinetic and statistical analyses.

The following pharmacokinetic parameters were calculated: peak concentration in plasma (Cₘₐₓ), time to peak concentration (Tₘₐₓ), elimination rate constant (λ_{z}), terminal half-life (T_{1/2}), area under the concentration-time curve from time-zero to the time of the last quantifiable concentration (AUCₗₐₛₜ), and area under the plasma concentration time curve from time-zero extrapolated to infinity (AUC_{inf}). Additionally, the following partial AUCs were calculated for promethazine and hydrocodone: area under the plasma concentration time curve from time-zero to 0.25 h (AUC_{0-0.25}), area under the plasma concentration time curve from time-zero to 0.50 h (AUC_{0-0.5}), area under the plasma concentration time curve from time-zero to 0.75 h (AUC_{0-0.75}), area under the plasma concentration time curve from time-zero to 1.00 h (AUC_{0-1.0}), area under the plasma concentration time curve from time-zero to 1.50 h (AUC_{0-1.5}), area under the plasma concentration time curve from time-zero to 2.00 h (AUC_{0-2.0}), and area under the plasma concentration time curve from time-zero to 4.00 h (AUC_{0-4.0}).

Analysis of variance (ANOVA) and the Schuirmann's two one-sided t-test procedures at the 5% significance level were applied to the log-transformed pharmacokinetic exposure parameters, Cₘₐₓ, AUCₗₐₛₜ, and AUC_{inf}. Partial AUCs [AUC_{0-0.25}, AUC_{0-0.5}, AUC_{0-0.75}, AUC_{0-1.0}, AUC_{0-1.5}, AUC_{0-2.0}, and AUC_{0-4.0}] for promethazine and hydrocodone were included in the analysis for comparisons of early systemic exposure across treatments. The 90% confidence interval for the ratio of the geometric means (Test/Reference) was calculated.

Results.

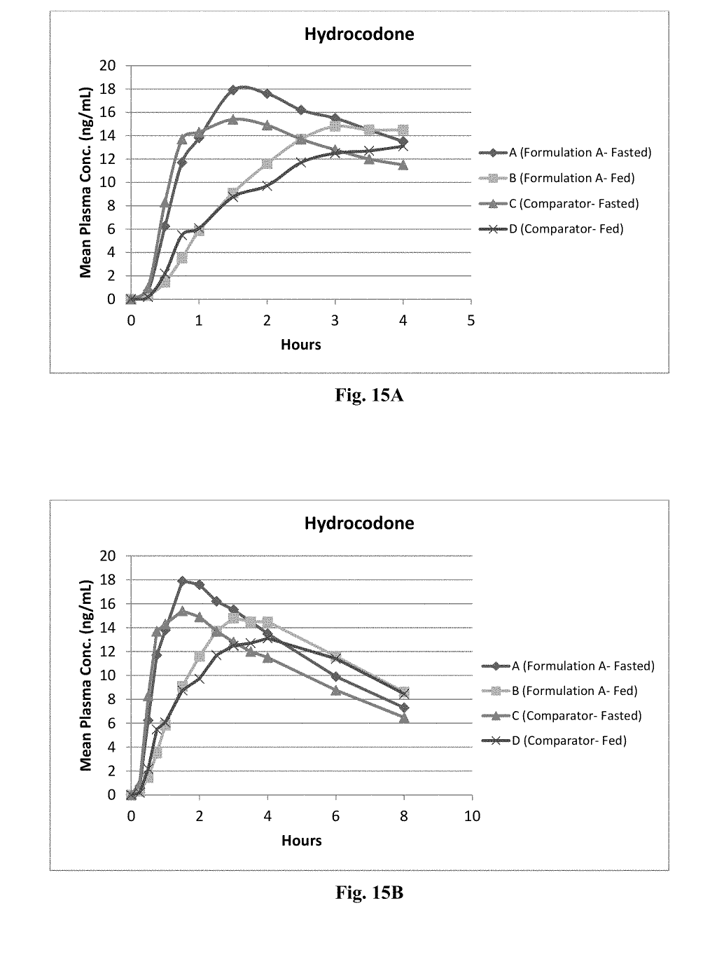

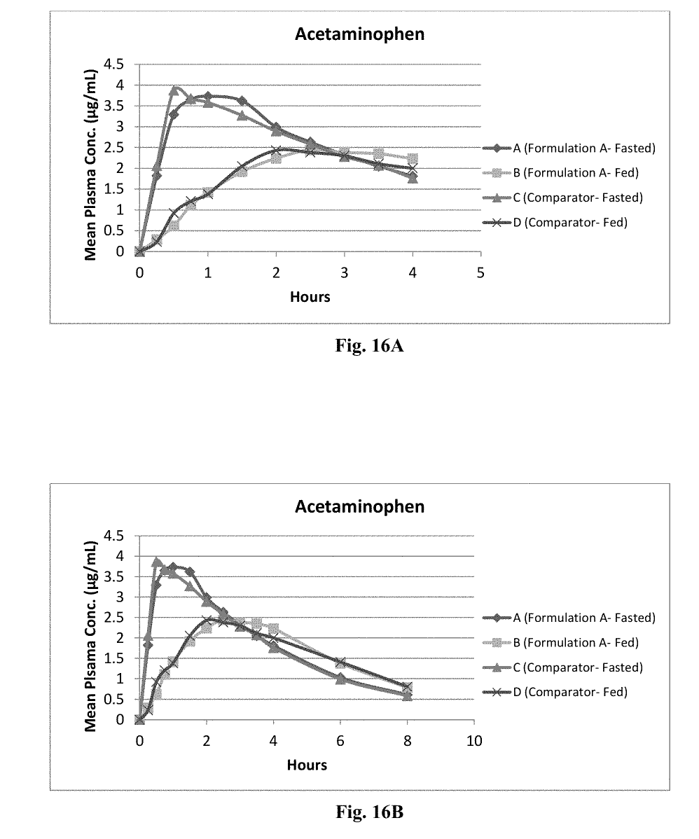

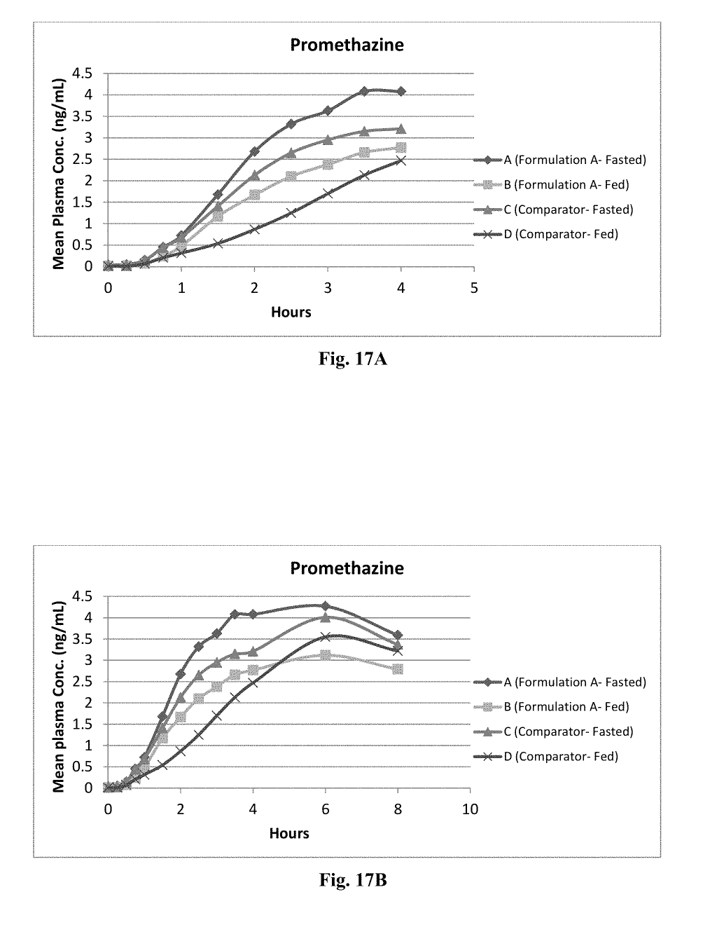

19 of the 20 subjects who enrolled completed the study. Data from 20 subjects who at least completed one study period were included in the pharmacokinetic and statistical analyses. Mean concentration-time data are shown in FIGS. 15A through 17B. Results of the pharmacokinetic and statistical analyses are shown below in Tables 5 through 13.

TABLE 4
Pharmacokinetic parameters of Formulation A compared to RLD.
(Results from Tables 5-7)

| | | | | | |
|---|---|---|---|---|---|
| HC | | APAP | | PMZ | |
| Formu- | HC | Formu- | APAP | Formu- | PMZ |
| lation A | RLD | lation A | RLD | lation A | RLD |

Fasted

| | | | | | | |
|---|---|---|---|---|---|---|
| Tₘₐₓ (h) | 1.55 | 1.39 | 0.91 | 0.80 | 4.35 | 4.74 |
| Cₘₐₓ | 20.00 | 18.40 | 4.71 | 5.02 | 4.79 | 4.43 |
| (HC, PMZ, | | | | | | |
| ng/mL; | | | | | | |
| APAP | | | | | | |
| μg/mL) | | | | | | |
| T_{1/2} (h) | 4.93 | 5.13 | 4.59 | 4.52 | 17.49 | 17.92 |

Fed

| | | | | | | |
|---|---|---|---|---|---|---|
| Tₘₐₓ (h) | 3.38 | 3.09 | 2.84 | 2.64 | 5.24 | 6.45 |
| Cₘₐₓ | 17.90 | 17.50 | 3.27 | 3.03 | 3.66 | 4.08 |
| (HC, PMZ, | | | | | | |
| ng/mL; | | | | | | |
| APAP | | | | | | |
| μg/mL) | | | | | | |
| T_{1/2} (h) | 5.22 | 5.26 | 5.22 | 5.03 | 15.95 | 16.24 |

PMZ in Formulation A has a shorter Tmax, e.g., 49 minutes less, than that in RLD. The comparisons show slower absorption (later Tmax and lower Cmax) in Fed subjects for HC, APAP, and PMZ in both Formulation A and RLDs. Comparison of the partial AUCs over the first 2 hours for PMZ in Formulation A to RLD (Summary of Table 12).

| | |
|---|---|
| AUC_{0-0.75} | 143% |
| AUC_{0-1.0} | 159% |
| AUC_{0-1.5} | 150% |
| AUC_{0-2.0} | 147% |

Over the first hour there was 59% greater absorption of promethazine from Formulation A than a commercial product. Detailed results of pharmacokinetic parameters for Formulation A to reference drugs are shown below.

TABLE 5
Pharmacokinetic Parameters of Hydrocodone

| | Treatment A: | | | | Treatment B: | | | |
|---|---|---|---|---|---|---|---|---|
| | Test Formulation-Fasted | | | | Test Formulation-Fed | | | |
| Parameter | n | Mean | SD | CV % | n | Mean | SD | CV % |
| Tₘₐₓ (h) | 19 | 1.55 | 0.71 | 45.61 | 20 | 3.38 | 1.58 | 46.77 |
| Cₘₐₓ | 19 | 20.0 | 5.48 | 27.42 | 20 | 17.9 | 5.80 | 32.41 |
| (ng/mL) | | | | | | | | |
| AUC_{0-0.25} | 19 | 0.08870 | 0.1318 | 148.54 | 20 | 0.03786 | 0.1269 | 335.29 |
| (h*ng/mL) | | | | | | | | |
| AUC_{0-0.5} | 19 | 0.9532 | 0.9498 | 99.64 | 20 | 0.2566 | 0.6382 | 248.69 |
| (h*ng/mL) | | | | | | | | |
| AUC_{0-0.75} | 19 | 3.194 | 2.379 | 74.49 | 20 | 0.8759 | 1.645 | 187.87 |
| (h*ng/mL) | | | | | | | | |
| AUC_{0-1.0} | 19 | 6.383 | 3.637 | 56.97 | 20 | 2.046 | 3.244 | 158.56 |
| (h*ng/mL) | | | | | | | | |
| AUC_{0-1.5} | 19 | 14.33 | 4.971 | 34.69 | 20 | 5.782 | 7.049 | 121.91 |
| (h*ng/mL) | | | | | | | | |
| AUC_{0-2.0} | 19 | 23.21 | 6.240 | 26.88 | 20 | 10.95 | 10.34 | 94.40 |
| (h*ng/mL) | | | | | | | | |
| AUC_{0-4.0} | 19 | 54.04 | 13.77 | 25.47 | 20 | 38.97 | 18.47 | 47.39 |
| (h*ng/mL) | | | | | | | | |
| AUCₗₐₛₜ | 19 | 146.2 | 56.77 | 38.84 | 20 | 151.9 | 58.79 | 38.69 |
| (h*ng/mL) | | | | | | | | |
| AUC_{inf} | 19 | 150.2 | 57.30 | 38.16 | 20 | 155.8 | 58.75 | 37.70 |
| (h*ng/mL) | | | | | | | | |
| AUC_{Extrap} | 19 | 2.72 | 1.72 | 63.33 | 20 | 2.68 | 1.97 | 73.45 |
| (%) | | | | | | | | |
| λ_{z} (h⁻¹) | 19 | 0.1468 | 0.0293 | 19.94 | 20 | 0.1373 | 0.0258 | 18.78 |
| T_{1/2} (h) | 19 | 4.93 | 1.11 | 22.61 | 20 | 5.22 | 0.99 | 19.03 |
| Tₗₐₛₜ (h) | 19 | 27.80 | 8.99 | 32.32 | 20 | 31.21 | 11.28 | 36.13 |
| Cₗₐₛₜ | 19 | 0.556 | 0.354 | 63.60 | 20 | 0.523 | 0.412 | 78.77 |
| (ng/mL) | | | | | | | | |

| | Treatment C: | | | | Treatment D: | | | |
|---|---|---|---|---|---|---|---|---|
| | Comparator-Fasted | | | | Comparator-Fed | | | |
| Parameter | n | Mean | SD | CV % | n | Mean | SD | CV % |
| Tₘₐₓ (h) | 20 | 1.39 | 1.22 | 87.61 | 19 | 3.09 | 1.89 | 61.11 |
| Cₘₐₓ | 20 | 18.4 | 5.62 | 30.63 | 19 | 17.5 | 3.63 | 20.75 |
| (ng/mL) | | | | | | | | |
| AUC_{0-0.25} | 20 | 0.1219 | 0.2151 | 176.53 | 19 | 0.02250 | 0.04577 | 203.43 |
| (h*ng/mL) | | | | | | | | |
| AUC_{0-0.5} | 20 | 1.280 | 1.271 | 99.28 | 19 | 0.3163 | 0.4305 | 136.11 |
| (h*ng/mL) | | | | | | | | |
| AUC_{0-0.75} | 20 | 4.027 | 3.092 | 76.78 | 19 | 1.276 | 1.629 | 127.63 |
| (h*ng/mL) | | | | | | | | |
| AUC_{0-1.0} | 20 | 7.524 | 4.678 | 62.17 | 19 | 2.722 | 3.329 | 122.32 |
| (h*ng/mL) | | | | | | | | |
| AUC_{0-1.5} | 20 | 14.95 | 6.822 | 45.62 | 19 | 6.424 | 6.742 | 104.95 |
| (h*ng/mL) | | | | | | | | |
| AUC_{0-2.0} | 20 | 22.53 | 8.183 | 36.32 | 19 | 11.04 | 10.12 | 91.66 |
| (h*ng/mL) | | | | | | | | |
| AUC_{0-4.0} | 20 | 48.35 | 12.87 | 26.62 | 19 | 35.14 | 17.97 | 51.14 |
| (h*ng/mL) | | | | | | | | |
| AUCₗₐₛₜ | 20 | 130.9 | 47.94 | 36.63 | 19 | 148.5 | 56.84 | 38.27 |
| (h*ng/mL) | | | | | | | | |
| AUC_{inf} | 20 | 134.3 | 48.20 | 35.88 | 19 | 152.4 | 56.83 | 37.29 |
| (h*ng/mL) | | | | | | | | |
| AUC_{Extrap} | 20 | 2.65 | 1.78 | 67.24 | 19 | 2.71 | 2.06 | 76.01 |
| (%) | | | | | | | | |
| λ_{z} (h⁻¹) | 20 | 0.1415 | 0.0292 | 20.61 | 19 | 0.1371 | 0.0271 | 19.79 |
| T_{1/2} (h) | 20 | 5.13 | 1.18 | 22.94 | 19 | 5.26 | 1.08 | 20.59 |
| Tₗₐₛₜ (h) | 20 | 28.81 | 9.84 | 34.17 | 19 | 31.58 | 11.46 | 36.28 |
| Cₗₐₛₜ | 20 | 0.465 | 0.315 | 67.73 | 19 | 0.527 | 0.424 | 80.46 |
| (ng/mL) | | | | | | | | |

TABLE 6
Pharmacokinetic Parameters of Acetaminophen

| | Treatment A: | | | | Treatment B: | | | |
|---|---|---|---|---|---|---|---|---|
| | Test Formulation-Fasted | | | | Test Formulation-Fed | | | |
| Parameter | n | Mean | SD | CV % | n | Mean | SD | CV % |
| Tₘₐₓ (h) | 19 | 0.91 | 0.44 | 48.75 | 20 | 2.84 | 1.49 | 52.33 |
| Cₘₐₓ | 19 | 4.71 | 1.49 | 31.55 | 20 | 3.27 | 1.00 | 30.71 |
| (μg/mL) | | | | | | | | |
| AUCₗₐₛₜ | 19 | 19.19 | 5.661 | 29.51 | 20 | 17.85 | 4.621 | 25.88 |
| (h*μg/mL) | | | | | | | | |
| AUC_{inf} | 19 | 19.69 | 5.785 | 29.38 | 20 | 18.56 | 4.875 | 26.27 |
| (h*μg/mL) | | | | | | | | |
| AUC_{Extrap} | 19 | 2.63 | 1.52 | 58.04 | 20 | 3.67 | 1.23 | 33.46 |
| (%) | | | | | | | | |
| λ_{z} (h⁻¹) | 19 | 0.1577 | 0.0310 | 19.65 | 20 | 0.1359 | 0.0223 | 16.38 |
| T_{1/2} (h) | 19 | 4.59 | 1.06 | 23.18 | 20 | 5.22 | 0.79 | 15.17 |
| Tₗₐₛₜ (h) | 19 | 23.38 | 2.76 | 11.79 | 20 | 24.01 | 0.04 | 0.15 |
| Cₗₐₛₜ | 19 | 0.0741 | 0.0297 | 40.11 | 20 | 0.0917 | 0.0378 | 41.23 |
| (μg/mL) | | | | | | | | |
| | Treatment C: | | | | Treatment D: | | | |
| | Comparator-Fasted | | | | Comparator-Fed | | | |
| Parameter | n | Mean | SD | CV % | n | Mean | SD | CV % |
| Tₘₐₓ (h) | 20 | 0.80 | 0.55 | 69.21 | 19 | 2.64 | 1.44 | 54.51 |
| Cₘₐₓ | 20 | 5.02 | 1.66 | 33.06 | 19 | 3.03 | 0.919 | 30.29 |
| (μg/mL) | | | | | | | | |
| AUCₗₐₛₜ | 20 | 18.69 | 5.186 | 27.75 | 19 | 17.85 | 4.742 | 26.57 |
| (h*μg/mL) | | | | | | | | |
| AUC_{inf} | 20 | 19.16 | 5.331 | 27.82 | 19 | 18.53 | 4.988 | 26.92 |
| (h*μg/mL) | | | | | | | | |
| AUC_{Extrap} | 20 | 2.48 | 1.04 | 41.99 | 19 | 3.55 | 1.54 | 43.27 |
| (%) | | | | | | | | |
| λ_{z} (h⁻¹) | 20 | 0.1600 | 0.0355 | 22.20 | 19 | 0.1412 | 0.0218 | 15.41 |
| T_{1/2} (h) | 20 | 4.52 | 0.93 | 20.48 | 19 | 5.03 | 0.86 | 16.99 |
| Tₗₐₛₜ (h) | 20 | 23.41 | 2.69 | 11.47 | 19 | 24.01 | 0.02 | 0.10 |
| Cₗₐₛₜ | 20 | 0.0724 | 0.0291 | 40.26 | 19 | 0.0907 | 0.0428 | 47.17 |
| (μg/mL) | | | | | | | | |

TABLE 7
Pharmacokinetic Parameters of Promethazine

| | Treatment A: | | | | Treatment B: | | | |
|---|---|---|---|---|---|---|---|---|
| | Test Formulation-Fasted | | | | Test Formulation-Fed | | | |
| Parameter | n | Mean | SD | CV % | n | Mean | SD | CV % |
| Tₘₐₓ (h) | 19 | 4.35 | 1.37 | 31.60 | 19 | 5.24 | 2.37 | 45.26 |
| Cₘₐₓ | 19 | 4.79 | 3.75 | 78.24 | 19 | 3.66 | 2.16 | 59.08 |
| (ng/mL) | | | | | | | | |
| AUC_{0-0.25} | 19 | 0.01086 | 0.03169 | 291.92 | 19 | 0.003941 | 0.01413 | 358.62 |
| (h*ng/mL) | | | | | | | | |
| AUC_{0-0.5} | 19 | 0.03535 | 0.06239 | 176.52 | 19 | 0.01570 | 0.03508 | 223.44 |
| (h*ng/mL) | | | | | | | | |
| AUC_{0-0.75} | 19 | 0.1108 | 0.1090 | 98.38 | 19 | 0.05549 | 0.08128 | 146.49 |
| (h*ng/mL) | | | | | | | | |
| AUC_{0-1.0} | 19 | 0.2578 | 0.1999 | 77.56 | 19 | 0.1457 | 0.1649 | 113.19 |
| (h*ng/mL) | | | | | | | | |
| AUC_{0-1.5} | 19 | 0.8524 | 0.5410 | 63.47 | 19 | 0.5573 | 0.4798 | 86.08 |
| (h*ng/mL) | | | | | | | | |
| AUC_{0-2.0} | 19 | 1.938 | 1.318 | 68.04 | 19 | 1.267 | 0.9936 | 78.43 |
| (h*ng/mL) | | | | | | | | |
| AUC_{0-4.0} | 19 | 9.124 | 6.271 | 68.72 | 19 | 5.943 | 3.676 | 61.84 |
| (h*ng/mL) | | | | | | | | |
| AUCₗₐₛₜ | 19 | 75.69 | 78.87 | 104.20 | 19 | 66.63 | 67.37 | 101.11 |
| (h*ng/mL) | | | | | | | | |
| AUC_{inf} | 19 | 96.92 | 119.5 | 123.25 | 18 | 60.72 | 29.74 | 48.98 |
| (h*ng/mL) | | | | | | | | |
| AUC_{Extrap} | 19 | 14.35 | 7.03 | 49.02 | 18 | 13.54 | 4.64 | 34.31 |
| (%) | | | | | | | | |
| λ_{z} (h⁻¹) | 19 | 0.0429 | 0.0129 | 30.00 | 18 | 0.0451 | 0.0094 | 20.89 |
| T_{1/2} (h) | 19 | 17.49 | 5.21 | 29.81 | 18 | 15.95 | 3.04 | 19.07 |
| Tₗₐₛₜ (h) | 19 | 46.76 | 5.51 | 11.79 | 19 | 46.77 | 5.52 | 11.79 |
| Cₗₐₛₜ | 19 | 0.611 | 0.936 | 153.18 | 19 | 0.586 | 1.04 | 177.98 |
| (ng/mL) | | | | | | | | |
| | Treatment C: | | | | Treatment D: | | | |
| | Comparator-Fasted | | | | Comparator-Fed | | | |
| Parameter | n | Mean | SD | CV % | n | Mean | SD | CV % |
| Tₘₐₓ (h) | 19 | 4.74 | 1.60 | 33.82 | 19 | 6.45 | 4.59 | 71.16 |
| Cₘₐₓ | 19 | 4.43 | 2.99 | 67.48 | 19 | 4.08 | 1.95 | 47.73 |
| (ng/mL) | | | | | | | | |
| AUC_{0-0.25} | 19 | 0.006618 | 0.02675 | 404.16 | 19 | 0.002612 | 0.01138 | 435.89 |
| (h*ng/mL) | | | | | | | | |
| AUC_{0-0.5} | 19 | 0.02833 | 0.06229 | 219.85 | 19 | 0.01220 | 0.02452 | 201.04 |
| (h*ng/mL) | | | | | | | | |
| AUC_{0-0.75} | 19 | 0.1015 | 0.1504 | 148.17 | 19 | 0.04610 | 0.06227 | 135.08 |
| (h*ng/mL) | | | | | | | | |
| AUC_{0-1.0} | 19 | 0.2415 | 0.3102 | 128.48 | 19 | 0.1109 | 0.1471 | 132.56 |
| (h*ng/mL) | | | | | | | | |
| AUC_{0-1.5} | 19 | 0.7645 | 0.8503 | 111.23 | 19 | 0.3239 | 0.3643 | 112.47 |
| (h*ng/mL) | | | | | | | | |
| AUC_{0-2.0} | 19 | 1.650 | 1.708 | 103.56 | 19 | 0.6735 | 0.6328 | 93.96 |
| (h*ng/mL) | | | | | | | | |
| AUC_{0-4.0} | 19 | 7.363 | 6.212 | 84.37 | 19 | 4.045 | 2.497 | 61.74 |
| (h*ng/mL) | | | | | | | | |
| AUCₗₐₛₜ | 19 | 69.80 | 64.72 | 92.73 | 19 | 74.76 | 57.53 | 76.95 |
| (h*ng/mL) | | | | | | | | |
| AUC_{inf} | 19 | 90.51 | 102.6 | 113.35 | 18 | 84.13 | 76.72 | 91.19 |
| (h*ng/mL) | | | | | | | | |
| AUC_{Extrap} | 19 | 15.31 | 8.04 | 52.51 | 18 | 14.56 | 6.28 | 43.13 |
| (%) | | | | | | | | |
| λ_{z} (h⁻¹) | 19 | 0.0419 | 0.0123 | 29.24 | 18 | 0.0458 | 0.0145 | 31.53 |
| T_{1/2} (h) | 19 | 17.92 | 5.68 | 31.66 | 18 | 16.24 | 4.09 | 25.16 |
| Tₗₐₛₜ (h) | 19 | 46.75 | 5.51 | 11.78 | 19 | 46.74 | 5.51 | 11.78 |
| Cₗₐₛₜ | 19 | 0.589 | 0.848 | 143.94 | 19 | 0.701 | 0.912 | 130.12 |
| (ng/mL) | | | | | | | | |

TABLE 8
Statistical Analysis of the Log-Transformed Systemic Exposure
Parameters of Hydrocodone for Test Formulation-Fasted (Treatment
A) vs. Comparator Product-Fasted (Treatment C)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 19.1416 | 17.6351 | 108.54 | 97.30 | 121.09 | 0.9563 | 20.45 |
| ln(AUC_{0-0.25}) | 0.0813 | 0.1295 | 62.83 | 30.97 | 127.43 | 0.1418 | 98.76 |
| ln(AUC_{0-0.5}) | 0.5196 | 0.6313 | 82.31 | 40.19 | 168.58 | 0.1432 | 218.05 |
| ln(AUC_{0-0.75}) | 2.2732 | 2.8561 | 79.59 | 41.53 | 152.54 | 0.1524 | 179.91 |
| ln(AUC_{0-1.0}) | 5.3640 | 6.0985 | 87.96 | 45.90 | 168.56 | 0.1525 | 180.14 |
| ln(AUC_{0-1.5}) | 13.4272 | 13.5706 | 98.94 | 56.15 | 174.36 | 0.1691 | 141.44 |
| ln(AUC_{0-2.0}) | 22.2070 | 21.3215 | 104.15 | 66.01 | 164.34 | 0.2061 | 101.89 |
| ln(AUC_{0-4.0}) | 52.0387 | 46.9004 | 110.96 | 90.47 | 136.08 | 0.5622 | 39.15 |
| ln(AUCₗₐₛₜ) | 136.1000 | 123.2791 | 110.40 | 105.54 | 115.48 | 1.0000 | 8.34 |
| ln(AUC_{inf}) | 139.9624 | 126.6536 | 110.51 | 105.85 | 115.37 | 1.0000 | 7.98 |

^{a}Geometric Mean for Test Formulation-Fasted (Test) and Comparator Product-Fasted (Ref) based on Least Squares Mean of log-transformed parameter values
^{b}Ratio(%) = Geometric Mean (Test)/Geometric Mean (Ref)
^{c}90% Confidence Interval

TABLE 9
Statistical Analysis of the Log-Transformed Systemic Exposure
Parameters of Hydrocodone for Test Formulation-Fed (Treatment
B) vs. Comparator Product-Fed (Treatment D)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 17.0949 | 17.0793 | 100.09 | 89.72 | 111.66 | 0.9563 | 20.45 |
| ln(AUC_{0-0.25}) | 0.0301 | 0.0599 | 50.26 | 16.28 | 155.16 | 0.1164 | 98.76 |
| ln(AUC_{0-0.5}) | 0.1007 | 0.1947 | 51.73 | 23.07 | 116.02 | 0.1340 | 218.05 |
| ln(AUC_{0-0.75}) | 0.3736 | 0.5842 | 63.96 | 31.58 | 129.51 | 0.1446 | 179.91 |
| ln(AUC_{0-1.0}) | 0.9916 | 0.9043 | 109.66 | 55.73 | 215.76 | 0.1485 | 180.14 |
| ln(AUC_{0-1.5}) | 3.3736 | 2.4286 | 138.91 | 78.83 | 244.79 | 0.1691 | 141.44 |
| ln(AUC_{0-2.0}) | 7.9442 | 5.3848 | 147.53 | 93.50 | 232.78 | 0.2061 | 101.89 |
| ln(AUC_{0-4.0}) | 35.1589 | 29.6010 | 118.78 | 96.85 | 145.67 | 0.5622 | 39.15 |
| ln(AUCₗₐₛₜ) | 141.2952 | 139.1236 | 101.56 | 97.09 | 106.23 | 1.0000 | 8.34 |
| ln(AUC_{inf}) | 145.2129 | 143.1030 | 101.47 | 97.20 | 105.94 | 1.0000 | 7.98 |

^{a}Geometric Mean for Test Formulation-Fed (Test) and Comparator Product-Fed (Ref) based on Least Squares Mean of log-transformed parameter values
^{b}Ratio(%) = Geometric Mean (Test)/Geometric Mean (Ref)
^{c}90% Confidence Interval

TABLE 10
Statistical Analysis of the Log-Transformed Systemic Exposure
Parameters of Acetaminophen for Test Formulation-Fasted (Treatment
A) vs. Comparator Product-Fasted (Treatment C)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 4.4880 | 4.7934 | 93.63 | 83.08 | 105.52 | 0.9236 | 22.39 |
| ln(AUCₗₐₛₜ) | 18.3460 | 17.9344 | 102.29 | 98.13 | 106.64 | 1.0000 | 7.70 |
| ln(AUC_{inf}) | 18.8457 | 18.3907 | 102.47 | 98.40 | 106.71 | 1.0000 | 7.51 |

^{a}Geometric Mean for Test Formulation-Fasted (Test) and Comparator Product-Fasted (Ref) based on Least Squares Mean of log-transformed parameter values
^{b}Ratio(%) = Geometric Mean (Test)/Geometric Mean (Ref)
^{c}90% Confidence Interval

TABLE 11
Statistical Analysis of the Log-Transformed Systemic Exposure
Parameters of Acetaminophen for Test Formulation-Fed (Treatment
B) vs. Comparator Product-Fed (Treatment D)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 3.1074 | 2.9065 | 106.91 | 94.86 | 120.49 | 0.9236 | 22.39 |
| ln(AUCₗₐₛₜ) | 17.2244 | 17.2805 | 99.68 | 95.62 | 103.91 | 1.0000 | 7.70 |
| ln(AUC_{inf}) | 17.8828 | 17.9172 | 99.81 | 95.84 | 103.94 | 1.0000 | 7.51 |

^{a}Geometric Mean for Test Formulation-Fed (Test) and Comparator Product-Fed (Ref) based on Least Squares Mean of log-transformed parameter values
^{b}Ratio(%) = Geometric Mean (Test)/Geometric Mean (Ref)
^{c}90% Confidence Interval

TABLE 12
Statistical Analysis of the Log-Transformed Systemic Exposure
Parameters of Promethazine for Test Formulation-Fasted (Treatment
A) vs. Comparator Product-Fasted (Treatment C)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 3.6763 | 3.6145 | 101.71 | 89.15 | 116.04 | 0.8746 | 24.24 |
| ln(AUC_{0-0.5}) | 0.0287 | 0.0280 | 102.75 | 56.63 | 186.42 | 0.1597 | 73.29 |
| ln(AUC_{0-0.75}) | 0.0716 | 0.0500 | 143.22 | 75.41 | 271.99 | 0.1537 | 164.57 |
| ln(AUC_{0-1.0}) | 0.1829 | 0.1152 | 158.76 | 88.39 | 285.16 | 0.1645 | 143.87 |
| ln(AUC_{0-1.5}) | 0.6920 | 0.4614 | 149.98 | 85.19 | 264.04 | 0.1693 | 136.46 |
| ln(AUC_{0-2.0}) | 1.5615 | 1.0609 | 147.19 | 91.12 | 237.74 | 0.1961 | 106.26 |
| ln(AUC_{0-4.0}) | 7.2596 | 5.4269 | 133.77 | 93.98 | 190.41 | 0.2744 | 71.16 |
| ln(AUCₗₐₛₜ) | 51.9433 | 52.0597 | 99.78 | 91.26 | 109.09 | 0.9924 | 16.29 |
| ln(AUC_{inf}) | 60.7200 | 61.7188 | 98.38 | 90.60 | 106.83 | 0.9969 | 15.00 |

^{a}Geometric Mean for Test Formulation-Fasted (Test) and Comparator Product-Fasted (Ref) based on Least Squares Mean of log-transformed parameter values
^{b}Ratio(%) = Geometric Mean (Test)/Geometric Mean (Ref)
^{c}90% Confidence Interval
Note:
Due to limited AUC_{0-0.25} data, no statistical analysis could be performed for ln(AUC_{0-0.25})

TABLE 13
Statistical Analysis of the Log-Transformed Systemic Exposure
Parameters of Promethazine for Test Formulation-Fed (Treatment
B) vs. Comparator Product-Fed (Treatment D)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 3.3595 | 3.6441 | 92.19 | 80.84 | 105.14 | 0.8762 | 24.24 |
| ln(AUC_{0-0.5}) | 0.0282 | 0.0236 | 119.35 | 62.05 | 229.53 | 0.1495 | 73.29 |
| ln(AUC_{0-0.75}) | 0.0410 | 0.0369 | 111.12 | 50.58 | 244.12 | 0.1357 | 164.57 |
| ln(AUC_{0-1.0}) | 0.1140 | 0.0616 | 185.13 | 97.60 | 351.15 | 0.1541 | 143.87 |
| ln(AUC_{0-1.5}) | 0.3596 | 0.1674 | 214.84 | 122.23 | 377.59 | 0.1697 | 136.46 |
| ln(AUC_{0-2.0}) | 0.9227 | 0.4225 | 218.38 | 135.39 | 352.24 | 0.1966 | 106.26 |
| ln(AUC_{0-4.0}) | 5.1208 | 3.1295 | 163.63 | 115.07 | 232.67 | 0.2754 | 71.16 |
| ln(AUCₗₐₛₜ) | 55.5554 | 59.1074 | 93.99 | 85.99 | 102.74 | 0.9926 | 16.29 |
| ln(AUC_{inf}) | 65.8925 | 72.1654 | 91.31 | 83.91 | 99.36 | 0.9958 | 15.00 |

^{a}Geometric Mean for Test Formulation-Fed (Test) and Comparator Product-Fed (Ref) based on Least Squares Mean of log-transformed parameter values
^{b}Ratio(%) = Geometric Mean (Test)/Geometric Mean (Ref)
^{c}90% Confidence Interval
Note:
Due to limited AUC_{0-0.25} data, no statistical analysis could be performed for ln(AUC_{0-0.25})

### Example 8: Design of Clinical Phase III Study of Formulation a (Third Molar Extraction)

A Clinical Phase III study was performed for Formulation A in the form of a bi-layer tablet in the treatment of moderate to severe pain. This was a multi-center, double-blind, randomized, multiple-dose, placebo- and positive-controlled study of Formulation A in subjects with moderate to severe pain following surgical removal of impacted third molar teeth. A positive control (a commercial formulation-NORCO (hydrocodone 7.5 mg/acetaminophen 325 mg)) was included to determine the antiemetic effects of Formulation A.

Human adults 18 years of age or older with impacted third molars had a screening visit during which a panoramic dental x-ray and admission criteria were reviewed and a pre-operative physical examination was performed. The medical history and medication history was obtained, including a “Nausea-Prone Questionnaire,” or NPQ, to identify subjects who were likely or possibly “nausea-prone,” i.e., likely or possibly at risk of opioid-induced nausea or vomiting (OINV). During a Hydrocodone Challenge, subjects received a single dose of NORCO (hydrocodone 7.5 mg/acetaminophen 325 mg) and were observed for 2 hours for all effects of the medication, in particular, for any nausea or vomiting.

Subjects who were considered “Likely Nausea-Prone” or “Possibly Nausea-Prone” based on their responses on the NPQ and subjects who actually experienced nausea or vomiting after the Hydrocodone Challenge were scheduled for surgery (removal of at least 2 third molar teeth, one of which required either partial or full bony extraction from the mandible) not earlier than 1 week later.

Oral Surgery was performed using only intravenous conscious sedation (midazolam), nitrous oxide, and local anesthesia (3% mepivacaine plain for blocks, lidocaine with epinephrine 1:100,000 for local infiltration anesthesia). Prophylactic antiemetic and analgesic medications were not permitted. Surgery involved the removal of at least 2 third molar teeth, one of which required either partial or full bony extraction from the mandible

Subjects were observed post-surgery to determine if they developed at least moderate pain on a 4-point categorical pain intensity scale (PI-CAT), confirmed by a score of at least 50 mm on a 100-mm visual analog pain intensity scale (PI-VAS). Four study questionnaires concerning common opioid-related side effects were administered to each eligible subject before receiving study medication: Opioid Symptoms Scales (OSS) to evaluate other common opioid-related side effects such as sedation, dizziness, itch; a Nausea Intensity Scale (NIS); a Vomiting Frequency Scale (VFS); and a Stomach Scale (StomS) to assess stomach status, ranging from normal to vomiting.

Under double-blind conditions qualifying subjects (n=466) were randomized (4:4:1) within the two nausea-prone stratifications to use one of the three treatments: Formulation A (hydrocodone 7.5 mg/acetaminophen 325 mg and promethazine 12.5 mg), NORCO, or Placebo.

Subjects remained at the research site for the first 6 hours post-treatment observations. Vital signs (pulse, respiratory rate), blood pressure, and pulse oximetry were obtained immediately prior to dosing and hourly until 6 hours post-dosing. Temperature was measured at Baseline and at 6 hours post-dosing.

At each half-hourly time point over 6 hours, subjects assessed pain intensity (PI-CAT) and pain relief (REL). After each pair of pain intensity and pain relief assessments, the Nausea Intensity Scale (NIS) was completed each half-hour in order to assess the occurrence and severity of nausea. The Vomiting Frequency Scale (VFS) was used to document the occurrence and frequency of vomiting at hourly intervals, when subjects also used the Stomach Scale (StomS) to assess stomach status. At the end of this 6-hour treatment evaluation period, subjects repeated the PI-VAS and the Opioid Symptoms Scales (OSS) and assessed their satisfaction with the study medication on a Subject Satisfaction Scale. Subjects were then discharged home.

For hours 7-24 (while awake), subjects made hourly assessments of pain (on the PI-CAT) and nausea/vomiting (on the NIS and VFS). At bed-time, subjects also documented other opioid symptoms experienced over the day (on the OSS). Subjects self-dosed with assigned study medication as needed for pain every 4-6 hours, for a maximum of 6 doses in a 24-hour period, documenting the date and the times in an Subject Diary. Subjects also recorded the date and times of taking supplemental medications for pain (ibuprofen 400 mg every 4-6 hrs) and/or for nausea/vomiting (as prescribed according to standard clinical practice).

After the initial 24 hours and for all 5 study days, subjects documented each self dosing. Immediately before each self-dosing they made assessments on the PI-CAT and NIS; at 1 and 2 hours after each self-dosing subjects made assessments on the PI-CAT, NIS, and VFS. Every night for 5 nights, subjects also assessed other opioid-related side effects on the OSS. Within approximately 1 week after surgery, subjects returned to the clinic for a post-op visit and were then discharged from the study.

Study participants were excluded for any of the following reasons: medical condition; infection; drug allergy (history of hypersensitivity to an opioid drug, promethazine, acetaminophen, NSAID (such as ibuprofen) or ketorolac or history of a dystonic/dyskinetic reaction to prior antiemetic or anti-psychotic medication; confounding and contraindicated Drugs (use (within 24 hours of the surgical procedure) of any confounding prescription or non-prescription drug (e.g., analgesic, antiemetic, sedating antihistamine, sedative, alcohol, CNS/psychotropic agent, including sleep aides, benzodiazepines, performance/attention enhancers, marijuana, anti-depressants) or any drug contraindicated with hydrocodone, acetaminophen, or promethazine (except for pre-op medications); antibiotic prophylaxis for endocarditis is permitted (except if known to cause nausea); caffeine use since mid-night before the operation; investigational drug use (use of an investigational drug within the past 30 days); previous participation in the study; present pregnancy or lactation; and participant relationships with an employee or relative of an employee who is directly involved in study oversight or funding.

Subjects were observed post-op in the clinic for up to 4 hours to determine if they became eligible for the study by reporting moderate or severe post-operative pain on the 4-point categorical pain intensity scale (PI-CAT). All prospective subjects had to have a score of at least 50 mm on the 100-mm visual analog pain intensity scale (PI-VAS) for admission to the study. Qualitative dimensions of pain (sensory, affective, and evaluative) were also assessed on a Dental Pain Qualities Index (QDPI). Subjects who did not qualify were screen failures. Qualifying subjects (n=466) were randomized within the two nausea-prone risk stratifications (Likely Nausea-Prone or, Possibly Nausea-Prone) to assure equal distribution of predisposition to OINV within the treatment groups. A computer-generated randomization code was used by the statistician to allocate subjects to: Formulation A (n=211), NORCO (n=205), or placebo (n=50).

Before receiving study medication, all qualifying subjects completed several different side effect scales: a Nausea Intensity Scale (NIS) from the Edmonton Symptom Assessment Scale; Opioid Symptoms Scales Opioid Symptoms Scales (OSS) derived from the Symptom Distress Scale; a Vomiting Frequency Scale (VFS); a Stomach Scale (StomS); a Symptom Checklist. The purpose of administering these side effect scales was to document if the subject had nausea or vomiting (e.g., post-operative nausea/vomiting, or PONV) or other opioid-related symptoms (e.g., drowsiness, dizziness) prior to opioid exposure. These measurements thus served as pre-treatment measures for “adverse non-drug reactions.” Study medication was then administered to subjects under double-blind conditions, and the subjects all remained in the clinic for the first 6 hours post initial treatment.

After discharge from the clinic, subjects used a separate Outpatient Diary. Subjects were permitted to take another single capsule of study medication at 4 hours after the initial dose, and at every 4-6 hours as needed. During the initial 24-hour treatment period of the study (and for every day in the study), subjects were allowed to self-medicate with a single capsule of study medication every 4-6 hours, up to a maximum of 6 doses of study medication over a 24-hour period as needed for pain.

After discharge from the clinic, subjects used the Outpatient Diary to record their hourly ratings on the PI-CAT, NIS, and VFS for hours 7-24 (while awake). The subjects were required to document all hourly evaluations of PICAT, NIS, VFS and side effects on the OSS throughout the entire initial 24-hour treatment evaluation period and all evaluations while self-dosing for up to 5 days Subjects were required to document the use of their assigned study medication as well as the use of any supplemental medication.

After the completion of ratings at 24 hours, subjects were required to use the Outpatient Diary to document the dates and times of each subsequent self-dosing (every 4-6 hours, as needed for pain, with up to 6 doses per 24-hour period). Before each self-dosing on Days 2-7, subjects were required to make entries into the Outpatient Diary which included their pre-treatment ratings on the PI-CAT, NIS, and VFS.

The subjects employed the Outpatient Diary at 1 and 2 hours after each dosing to rate PI-CAT, NIS, and VFS. Subjects were required to make entries into the same Outpatient Diary every night to note any other opioid-related side effects, such as itchiness, constipation, sedation, dizziness (on the OSS) and any other side effects or changes in their medical condition as might have occurred and to document the date and time of taking any supplemental medication(s). Subjects were required to document all protocol mandated evaluations while self-dosing for post-operative pain, as needed, for up to 5 days. Subjects were also required to document any other opioid related side effect on the OSS every evening for the 5 study days even if they had stopped taking medication for pain.

### Example 9: Analysis of Clinical Phase III Study for Formulation A (Third Molar Extraction)

Endpoint Categories.

Results from the study described in Example 8 were analyzed. The primary variables were pain and opioid-induced nausea/vomiting as experienced by subjects who had just undergone extraction of 2 or more impacted third molars. Two co-primary endpoints were used, one for the assessment of opioid induced nausea or vomiting (OINV) and the other for the assessment of pain. Both endpoints were defined over the initial 24 hours following randomization.

The co-primary endpoint for opioid-induced nausea or vomiting (OINV) for a subject was a binary assessment of response/no response, a composite index for the occurrence of opioid-induced nausea or vomiting (OINV) over 24 hours. A subject was considered a responder if he or she experienced at most mild nausea (as documented by a rating ≤3 on the NIS) during the 24 hours post randomization. A subject was considered a non-responder if he or she experienced moderate or severe nausea (as documented by NIS ratings of 4-6 or 7-10, respectively) or vomited (as documented on the Vomiting Frequency Scale), or received supplemental antiemetic medication at any point during the 24 hours post randomization.

The co-primary analgesia endpoint for a subject was the time weighted sum of pain intensity differences over 24 hours (SPID24), comparing Formulation A to placebo. The endpoint was calculated from the PI-CAT values at baseline, every 30 minutes until hour 6, and then every hour (while awake) until hour 24 as follows: (1) the baseline the PI-CAT value was subtracted from each of the subsequent PI-CAT values; (2) each difference was weighted by the elapsed time from the previous PI-CAT value to the current one; and (3) the weighed differences were summed to yield the SPID24. Subjects who received supplemental medication for pain, nausea, or vomiting (i.e., non-responders) had their subsequent PI-CAT values replaced by the baseline PI-CAT (yielding differences of 0). A total of 466 subjects had completed the study when it was stopped after the Data Safety Monitoring Board's interim analysis.

During the study, subjects assigned to Formulation A took, on average, 3.5 capsules on the first day (most commonly 3 capsules); subjects on NORCO took, on average, 3.6 capsules on the first day (most commonly 3 capsules); placebo, 3.3 capsules on the first day (most commonly 3 capsules). There use of study medications gradually decreased for each successive day during the post-operative period with no significant differences between the treatment groups.

Other than the dental conditions which had induced subjects to seek oral surgery there were no individual physical abnormalities that exceeded 2% of the population in any treatment group. Except for the prior use of hormonal and topical contraceptives in this predominately female population and the prior use of anti-inflammatory, anti-rheumatic, anti-pyretic and analgesic products before midnight on the day prior to surgery, the use of other prior medications did not exceed 5% for any other medication type.

Results from Nausea-Prone Assessment of Study Participants.

Table 14A-B provides the nausea-prone assessment for subjects in the subject population. The nausea-prone status for subjects as actually randomized was approximately ¾ of subjects who were likely nausea-prone and approximately ¼ of subjects who were possibly nausea prone in each of the three treatment groups. Five other pre-treatment nausea prone assessments also showed that the subjects in all three treatment groups were quite closely comparable.

TABLE 14A
Nausea Prone Assessment - Hydrocodone Challenge Result (Intent to Treat Population)

| | | Positive | | | Negative | | |
|---|---|---|---|---|---|---|---|
| | | Formulation A | NORCO | Placebo | Formulation A | NORCO | Placebo |
| | | (n = 143) | (n = 145) | (n = 34) | (n = 68) | (n = 60) | (n = 16) |
| Nausea Prone | Likely | 33 (23.1) | 29 (20.0) | 9 (26.5) | 18 (26.5) | 14 (23.3) | 3 (18.8) |
| Result Based | Possibly | 0 | 0 | 0 | 50 (73.5) | 46 (76.7) | 13 (81.3) |
| on Nausea Prone | Neither | 110 (76.9) | 116 (80.0) | 25 (73.5) | 0 | 0 | 0 |
| Questionnaire | Likely | | | | | | |
| (NPQ), n (%) | nor Possibly | | | | | | |

NPQ = Nausea Prone Questionnaire. Nausea prone status based on NPQ was not recorded on the case report form for all the subjects who had positive hydrocodone challenge results, and is indicated “Neither Likely nor Possibly Nausea Prone” in the report.

TABLE 14B
Nausea Prone Assessment (Intent to Treat Population)

| | | Formulation A | NORCO | Placebo |
|---|---|---|---|---|
| | | (n = 211) | (n = 205) | (n = 50) |
| Nausea prone | Likely | 160 (75.8) | 159 (77.6) | 37 (74.0) |
| status actually | Possibly | 51 (24.2) | 46 (22.4) | 13 (26.0) |
| randomized, | | | | |
| n (%) | | | | |
| Hydrocodone | Positive | 143 (67.8) | 145 (70.7) | 34 (68.0) |
| challenge, n (%) | Negative | 68 (32.2) | 60 (29.3) | 16 (32.0) |
| Nausea intensity | n | 211 | 205 | 50 |
| scale after | Mean (SD) | 2.8 (2.7) | 2.6 (2.6) | 2.7 (2.6) |
| hydrocodone | Median | 2.0 | 2.0 | 2.0 |
| challenge | Min, Max | (0, 10) | (0, 10) | (0, 10) |
| Nausea intensity | ≥7 | 27 (12.8) | 23 (11.2) | 4 (8.0) |
| scale after | <7 | 184 (87.2) | 182 (88.8) | 46 (92.0) |
| hydrocodone | | | | |
| challenge, n (%) | | | | |
| Nausea prone | Likely | 51 (24.2) | 43 (21.0) | 12 (24.0) |
| status based on | Possibly | 50 (23.7) | 46 (22.4) | 13 (26.0) |
| NPQ, n (%) | Neither | 110 (52.1) | 116 (56.6) | 25 (50.0) |
| | Likely | | | |
| | nor Possibly | | | |
| Nausea prone | Likely | 161 (76.3) | 159 (77.6) | 37 (74.0) |
| status based on | Possibly | 50 (23.7) | 46 (22.4) | 13 (26.0) |
| Investigator's | | | | |
| Assessment, | | | | |
| n (%) | | | | |

NPQ = Nausea Prone Questionnaire,
Min = minimum,
Max = maximum,
SD = standard deviation.
Nausea prone status based on Investigator's Assessment was collected at Visit 1. Nausea prone status based on NPQ was not recorded on the case form for all the subjects who had positive hydrocodone challenge results, and is indicated to be determined in the report.

Increased Pain Relief in Subjects Treated with Formulation A.

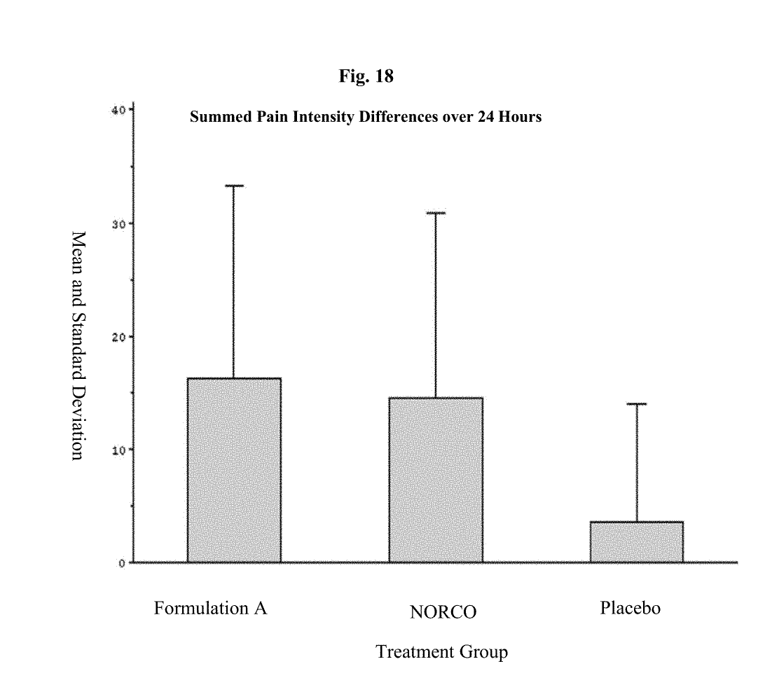

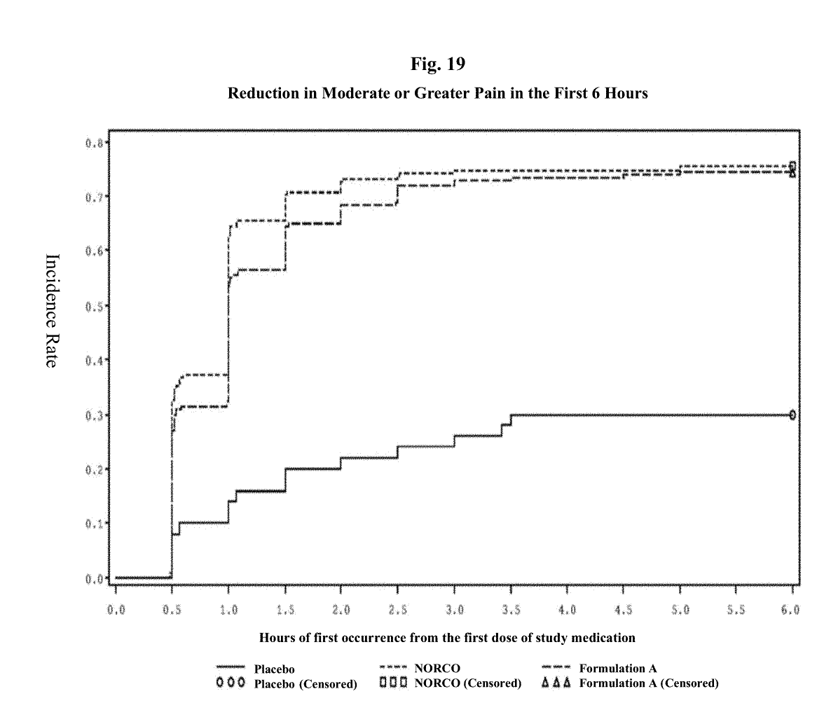

For one of the Co-Primary endpoints (the summed differences in pain intensity [on PI-CAT] over the initial 24 hours post randomization, SPID24), the results are provided in Table 15 and FIG. 18 which showed a significant difference in pain reduction between Formulation A and placebo (16.2 and 3.5, respectively; p<0.001).

TABLE 15
Co-Primary Endpoint Results (Intent to Treat Population)

| | | Formulation | | |
|---|---|---|---|---|
| | | A | NORCO | Placebo |
| | | (N = 211) | (N = 205) | (N = 50) |
| Summed pain | Mean (SD) | 16.2 (17.1) | 14.6 (16.3) | 3.5 (10.5) |
| intensity | Median | 11.5 | 8.1 | 0.0 |
| differences (on | Min, Max | (−19, 68) | (−3, 68) | (−13, 45) |
| PI-CAT) over | Type 3 p value | <0.001 | | |
| initial 24 hours | (Formulation | | | |
| | A vs Placebo) | | | |

PI-CAT = Categorical Pain Intensity Scale,
Min = minimum,
Max = maximum,
SD = standard deviation.
PI-CAT is based on: 0 = No Pain, 1 = Mild Pain, 2 = Moderate Pain, 3 = Severe Pain. The analgesia co-primary endpoint is defined as the summed pain intensity differences (on PI-CAT) subtracted from baseline over 24 hours post randomization (SPID24). The last observation result was used to replace any missing results prior to the first use of supplemental medication for pain or early withdrawal. The pain intensity result was set to the baseline result at all the time points post the first use of supplemental medication for pain or early withdrawal. The type 3 p-value for the analgesia endpoint is from the general linear model with factors of treatment, site and baseline pain intensity.

Formulation A is much more effective than NORCO in relieving severe pain (i.e., scale >=70 mm or >=7 in the scale of 10), in addition to reducing nausea/vomiting. For example, in Table 16, Formulation A increased pain relief by about 25% over initial 24 hours.

TABLE 16
Numeric Pain Intensity Rating scale >=70 mm at baseline.

| | | Formulation | NORCO | Placebo |
|---|---|---|---|---|
| | | A (N = 107) | (N = 118) | (N = 29) |
| Nausea/ | Yes, n (%) | 62 (57.9) | 44 (37.3) | 23 (79.3) |
| Vomiting | | | | |
| responder | | | | |
| (nausea | | | | |
| co-primary | | | | |
| endpoint) | | | | |
| Summed pain | Mean (SD) | 21.1 (19.0) | 16.9 (18.3) | 1.0 (7.1) |
| intensity | Median | 19.0 | 10.2 | 0.0 |
| differences (on | Min, Max | (−3, 68) | (−2, 68) | (−13, 34) |
| PI-CAT) over | | | | |
| initial 24 hours | | | | |

PI-CAT = Categorical Pain Intensity Scale.
Min = minimum,
Max = maximum,
SD = standard deviation.
PI-CAT is based on: 0 = No Pain, 1 = Mild Pain, 2 = Moderate Pain, 3 = Severe Pain.

Table 17A provides the linear difference in Pain Intensity (as measured on the PI-VAS) at 6 hours compared to baseline for the subject population. Subjects on Formulation A experienced a 36% reduction in pain intensity compared to a 15% reduction by subjects on placebo (p<0.001). This evidence of analgesic activity was also registered by the observation that 51% of subjects who took a single dose of Formulation A reported ≥30% reduction in pain intensity in the first 6 hours compared with 20% of subjects who used placebo (p<0.001), Table 17B.

TABLE 17A
Pain Intensity (PI-VAS) Results over 6 Hours (Intent to Treat
Population)

| | | Formulation A (n = | NORCO | Placebo |
|---|---|---|---|---|
| | | 211) | (n = 205) | (n = 50) |
| Pain intensity | Mean (SD) | 26.7 (27.2) | 28.9 (29.2) | 9.8 (19.2) |
| difference (on | Median | 24.0 | 24.0 | 0.0 |
| PI-VAS) at 6 | Min, Max | (−26, 95) | (−15, 93) | (0, 68) |
| hours | Type 3 p value | <0.001 | | |
| | (Formulation A | | | |
| | vs Placebo) | | | |
| Percent change | Mean (SD) | 36.33 (36.26) | 39.57 (39.14) | 14.68 (28.92) |
| in pain (on PI- | Median | 32.95 | 37.50 | 0.00 |
| VAS) at 6 hours | Min, Max | (−37.7, 100.0) | (−22.6, 100.0) | (0.0, 100.0) |

| | |
|---|---|
| Type 3 p value | <0.001 |
| (Formulation A | |
| vs Placebo) | |

PI-VAS = Visual Analog Pain Intensity Scale,
Min = minimum,
Max = maximum,
SD = standard deviation.
PI-VAS is based on current pain with a scale of 0-to-100, where 0 is no pain and 100 is severe pain. The baseline result (if baseline non-missing) or last observation (if baseline missing) was used for replacing the actual or missing result at 6 hours post baseline if a subject used supplemental medication for pain or had early withdrawal prior to 6 hours post baseline. Pain intensity difference is calculated as the pain intensity of a post-dose time point, divided by baseline. The type 3 p value is from the general linear model with factors of treatment, site and baseline pain intensity (PI-VAS).

TABLE 17B
Pain Intensity (PI-VAS) Results (Intent to Treat Population)

| | | Formulation | NORCO | Placebo |
|---|---|---|---|---|
| | | A (n = 211) | (n = 205) | (n = 50) |
| Subjects with | n (%) | 108 (51.2) | 104 (50.7) | 10 |
| >=30% reduction | | | | (20.0) |
| in pain (on PI- | Adjusted Odds Ratio and 95% | 5.1 (2.3, 11.3) | | |
| VAS) at 6 hours | CI | | | |
| | Type 3 p value | <0.001 | | |
| | (Formulation A vs Placebo) | | | |
| | NNT (95% CI) | −3 (−2, −5) | | |
| Subjects with >=20% | n (%) | 120 (56.9) | 112 (54.6) | 11 |
| reduction in | | | | (22.0) |

| | | |
|---|---|---|
| pain (on PI- | Adjusted Odds Ratio and 95% | 5.8 (2.7, 12.7) |
| VAS) at 6 hours | CI | |
| | Type 3 p value | <0.001 |
| | (Formulation A vs Placebo) | |
| | NNH (95% CI) | −3 (−2, −5) |

PI-VAS = Visual Analog Pain Intensity Scale,
CI = confidence interval,
NNH = number needed to harm,
Min = minimum,
Max = maximum,
SD = standard deviation.
PI-VAS is based on current pain with a scale of 0-to-100, where 0 is no pain and 100 is severe pain. The NNH and 95% CI are based on the inverse of the absolute risk difference between Formulation A and Placebo. The adjusted odds ratio, 95% CI, and type 3 p value are from the logistic regression model with factors of treatment, site and baseline pain intensity (PI-VAS).

The effects of each of the three treatments on Pain Intensity (PI-CAT) in the subject population are shown in Table 18. When treatment with Formulation A is compared with placebo, the differences in pain intensity are significant (Type 3 p-value <0.001) over 4 hours and 6 hours post randomization (i.e., as currently labeled).

TABLE 18
Pain Intensity (PI-CAT) Results (Intent to Treat Population): Summed
pain intensity differences (on PI-CAT)

| | | Formulation | NORCO | Placebo |
|---|---|---|---|---|
| | | A (n = 211) | (n = 205) | (n = 50) |
| Over initial | Mean (SD) | 2.3 (2.5) | 2.4 (2.3) | 0.2 (1.6) |
| 4 hours | Median | 2.1 | 2.1 | 0.0 |
| | Min, Max | (−3, 11) | (−3, 11) | (−3, 5) |
| | Type 3 p value | <0.001 | | |
| | (Formulation A vs | | | |
| | Placebo) | | | |
| Over initial | Mean (SD) | 3.5 (3.6) | 3.7 (3.5) | 0.7 (2.4) |
| 6 hours | Median | 2.8 | 3.3 | 0.0 |
| | Min, Max | (−4, 15) | (−3, 15) | (−5, 9) |

| | |
|---|---|
| Type 3 p value | <0.001 |
| (Formulation A vs | |
| Placebo) | |

Pain Relief Results in the ITT population are provided in Table 19. Comparing the various time interval strata for Formulation A and placebo show Formulation A to be strongly superior to placebo in providing pain relief at both 4 hours (p<0.001) and at 6 hours (p<0.001) when pain relief is measured either by Total Pain Relief at each time interval or by the % of Maximum Total Pain Relief at each time interval.

TABLE 19
Pain Relief Results (Intent to Treat Population)

| | | Formulation | NORCO | Placebo |
|---|---|---|---|---|
| | | A (n = 211) | (n = 205) | (n = 50) |
| Total pain relief over initial | Mean (SD) | 7.3 (5.3) | 7.8 (5.2) | 2.8 (4.1) |
| 4 hours (TOTPAR4) | Median | 6.0 | 7.3 | 1.0 |
| | Min, Max | (0, 23) | (0, 22) | (0, 17) |
| | Type 3 p value | <0.001 | | |
| | (Formulation A vs | | | |
| | Placebo) | | | |
| % of maximum total pain | Mean (SD) | 48.1 (23.7) | 49.1 (21.5) | 23.3 (22.7) |
| relief over initial 4 hours | | | | |
| (% maxTOPAR4) | Median | 50.0 | 52.4 | 18.8 |
| | Min, Max | (0, 92) | (0, 90) | (0, 78) |
| | Type 3 p value | <0.001 | | |
| | (Formulation A vs | | | |
| | Placebo) | | | |
| Total pain relief over initial | Mean (SD) | 11.3 (8.4) | 11.9 (8.6) | 4.3 (6.8) |
| 6 hours (TOTPAR6) | Median | 10.3 | 11.3 | 1.0 |
| | Min, Max | (0, 33) | (0, 33) | (0, 26) |
| | Type 3 p value | <0.001 | | |
| | (Formulation A vs | | | |
| | Placebo) | | | |
| % of maximum total pain | Mean (SD) | 44.6 (25.0) | 45.2 (24.4) | 20.7 (22.8) |
| relief over initial 6 hours | | | | |
| (% maxTOTPAR6) | Median | 45.8 | 50.0 | 12.5 |
| | Min, Max | (0, 91) | (0, 92) | (0, 72) |

| | |
|---|---|
| Type 3 p value | <0.001 |
| (Formulation A vs | |
| Placebo) | |

Min = minimum,
Max = maximum,
SD = standard deviation.
Pain relief is based on Categorical Relief Scale (How much pain relief do you have now?): 0 = No Relief, 1 = Slight Relief, 2 = Mild Relief, 3 = Moderate Relief, 4 = Considerable Relief, 5 = Almost Complete Relief, 6 = Complete Relief. The last observation result was used to replace any missing results prior to the first use of the supplemental medication for pain or early withdrawal. The pain relief scale was set to 0 (no relief) at baseline and at all the time points post the first use of supplemental medication for pain or early withdrawal. Total pain is calculated as the area under the curve (based on trapezoidal rule) of pain relief results across all the time points within the reporting period. Percent of maximum total pain relief is calculated as the total pain relief, multiplied by 100 and divided by the product of maximal result during the reporting period and duration of the reporting period. The type 3 p value is from the general linear model with factors of treatment and site.

Formulation A versus placebo in alleviating pain as experienced by the subject population was also assessed by means of the Summed Qualities of Dental Pain Index (QDPI), Table 16. The Summed QDPI at 6 hours showed Formulation A to be more effective than placebo (p<0.001).

TABLE 20
Qualities of Dental Pain (QDPI) Results (Intent to Treat Population)

| | | Formulation | NORCO | Placebo |
|---|---|---|---|---|
| | | A (N = 211) | (N = 205) | (N = 50) |
| Summed qualities of | Mean (SD) | 18.9 (26.4) | 21.4 (27.8) | 5.6 (19.1) |
| dental pain different (on | | | | |
| QDPI) at 6 hours from | Median | 8.5 | 10.5 | 0.0 |
| baseline | Min, Max | (−54, 91) | (−41, 114) | (−33, 81) |
| | Type 3 p value | <0.001 | | |
| | (Formulation A vs | | | |
| | Placebo) | | | |
| Differences in 11 | Mean (SD) | 11.8 (17.8) | 13.5 (19.3) | 3.7 (13.1) |
| sensory qualities of | | | | |
| dental pain (on QDPI) at | Median | 4.0 | 5.5 | 0.0 |
| 6 hours from baseline | Min, Max | (−35, 63) | (−33, 79) | (−19, 56) |
| | Type 3 p value | <0.001 | | |
| | (Formulation A vs | | | |
| | Placebo) | | | |
| Differences in the 2 | Mean (SD) | 3.4 (4.9) | 3.6 (4.9) | 0.9 (3.6) |
| affective qualities of | Median | 1.0 | 1.0 | 0.0 |
| dental pain (on QDPI) at | Min, Max | (−13, 17) | (−6, 19) | (−9, 13) |
| 6 hours from baseline | Type 3 p value | <0.001 | | |
| | (Formulation A vs | | | |
| | Placebo) | | | |
| Differences in the 2 | Mean (SD) | 3.7 (5.3) | 4.4 (5.3) | 0.9 (3.6) |
| evaluative qualities of | Median | 1.0 | 2.5 | 0.0 |
| dental pain (on QDPI) at | Min, Max | (−6, 19) | (−3, 19) | (−8, 13) |

| | | |
|---|---|---|
| 6 hours from baseline | 12 Type 3 p value | <0.001 |
| | (Formulation A vs | |
| | Placebo) | |

QDPI = QDPI = Qualities of Dental Pain Index,
Min = minimum,
Max = maximum,
SD = standard deviation.
QDPI is based on a pain scale of 0 (not at all) to 10 (very much) on each of 15 words (Aching, Throbbing, Hot, Annoying, Heavy, Pulling, Sharp, Radiating, Pressing, Hurting, Swollen, Agonizing, Tight, Stabbing, Stinging). The 11 sensory qualities of dental pain include throbbing, heavy, swollen, tight, sharp, hot, pulling, stabbing, pressing, stinging, radiating. The 2 affective qualities of dental pain include annoying, agonizing. The 2 evaluative qualities of dental pain include aching, hurting. For each quality of pain, the baseline result (if baseline non-missing) or last observation (if baseline missing) was used for replacing the actual or missing result at 6 hours post baseline if a subject used supplemental medication for pain or had early withdrawal prior to 6 hours post baseline. The type 3 p value is from the general linear model with factors of treatment, site and baseline result (QDPI) at summed level.

Differences in the 11 sensory qualities of dental pain (throbbing, heavy, swollen, tight, sharp, hot, pulling, stabbing, pressing, stinging, radiating) at 6 hours also showed Formulation A effectiveness (p<0.001). In addition, the differences between these two groups as measured for the 2 affective qualities of dental pain (annoying and agonizing) and for the 2 evaluative qualities of dental pain (aching and hurting) also demonstrated the effectiveness of Formulation A (both p<0.001).

Table 21A-B records the data for subjects in the subject population taking any supplemental analgesic medication (as permitted by the clinical protocol) beyond their randomized Treatment Assignment at 6 hours, 24 hours, 48 hours or at 5 days following randomization. The difference (which showed that subjects in the Formulation A stratum consistently depended less on supplementary medications than placebo subjects) was statistically significant at the p<0.001 level for all four of these comparisons (with the suggestion that especially over the initial 24-48 hours, fewer subjects on Formulation A took supplementary medication for pain, and did so later, than subjects on NORCO). The time in hours until use of a supplementary medication for pain also favored Formulation A over placebo at the p<0.001 level of significance.

TABLE 21A
Use of Supplement Medication for Pain (Intent to Treat Population):
Percent of subjects taking any supplemental medication for pain

| | | Formulation | NORCO | Placebo |
|---|---|---|---|---|
| | | A (N = 211) | (N = 205) | (N = 50) |
| Over initial | n (%) | 72 (34.1) | 76 (37.1) | 35 |
| 6 hours | | | | (70.0) |
| | Adjusted Odds | 0.2 (0.1, 0.4) | | |
| | Ratio and 95% CI | | | |
| | Type 3 P value | <0.001 | | |
| | (Formulation A vs | | | |
| | Placebo) | | | |
| Over initial | n (%) | 93 (44.1) | 109 (53.2) | 43 |
| 24 hours | | | | (86.0) |
| | Adjusted Odds | 0.1 (0.0, 0.2) | | |
| | Ratio and 95% CI | | | |
| | Type 3 P value | <0.001 | | |
| | (Formulation A vs | | | |
| | Placebo) | | | |
| Over initial | n (%) | 109 (51.7) | 123 (60.0) | 44 |
| 48 hours | | | | (88.0) |
| | Adjusted Odds | 0.1 (0.0, 0.3) | | |
| | Ratio and 95% CI | | | |
| | Type 3 P value | <0.001 | | |
| | (Formulation A vs | | | |
| | Placebo) | | | |
| Over 5 days | n (%) | 133 (63.0) | 136 (66.3) | 45 |
| | | | | (90.0) |

| | |
|---|---|
| Adjusted Odds | 0.2 (0.1, 0.5) |
| Ratio and 95% CI | |
| Type 3 P value | <0.001 |
| (Formulation A vs | |
| Placebo) | |

CI = Confidence interval.
The adjusted odds ratio, 95% CI, and type 3 p value are from the logistic regression model with factors of treatment and site.

TABLE 21B
Use of Supplement Medication for Pain (Intent to Treat Population):
Time (in hours) to supplemental analgesic medication

| | | Formulation A | NORCO | Placebo |
|---|---|---|---|---|
| | | (N = 211) | (N = 205) | (N = 50) |
| Median Onset and 95% CI | | 33.0 (14.7, 58.3) | 15.6 (8.0, 33.4) | 2.2 (20, |
| | | | | 2.3) |
| Adjusted Hazard Ratio and | 0.3 (0.2, 0.4) | | | |
| 95% CI | | | | |
| Type 3 p value | <0.001 | | | |
| (Formulation A vs | | | | |
| Placebo) | | | | |

CI = confidence interval.
The median onset and 95% CI are from the Kaplan-Meier survival analysis. The adjusted hazard ratio, 95% CI and type 3 p value are from the Cox regression model with factors of treatment and site.

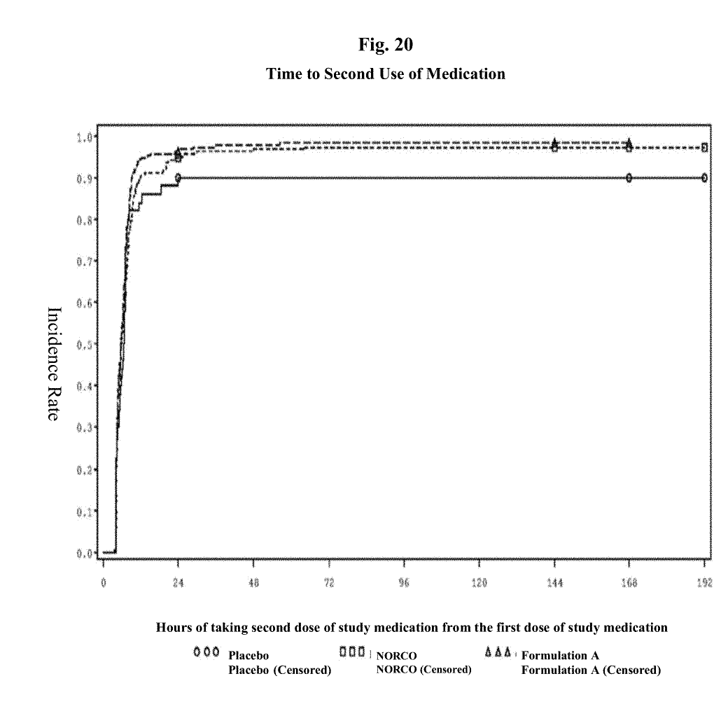

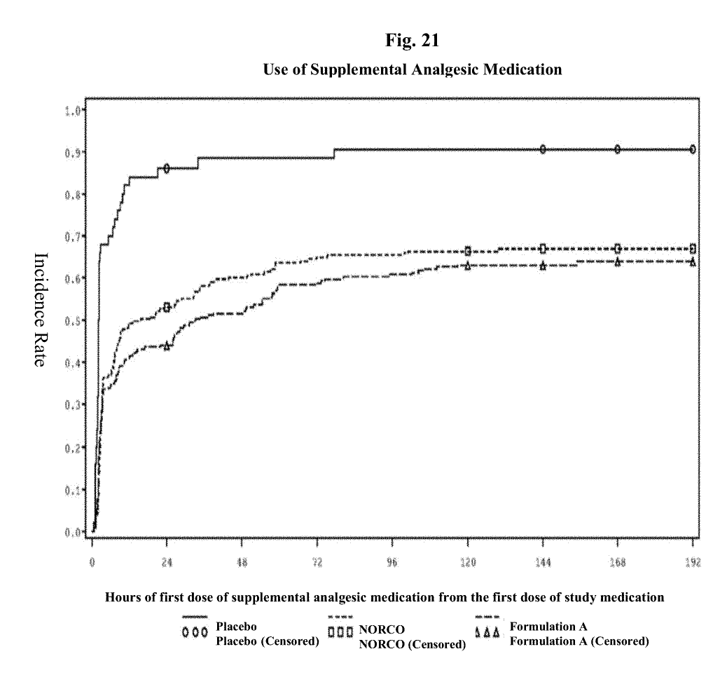

FIG. 19 provides the Kaplan Meier survival curves for the onset of pain reduction among subjects with at least moderate pain relief in the first 6 hours after randomization (i.e., subjects with meaningful relief). Subjects in the placebo group experienced the least amount of pain reduction, with onset by Formulation A-treated subjects beginning at 30 minutes (the first post-treatment time point) and the suggestion that Formulation A has a faster onset than NORCO detectable within the first hour. The time to Second Use of the assigned study medication is provided in FIG. 20 and as can be seen, there were very little differences between these groups in the ITT population. FIG. 21 demonstrates that the placebo group differed notably from both the Formulation A and the NORCO groups in the incidence and time at which supplemental analgesic medication was taken.

Several other descriptive strata within the Intent to Treat (ITT) population have also been presented, but have not had statistical treatment. These include Post-Operative Nausea at baseline, Subjects with a NIS Rating ≥70 mm at baseline, Categorical Pain Intensity Scale at baseline, Number of molar teeth extracted, Tobacco Use, and Subjects having NCS results above the upper tertile in the entire ITT population. Pain reduction measurements for subjects in the ITT population demonstrate differences between the Formulation A and placebo treatment groups. The QPI is sensitive to differences between active and placebo and, notably, that affective (and sensory) qualities of pain are especially fine discriminators for acute pain. In sum, the two Co-Primary Clinical End Points (reduction of Opioid-Induced Nausea or Vomiting and successful induction of Analgesia) were achieved. Statistically significant alleviation of the severity of OINV and of moderate to severe acute pain (as defined by numerous other well defined ITT population strata) was also demonstrated.

Increased Pain Relief and Reducing or Preventing Opioid Induced Nausea or Vomiting in Subjects Treated with Formulation A.

The OINV findings are shown in Table 22, which provides the data for subjects who experienced at most mild nausea, no vomiting, and no use of rescue medication during the initial 24 hours post randomization (“Nausea/Vomiting Responder” in the Table 22). For comparison, 58% of the subjects on NORCO experienced OINV (58%=100%−42%), compared with 36% of the subjects on Formulation A that experienced OINV (36%=100%−64%). The absolute difference in the incidence of OINV between Formulation A and NORCO was (64%−42%=22%), the relative risk reduction (0.22/0.58) was 38%, and the adjusted odds ratio was 2.7 (1.8, 4.1). The type 3 p-value of Formulation A vs NORCO was <0.001.

TABLE 22
Co-Primary Endpoint Results (Intent to Treat Population)

| | | Formulation | NORCO | Placebo |
|---|---|---|---|---|
| | | A (N = 211) | (N = 205) | (N = 50) |
| Nausea/Vomiting | n (%) | 135 (64.0) | 86 (42.0) | 41 (82.0) |

| | | |
|---|---|---|
| Responder | Adjusted Odds | 2.7 (1.8, 4.1) |
| (Nausea Co- | Ratio and 95% | |
| primary | CI | |
| Endpoint) | Type 3 p value | <0.001 |
| | (Formulation | |
| | A vs NORCO) | |
| | NNH (95% | −5 (−3, −8) |
| | CI) | |

CI = confidence interval,
NNH = number needed to harm.
The nausea/vomiting responder is defined as those who experience at most mild nausea, no vomiting, and no use of rescue medication for nausea during the 24 hours post randomization. The adjusted odds ratio, 95% CI, and type 3 p-value are from the logistic regression model with factors of treatment, site and randomized nausea-prone stratum. The NNH and 95% CI for nausea/vomiting endpoint are based on the inverse of the absolute risk difference between Formulation A and NORCO.

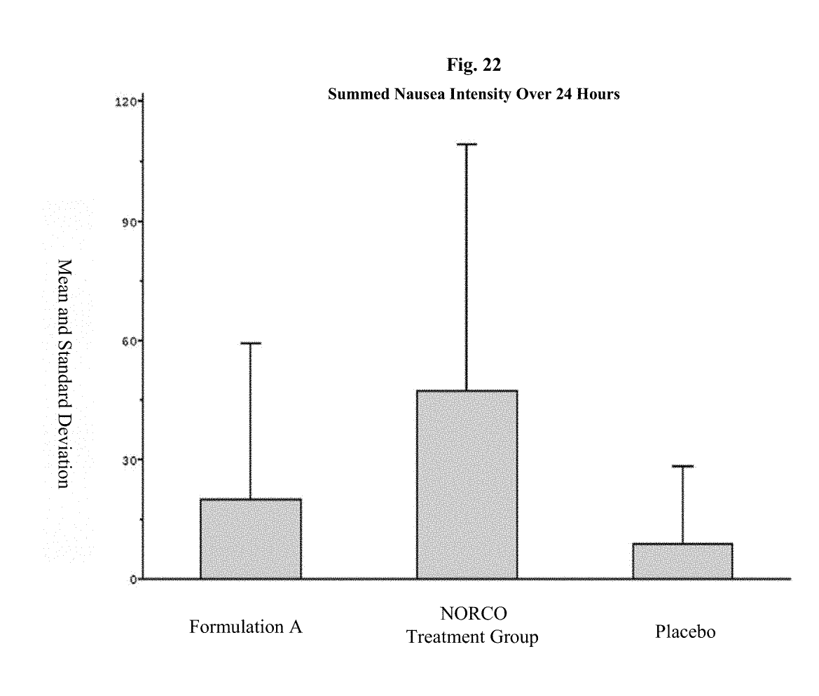

Overall severity of post-treatment nausea experienced over the first 24 hours is shown in FIG. 22. Summed nausea intensity shows the overall severity of post-treatment nausea experienced over the first 24 hours and was highest for subjects randomized to the NORCO group (who received hydrocodone). Summed nausea intensity for subjects in the Formulation A group (who received hydrocodone with promethazine) was substantially (60%) lower than that experienced by subjects in the NORCO group (p<0.001).

The peak intensity of nausea experienced by subjects treated with Formulation A was significantly lower compared to subjects treated with NORCO over the initial 6 hours (p=0.05), over 24 hours (33% less severe nausea, p<0.001), and over the 5 days of treatment (31% less severe nausea, p<0.001) (Table 23A). Comparing Formulation A to NORCO there was 4.9% lower absolute incidence of moderate or severe nausea over the initial 6 hours after the first dose, 14.3% lower absolute incidence over 24 hours (p<0.001), which represents a 62.7% relative risk reduction over the primary 24-hour treatment observation period, Table 23B. (This highly significant divergence between Formulation A and NORCO in terms of the severity of nausea demonstrates the antiemetic effectiveness of Formulation A. This is the period when subjects are most nausea vulnerable and most likely to seek additional analgesia by taking additional doses of opioids.) There was 14.5% lower absolute incidence of moderate/severe nausea over 5 days (p<0.001). The proportion of time that a subject experienced moderate or severe nausea over 24 hours was significantly lower in subjects treated by Formulation A compared to subjects treated with NORCO (p<0.001), Table 23C.

TABLE 23A
Peak Nausea Intensity Scale (NIS) Results (Intent to Treat Population):
Peak Intensity of Nausea (on NIS)

| | | Formulation | NORCO | Placebo |
|---|---|---|---|---|
| | | A (N = 211) | (N = 205) | (N = 50) |
| Over initial 6 | Mean (SD) | 2.5 (2.9) | 3.0 (3.2) | 1.6 (1.9) |
| hours | Median | 1.0 | 2.0 | 1.0 |
| | Min, Max | (0, 10) | (0, 10) | (0, 8) |
| | Type 3 p value | 0.049 | | |
| | (Formulation A vs | | | |
| | NORCO) | | | |
| Over initial | Mean (SD) | 3.0 (3.1) | 4.5 (3.4) | 1.7 (1.8) |
| 24 hours | Median | 2.0 | 5.0 | 1.0 |
| | Min, Max | (0, 10) | (0, 10) | (0, 8) |
| | Type 3 p value | <0.001 | | |
| | (Formulation A vs | | | |
| | NORCO) | | | |
| For days 1-5 | Mean (SD) | 3.4 (3.1) | 4.9 (3.4) | 1.8 (1.9) |
| | Median | 3.0 | 5.0 | 1.0 |
| | Min, Max | (0, 10) | (0, 10) | (0, 8) |

| | |
|---|---|
| Type 3 p value | <0.001 |
| (Formulation A vs | |
| NORCO) | |

NIS = Nausea Intensity Scale,
Min = minimum,
Max = maximum,
SD = standard deviation.
NIS is based on worst nausea a subject has had over the past hour with a scale of 0-to-10, where 0 is no nausea and 10 is severe nausea. The type 3 p value is from the general linear model with factors of treatment, site and randomized nausea-prone stratum.

TABLE 23B
Peak Nausea Intensity Scale (NIS) Results (Intent to Treat Population):
Subjects with any moderate or severe nausea (on NIS)

| | | Formulation | NORCO | Placebo |
|---|---|---|---|---|
| | | A (N = 211) | (N = 205) | (N = 50) |
| Over | n (%) | 61 (28.9) | 74 (36.1) | 8 (16.0) |
| initial 6 | Adjusted Odds Ratio | 0.7 (0.5, 1.1) | | |
| hours | and 95% CI | | | |
| | Type 3 p value | 0.100 | | |
| | (Formulation A vs | | | |
| | NORCO) | | | |
| Over | n (%) | 73 (34.6) | 117 (57.1) | 8 (16.0) |
| initial 24 | Adjusted Odds Ratio | 0.4 (0.2, 0.5) | | |
| hours | and 95% CI | | | |
| | Type 3 p value | <0.001 | | |
| | (Formulation A vs | | | |
| | NORCO) | | | |
| For days | n (%) | 84 (39.8) | 124 (60.5) | 10 (20.0) |

| | | |
|---|---|---|
| 1-5 | Adjusted Odds Ratio | 0.4 (0.3, 0.6) |
| | and 95% CI | |
| | Type 3 p value | <0.001 |
| | (Formulation A vs | |
| | NORCO) | |

Nausea Intensity Scale (NIS) is based on worst nausea a subject has had over the past hour with a scale of 0-to-10, where 0 is no nausea and 10 is severe nausea. Any moderate or severe nausea (on NIS) is defined by NIS result >=4 at any time point during the specific reporting period. The adjusted odds ratio, 95% CI, and type 3 p value from the logistic regression model with factors of treatment, site and randomized nausea prone stratum.

TABLE 23C
Peak Nausea Intensity Scale (NIS) Results (Intent to Treat Population):
Proportion of time that a subject experiences moderate or severe nausea (on NIS)

| | | Formulation A | NORCO | Placebo |
|---|---|---|---|---|
| | | (N = 211) | (N = 205) | (N = 50) |
| Initial 6 hours | Mean (SD) | 0.101 (0.219) | 0.150 (0.268) | 0.057 (0.173) |
| | Median | 0.000 | 0.000 | 0.000 |
| | Min, Max | (0.00, 1.11) | (0.00, 1.04) | (0.00, 1.02) |
| | Type 3 p value | 0.035 | | |
| | (Formulation A | | | |
| | vs NORCO) | | | |
| Initial 24 | Mean (SD) | 0.085 (0.206) | 0.228 (0.334) | 0.046 (0.137) |
| hours | | | | |
| | Median | 0.000 | 0.042 | 0.000 |
| | Min, Max | (0.00, 1.00) | (0.00, 1.01) | (0.00, 0.64) |

| | |
|---|---|
| Type 3 p value | <0.001 |
| (Formulation A | |
| vs NORCO) | |

NIS = Nausea Intensity Scale,
Min = minimum,
Max = maximum,
SD = standard deviation.
NIS is based on worst nausea a subject has had over the past hour with a scale of 0-to-10, where 0 is no nausea and 10 is severe nausea. The last observation result was used to replace any missing results prior to the first use of supplemental medication for nausea or early withdrawal. The worst result recorded up to the first use of supplemental medication for nausea was used to replace at all the time points through the end of study post the first use of supplemental medication for nausea. The results at all the time points post early withdrawal were set to the worst result recorded up to early withdrawal visit. A subject was considered experiencing moderate or severe nausea if the subject had any NIS result >=4 at a specific time point. The type 3 p value is from the general linear model with factors of treatment, site and randomized nausea-prone stratum.

Using the Stomach Scale for measuring the intensity of nausea over the first 6 hours (Table 24) confirmed results on the Nausea Intensity Scale: after the first dose of NORCO subjects reported substantially more severe nausea than subjects who took Formulation A (p=0.026).

TABLE 24
Stomach Scale (StomS) (Intent to Treat Population): Peak intensity of
nausea (on StomS)

| | | Formulation A | NORCO | Placebo |
|---|---|---|---|---|
| | | (n = 211) | (n = 205) | (n = 50) |
| Over | Mean (SD) | 2.0 (2.7) | 2.6 (3.1) | 1.1 (1.6) |
| initial | Median | 1.0 | 2.0 | 0.0 |
| 6 hours | Min, Max | (0, 10) | (0, 10) | (0, 6) |

| | |
|---|---|
| Type 3 p value | 0.026 |
| (Formulation A vs | |
| NORCO) | |

StomS = Stomach Scale,
Min = minimum,
Max = maximum,
SD = standard deviation.
StomS is based on worst stomach feeling a subject has had over the past hours with a scale of 0-to-10, where 0 is normal stomach and 10 is I vomited. The last observation result was used to replace any missing results prior to the first use of supplemental medication for nausea or early withdrawal. The worst result recorded up to the first use of supplemental medication for nausea was used to replace results at all the time points post the first use of supplemental medication for nausea. The results at all the time points post early withdrawal were set to the worst result recorded up to early withdrawal visit. The type 3 p value is from the general linear model with factors of treatment, site and randomized nausea-prone stratum.

As shown in Table 25A, over the initial 24 hours, subjects using NORCO experienced nausea or vomiting more frequently than subjects using Formulation A (p<0.001) and fewer subjects using NORCO had a complete response (no nausea, vomiting or use of antiemetics) than subjects using Formulation A (p=0.002), as shown in Table 25B. Subject satisfaction was measured 6 hours after the initial dose: subjects reported greater satisfaction with Formulation A than NORCO (p<0.001), as shown in Table 25C. By these measures, subjects receiving Formulation A reported significantly more relief of opioid-induced nausea and vomiting and greater satisfaction with their treatment.

TABLE 25A
OINV Endpoint Results (Intent to Treat Population): Proportion of
time that a subject experiences nausea/vomiting

| | | Formulation A | NORCO | Placebo |
|---|---|---|---|---|
| | | (n = 211) | (n = 205) | (n = 50) |
| Initial | Mean (SD) | 0.088 (0.208) | 0.233 (0.335) | 0.055 (0.171) |
| 24 | Median | 0.000 | 0.042 | 0.000 |
| hours | Min, Max | (0.00, 1.00) | (0.00, 1.01) | (0.00, 0.86) |

| | |
|---|---|
| Type 3 p value | <0.001 |
| (Formulation | |
| A vs NORCO) | |

Min = minimum,
Max = maximum,
SD = standard deviation.
A subject was considered experiencing nausea/vomiting if the subject had any Nausea Intensity Scale result >0 or Vomiting Frequency Scale result >0 at a specific time point. The type 3 p value is from the general linear model with factors of treatment, site and randomized nausea-prone stratum.

TABLE 25B
OINV Endpoint Results (Intent to Treat Population): Subjects with
complete response (no nausea, vomiting, or supplemental medication
for nausea/vomiting)

| | | Formulation | NORCO | Placebo |
|---|---|---|---|---|
| | | A (n = 211) | (n = 205) | (n = 50) |
| Over initial 6 | n (%) | 84 (39.8) | 64 (31.2) | 20 (40.0) |
| hours | Adjusted Odds | 1.5 (1.0, 2.3) | | |
| | Ratio and 95% CI | | | |
| | Type 3 p value | 0.063 | | |
| | (Formulation A vs | | | |
| | NORCO) | | | |
| Over initial | n (%) | 68 (32.2) | 40 (19.5) | 17 (34.0) |

| | | |
|---|---|---|
| 24 hours | Adjusted Odds | 2.1 (1.3, 3.4) |
| | Ratio and 95% CI | |
| | Type 3 p value | 0.002 |
| | (Formulation A vs | |
| | NORCO) | |

CI = confidence interval.
The adjusted odds ratio, 95% CI, and type 3 p value are from the logistic regression model with factors of treatment, site and randomized nausea-prone stratum.

Table 25C provides additional data measured by the Subject Satisfaction Scale at 6 hours after the initial dose. The entire subject population is represented by this measure and it shows that subjects in the Formulation A group were more satisfied after 1 dose than subjects randomized to the placebo group with a p-value also of <0.001.

TABLE 25C
OINV Endpoint Results (Intent to Treat Population): Subject
Satisfaction Scale

| | | Formulation A | NORCO | Placebo |
|---|---|---|---|---|
| | | (n = 211) | (n = 205) | (n = 50) |
| Visit 2 6.0 hour | Mean (SD) | 3.8 (2.2) | 3.4 (2.3) | 2.1 (1.8) |
| from baseline | Median | 5.0 | 4.0 | 1.0 |
| | Min, Max | (1, 7) | (1, 7) | (1, 7) |

| | |
|---|---|
| Type 3 p value | <0.001 |
| (Formulation A | |
| vs Placebo) | |

Subject Satisfaction Scale is based on a scale of 1-to-7:
1 = Extremely Dissatisfied,
2 = Very Dissatisfied,
3 = Dissatisfied,
4 = Somewhat Satisfied,
5 = Satisfied,
6 = Very Satisfied,
7 = Extremely Satisfied.
The subject satisfaction scale at 6 hours post baseline was set to 1 (Extremely Dissatisfied) if a subject used supplemental medication for pain or nausea or had early withdrawal prior to 6 hours post baseline.
The type 3 p value is from the general linear model with factors of treatment and site.

The incidences of vomiting over various time periods following randomization are reported in Tables 26A-B. After the first dose of study medication, fewer subjects in the Formulation A group (6.2%) had any vomiting over the initial 6 hours than in the NORCO group (10.2%). Over 24 hours (i.e., after repeated doses of hydrocodone for pain), 21.0% of subjects who used NORCO vomited, compared to 10.0% of subjects who used Formulation A (p=0.002), representing a 52% relative reduction in the risk of vomiting over 24 hours.

TABLE 26A
Vomiting Frequency (VFS) Results (Intent to Treat Population):
Subjects with any vomiting (on VFS)

| | | Formulation A | NORCO | Placebo |
|---|---|---|---|---|
| | | (N = 211) | (N = 205) | (N = 50) |
| Over | n (%) | 13 (6.2) | 21 (10.2) | 1 (2.0) |
| initial | Adjusted Odds | 0.6 (0.3, 1.2) | | |
| 6 hours | Ratio and 95% CI | | | |
| | Type 3 p value | 0.137 | | |
| | (Formulation A vs | | | |
| | NORCO) | | | |
| Over | n (%) | 21 (10.0) | 43 (21.0) | 2 (4.0) |
| initial | Adjusted Odds | 0.4 (0.2, 0.7) | | |
| 24 hours | Ratio and 95% CI | | | |
| | Type 3 p value | 0.002 | | |
| | (Formulation A vs | | | |
| | NORCO) | | | |
| Over days | n (%) | 28 (13.3) | 54 (26.3) | 3 (6.0) |

| | | |
|---|---|---|
| 1-5 | Adjusted Odds | 0.4 (0.2, 0.7) |
| | Ratio and 95% CI | |
| | Type 3 p value | <0.001 |
| | (Formulation A vs | |
| | NORCO) | |

VFS = Vomiting Frequency Scale,
CI = Confidence Interval.
VFS is based on question, ‘How often did you vomit over the past hour?’.
0 = Not at All,
1 = One time,
2 = Two times,
3 = Three or More Times.
The type 3 p value is from the logistic regression model with factors of treatment, site and randomized nausea-prone stratum.

TABLE 26B
Vomiting Frequency (VFS) Results (Intent to Treat Population):
Frequency of vomiting (on VFS)

| | | Formulation | | |
|---|---|---|---|---|
| | | A | NORCO | Placebo |
| | | (N = 211) | (N = 205) | (N = 50) |
| Over initial 6 | Mean (SD) | 0.1 (0.6) | 0.3 (1.1) | 0.1 (0.6) |
| hours | Median | 0.0 | 0.0 | 0.0 |
| | Min, Max | (0, 5) | (0, 9) | (0, 4) |
| | Type 3 p value | <0.001 | | |
| | (Formulation A | | | |
| | vs NORCO) | | | |
| For days 1-5 | Mean (SD) | 1.4 (7.6) | 4.6 (15.0) | 0.5 (3.3) |
| | Median | 0.0 | 0.0 | 0.0 |
| | Min, Max | (0, 58) | (0, 118) | (0, 23) |

| | |
|---|---|
| Type 3 p value | <0.001 |
| (Formulation A | |
| vs NORCO) | |

VFS = Vomiting Frequency Scale,
Min = minimum,
Max = maximum,
SD = standard deviation.
VFS is based on question, ‘How often did you vomit over the past hour?’:
0 = Not at All,
1 = One time,
2 = Two times,
3 = Three or More Times.
The last observance result was used to replace any missing results prior to the first use of supplemental medication for nausea or early withdrawal.
The worst result recorded up to the first use of supplemental medication for nausea was used to replace results at all the time points through the end of study post the first use of supplemental medication for nausea.
The results at all the time points post early withdrawal were set to the worst result recorded up to early withdrawal visit.
The type 3 p value is from the Poisson model with factors of treatment, site and randomized nausea-prone stratum.

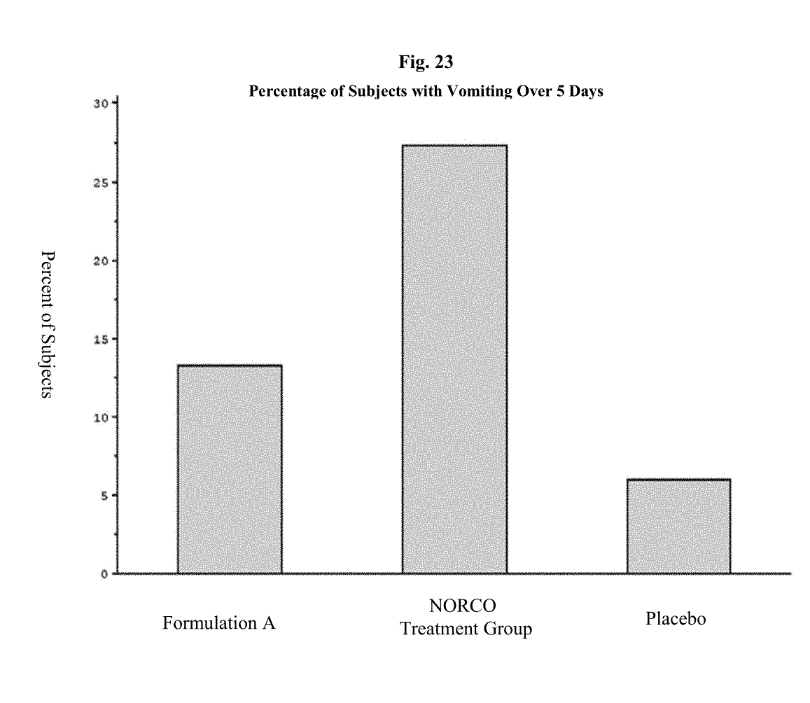

Subjects did not stop vomiting with continued opioid exposure (as seen in the frequency of vomiting after the first dose and over all 5 post-op days in FIG. 23): some additional subjects vomited later in the course of treatment as their opioid exposure increased and some subjects vomited yet again. With ongoing opioid use for pain over the 5 post-op days, 26.3% of subjects who used NORCO vomited compared with 13.3% of subjects who used Formulation A (p<0.001), representing a 49% relative reduction in the risk of vomiting.

Other opioid side effects were also measured prospectively as endpoints in the OSS. There was no significant difference between Formulation A and NORCO for any of these side effects after the first dose, the initial 24 hours, or over the entire 5-day treatment period, Table 27. There was no evidence of greater drowsiness, dizziness, itch, constipation, etc. in Formulation A-treated subjects compared with NORCO-treated subjects.

TABLE 27
Number and percent of subjects with moderate or severe opioid-related
side effects other than nausea or vomiting

| | | Formulation A | NORCO | Placebo |
|---|---|---|---|---|
| | | (N = 211) | (N = 205) | (N = 50) |
| At 6 hours after | n/D (%) | 55/209 (26.3) | 52/205 (25.4) | 7/48 (14.6) |
| baseline | Adjusted Odds Ratio and | 1.0 (0.7, 1.6) | | |
| | 95% CI | | | |
| | Type 3 p value | 0.848 | | |
| | (Formulation A vs | | | |
| | NORCO) | | | |
| On Day 1 | n/D (%) | 80/209 (38.3) | 83/205 (40.5) | 13/48 (27.1) |
| | Adjusted Odds Ratio and | 0.9 (0.6, 1.3) | | |
| | 95% CI | | | |
| | Type 3 p value | 0.625 | | |
| | (Formulation A vs | | | |
| | NORCO) | | | |
| For Days 1-5 | n/D (%) | 130/209 (62.2) | 121/205 (59.0) | 19/48 (39.6) |

| | |
|---|---|
| Adjusted Odds Ratio and | 1.1 (0.8, 1.7) |
| 95% CI | |
| Type 3 p value | 0.521 |
| (Formulation A vs | |
| NORCO) | |

CI = confidence interval.
Opioid Symptoms Scales (OSS) are based on a set of 11 symptoms with a scale of 0-to-10, where 0 is none and 10 is severe.
n and % are number and percentage of subjects who had OSS score ≥4 within the reporting period and had none (score = 0) at baseline for any opioid symptoms.
Percentages are based on number of subjects (D) who had non-missing results both within the reporting period and at baseline for any opioid symptoms.
The adjusted odds ratio, 95% CI, and type 3 p value are from the logistic regression model with factors of treatment, site and randomized nause-prone stratum

Evaluation of Formulation A in preventing OINV compared to NORCO in the subject population is provided in Table 28. This objective endpoint shows statistically significant differences between Formulation A and NORCO for the primary 24-hour observation period (the day of most pain, when subjects used the most analgesics), showing an 11.4% incidence of OINV among Formulation A-treated subjects compared to a 31.7% incidence among subjects treated with NORCO (p<0.001). This represents a relative risk reduction of 64%. Significantly less OINV by this endpoint definition for Days 2, 3 and 4 confirmed the treatment effects of Formulation A compared to NORCO. The evaluation of OINV over all five days of opioid treatment as measured on this endpoint demonstrated significantly less OINV experienced by the 7.1% subjects who used Formulation A compared to the 17.2% of subjects who used NORCO (p=0.002), or a 58% relative risk reduction.

TABLE 28
Incidence of Vomiting or Use of Antiemetic Medication by Day (Intent
to Treat Population)

| | Formulation A (n = | NORCO | |
|---|---|---|---|
| | 211) | (n = 205) | |
| | n/D (%) | n/D (%) | p-value |
| Day One | 24/211 (11.4) | 65/205 (31.7) | <0.001 |
| Day Two | 8/210 (3.8) | 21/204 (10.3) | 0.010 |
| Day Three | 7/209 (3.3) | 16/204 (7.8) | 0.046 |
| Day Four | 3/209 (1.4) | 14/204 (6.9) | 0.006 |
| Day Five+ | 3/208 (1.4) | 3/204 (1.5) | 0.981 |
| Post Day 1 | 15/210 (7.1) | 35/204 (17.2) | 0.002 |

Day 1, 2, 3, 4 reporting windows are based on consecutive 24-hr interval from the first dose of study medication.
Day 5+ reporting window is defined to be 96 hours or more from the first dose of study medication.
Post Day 1 reporting window is defined to be 24 hours or more from the first dose of study medication.
n and % are number and percentage of subjects who had at least one occurrence of vomiting or use of antiemetic medication within each reporting period.
Percentages are based on number of subjects (D) who took at least one study medication within each reporting period.
Nominal p-value is based on Chi-squared test.

Increased Pain Relief without the Need for a Rescue Drug in Subjects Treated with Formulation A.

The use of antiemetics as rescue medications for opioid-induced nausea/vomiting is another strong measure of Formulation A to impact OINV. Table 29 compares this rescue utilization in the subjects receiving Formulation A and NORCO over all 5 study days and over the initial 24 hours. The difference in the percentages of subjects using antiemetic medication was significantly lower for Formulation A than for NORCO over both time intervals (p<0.001).

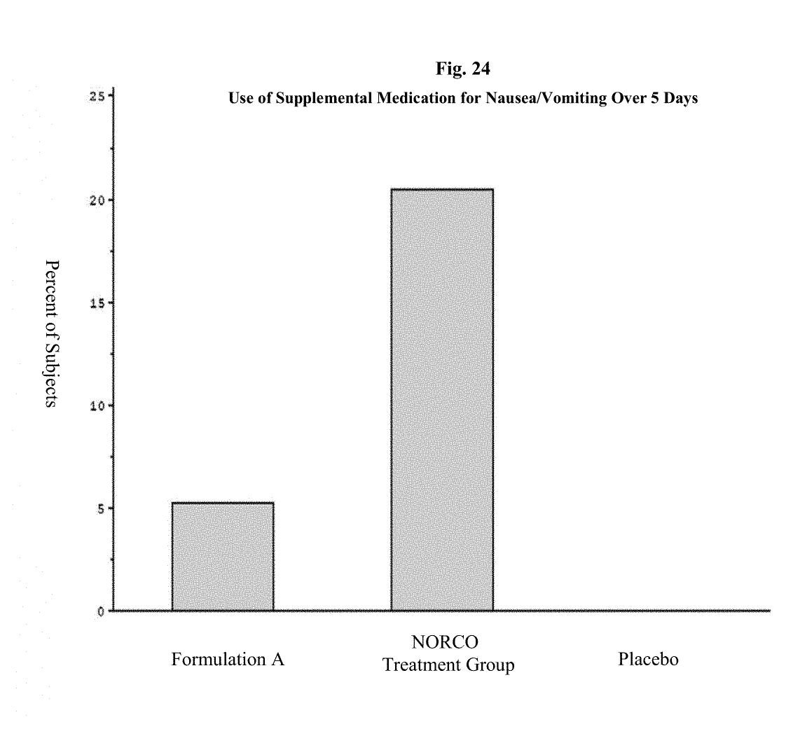

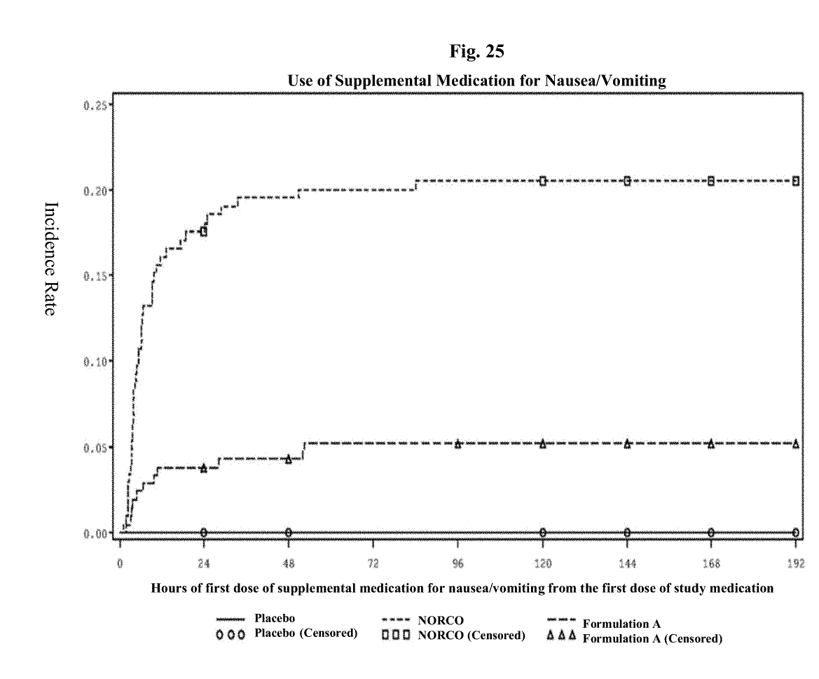

Over the entire study period 5.2% of subjects on Formulation A used antiemetics, compared to 20.5% of subjects on NORCO, representing a substantial reduction in the use of antiemetics: 75% fewer subjects in the Formulation A treatment group used antiemetic medication compared with subjects in the NORCO group (FIG. 24). The time before resorting to antiemetic medication also favored treatment with Formulation A (p<0.001). FIG. 25, a Kaplan Meier survival curve, shows that subjects in the NORCO began using rescue antiemetic medication earlier than subjects in the Formulation A group.

TABLE 29
Use of Supplement Medication for Nausea/Vomiting (Intent to Treat
Population)

| | | Formu- | | |
|---|---|---|---|---|
| | | lation A | NORCO | Placebo |
| | | (N = 211) | (N = 205) | (N = 50) |
| Subjects taking | n (%) | 11 (5.2) | 42 (20.5) | 0 |
| supplemental | Adjusted Odds | 0.2 (0.1, 0.4) | | |
| medication for | Ratio and 95% | | | |
| nausea/vomiting | CI | | | |
| over 5 days | Type 3 p value | <0.001 | | |
| | (Formulation A | | | |
| | vs NORCO) | | | |
| Number of | Mean (SD) | 0.0 (0.3) | 0.2 (0.5) | 0.0 (0.0) |
| antiemetic doses | Median | 0.0 | 0.0 | 0.0 |
| in first 24 hours | Min, Max | (0, 2) | (0, 3) | (0, 0) |

| | |
|---|---|
| Type 3 p value | <0.001 |
| (Formulation A | |
| vs NORCO) | |

CI = confidence interval.
The adjusted odds ratio, 95% CI, and type 3 p value are from the logistic regression model with factors of treatment, site, and randomized nausea-prone stratum.
Min = minimum,
Max = maximum,
SD = standard deviation.
The type 3 p value is from the Poisson model with factors of treatment, site, and randomized nausea-prone stratum.

Table 30 provides comparisons of the summed intensity of nausea (on the NIS) over the initial 24 hours showing Formulation A and NORCO were significantly different at the <0.001 level. The summed intensity comparing the same two groups over the initial 6 hours was significant at the 0.013 level. The summed intensity difference of nausea for the same two groups on the Stomach Scale was significant at the 0.020 level, and a comparison of the frequency of vomiting over 24 hours (on VFS) for the same two groups was significant at the <0.001 level. From these four secondary endpoints, the severity of nausea and the frequency of vomiting were significantly reduced by Formulation A compared to NORCO.

TABLE 30
Secondary Endpoint Results (Per-Protocol Population): Summed
intensity of nausea

| | | Formulation | NORCO | Placebo |
|---|---|---|---|---|
| | | A (n = 200) | (n = 191) | (n = 46) |
| (on NIS) over | Mean (SD) | 20.3 (39.0) | 48.4 (62.3) | 7.9 (19.7) |
| initial 24 hours | Median | 4.9 | 19.8 | 1.4 |
| | Min, Max | (0, 217) | (0, 240) | (0, 102) |
| | Type 3 p value | <0.001 | | |
| | (Formulation A vs | | | |
| | NORCO) | | | |
| (on NIS) over | Mean (SD) | 5.7 (8.9) | 8.4 (12.0) | 3.8 (6.6) |
| initial 6 hours | Median | 1.9 | 3.7 | 1.3 |
| | Min, Max | (0, 41) | (0, 60) | (0, 35) |
| | Type 3 p value | 0.013 | | |
| | (Formulation A vs | | | |
| | NORCO) | | | |
| (on StomS) over | Mean (SD) | 5.8 (8.9) | 8.2 (11.7) | 3.8 (6.3) |
| initial 6 hours | Median | 1.7 | 3.5 | 1.1 |
| | Min, Max | (0, 42) | (0, 62) | (0, 26) |
| | Type 3 p value | 0.020 | | |
| | (Formulation A vs | | | |
| | NORCO) | | | |
| (on VFS) over | Mean (SD) | 0.8 (3.4) | 2.4 (8.0) | 0.0 (0.1) |
| initial 24 hours | Median | 0.0 | 0.0 | 0.0 |
| | Min, Max | (0, 23) | (0, 61) | (0, 1) |

| | |
|---|---|
| Type 3 p value | <0.001 |
| (Formulation A | |
| vs NORCO) | |

NIS = Nausea Intensity Scale,
StomS = Stomach Scale,
VFS = Vomiting Frequency Scale,
Min = minimum,
Max = maximum,
SD = standard deviation.
NIS is based on worst nausea a subject has had over the past hour with a scale of 0-to-10, where 0 is ‘normal stomach’ and 10 is ‘I vomited’.
VFS is based on question ‘How often did you vomit over the past hour?’:
0 = Not at All,
1 = One time,
2 = Two times,
3 = Three or more times.
The last observation result was used to replace any missing results prior to the first use of supplemental medication for nausea or early withdrawal.
The worst result recorded up to the first use of supplemental medication for nausea was used to replace results at all the time points post the first use of supplemental medication for nausea.
The results at all the time points post early withdrawal were set to the worst result recorded up to early withdrawal visit.
The type 3 p value for the NIS and StomS endpoints is from the general linear model with factors of treatment, site and nausea prone status.
The type 3 p value for the frequency of vomiting endpoint is from the Poisson regression model with factors of treatment, site and randomized nausea-prone stratum.

In this study the incidence of OINV among subjects who took Formulation A was 0.36; among subjects who took NORCO the incidence of OINV was 0.58. Thus the relative reduction of OINV by Formulation A compared to NORCO was 0.58−0.36/0.58, or 38%. The relative reduction of OINV by Formulation A (0.11) compared to NORCO (0.32) was determined to be 0.32−0.11/0.32, or 64%.

In this study, nausea, vomiting, dizziness, drowsiness, constipation, difficulty voiding, confusion, headache, itch, difficulty concentrating, and dry mouth were not recorded as adverse events. There were no subject deaths, and no subjects reporting any serious treatment emergent adverse events. Other treatment-emergent adverse events (TEAEs) assessed by the investigators averaged 6.6%, 5.4% and 4.0% in the Formulation A, NORCO and placebo treatment groups respectively. Subjects reporting at least one AE ranged between 27% and 28% and subjects reporting at least one TEAE ranged from 26.5% to 28%.

The data show that Formulation A use for periods of up to 5 days is equally as safe as that of NORCO, an already approved hydrocodone analgesic, with no clinically important hemodynamic or respiratory effects. Formulation A has the additional value of providing a safe level of antiemetic medication (promethazine) without triggering any meaningful additional adverse events. Using the protocol-defined criteria for OINV, the risk of OINV was reduced 38% by Formulation A relative to NORCO. This risk was reduced by 64%, both outcomes exceeding the maximum relative risk reduction observed by a parenteral antiemetic in the post-operative setting.

### Example 10: Design of Clinical Phase III Study for Formulation A (Bunionectomy)

A Clinical Phase III study was performed for Formulation A in the form of a bi-layer tablet in the treatment of moderate to severe pain. This was a double-blind, randomized, multiple-dose, placebo- and positive-controlled study of Formulation A in subjects with moderate to severe pain following bunionectomy (with osteotomy and internal fixation) without collateral procedures. A positive control (NORCO, a commercial formulation of hydrocodone 7.5 mg/acetaminophen 325 mg) was included to determine the antiemetic effects of Formulation A. Analgesia was determined by comparing Formulation A to placebo.

Human adults 18 years of age and older with a first metatarsal bunion had a screening visit (Visit 1) where a foot x-ray was obtained and reviewed by the surgeon (between Visit 1 and Visit 2 prior to surgery). The medical history and medication history was obtained, including a “Nausea-Prone Questionnaire,” or NPQ, to increase the sensitivity of the assay by enriching the study sample with subjects at risk of developing opioid induced nausea and vomiting (OINV). Potential subjects who were considered at risk of OINV according to their responses on the NPQ were scheduled for surgery within 3 months.

After sedation (midazolam and/or propofol) was achieved, regional anesthesia was established with a popliteal sciatic nerve block (PSB) using ropivacaine initially, then mepivacaine, after which subjects underwent primary, unilateral, first metatarsal bunionectomy surgery (osteotomy and internal fixation) without collateral procedures. Surgery was performed under regional anesthesia and propofol sedation. If the PSB was not sufficient to provide adequate anesthesia, a standard Mayo block could be established. Prophylactic or post-operative antiemetic or opioid medications other than protocol-allocated medications were permitted. After surgery the regional anesthesia was continued via a continuous anesthetic infusion until approximately 3 am of the morning after surgery. Supplementary analgesic medication (IV or IM ketorolac) could be administered as needed for breakthrough pain. Subject were observed in the research center for up to 9 hours after the PSB was discontinued to determine if they developed at least moderate pain as measured on a categorical pain intensity scale (PI-CAT).

Four study questionnaires concerning common opioid-related side effects were administered to each eligible subject before receiving the study medication: a nausea intensity scale (NIS), a nausea scale (N-CAT) to assess severity of nausea on a standard categorical scale, a retching/vomiting Index (RVI) to assess retching and/or vomiting (i.e., “emetic episodes”) on an ordinal scale, and an opioid symptoms scales (OSS) to assess other opioid-related side effects (e.g., drowsiness, dizziness, ability to concentrate, itch, constipation, etc.). A Symptom Checklist was administered to identify other symptoms present before drug exposure. To characterize the sensory, evaluative, and affective dimensions of pain, a Qualities of Pain Index (QPI) was then administered. Baseline pain intensity was measured on a 0-10 numerical pain intensity scale (PINRS). Subjects must have had moderate (4-6) or severe pain (7-10) on the PI-NRS in order to be eligible for randomization.

Qualifying subjects (n=550) undergoing a primary, unilateral metatarsal bunionectomy (osteotomy and internal fixation without collateral procedures) were randomized to one of the three treatment groups: Formulation A (n=250), NORCO (n=250), or placebo (n=50). Study medication was administered to subjects under double-blind conditions. Randomized subjects remained at the research site for the initial 54 hours. Pulse, respiratory rate, systolic and diastolic blood pressure and pulse oximetry were obtained immediately prior to Baseline assessments, then hourly (while awake) until 48 hours post-dosing. Temperature was measured at Baseline and every 6 hours post-dosing (while awake) until 48 hours post-dosing. Pain intensity was assessed on the PI-NRS at 10-minute time points over 2 hours after the first dose, then every 30 minutes over the next 4 hours. A Likert pain relief scale (REL) was also completed every 30 minutes over this initial 6-hour observation period. After the PI-NRS and REL assessments at each 30-minute time point during the initial 6-hour period, the Nausea Intensity Scale (NIS) was also completed in order to assess the occurrence and severity of nausea.

Beginning at 1 hour after the first dose, the Retching/Vomiting Index (RVI) was completed hourly to assess the occurrence and frequency of retching and/or vomiting (i.e., emetic episodes). At the end of the initial 6-hour treatment evaluation period (or before subjects took a second dose of study medication), the subjects completed the Qualities of Pain Index (QPI) as well as the Opioid Symptoms Scales (OSS) to evaluate other beneficial or adverse effects after the first dose of study medication. After 6 hours, the PI-NRS, REL, NIS and RVI were completed every hour (while awake) until 12 hours, when the QPI was also be completed (i.e., after the first 2-3 doses of study medication). After 12 hours, the subject continued completing only the PI-NRS, NIS and RVI every hour (while awake) until 48 hours. The same assigned study medication was administered every 4-6 hours (for a maximum of 6 doses in a 24-hour period). To provide evaluations of other opioid side effects associated with multiple doses of study medication, subjects repeated the OSS at 24 hours and 48 hours in the clinic. To provide evaluations of beneficial effects of multiple doses of study medication, subjects repeated the QPI at 24 hours and 48 hours while in the clinic. At 48 hours the subjects assessed his/her overall satisfaction with the study medication on a Patient Satisfaction Scale (PSS). Upon reviewing the subject's course over 48 hours, the Investigator provided a Physician's Global Evaluation (PGE) of the study medication as a treatment for pain. The next dose of study medication after 48 hours was then administered while the subject remained in the research center. Directly before this dose (and hourly for 6 hours) the subject provided evaluations on the PI-NRS, NIS, N-CAT and RVI. At the end of this 6-hour evaluation, adverse events that may have occurred in-clinic were reviewed and subjects were discharged home.

For subjects receiving administrations outside of a hospital on Days 3, 4 and 5, the subjects self-dosed with the same assigned study medication as needed for pain every 4-6 hours (for a maximum of 6 doses in a 24-hour period), documenting the date and time of each dosing in a Subject Diary. At approximately 9 pm each evening of Days 3, 4, and 5, subjects used the diary to make assessments of: the worst severity of nausea they experienced in the past 24 hours on the WNS (Worst Nausea Scale); retching and/or vomiting over the past 24 hours (on the 24-hour RVI); other opioid symptoms over the past 24 hours (on the OSS); other adverse events they experienced over the past 24 hours. Subjects also recorded the date and time of taking supplemental analgesic medication (ibuprofen 400 mg) and/or antiemetic. Cigarette smokers also recorded in the Subject Diary the number of cigarettes smoked on Days 3, 4 and 5. Subjects who used supplemental medication for pain and/or for nausea/vomiting continued regularly scheduled evaluations of pain and nausea/vomiting and other side effects throughout the entire 5-day treatment observation period. For all 5 study days subjects continued using the Subject Diary to document each self-dosing and each use of a supplementary analgesic or antiemetic medication.

### Example 11: Analysis of Clinical Phase III Study for Formulation A (Bunionectomy)

Results from the study described in Example 10 were analyzed.

### Endpoints.

The two co-primary endpoints were (1) the occurrence of opioid-induced nausea and vomiting (OINV), a composite (2-component) endpoint for OINV (any vomiting or use of antiemetic medication), comparing Formulation A to NORCO over 48 hours; and (2) the relief of moderate to severe acute pain, comparing Formulation to placebo over 48 hours (summed pain intensity differences over 48 hours, or SPID48).

Secondary endpoints included: the occurrence of opioid-induced nausea and vomiting (OINV) as a 3-component endpoint (a composite (3-component) endpoint for OINV including the severity of nausea: any moderate or severe nausea, any vomiting or use of antiemetics, (comparing Formulation A to NORCO over 48 hours); the severity of opioid-induced nausea (comparing Formulation A to NORCO over 48 hours); the use of antiemetics (comparing Formulation A to NORCO over 5 days); the occurrence of vomiting (comparing Formulation A to NORCO over 5 days), the relief of severe pain (comparing Formulation A to NORCO over 24 hours (SPID24)); the percent change in QPI (comparing Formulation A to NORCO over 24 hours); Global Evaluation (comparing Formulation A to NORCO over 48 hours); and the incidence of post-discharge nausea and vomiting (PDNV) (comparing Formulation A to NORCO over days 3-5).

Reduction of OINV in Subjects Treated with Formulation A.

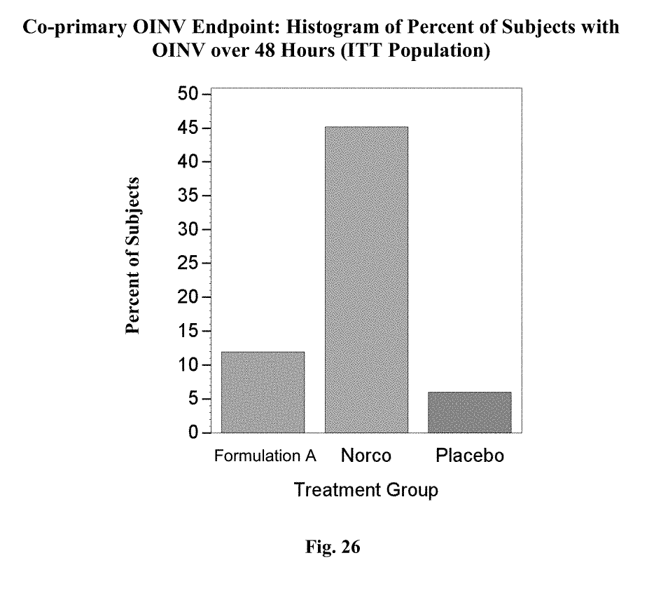

Table 31 summarizes the occurrence of opioid-induced nausea and vomiting (OINV) co-primary endpoint result. Subjects administered Formulation A experienced a 74% reduction in OINV (as measured by the composite (2-component) endpoint for OINV (p<0.001) (FIG. 26). “Reduction” is based on the relative risk reduction (RRR). For example, RRR of 0.737 means there is 74% less chance (74% reduced risk) of developing OINV when treated with Formulation A than (“relative to”) subjects treated with NORCO (ie, RRR=0.452−0.119=0.333; 0.333/0.452=0.737=73.7%).

TABLE 31
Co-primary OINV Endpoint Results (Intend to Treat Population).

| | | Formulation A | NORCO | Placebo |
|---|---|---|---|---|
| | | (n = 252) | (n = 250) | (n = 50) |
| OINV Endpoint: | n (%) | 30 (11.9%) | 113 (45.2%) | 3 (6.0%) |

| | | |
|---|---|---|
| Occurrence of | Adjusted Odds Ratio and | 0.16 (0.10, 0.26) |
| Nausea/Vomiting | 95% CI | |
| over 48 Hours | Type III P Value | <.001 |
| | (Formulation A vs NORCO) | |
| | Relative Risk Reduction and | 73.7 (57.4, 89.9) |
| | 95% CI (%) | |
| | NNT and 95% CI | 4 (3, 4) |

The OINV endpoint is defined a composite (2-component) endpoint indicating any vomiting or use of antiemetic medication (indicative of nausea) over 48 hrs.
CI = confidence interval.
The Adjusted Odds Ratio, 95% CI, and Type III P Value are from the logistic regression model with factors of treatment and investigator as main effects.
NNT = number needed to treat.
The NNT and 95% CI for nausea/vomiting endpoint are based on the inverse of the absolute risk difference between Formulation A and NORCO.

Reduction in the Use of Antiemetics and Number of Vomiting Episodes in Subjects Treated with Formulation A.

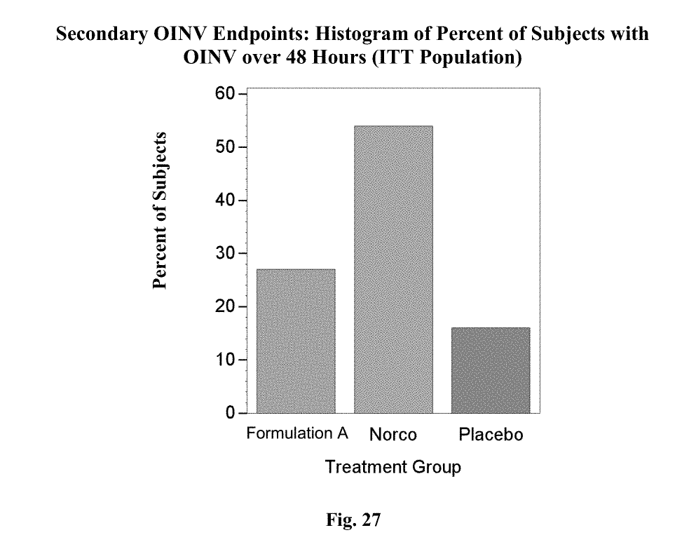

Table 33 provides the results for a subset of the secondary endpoints (occurrence of nausea/vomiting and use of antiemetic results). Subjects on Formulation A experienced a 71% reduction in the use of antiemetics (13.5%) and a 66% reduction in the number of vomiting episodes (8.3%) compared to subjects treated with NORCO (46.0% and 24.4% respectively, p<0.001) over the 5-day observation period. A 50% reduction in the incidence of moderate/severe nausea, vomiting and use of antiemetics among subjects treated with Formulation A compared to subjects treated with NORCO over 48 hours (p<0.001) was also observed (FIG. 27).

TABLE 33
Occurrence of Nausea/Vomiting and use of Antiemetic Results (Intend
to Treat population).

| | | Formulation A | NORCO | Placebo |
|---|---|---|---|---|
| | | (N = 252) | (N = 250) | (N = 50) |
| Occurrence of | n (%) | 68 (27.0%) | 135 (54.0%) | 8 (16.0%) |
| Nausea/Vomiting | Adjusted Odds Ratio and | 0.31 (0.21, 0.45) | | |
| over 48 Hours | 95% CI | | | |
| | Type III P Value | <.001* | | |
| | (Formulation A vs | | | |
| | NORCO) | | | |
| | Relative Risk Reduction | 50.0 (34.7, 65.3) | | |
| | and 95% CI (%) | | | |
| Use of any | n (%) | 34 (13.5%) | 115 (46.0%) | 5 (10.0%) |
| AntiEmetics over | Adjusted Odds Ratio and | 0.18 (0.12, 0.28) | | |
| 5 Days | 95% CI | | | |
| | Type III P Value | <.001* | | |
| | (Formulation A vs | | | |
| | NORCO) | | | |
| | Relative Risk Reduction | 70.7 (54.4, 86.9) | | |
| | and 95% CI (%) | | | |
| Occurrence of | n (%) | 21 (8.3%) | 61 (24.4%) | 1 (2.0%) |

| | | |
|---|---|---|
| any Vomiting | Adjusted Odds Ratio and | 0.28 (0.16, 0.48) |
| over 5 Days | 95% CI | |
| | Type III P Value | <.001* |
| | (Formulation A vs | |
| | NORCO) | |
| | Relative Risk Reduction | 65.8 (39.9, 91.8) |
| | and 95% CI (%) | |

CI = confidence interval.
The Type III P Value is from likelihood ratio test in logistic regression model with factors of treatment and investigator as main effects (*following a Type III P Value indicates the statistical significance using Hochberg step-up test procedure for controlling family-wise type 1 error at the 0.05 level).

Reduction in Severity of Nausea in Subjects Treated with Formulation A.

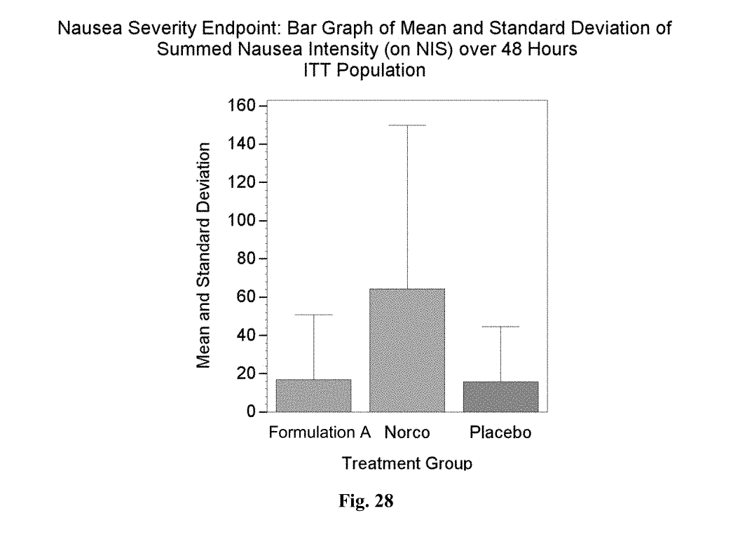

Table 34 provides the results for another secondary endpoint. Over the 5-day observation period there was 70% less severe nausea experienced by subjects treated with Formulation A (40.8) compared to subjects treated with NORCO (136.1, p<0.001) (FIG. 28).

TABLE 34
Severity of Opioid-Induced Nausea Results (Intend to Treat population).

| | | Formulation A | NORCO | Placebo |
|---|---|---|---|---|
| | | (N = 252) | (N = 250) | (N = 50) |
| Severity of | n | 252 | 250 | 50 |
| Opioid-Induced | Mean (SD) | 40.8 (70.24) | 136.1 (179.31) | 28.5 (50.86) |
| Nausea (NIS | median | 5.8 | 59.5 | 1.0 |
| and WNS) over | Min, Max | 0, 420 | 0, 1073 | 0, 249 |

| | | |
|---|---|---|
| 5 Days | LS-Means and 95% CI | −95.5 (−119.2, −71.7) |
| | (Difference between | |
| | Treatment Groups) | |
| | Type III P Value | <.001* |
| | (Formulation A vs | |
| | NORCO) | |

SD = standard deviation.
Min = minimum.
Max = maximum.
The Type III P Value is from the general linear model with factors of treatment and investigator as main effects (*following a Type III P Value indicates the statistical significance using Hochberg step-up test procedure for controlling family-wise type 1 error at the 0.05 level)

Reduction in the Use of Antiemetics in Subjects Treated with Formulation A.

Tables 35A and 35B provides summaries of the supplemental medications used for nausea and vomiting (NVSM). Over the 5-day observation period six times as many subjects on NORCO (30.8%) used antiemetics repeatedly compared to subjects on Formulation A (4.8%, p<0.001), which was indistinguishable from placebo (6.0%). Additionally, four times as many subjects on NORCO (21.2%) used parenteral antiemetics over 48 hours compared to subjects on Formulation A (5.2%, p<0.001).

TABLE 35A
Summary of Nausea and Vomiting Supplemental Medications (NVSM)
(Intend to Treat Population).

| | | Formulation A | NORCO | Placebo |
|---|---|---|---|---|
| | | (N = 252) | (N = 250) | (N = 50) |
| Percentage of | n (%) | 27 (10.7%) | 106 (42.4%) | 3 (6.0%) |
| Subjects Using | | | | |
| Antiemetics over 48 | Adjusted Odds Ratio | 0.16 (0.10, 0.25) (vs NORCO), 1.89 | | |
| Hours | and 95% CI | (0.55, 6.54) (vs Placebo) | | |
| | Type III P Value | <.001 (vs NORCO), 0.313 (vs Placebo) | | |
| Percentage of | n (%) | 21 (8.3%) | 72 (28.8%) | 2 (4.0%) |
| Subjects Using | | | | |
| Antiemetics over 24 | Adjusted Odds Ratio | 0.22 (0.13, 0.37) (vs NORCO), 2.18 | | |
| Hours | and 95% CI | (0.49, 9.71) (vs Placebo) | | |
| | Type III P Value | <.001 (vs NORCO), 0.304 (vs Placebo) | | |
| Percentage of | n (%) | 34 (13.5%) | 115 (46.0%) | 5 (10.0%) |
| Subjects Using | | | | |
| Antiemetics over 5 | Adjusted Odds Ratio | 0.18 (0.12, 0.28) (vs NORCO), 1.42 | | |
| Days | and 95% CI | (0.52, 3.84) (vs Placebo) | | |
| | Type III P Value | <.001 (vs NORCO), 0.495 (vs Placebo) | | |
| Percentage of | n (%) | 12 (4.8%) | 77 (30.8%) | 3 (6.0%) |
| Subjects with | Adjusted Odds Ratio | 0.11 (0.06, 0.21) (vs NORCO), 0.78 | | |
| Repeat Use of | and 95% CI | (0.21, 2.88) (vs Placebo) | | |
| Antiemetics over 5 | Type III P Value | <.001 (vs NORCO), 0.708 (vs Placebo) | | |
| Days | | | | |
| Percentage of | n (%) | 13 (5.2%) | 53 (21.2%) | 0 |
| Subjects Using | Adjusted Odds Ratio | 0.20 (0.11, 0.38) (vs NORCO), >999.99 | | |
| Parenteral (IV or | and 95% CI | (<0.01, >999.99) (vs Placebo) | | |
| IM) Antiemetics | Type III P Value | <.001 (vs NORCO), 0.963 (vs Placebo) | | |
| over 48 Hours | | | | |
| Percentage of | n (%) | 9 (3.6%) | 42 (16.8%) | 0 |

| | | |
|---|---|---|
| Subjects Using | Adjusted Odds Ratio | 0.18 (0.09, 0.38) (vs NORCO), >999.99 |
| Parenteral (IV or | and 95% CI | (<0.01, >999.99) (vs Placebo) |
| IM) Antiemetics | Type III P Value | <.001 (vs NORCO), 0.968 (vs Placebo) |
| over 24 Hours | | |

IV = intravenous.
IM = intramuscular.
CI = confidence interval.
P Value tested using type III likelihood ratio test in logistic regression model with factors of treatment and investigator as main effects.

The number of doses of antiemetic medications, used by subjects taking Formula A compared to subjects taking NORCO is provided in Table 35B. Over the 5-day observation period five times as many doses of antiemetics were used by subjects taking NORCO compared to subjects taking Formulation A (p<0.001). Over the 48-hour in-hospital period, five times as many doses of parenteral antiemetics were administered to subjects taking NORCO compared to subjects taking Formulation A (p<0.001).

TABLE 35B
Summary of Nausea and Vomiting Supplemental Medications (NVSM)
(Intend to Treat Population).

| | | Formulation A | NORCO | Placebo |
|---|---|---|---|---|
| | | (N = 252) | (N = 250) | (N = 50) |
| Number of Doses of | n | 252 | 250 | 50 |
| Antiemetics Used | Mean (SD) | 0.2 (0.67) | 1.0 (1.66) | 0.1 (0.34) |
| over 48 Hours | Median | 0.0 | 0.0 | 0.0 |
| | Min, Max | 0, 5 | 0, 8 | 0, 2 |
| | Type III P Value | <.001 (vs NORCO), 0.094 (vs Placebo) | | |
| Number of Doses of | n | 252 | 250 | 50 |
| Antiemetics Used | Mean (SD) | 0.1 (0.40) | 0.5 (0.95) | 0.1 (0.31) |
| over 24 Hours | Median | 0.0 | 0.0 | 0.0 |
| | Min, Max | 0, 3 | 0, 4 | 0, 2 |
| | Type III P Value | <.001 (vs NORCO), 0.344 (vs Placebo) | | |
| Number of Doses of | n | 252 | 250 | 50 |
| Antiemetics Used | Mean (SD) | 0.3 (0.86) | 1.5 (2.51) | 0.2 (0.60) |
| over 5 Days | Median | 0.0 | 0.0 | 0.0 |
| | Min, Max | 0, 7 | 0, 14 | 0, 3 |
| | Type III P Value | <.001 (vs NORCO), 0.291 (vs Placebo) | | |
| Number of Doses of | n | 252 | 250 | 50 |
| Parenteral (IV or | Mean (SD) | 0.1 (0.43) | 0.5 (1.22) | 0.0 (0.00) |
| IM) Antiemetics | median | 0.0 | 0.0 | 0.0 |
| Used over 48 Hours | Min, Max | 0, 5 | 0, 7 | 0, 0 |
| | Type III P Value | <.001 (vs NORCO), <.001 (vs Placebo) | | |
| Number of Doses of | n | 252 | 250 | 50 |
| Parenteral (IV or | Mean (SD) | 0.0 (0.26) | 0.3 (0.75) | 0.0 (0.00) |
| IM) Antiemetics | median | 0.0 | 0.0 | 0.0 |
| Used over 24 Hours | Min, Max | 0, 3 | 0, 4 | 0, 0 |

| | |
|---|---|
| Type III P Value | <.001 (vs NORCO), <.001 (vs Placebo) |

IV = intravenous.
IM = intramuscular.
SD = standard deviation.
Min = minimum.
Max = maximum.
P Value tested using the Poisson regression model with factors of treatment and investigator as main effects.

Reduction in Vomiting and Retching in Subjects Treated with Formulation A.

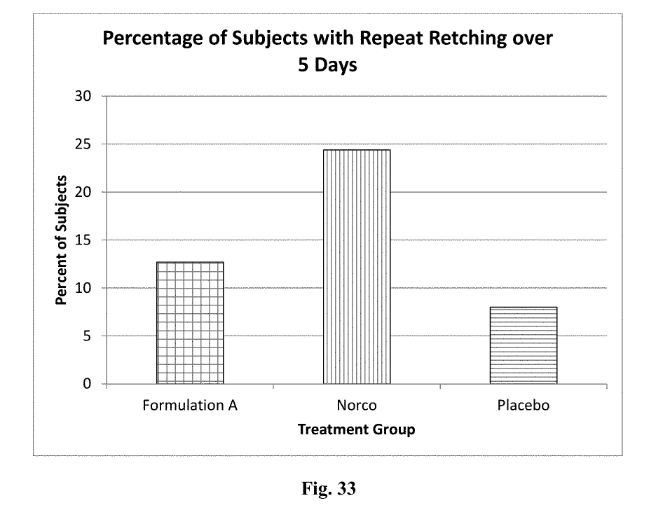

The incidences of vomiting and retching over various time periods are reported in Table 36(A-B). Three times as many subjects on NORCO (21.2%) vomited repeatedly over 5 days compared to subjects on Formulation A (7.1%, p<0.001). Over the 5-day observation period, more subjects treated with NORCO were retching (29.6%) than subjects who used Formulation A (17.9%, p<0.01) and nearly twice as many subjects who used NORCO (24.4%) were retching repeatedly over 5 days than subjects who used Formulation A (12.7%, p<0.001) (FIG. 33). Over the 5-day observation period almost twice as many subjects using NORCO (36.8%) reported “emetic episodes” (i.e., retching or vomiting) as subjects who used Formulation A (19.8%, p<0.001).

TABLE 36A
Summary of Retching/Vomiting Index (RVI) (Intend To Treat
Population).

| | | Formulation A | NORCO | Placebo |
|---|---|---|---|---|
| | | (N = 252) | (N = 250) | (N = 50) |
| Percentage of Subjects | n (%) | 15 (6.0%) | 56 (22.4%) | 0 |
| with Occurrence of | Adjusted Odds Ratio | 0.21 (0.12, 0.39) (vs NORCO), >999.99 | | |
| Vomiting over 48 Hours | and 95% CI | (<0.01, >999.99) (vs Placebo) | | |
| | Type III P Value | <.001 (vs NORCO), 0.954 (vs Placebo) | | |
| Percentage of Subjects | n (%) | 11 (4.4%) | 37 (14.8%) | 0 |
| with Occurrence of | Adjusted Odds Ratio | 0.26 (0.13, 0.52) (vs NORCO), >999.99 | | |
| Vomiting over 24 Hours | and 95% CI | (<0.01, >999.99) (vs Placebo) | | |
| | Type III P Value | <.001 (vs NORCO), 0.958 (vs Placebo) | | |
| Percentage of Subjects | n (%) | 14 (5.6%) | 51 (20.4%) | 0 |
| with Repeat Vomiting | Adjusted Odds Ratio | 0.22 (0.12, 0.42) (vs NORCO), >999.99 | | |
| over 48 Hours | and 95% CI | (<0.01, >999.99) (vs Placebo) | | |
| | Type III P Value | <.001 (vs NORCO), 0.954 (vs Placebo) | | |
| Percentage of Subjects | n (%) | 18 (7.1%) | 53 (21.2%) | 1 (2.0%) |
| with Repeat Vomiting | Adjusted Odds Ratio | 0.28 (0.16, 0.50) (vs NORCO), 3.90 | | |
| over 5 Days | and 95% CI | (0.50, 30.29) (vs Placebo) | | |
| | Type III P Value | <.001 (vs NORCO), 0.194 (vs Placebo) | | |
| Percentage of Subjects | n (%) | 45 (17.9%) | 74 (29.6%) | 6 (12.0%) |
| with Any Retching over | Adjusted Odds Ratio | 0.52 (0.34, 0.79) (vs NORCO), 1.61 | | |
| 5 Days | and 95% CI | (0.65, 4.03) (vs Placebo) | | |
| | Type III P Value | .002 (vs NORCO), 0.306 (vs Placebo) | | |
| Percentage of Subjects | n (%) | 32 (12.7%) | 61 (24.4%) | 4 (8.0%) |
| with Repeat Retching | Adjusted Odds Ratio | 0.45 (0.28, 0.72) (vs NORCO), 1.69 | | |
| over 5 Days | and 95% CI | (0.57, 5.06) (vs Placebo) | | |
| | Type III P Value | <.001 (vs NORCO), 0.347 (vs Placebo) | | |
| Percentage of Subjects | n (%) | 40 (15.9%) | 84 (33.6%) | 5 (10.0%) |
| with Retching or | Adjusted Odds Ratio | 0.37 (0.24, 0.57) (vs NORCO), 1.70 | | |
| Vomiting over 48 Hours | and 95% CI | (0.63, 4.59) (vs Placebo) | | |
| | Type III P Value | <.001 (vs NORCO), 0.292 (vs Placebo) | | |
| Percentage of Subjects | n (%) | 50 (19.8%) | 92 (36.8%) | 6 (12.0%) |

| with Retching or | Adjusted Odds Ratio | 0.42 (0.28, 0.63) (vs NORCO), 1.83 |
|---|---|---|
| Vomiting over 5 Days | and 95% CI | (0.74, 4.56) (vs Placebo) |
| | Type III P Value | <.001 (vs NORCO), 0.194 (vs Placebo) |

CI = confidence interval.
P Value tested using type III likelihood ratio test in logistic regression model with factors of treatment and investigator as main effects.

TABLE 36B
Retching or Vomiting Episodes Numbers (Intend To Treat Population).

| | | Formulation A | NORCO | Placebo |
|---|---|---|---|---|
| | | (N = 252) | (N = 250) | (N = 50) |
| over | n | 252 | 250 | 50 |
| 5 Days | Mean (SD) | 1.7 (5.75) | 6.4 (14.22) | 0.8 (3.12) |
| | Median | 0.0 | 0.0 | 0.0 |
| | Min, Max | 0, 43 | 0, 100 | 0, 19 |
| | Type III P Value | <.001 (vs NORCO), <.001 (vs Placebo) | | |
| over | n | 252 | 250 | 50 |
| 48 Hours | Mean (SD) | 1.5 (5.50) | 5.8 (12.50) | 0.7 (2.94) |
| | Median | 0.0 | 0.0 | 0.0 |
| | Min, Max | 0, 43 | 0, 82 | 0, 19 |

| | |
|---|---|
| Type III P Value | <.001 (vs NORCO), <.001 (vs Placebo) |

SD = standard deviation.
Min = minimum.
Max = maximum.
P values tested using Poisson regression model with factors of treatment and investigator as main effects.

Reduction in Peak Intensity of Nausea in Subjects Treated with Formulation A.

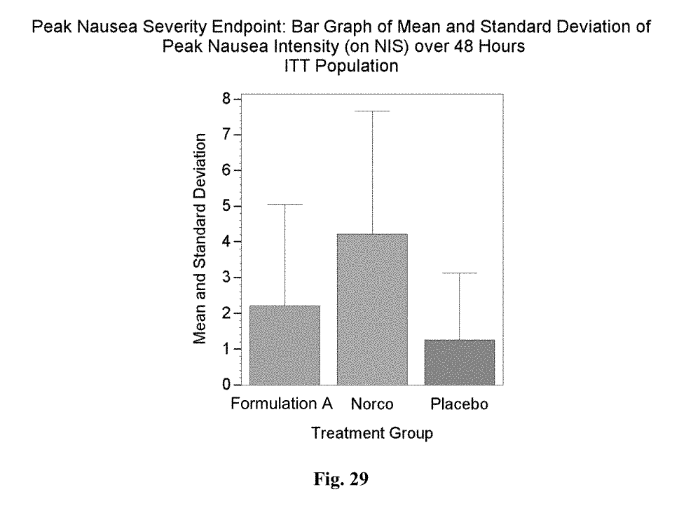

The peak intensity of nausea experienced by subjects treated with Formulation A was significantly lower compared to subjects treated with NORCO over 48 hours and over 5 days of treatment (Table 37). Over the 48 hour treatment period there was a 48% difference in the worst (peak) severity of nausea experienced by subjects treated with Formulation A compared to subjects treated with NORCO (p<0.001) and over the 5-day treatment period there was a 42% difference in the worst (peak) severity of nausea experienced by subjects treated with Formulation A compared to subjects treated with NORCO (p<0.001) (FIG. 29).

TABLE 37
Nausea Intensity Scale (NIS) results (Intend To Treat Population).

| | | Formulation A | NORCO | Placebo |
|---|---|---|---|---|
| | | (N = 252) | (N = 250) | (N = 50) |
| Peak | n | 252 | 250 | 50 |
| Nausea | mean (SD) | 2.2 (2.85) | 4.2 (3.45) | 1.3 (1.86) |
| over | Median | 1.0 | 3.5 | 0.5 |
| 48 Hours | Min, Max | 0, 10 | 0, 10 | 0, 9 |
| | Type III P Value | <.001 (vs NORCO), 0.046 (vs Placebo) | | |
| Peak | n | 252 | 250 | 50 |
| Nausea | mean (SD) | 2.6 (2.93) | 4.5 (3.42) | 1.5 (2.01) |
| over | Median | 2.0 | 4.0 | 1.0 |
| 5 Days | Min, Max | 0, 10 | 0, 10 | 0, 9 |

| | |
|---|---|
| Type III P Value | <.001 (vs NORCO), 0.020 (vs Placebo) |

SD = standard deviation.
Min = minimum.
Max = maximum.
P values tested using type III test in general linear model with factors treatment and investigator as the main effects.

Table 38(A-B) summarizes the data for subjects with complete response. Complete response is either defined as no nausea, vomiting or use of antiemetics (Table 38A) or as no vomiting or use of antiemetics (Table 38B). Over the 5-day observation period more than twice as many subjects who used Formulation A (35.3%) had no nausea, no vomiting and no use of antiemetics (“complete response”) than subjects who used NORCO (16.0%, p<0.001) (Table 38A). Over the 5-day observation period more subjects who used Formulation A (84.1%) did not vomit or use an antiemetic than subjects who used NORCO (50.8%, p<0.001) (Table 38B), an incidence that was indistinguishable from subjects treated with placebo (90.0%, p=0.283).

TABLE 38A
Summary of Subjects with Complete Response (Intend To Treat
Population).

| | | Formulation | | |
|---|---|---|---|---|
| | | A | NORCO | Placebo |
| | | (N = 252) | (N = 250) | (N = 50) |
| over | n (%) | 89 (35.3%) | 40 (16.0%) | 22 (44.0%) |
| 5 Days | Adjusted Odds | 2.88 (1.88, 4.42) (vs NORCO), 0.69 | | |
| | Ratio and 95% CI | (0.37, 1.28) (vs Placebo) | | |
| | Type III P Value | <.001 (vs NORCO), 0.244 (vs Placebo) | | |
| over | n (%) | 108 (42.9%) | 48 (19.2%) | 24 (48.0%) |

| | | |
|---|---|---|
| 48 | Adjusted Odds | 3.21 (2.14, 4.81) (vs NORCO), 0.81 |
| Hours | Ratio and 95% CI | (0.44, 1.50) (vs Placebo) |
| | Type III P Value | <.001 (vs NORCO), 0.498 (vs Placebo) |

CI = confidence interval.
P values tested using type III likelihood ratio test in logistic regression model with factors of treatment and investigator as main effects. Complete Response is defined as: no Nausea, vomiting or use of Antiemetics.

TABLE 38B
Summary of Subjects with Complete Response (Intend To Treat
Population).

| | | Formulation A | NORCO | Placebo |
|---|---|---|---|---|
| | | (N = 252) | (N = 250) | (N = 50) |
| over | n (%) | 212 (84.1%) | 127 (50.8%) | 45 (90.0%) |
| 5 Days | Adjusted Odds | 5.13 (3.38, 7.81) (vs NORCO), 0.58 (0.22, | | |
| | Ratio and 95% CI | 1.57) (vs Placebo) | | |
| | Type III P Value | <.001 (vs NORCO), 0.283 (vs Placebo) | | |
| over | n (%) | 222 (88.1%) | 137 (54.8%) | 47 (94.0%) |

| | | |
|---|---|---|
| 48 | Adjusted Odds | 6.19 (3.92, 9.79) (vs NORCO), 0.47 (0.14, |
| Hours | Ratio and 95% CI | 1.61) (vs Placebo) |
| | Type III P Value | <.001 (vs NORCO), 0.229 (vs Placebo) |

CI = confidence Interval.
P values tested using type III likelihood ratio test in logistic regression model with factors of treatment and investigator as main effects. Complete response defined as: no vomiting or use of antiemetics.

Decreased PONV (Post-Operative Nausea and Vomiting) in Subjects Treated with Formulation A.

Almost four times as many subjects with post-operative nausea and vomiting, or PONV (i.e., nausea and vomiting present before taking study medication) reported improvement in PONV after administration of Formulation A compared to subjects who administered NORCO-most (80.6%) of whom reported worsening of PONV after taking NORCO (p<0.001) after 5 days treatment the period (Table 39).

TABLE 39
14.2.3.14 Post-Operative Nausea and Vomiting (PONV) results (Intend
To Treat Population)

| Relief | | Formulation A | NORCO | Placebo |
|---|---|---|---|---|
| of PONV | | (N = 69) | (N = 67) | (N = 11) |
| over | Improved | 34 (49.3%) | 19 (28.4%) | 7 (63.6%) |
| 6 Hours | Unchanged | 18 (26.1%) | 20 (29.9%) | 3 (27.3%) |
| | Worsened | 17 (24.6%) | 28 (41.8%) | 1 (9.1%) |
| | Type III P Value | 0.009 (vs NORCO), 0.258 (vs Placebo) | | |
| over | Improved | 32 (46.4%) | 15 (22.4%) | 6 (54.5%) |
| 12 Hours | Unchanged | 16 (23.2%) | 18 (26.9%) | 3 (27.3%) |
| | Worsened | 21 (30.4%) | 34 (50.7%) | 2 (18.2%) |
| | Type III P Value | 0.003 (vs NORCO), 0.464 (vs Placebo) | | |
| over | Improved | 27 (39.1%) | 11 (16.4%) | 6 (54.5%) |
| 24 Hours | Unchanged | 17 (24.6%) | 16 (23.9%) | 3 (27.3%) |
| | Worsened | 25 (36.2%) | 40 (59.7%) | 2 (18.2%) |
| | Type III P Value | 0.002 (vs NORCO), 0.235 (vs Placebo) | | |
| over | Improved | 23 (33.3%) | 6 (9.0%) | 6 (54.5%) |
| 48 Hours | Unchanged | 14 (20.3%) | 9 (13.4%) | 2 (18.2%) |
| | Worsened | 32 (46.4%) | 52 (77.6%) | 3 (27.3%) |
| | Type III P Value | <.001 (vs NORCO), 0.166 (vs Placebo) | | |
| over | Improved | 23 (33.3%) | 6 (9.0%) | 6 (54.5%) |
| 3 Days | Unchanged | 13 (18.8%) | 8 (11.9%) | 2 (18.2%) |
| | Worsened | 33 (47.8%) | 53 (79.1%) | 3 (27.3%) |
| | Type III P Value | <.001 (vs NORCO), 0.154 (vs Placebo) | | |
| over | Improved | 23 (33.3%) | 6 (9.0%) | 6 (54.5%) |
| 4 Days | Unchanged | 13 (18.8%) | 7 (10.4%) | 2 (18.2%) |
| | Worsened | 33 (47.8%) | 54 (80.6%) | 3 (27.3%) |
| | Type III P Value | <.001 (vs NORCO), 0.154 (vs Placebo) | | |
| over | Improved | 23 (33.3%) | 6 (9.0%) | 6 (54.5%) |
| 5 Days | Unchanged | 13 (18.8%) | 7 (10.4%) | 2 (18.2%) |
| | Worsened | 33 (47.8%) | 54 (80.6%) | 3 (27.3%) |

| | |
|---|---|
| Type III P Value | <.001 (vs NORCO), 0.154 (vs Placebo) |

PONV = post-operative nausea and vomiting. Select subjects who had nausea >0 on the NIS or had vomiting (Score of (7), (6), (5), (4), or (3) on RVI) at baseline, prior to administration of study drug. The NIS and RVI scores post treatment are compared to baseline for improvement, worsening or no change in PONV: if both scores decrease (or one decreases and the other is 0 at baseline) without use of antiemetic, the subject is categorized as ‘improved’ at that visit; if they both remain the same without the use of antiemetic, they are categorized as ‘unchanged’. If either score increases or the subject uses an antiemetic, the subject is categorized as having ‘worsened’. Display the % of subjects who are categorized ‘improved’, ‘unchanged’ and ‘worsened’.
P values tested using a Mantel-Haenszel Chi-Square test.

Decreased PDNV (Post-Discharge Nausea and Vomiting) in Subjects Treated with Formulation A.

Over the three days when subjects were at home, three times as many subjects on NORCO experienced post-discharge nausea and vomiting (PDNV) as subjects on Formulation A, which was indistinguishable from placebo, representing a 63% reduction in the risk of developing PDNV (p<0.001) (as shown in Table 40).

TABLE 40
Incidents of PDNV Results (Intend To Treat Population)

| | | Formulation | | |
|---|---|---|---|---|
| | | A | NORCO | Placebo |
| | | (N = 252) | (N = 250) | (N = 50) |
| Number of | n | 252 | 250 | 50 |
| Incidents of | Mean (SD) | 1.1 (2.05) | 3.0 (2.83) | 0.8 (1.82) |
| Post- | Median | 0.0 | 2.0 | 0.0 |
| Discharge | Min, Max | 0, 6 | 0, 6 | 0, 6 |

| | | |
|---|---|---|
| Nausea and | LS-Means and | 0.37 (0.27, 0.49) |
| Vomiting | 95% CI (Ratio | |
| (PDNV) over | between | |
| Days 3-5 | Treatment | |
| | Groups) | |
| | Type III P Value | <.001 |
| | (Formulation A | |
| | vs NORCO), | |
| | using Negative | |
| | Binomial | |
| | Distribution | |

PDNV = post-discharge nausea and vomiting.
CI = confidence interval.
SD = standard deviation.
Min = minimum.
Max = maximum.
The Type III P Value for the frequency of vomiting endpoint is from the Poisson regression model with factors of treatment and investigator as main effects.

Reduction in Moderate to Severe Pain in Subjects Treated with Formulation A

Table 41 provides the relief of moderate to severe acute pain co-primary endpoint result. Subjects on Formulation A experienced a significant difference in the reduction of moderate to severe pain over 48 hours (SPID48) compared to subjects treated with placebo (p<0.001).

TABLE 41
Co-Primary Analgesia Endpoint Results (Intend to Treat Population)

| | | Formulation A | NORCO | Placebo |
|---|---|---|---|---|
| | | (n = 252) | (n = 250) | (n = 50) |
| Analgesia | n | 252 | 250 | 50 |
| Endpoint: (Sum | Mean (SD) | 118.4 (80.02) | 107.0 (83.60) | 53.1 (69.69) |
| of Pain Intensity | Median | 109.5 | 88.0 | 32.3 |
| Differences over | Min, Max | −23, 399 | −12, 380 | −76, 313 |
| 48 Hours) | | | | |

| | |
|---|---|
| LS-Means and 95% CI | 69.4 (46.5, 92.3) |
| (Difference between | |
| Treatment Groups) | |
| Type III P Value | <.001 |
| (Formulation A vs Placebo) | |

SD = standard deviation.
Min = minimum.
Max = maximum.
CI = confidence interval.
The Adjusted Odds Ratio, 95% CI, and Type III P Value are from the logistic regression model with factors of treatment and investigator as main effects. The analgesia endpoint is defined as the time-weighted sum of pain intensity differences from baseline over 48 hours post randomization. The Type III P Value for the analgesia endpoint is from the general linear model with factors of treatment, investigator and baseline pain intensity as main effects.

Reduction of Pain in Subjects Treated with Formulation A.

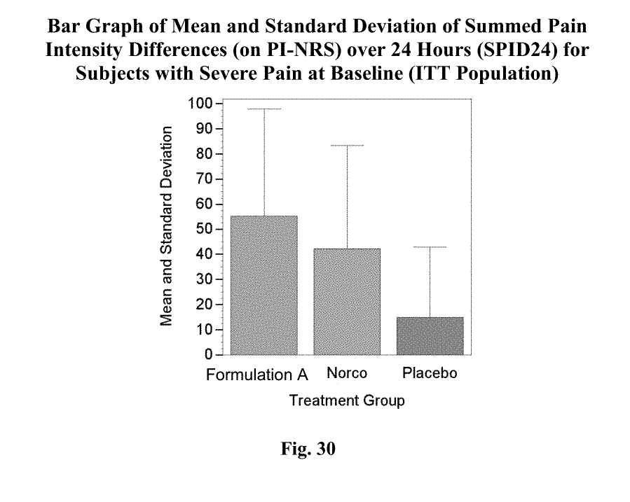

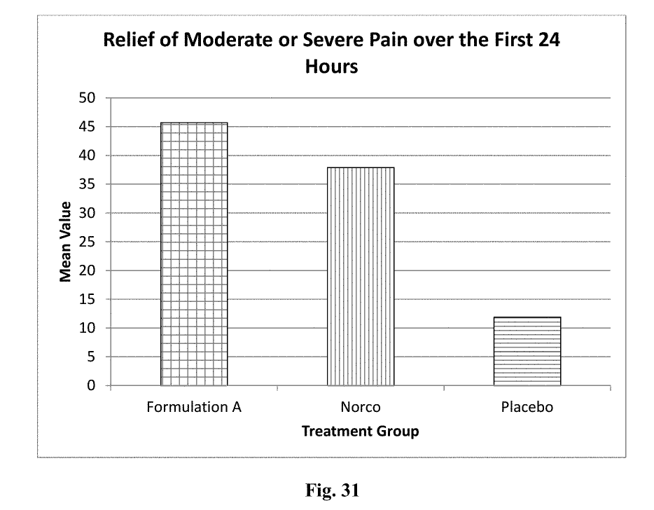

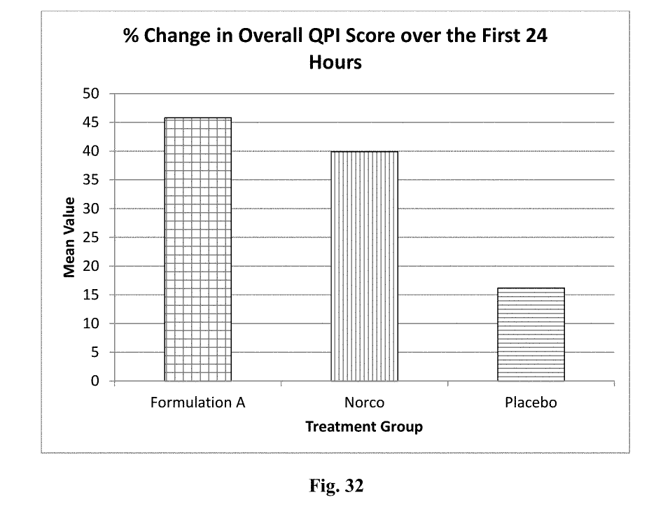

Table 42 provides the results for another subset of the secondary endpoints. There was 31% greater reduction of pain over 24 hours (SPID24) for subjects with severe pain who were treated with Formulation A than subjects treated with NORCO (p=0.01) (FIG. 30). There was also a 15% greater reduction of qualitative features of pain over 24 hours for subjects treated with Formulation A than subjects treated with NORCO (p=0.12) (FIG. 32).

TABLE 42
Secondary Results (Intend to Treat population).

| | | Formulation A | NORCO | Placebo |
|---|---|---|---|---|
| | | (N = 252) | (N = 250) | (N = 50) |
| Relief of Severe | n | 127 | 126 | 25 |
| Pain over 24 | Mean (SD) | 55.2 (42.71) | 42.2 (41.18) | 14.9 (28.01) |
| Hours (SPID24) | Median | 48.3 | 32.4 | 2.3 |
| for Subjects with | Min, Max | −2, 192 | −5, 193 | −3, 121 |
| Severe Pain (PI- | LS-Means and 95% CI | 13.4 (3.0, 23.8) | | |
| NRS >=7) at | (Difference between | | | |
| Baseline | Treatment Groups) | | | |
| | Type III P Value | 0.012* | | |
| | (Formulation A vs | | | |
| | NORCO) | | | |
| % Change in | n | 252 | 250 | 49 |
| Overall QPI | Mean (SD) | −45.8 (43.25) | −39.9 (40.01) | −16.2 (40.95) |
| Score over 24 | Median | −56.0 | −39.2 | 0.0 |
| Hours | Min, Max | −100, 108 | −100, 58 | −100, 83 |

| | |
|---|---|
| LS-Means and 95% CI | −5.8 (−13.2, 1.5) |
| (Difference between | |
| Treatment Groups) | |
| Type III P Value | 0.117 |
| (Formulation A vs | |
| NORCO) | |

PI-NRS = Numerical Pain Intensity Rating Scale.
QPI = Quality of Pain Index.
SPIDx = Summed Pain Intensity Differences over x hours.
SD = standard deviation.
Min = minimum.
Max = maximum.
The Type III P Value is from the general linear model with factors of treatment and investigator as main effects (* following a Type III P Value indicates the statistical significance using Hochberg step-up test procedure for controlling family-wise type 1 error at the 0.05 level).

Increased Pain Relief in Subjects Treated with Formulation A.

There was a 21% (p=0.01) and a 11% (p=0.11) greater reduction of pain over 24 hours (SPID24) and over 48 hours (SPID48), respectively, for subjects with moderate or severe pain who were treated with Formulation A than subjects treated with NORCO (p=0.01) as shown in Table 43. (See FIG. 31 for the SPID24 data)

TABLE 43
Summary of Pain Intensity Numerical Rating Scale (PI-NRS) (Intend To
Treat Population).

| Relief of | | | | |
|---|---|---|---|---|
| Moderate | | | | |
| or Severe | | Formulation A | NORCO | Placebo |
| Pain | Endpoint | (N = 252) | (N = 250) | (N = 50) |
| over | n | 252 | 250 | 50 |
| 24 Hours | Mean (SD) | 45.7 (38.07) | 37.9 (35.45) | 11.9 (22.49) |
| (SPID24) | Median | 39.3 | 32.0 | 3.2 |
| | Min, Max | −18, 192 | −10, 193 | −27, 121 |
| | Type III | 0.015 (vs NORCO), <.001 (vs Placebo) | | |
| | P Value | | | |
| over | n | 252 | 250 | 50 |
| 48 Hours | Mean (SD) | 118.4 (80.02) | 107.0 (83.60) | 53.1 (69.69) |
| (SPID48) | Median | 109.5 | 88.0 | 32.3 |
| | Min, Max | −23, 399 | −12, 380 | −76, 313 |

| | |
|---|---|
| Type III | 0.112 (vs NORCO), <.001 (vs Placebo) |
| P Value | |

SPIDx = Summed Pain Intensity Differences over x hours.
SD = standard deviation.
Min = minimum.
Max = maximum.
P values tested using type III test in general linear model with factors treatment and investigator as the main effects.

Comparing the various time interval strata for Formulation A, NORCO, and placebo show that over the first 12 hours (ie, over the first 2-3 doses) subjects treated with Formulation A experienced greater relief of moderate to severe pain than subjects treated with NORCO (p=0.01). Table 44. The comparison also shows that the first dose of Formulation A relieved moderate to severe pain compared to placebo: total pain relief over 4 and 6 hours was greater for subjects treated with Formulation A compared to subjects treated with placebo (p<0.001).

TABLE 44
Pain Relief (REL) Results (Intend To Treat Population).

| Total Pain | | | | |
|---|---|---|---|---|
| Relief | | Formulation A | NORCO | Placebo |
| (TOTPAR) | | (N = 252) | (N = 250) | (N = 50) |
| over | n | 252 | 250 | 50 |
| 12 Hours | Mean (SD) | 38.0 (29.45) | 31.6 (29.38) | 8.6 (14.09) |
| | Median | 34.9 | 24.0 | 1.8 |
| | Min, Max | 0, 109 | 0, 115 | 0, 58 |
| | Type III | 0.012 (vs NORCO), <.001 (vs Placebo) | | |
| | P Value | | | |
| over | n | 252 | 250 | 50 |
| 6 Hours | Mean (SD) | 17.5 (15.36) | 16.2 (15.15) | 4.3 (8.65) |
| | Median | 15.6 | 12.8 | 1.0 |
| | Min, Max | 0, 52 | 0, 57 | 0, 46 |
| | Type III | 0.326 (vs NORCO), <.001 (vs Placebo) | | |
| | P Value | | | |
| over | n | 252 | 250 | 50 |
| 4 Hours | Mean (SD) | 11.8 (9.99) | 11.7 (10.35) | 3.2 (6.06) |
| | Median | 11.3 | 9.0 | 1.0 |
| | Min, Max | 0, 35 | 0, 37 | 0, 33 |

| | |
|---|---|
| Type III | 0.951 (vs NORCO), <.001 (vs Placebo) |
| P Value | |

REL = Likert Pain Relief Scale.
SD = standard deviation.
Min = minimum.
Max = maximum.
P values tested using type III test in general linear model with factors treatment and investigator as the main effects.

Over the first 12 hours (ie, over the first 2-3 doses) more subjects who used Formulation A (76.6%) reported clinically meaningful relief (ie, at least moderate relief) than subjects who used NORCO (62.8%, p<0.001) (Table 45).

TABLE 45
Number of Subject with Pain relief Results (Intend To Treat
Population).

| | | Formulation | | |
|---|---|---|---|---|
| | | A | NORCO | Placebo |
| | | (N = 252) | (N = 250) | (N = 50) |
| Subjects with | n (%) | 193 (76.6%) | 157 (62.8%) | 19 (38.0%) |

| | | |
|---|---|---|
| Moderate or | Adjusted | 1.95 (1.32, 2.88) (vs NORCO), 5.51 |
| Better Relief | Odds Ratio | (2.89, 10.52) (vs Placebo), 2.79 (1.49, |
| over 12 Hours | and 95% CI | 5.25) (NORCO vs Placebo) |
| from Baseline | Type III | <.001 (vs NORCO), <.001 (vs Placebo), |
| | P Value | 0.001 (NORCO vs Placebo) |
| | NNT and | 8 (5, 18) (vs NORCO), 3 (2, 5) (vs |
| | 95% CI | Placebo), 5 (3, 10) (NORCO vs Placebo) |

P values tested using type III likelihood ratio test in logistic regression model with factors of treatment and investigator as main effects. Estimated as the inverse of the absolute risk difference in moderate or better relief (REL>=3) in the first 12 hours since baseline, i.e., the inverse of probability of moderate or better relief on Formulation A minus the probability of moderate or better relief on placebo. Subjects who received rescue medication for pain in the first 12 hours will be counted as having failed to achieve relief.

Formulation A versus placebo alleviating pain as experienced by the subjects population was also assessed by means of the QPI (Quality of Pain Index) as shown in Table 46. Formulation A was shown to reduce affective, sensory and evaluative qualities of pain after the first dose (over 6 hours) and after multiple doses (over 12 hours and over 24 hours) compared to placebo (all p<0.01).

TABLE 46
Qualities of Pain Index (QPI) Results (Intend To Treat Population).

| % Change in | | Formulation A | NORCO | Placebo |
|---|---|---|---|---|
| Overall | | (N = 252) | (N = 250) | (N = 50) |
| Sensory QPI Items | n | 251 | 248 | 49 |
| over 6 Hours | Mean (SD) | −11.7 (45.20) | −6.3 (47.15) | 3.7 (20.46) |
| | Median | 0.0 | 0.0 | 0.0 |
| | Min, Max | −100, 420 | −100, 500 | −49, 77 |
| | Type III P Value | 0.178 (vs NORCO), 0.026 (vs Placebo) | | |
| Sensory QPI Items | n | 251 | 248 | 49 |
| over 12 Hours | Mean (SD) | −23.0 (46.04) | −17.3 (52.83) | −2.8 (20.23) |
| | Median | −13.9 | 0.0 | 0.0 |
| | Min, Max | −100, 225 | −100, 600 | −73, 78 |
| | Type III P Value | 0.176 (vs NORCO), 0.007 (vs Placebo) | | |
| Sensory QPI Items | n | 251 | 248 | 49 |
| over 24 Hours | Mean (SD) | −43.4 (59.18) | −39.3 (42.66) | −12.5 (47.58) |
| | Median | −59.3 | −40.0 | 0.0 |
| | Min, Max | −100, 500 | −100, 100 | −100, 142 |
| | Type III P Value | 0.372 (vs NORCO), <.001 (vs Placebo) | | |
| Evaluative QPI | n | 252 | 249 | 48 |
| Items over 6 Hours | Mean (SD) | −7.8 (26.91) | −8.5 (26.68) | −1.2 (10.85) |
| | Median | 0.0 | 0.0 | 0.0 |
| | Min, Max | −100, 100 | −100, 75 | −50, 27 |
| | Type III P Value | 0.765 (vs NORCO), 0.098 (vs Placebo) | | |
| Evaluative QPI | n | 252 | 249 | 48 |
| Items over 12 | Mean (SD) | −21.8 (34.62) | −17.5 (32.32) | −4.6 (17.57) |
| Hours | Median | −10.0 | 0.0 | 0.0 |
| | Min, Max | −100, 100 | −100, 100 | −67, 67 |
| | Type III P Value | 0.141 (vs NORCO), <.001 (vs Placebo) | | |
| Evaluative QPI | n | 252 | 249 | 48 |
| Items over 24 | Mean (SD) | −44.0 (39.13) | −36.7 (40.80) | −17.3 (29.29) |
| Hours | Median | −50.0 | −33.3 | 0.0 |
| | Min, Max | −100, 89 | −100, 100 | −100, 25 |
| | Type III P Value | 0.037 (vs NORCO), <.001 (vs Placebo) | | |
| Affective QPI | n | 236 | 227 | 44 |
| Items over 6 Hours | Mean (SD) | −8.6 (72.17) | −7.0 (71.62) | 3.8 (33.20) |
| | Median | 0.0 | 0.0 | 0.0 |
| | Min, Max | −100, 900 | −100, 700 | −58, 160 |
| | Type III P Value | 0.802 (vs NORCO), 0.278 (vs Placebo) | | |
| Affective QPI | n | 236 | 227 | 44 |
| Items over 12 | Mean (SD) | −24.7 (58.21) | −21.2 (39.49) | −7.6 (24.79) |
| Hours | Median | −13.4 | 0.0 | 0.0 |
| | Min, Max | −100, 500 | −100, 150 | −100, 67 |
| | Type III P Value | 0.428 (vs NORCO), 0.030 (vs Placebo) | | |
| Affective QPI | n | 236 | 227 | 44 |
| Items over 24 | Mean (SD) | −46.9 (59.94) | −42.6 (52.97) | −14.0 (53.33) |
| Hours | Median | −60.0 | −47.1 | 0.0 |
| | Min, Max | −100, 300 | −100, 300 | −100, 200 |

| | |
|---|---|
| Type III P Value | 0.413 (vs NORCO), <.001 (vs Placebo) |

P values tested using type III test in general linear model with factors treatment and investigator as the main effects. Sensory items are the following 11 QPI items pooled: Throbbing, Hot, Heavy, Pulling, Sharp, Radiating, Pressing, Swollen, Tight, Stabbing, Stinging. Evaluative items are the following 2 QPI items pooled: Aching, Hurting. Affective items are the following 2 QPI items pooled: Annoying, Agonizing.

Reduction in Sleep Disturbance in Subjects Treated with Formulation A.

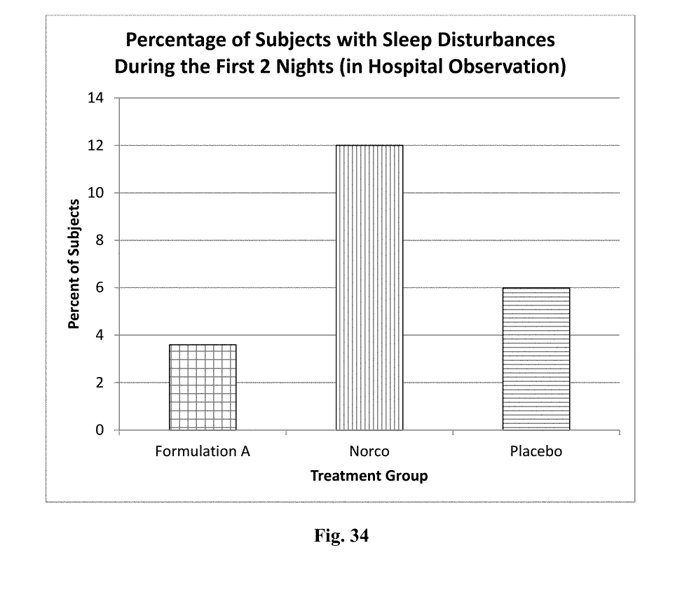

Table 47 summarizes the percentage of subjects who experienced sleep disturbances. During the 2 nights of in-hospital observation there was more than 3-fold greater incidence of sleep disturbance for subjects treated with NORCO (12.0%) than subjects treated with Formulation A (3.6%, p<0.001), which was indistinguishable from sleep disturbance among subjects taking placebo (6.0%). This represents a 70% reduction in the risk of disturbing sleep (FIG. 34).

TABLE 47
Sleep Disturbances Results (Intend To Treat Population).

| % of Subjects | | | | |
|---|---|---|---|---|
| with Sleep | | Formulation A | NORCO | Placebo |
| Disturbances | | (N = 252) | (N = 250) | (N = 50) |
| on Day 1 | n (%) | 5 (2.0%) | 21 (8.4%) | 1 (2.0%) |
| | Adjusted | (vs NORCO), (vs Placebo) | | |
| | Odds Ratio | | | |
| | and 95% CI | | | |
| | Type III | 0.003 (vs NORCO), 0.990 (vs Placebo) | | |
| | P Value | | | |
| on Day 2 | n (%) | 6 (2.4%) | (6.8%) | 2 (4.0%) |
| | Adjusted | 0.33 (0.13, 0.86) (vs NORCO), 0.57 | | |
| | Odds Ratio | (0.11, 2.92) (vs Placebo) | | |
| | and 95% CI | | | |
| | Type III | 0.023 (vs NORCO), 0.497 (vs Placebo) | | |
| | P Value | | | |
| on Day 1 | n (%) | 9 (3.6%) | 30 (12.0%) | 3 (6.0%) |

| | | |
|---|---|---|
| and 2 | Adjusted | 0.27 (0.12, 0.58) (vs NORCO), 0.57 |
| Combined | Odds Ratio | (0.15, 2.21) (vs Placebo) |
| | and 95% CI | |
| | Type III | <.001 (vs NORCO), 0.418 (vs Placebo) |
| | P Value | |

CI = confidence interval.
P values tested using type III likelihood ratio test in logistic regression model with factors of treatment and investigator as main effects.

Relief in Agnozing Pain in Subjects Treated with Formulation A

When qualities of pain are extremely painful, subjects often describe their pain as “agonizing,” signaling significant distress. Measurements of agonizing pain (i.e., how subjects actually describe their most distressing pain) provides a sensitive indicator of therapeutic efficacy. This study investigated the word which subjects use to describe their most bothersome pain (“agonizing”) and evaluated changes in this affective quality of pain before and after treatment.

Here multiple doses of Formulation A are effective on agonizing pain compared to placebo following orthopedic surgery. At baseline and intervals over 48 hours subjects rated pain intensity and other qualities of pain on 0-10 numeric rating scales (NRS) while re-dosing every 4-6 hours. There were significant differences between Formulation A and placebo on all rating scales in 152 subjects who had moderate/severe pain intensity and agonizing pain. At 24 hours, for example, subjects treated with Formulation A reported 57% reduction of agonizing pain compared to baseline, 37% greater than subjects treated with placebo (p<0.001); 69% of Formulation A-treated subjects had >30% reduction in agonizing pain intensity (compared to 23% of placebo-treated subjects, p<0.001), surpassing the 2-integer criterion for the raw intensity difference on a 0-10 NRS that is considered clinically important. Tables 48 and 49 illustrate a summary of qualities of pain index (QPI) agonizing in ITT population—subjects with agonizing intensity >=4 at baseline over 24 hours. Tables 50 and 51 illustrate a summary of qualities of pain index (QPI) agonizing in ITT population—subjects with agonizing intensity >=4 at baseline over 12 hours.

TABLE 48
Percentage Change in QPI Assessment (Agonizing) over 24 Hours

| | Estimate | Estimate | | | |
|---|---|---|---|---|---|
| | P Value vs | P Value vs | Formulation A | NORCO | Placebo |
| Endpoint | NORCO | Placebo | (N = 122) | (N = 138) | (N = 30) |
| n | | | 122 | 138 | 30 |
| Mean (SD) | | | −57.2 (41.71) | −50.0 (45.79) | −20.5 (36.61) |
| Median | | | −71.4 | −50.0 | 0.0 |
| Min, Max | | | −100, 33 | −100, 25 | −100, 13 |
| Type III P | 0.125 | <0.001 | | | |
| Value | | | | | |
| (Formulation A | | | | | |
| vs NORCO and | | | | | |
| Placebo)[1] | | | | | |

[1]Tested using type III test in general linear model with factors treatment and investigator as the main effects.

TABLE 49
Percentage of Subjects with >=30% Reduction in Agonizing over 24 Hours

| | Estimate | Estimate | | | |
|---|---|---|---|---|---|
| | P Value vs | P Value vs | Formulation A | NORCO | Placebo |
| Endpoint | NORCO | Placebo | (N = 122) | (N = 138) | (N = 30) |
| n (%) | | | 84 (68.9%) | 79 (57.2%) | 7 (23.3%) |
| Adjusted Odds | 1.71 (1.02, | 7.51 (2.89, | | | |
| Ratio and 95% | 2.87) | 19.48) | | | |
| CI | | | | | |
| Type III P | 0.043 | <0.001 | | | |
| Value | | | | | |
| (Formulation A | | | | | |
| vs NORCO and | | | | | |
| Placebo)[1] | | | | | |

[1]Tested using type III likelihood ratio test in logistic regression model with factors treatment and investigator as the main effects.

TABLE 50
Percentage Change in QPI Assessment (Agonizing) over 12 Hours

| | Estimate | Estimate | | | |
|---|---|---|---|---|---|
| | P Value vs | P Value vs | Formulation A | NORCO | Placebo |
| Endpoint | NORCO | Placebo | (N = 122) | (N = 138) | (N = 30) |
| n | | | 122 | 138 | 30 |
| Mean (SD) | | | −34.2 (42.38) | −29.3 (40.13) | −15.6 (30.12) |
| Median | | | −11.3 | 0.0 | 0.0 |
| Min, Max | | | −100, 40 | −100, 40 | −100, 0 |
| Type III P | 0.263 | 0.029 | | | |
| Value | | | | | |
| (Formulation A | | | | | |
| vs NORCO and | | | | | |
| Placebo)[1] | | | | | |

[1]Tested using type III test in general linear model with factors treatment and investigator as the main effects.

TABLE 51
Percentage of Subjects with >=30% Reduction in Agonizing over 12 Hours

| | Estimate | Estimate | | | |
|---|---|---|---|---|---|
| | P Value vs | P Value vs | Formulation A | NORCO | Placebo |
| Endpoint | NORCO | Placebo | (N = 122) | (N = 138) | (N = 30) |
| n (%) | | | 51 (41.8%) | 51 (37.0%) | 6 (20.0%) |
| Adjusted Odds | 1.27 (0.77, | 2.91 (1.08, | | | |
| Ratio and 95% | 2.11) | 7.85) | | | |
| CI | | | | | |
| Type III P | 0.348 | 0.034 | | | |
| Value | | | | | |
| (Formulation A | | | | | |
| vs NORCO and | | | | | |
| Placebo)[1] | | | | | |

[1]Tested using type III likelihood ratio test in logistic regression model with factors treatment and investigator as the main effects.

While particular embodiments described herein have been shown and described herein, such embodiments are provided by way of example only. Numerous variations, changes, and substitutions can now occur to those skilled in the art without departing from what is disclosed herein. It should be understood that various alternatives to the embodiments described herein can be employed in practicing embodiments disclosed herein. It is intended that the following claims define the scope of some embodiments of the invention and that methods and structures within the scope of these claims and their equivalents be covered thereby.

What is claimed is:

1. A method for providing treatment or reduction of agonizing pain in a subject, comprising:
administering to a subject in need thereof a pharmaceutical composition, wherein the pharmaceutical composition comprises:
an effective amount of an opioid analgesic to treat agonizing pain; and
an effective amount of an antiemetic to reduce or prevent nausea associated the opioid analgesic or to reduce or prevent vomiting associated the opioid analgesic, wherein administration of the pharmaceutical composition provides for a reduction or prevention of agonizing pain in the subject.

2. The method of claim 1, wherein the agonizing pain is measured by the subject on a Likert-type agonizing pain scale.

3. The method of any one of claims 1 to 2, wherein the method reduces severity of the agonizing pain.

4. The method of any one of claims 1 to 3, wherein the method reduces occurrence of the agonizing pain.

5. The method of any one of claims 1 to 4, wherein administration of the pharmaceutical composition provides for about 60% reduction of agonizing pain over 24 hours.

6. The method of any one of claims 1 to 4, wherein administration of the pharmaceutical composition provides for about 30% reduction in pain intensity over 24 hours.

7. The method of any one of claims 1 to 4, wherein administration of the pharmaceutical composition provides increased agonizing pain relief in the subject compared to administration of the opioid analgesic without the antiemetic.

8. The method of any one of claims 1 to 7, wherein the pharmaceutical composition further comprises an effective amount of a non-opioid analgesic to treat pain.

9. The method of any one of claims 1 to 8, wherein the pharmaceutical composition is a solid oral pharmaceutical composition.

10. A method for providing pain relief and reducing or preventing retching in a subject in need thereof, comprising:
administering to a subject in need thereof a pharmaceutical composition, wherein the pharmaceutical composition comprises:
an effective amount of an opioid analgesic to treat pain; and
an effective amount of an antiemetic to reduce or prevent retching associated the opioid analgesic, wherein administration of the pharmaceutical composition provides for a reduction or prevention of retching in the subject.

11. The method of claim 10, wherein the pharmaceutical composition further comprises an effective amount of a non-opioid analgesic to treat pain.

12. The method of any one of claims 10 to 11, wherein the pharmaceutical composition is a solid oral pharmaceutical composition.

13. The method of any one of claims 10 to 12, wherein the administration of the pharmaceutical composition provides for prevention of retching.

14. The method of any one of claims 10 to 12, wherein the administration of the pharmaceutical composition provides for reduction in severity of retching.

15. The method of any one of claims 10 to 12, wherein the administration of the pharmaceutical composition provides for reduction in severity of retching.

16. The method of any one of claims 10 to 12, wherein the retching is dry heaving.

17. The method of any one of claims 10 to 12, wherein administration of the pharmaceutical composition provides for less vomiting than observed for a subject administered the opioid without the antiemetic.

18. The method of claim 17, wherein administration of the pharmaceutical composition provides for three times less vomiting that observed for a subject administered the opioid without the antiemetic.

19. The method of claim 17, wherein administration of the pharmaceutical composition provides for two times less vomiting that observed for a subject administered the opioid without the antiemetic over about 5 days following administration of the pharmaceutical composition.

20. The method of any one of claims 10 to 12, wherein the subject has a reduction of at least 35% in occurrence of retching or vomiting over about 5 days or more following administration of the pharmaceutical composition.

21. The method of any one of claims 10 to 12, wherein the subject has a reduction of at least 45% in occurrence of retching or vomiting over about 5 days or more following administration of the pharmaceutical composition.

22. The method of any one of claims 10 to 12, wherein the subject has a reduction of about 50% in occurrence of retching over about 5 days following administration of the pharmaceutical composition.

23. The method of any one of claims 10 to 22, wherein administration of the pharmaceutical composition provides increased pain relief in the subject compared to administration of the opioid analgesic without the antiemetic.

24. A method for providing pain relief and reducing or preventing sleep disturbance in a subject in need thereof, comprising administering to a subject a pharmaceutical composition, wherein the pharmaceutical composition comprises:
an effective amount of an opioid analgesic to treat pain; and
an effective amount of an antiemetic to reduce or prevent sleep disturbance associated the opioid analgesic, wherein administration of the pharmaceutical composition provides for a reduction or prevention of sleep disturbance in the subject.

25. The method of claim 24, wherein administration of the pharmaceutical composition provides for about 3.5% chance of sleep disturbance over a 48 hour period in the subject.

26. The method of any one of claims 24 or 25, wherein the subject has a reduction of about 40% in occurrence of retching over about 5 days following administration of the pharmaceutical composition.

27. The method of claim 24, wherein the pharmaceutical composition further comprises an effective amount of a non-opioid analgesic to treat pain.

28. The method of claim 24, wherein the pharmaceutical composition is a solid oral pharmaceutical composition.

29. The method of any one of claims 24 to 28, wherein the subject has a reduction of at least 70% in the occurrence of sleep disturbance over 2 nights or more following administration of the pharmaceutical composition.

30. The method of any one of claims 24 to 29, wherein the sleep disturbance is measured by the number of awakenings in an observation period.

31. The method of any one of claims 24 to 30, wherein the subject awakens due to nausea, retching, or vomiting.

32. The method of any one of claims 24 to 31, wherein administration of the pharmaceutical composition provides for less sleep disturbance for the subject compared to administration of the opioid analgesic without the antiemetic.

33. The method of any preceding claim, wherein the method provides the subject a decrease in pain occurrence.

34. The method of any preceding claim, wherein the method provides the subject a decrease in pain severity.

35. The method of claim 34, wherein the pain severity is reduced by more than 30% following first administration of the pharmaceutical composition.

36. The method of any preceding claim, wherein the subject has a reduction of at least 30% in severe pain over about 24 hours or more following administration of the pharmaceutical composition.

37. The method of any preceding claim, wherein the subject has a reduction of at least 10% in moderate to severe pain over about 48 hours or more following administration of the pharmaceutical composition.

38. The method of any preceding claim, wherein the subject has a reduction of at least 20% in moderate to severe pain over about 24 hours or more following administration of the pharmaceutical composition.

39. The method of any preceding claim, wherein the subject has a reduction in moderate to severe pain over about 4 to 6 hours or more following administration of the pharmaceutical composition.

40. The method of any preceding claim, wherein the subject has a reduction in moderate to severe pain over about 12 hours or more following administration of the pharmaceutical composition.

41. The method of any preceding claim, wherein the subject has a reduction in moderate to severe pain over about 2 or 3 doses following administration of the pharmaceutical composition.

42. The method of any preceding claim, wherein the subject has an increase of at least 20% in pain relief over about 12 hours or more following administration of the pharmaceutical composition.

43. The method of any preceding claim, wherein the subject has an increase of at least 20% in pain relief over about the first 2 or 3 doses or more following administration of the pharmaceutical composition.

44. The method of any preceding claim, wherein the subject has a reduction in affective, sensory, or evaluative qualities of pain over about 6 hours or more following initial administration of the pharmaceutical composition.

45. The method of any preceding claim, wherein the subject has a reduction in affective, sensory, or evaluative qualities of pain over two or more doses or about 12 hours, about 24 hours, or more following administration of the pharmaceutical composition.

46. The method of any preceding claim, wherein the subject has a reduction of at least 15% in affective, sensory, or evaluative qualities of pain over about 24 hours or more following administration of the pharmaceutical composition.

47. The method of any preceding claim, wherein the subject has a reduction of at least 50% in the occurrence of vomiting over about 24 hours or more following administration of the pharmaceutical composition.

48. The method of any preceding claim, wherein the subject is nausea-prone.

49. The method of any preceding claim, wherein the subject is susceptible to opioid induced nausea or vomiting (OINV).

50. The method of any preceding claim, wherein the subject has increased pain relief compared to a subject administered a pharmaceutical composition that comprises the opioid analgesic and the non-opioid analgesic without the antiemetic.

51. The method of any preceding claim, wherein the subject has increased pain relief during the first 6 hours post administration.

52. The method of any preceding claim, wherein the administration of the pharmaceutical composition further provides for reduction or prevention of opioid induced nausea or vomiting (OINV) in the subject.

53. The method of any preceding claim, wherein the subject has reduced severity of nausea or vomiting.

54. The method of any preceding claim, wherein the subject has reduced severity of nausea or vomiting over about 6 hours following initial administration of the pharmaceutical composition.

55. The method of any preceding claim, wherein the subject has reduced severity of nausea or vomiting over about 24 hours following initial administration of the pharmaceutical composition.

56. The method of any preceding claim, wherein the subject has reduced occurrence of nausea or vomiting.

57. The method of any preceding claim, wherein the subject has a reduction of at least 70% in the occurrence of ONIV over about 48 hours or more following administration of the pharmaceutical composition.

58. The method of any preceding claim, wherein the subject has a reduction of at least 50% in occurrence of ONIV for about 24 hours or more following administration of the pharmaceutical composition.

59. The method of any preceding claim, wherein reducing nausea or vomiting in the subject is in comparison to a subject administered a pharmaceutical composition that comprises the opioid analgesic and the non-opioid analgesic without the antiemetic.

60. The method of any preceding claim, wherein the method reduces occurrence or severity of nausea or vomiting over about 24 hours following administration of the pharmaceutical composition.

61. The method of any preceding claim, wherein the subject has a reduction of at least 50% in occurrence of moderate to severe nausea or vomiting over about 48 hours or more following administration of the pharmaceutical composition.

62. The method of any preceding claim, wherein the subject has a reduction of at least 60% in occurrence of vomiting over about 5 days or more following administration of the pharmaceutical composition.

63. The method of any preceding claim, wherein the subject has a reduction of at least 50% in doses of an antiemetic over about 48 hours or more following administration of the pharmaceutical composition.

64. The method of any preceding claim, wherein the subject has a reduction of at least 75% in doses of a parenteral antiemetic over about 48 hours or more following administration of the pharmaceutical composition.

65. The method of any preceding claim, wherein the subject has a reduction of at least 70% in doses of an antiemetic over about 5 days or more following administration of the pharmaceutical composition.

66. The method of any preceding claim, wherein the subject has a reduction of at least 80% in doses of an antiemetic over about 5 days or more following administration of the pharmaceutical composition.

67. The method of any preceding claim, wherein the subject has a reduction of at least 70% in severe nausea over about 5 days or more following administration of the pharmaceutical composition.

68. The method of any preceding claim, wherein the subject has a reduction of at least 40% in peak severity of nausea over about 5 days or more following administration of the pharmaceutical composition.

69. The method of any preceding claim, wherein the subject has an increase of 60% in the likelihood of no nausea, no vomiting, or no use of an antiemetic over about 5 days or more following administration of the pharmaceutical composition.

70. The method of any preceding claim, wherein the subject has an increase of 100% in the likelihood of no nausea, no vomiting, or no use of an antiemetic over about 5 days or more following administration of the pharmaceutical composition.

71. The method of any preceding claim, wherein the administration occurs more than once over about 24 hours or longer.

72. The method of any preceding claim, further comprising administering an antiemetic when awake.

73. The method of any preceding claim, further comprising assessing the subject by a Nausea Severity Scale (NIS).

74. The method of any preceding claim, further comprising assessing the subject by a Retching or Vomiting Index (RVI).

75. The method of any preceding claim, further comprising assessing the subject by a Vomiting Supplemental Medications (NVSM).

76. The method of any preceding claim, wherein the administration occurs every four to six hours.

77. The method of any preceding claim, wherein the administration occurs two to six times over the first 24 hours.

78. The method of any preceding claim, wherein the administration occurs one to six times after the first 24 hours.

79. The method of any preceding claim, wherein the administration occurs no more than six times every 24 hours.

80. The method of any preceding claim, wherein the administration occurs periodically over 1-28 days.

81. The method of any preceding claim, wherein the administration occurs periodically over 1-21 days.

82. The method of any preceding claim, wherein the administration occurs periodically over 1-14 days.

83. The method of any preceding claim, wherein the administration occurs periodically over 1-7 days.

84. The method of any preceding claim, wherein the administration occurs periodically over 1-5 days.

85. The method of any preceding claim, wherein the administration occurs periodically over 1-3 days.

86. The method of any preceding claim, wherein the administration occurs periodically over 1-2 days.

87. The method of any preceding claim, wherein the administration occurs periodically over 24 hours.

88. The method of any preceding claim, wherein the administration is an oral administration.

89. The method of any preceding claim, wherein the administration begins from 0 to 6 hours before a surgery.

90. The method of any preceding claim, wherein the administration begins from 0 to 6 hours after a surgery.

91. The method of any preceding claim, wherein the administration is from 0 to 6 hours after an injury.

92. The method of any preceding claim, wherein the subject is a post-operative subject.

93. The method of any preceding claim, wherein the subject is a post-discharge subject.

94. The method of any preceding claim, wherein the pain is pain from an operation or post-operative pain.

95. The method of any preceding claim, wherein the pain is acute pain.

96. The method of any preceding claim, wherein the pain is chronic pain.

97. The method of any preceding claim, wherein the pain is severe.

98. The method of any preceding claim, wherein the pain is moderate.

99. The method of any preceding claim, wherein the pain is moderate to severe.

100. The method of any preceding claim, wherein the pain is agonizing pain.

101. The method of any preceding claim, wherein the administration is prophylactic.

102. The method of any preceding claim, wherein the subject has one or more conditions or diseases.

103. The method of claim 102, wherein the one or more conditions or diseases comprise cancer, surgical procedure, acute physical injury, chronic physical injury, bone fracture, crush injury, spinal cord injury, inflammatory disease, non-inflammatory neuropathic condition, photophobia, or any combination thereof.

104. The method of any preceding claim, wherein the pharmaceutical composition does not cause increased sedation in comparison to a pharmaceutical composition with the same amount of the opioid analgesic, in the absence of the antiemetic.

105. The method of any preceding claim, wherein the pharmaceutical composition is a solid dosage form.

106. The method of claim 105, wherein the solid dosage form comprises an immediate-release matrix, wherein the immediate release matrix comprises an effective amount of the antiemetic.

107. The method of claim 105 or 106, wherein the solid dosage form comprises a controlled-release matrix, wherein the controlled-release matrix comprises an effective amount of the opioid analgesic.

108. The method of claim 107, wherein the controlled-release matrix comprises an effective amount of the non-opioid analgesic.

109. The method of any one of claims 105 to 108, wherein the solid dosage form is a tablet, particle or capsule.

110. The method of claim 109, wherein the tablet is a multi-layer tablet.

111. The method of claim 109, wherein the tablet is a bi-layer tablet.

112. The method of any preceding claim, wherein the antiemetic is promethazine or a pharmaceutically acceptable salt thereof.

113. The method of any preceding claim, wherein the opioid analgesic is hydrocodone or a pharmaceutically acceptable salt thereof.

114. The method of any preceding claim, wherein the opioid analgesic is oxycodone or a pharmaceutically acceptable salt thereof.

115. The method of any preceding claim, wherein the non-opioid analgesic is acetaminophen or a pharmaceutically acceptable salt thereof.

116. The method of any preceding claim, wherein: the opioid analgesic is hydrocodone or a pharmaceutically acceptable salt thereof that is present in an amount of from about 6.5 mg to about 8.5 mg; and the antiemetic is promethazine or a pharmaceutically acceptable salt thereof that is present in an amount of from about 12.5 mg to about 25 mg.

117. The method of any preceding claim, wherein: the opioid analgesic is hydrocodone, or a pharmaceutically acceptable salt thereof that is present in an amount of from about 6.5 mg to about 8.5 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg.

118. The method of any preceding claim, wherein: the opioid analgesic is oxycodone or a pharmaceutically acceptable salt thereof that is present in an amount of from about 5 mg to about 15 mg; and the antiemetic is promethazine or a pharmaceutically acceptable salt thereof that is present in an amount of from about 12.5 mg to about 25 mg.

119. The method of any preceding claim, wherein: the opioid analgesic is hydrocodone, or a pharmaceutically acceptable salt thereof that is present in an amount of from 5 mg to about 15 mg; the antiemetic is promethazine or a pharmaceutically acceptable salt thereof that is present in an amount of from about 12.5 mg to about 25 mg; and the non-opioid analgesic is the acetaminophen or the pharmaceutically acceptable salt thereof that is present in an amount of from about 290 to about 360 mg.

120. The method of any preceding claim, wherein the non-opioid analgesic is present in an amount of about 200 mg to about 600 mg, about 200 mg to about 1000 mg, about 200 mg to about 325 mg, about 325 mg to about 330 mg, about 330 mg to about 335 mg, about 335 mg to about 340 mg, about 340 mg to about 345 mg, about 345 mg to about 350 mg, about 325 mg to about 350 mg, about 350 mg to about 400 mg, about 400 mg to about 1000 mg, or any combination thereof.

121. The method of any preceding claim, wherein: the antiemetic is promethazine or a pharmaceutically acceptable salt thereof that is present in amount of about 12.5 mg, the opioid analgesic is hydrocodone or a pharmaceutically acceptable salt thereof that is present in an amount of about 7.5 mg; and the non-opioid analgesic is acetaminophen or a pharmaceutically acceptable salt thereof that is present in an amount of about 325 mg.

122. The method of any preceding claim, wherein: the antiemetic is promethazine hydrochloride that is present in an amount of about 12.5 mg; the opioid analgesic is hydrocodone bitartrate that is present in an amount of about 7.5 mg; and the non-opioid analgesic is acetaminophen that is present in an amount of about 325 mg.

123. The method of any preceding claim, wherein: the antiemetic is promethazine or a pharmaceutically acceptable salt thereof that is present in amount of about 12.5 mg, and the opioid analgesic is oxycodone or a pharmaceutically acceptable salt thereof that is present in an amount of about 5.0 mg.

124. The method of any preceding claim, wherein: the antiemetic is promethazine hydrochloride that is present in an amount of about 12.5 mg; and the opioid analgesic is oxycodone hydrochloride that is present in an amount of about 5.0 mg.

125. The method of any preceding claim, wherein the subject is human.

126. The method of any preceding claim, wherein the pharmaceutical composition further comprises one or more excipients.

127. The method of claim 126, wherein the one or more excipients comprise an antioxidant agent, a binder, a coating material, a colorant agent, a diluent, a disintegrant, a dispersant, an emulsifying agent, a flavoring agent, a glidant, a lubricant, a pH modifying agent, a plasticizer, a preservative agent, a solubilizing agent, a stabilizer, a surfactant, a sweetening agent, a thickening agent, a pharmaceutically inert material, or any combination thereof.

128. The method of any preceding claim, wherein the antiemetic has a Tmax that is about 3-6 hours.

129. The method of any preceding claim, wherein the antiemetic has a Tmax that is about 20-100 minutes shorter than a Tmax of a corresponding standard-release antiemetic.

130. The method of any preceding claim, wherein the antiemetic has a Tmax that is about 3-5 hours, wherein the subject is fasted.

131. The method of claim 130, wherein the antiemetic has a Tmax of about 4 hours.

132. The method of any preceding claim, wherein the antiemetic has a Tmax that is about 20-80 minutes shorter than a Tmax of a corresponding standard-release antiemetic, wherein the subject is fasted.

133. The method of claim 132, wherein the antiemetic has a Tmax that is about 50 minutes shorter than a Tmax of a corresponding standard-release antiemetic.

134. The method of any preceding claim, wherein the antiemetic has a Tmax that is about 4-6 hours, wherein the subject is fed.

135. The method of claim 134, wherein the antiemetic has a Tmax of about 5 hours.

136. The method of any preceding claim, wherein the antiemetic has a Tmax that is about 40-100 minutes shorter than a Tmax of a corresponding standard-release antiemetic, wherein the subject is fed.

137. The method of claim 136, wherein the antiemetic has a Tmax that is about 70 minutes shorter than a Tmax of a corresponding standard-release antiemetic.

138. The method of any preceding claim, wherein the antiemetic has an about 20-200% greater absorption during in about two hours than a corresponding standard-release antiemetic.

139. The method of any preceding claim, wherein the antiemetic has an about 20-200% greater absorption in about 90 minutes than a corresponding standard-release antiemetic.

140. The method of any preceding claim, wherein the antiemetic has an about 20-200% greater absorption in about an hour than a corresponding standard-release antiemetic.

141. The method of any preceding claim, wherein the antiemetic has an about 20-100% greater absorption in about an hour than a corresponding standard-release antiemetic.

142. The method of any preceding claim, wherein the antiemetic has an about 60% greater absorption in about an hour than a corresponding standard-release antiemetic.

143. The method of any preceding claim, wherein the antiemetic has an about 20-60% greater absorption in about 45 minutes than a corresponding standard-release antiemetic.

144. The method of any preceding claim, wherein the antiemetic has an about 40% greater absorption in about 45 minutes than a corresponding standard-release antiemetic.

145. The method of any preceding claim, wherein the antiemetic has an about 20-60% greater absorption in about 30 minutes than a corresponding standard-release antiemetic.

146. The method of any preceding claim, wherein the antiemetic has an about 40% greater absorption in about 30 minutes than a corresponding standard-release antiemetic.

147. The method of any preceding claim, wherein the antiemetic is promethazine or a pharmaceutically acceptable salt thereof.

148. The method of claim 147, wherein the antiemetic is promethazine hydrochloride.

149. The method of any preceding claim, wherein the administration occurs multiple times per day.

150. The method of any preceding claim, wherein the administration occurs at least twice daily.

151. The method of any preceding claim, wherein the administration occurs 2 to 6 times daily.
